(19) United States
(12) Patent Application Publication
(10) Pub. No.: US 2015/0038348 A1
(43) Pub. Date: Feb. 5, 2015

(54) MICROBIAL BIOINDICATORS OF HYDROCARBONS IN WATER AND IN MARINE SEDIMENTS AND METHODS FOR MAKING AND USING THEM

(71) Applicants: Matthew Ashby, Mill Valley, CA (US); Dago Dimster-Denk, San Anselmo, CA (US); Ulrika Elisa Lidstrom, San Francisco, CA (US)

(72) Inventors: Matthew Ashby, Mill Valley, CA (US); Dago Dimster-Denk, San Anselmo, CA (US); Ulrika Elisa Lidstrom, San Francisco, CA (US)

(73) Assignee: TAXON BIOSCIENCES, INC., Tiburon, CA (US)

(21) Appl. No.: 13/696,954

(22) PCT Filed: Jul. 29, 2011
(86) PCT No.: PCT/US11/46015

§ 371 Date: Apr. 1, 2013

Related U.S. Application Data
(60) Provisional application No. 61/369,616, filed on Jul. 30, 2010.

(51) Int. Cl. C12Q 1/68

(52) U.S. Cl. CPC C12Q 1/6888; C12Q 2600/16
USPC 506/9; 536/24.33; 536/24.32; 506/16; 435/6.11; 435/6.12

(57) ABSTRACT

In alternative embodiments, the invention provides products of manufacture and compositions, e.g., nucleic acid probes, for use as identifying agents or indicators to detect the presence of a hydrocarbon in a sample, e.g., in marine sediments, muds, sands and the like, or in a solution, e.g., an aqueous solution, such as a production water, fresh water, underground water or seawater. In alternative embodiments, the invention provides compositions, e.g., nucleic acid probes or primers or primer pairs, for use as sensors and/or identifying agents to detect the presence of a hydrocarbon in a sample (e.g., in fresh water, underground water or seawater, or a marine mud, sand or sediment), where the presence of the hydrocarbon indicates e.g., the presence of a subsurface oil, petroleum or gas accumulation or deposit. In alternative embodiments, the invention provides compositions and methods for use as tools for offshore oil exploration activities.

### RELATED APPLICATIONS

This International (PCT) patent application claims benefit of priority to U.S. Provisional Patent application Ser. No. 61/369,616, filed Jul. 30, 2010, which is expressly incorporated by reference herein in its entirety for all purposes.

### TECHNICAL FIELD

This invention generally relates to hydrocarbon exploration, e.g., oil and gas exploration, oil pollution monitoring and management, and microbiology. In alternative embodiments, the invention provides products of manufacture and compositions, e.g., nucleic acid probes, for use as identifying agents or indicators to detect the presence of a hydrocarbon in a sample, e.g., in marine sediments, muds, sands and the like, or in a solution, e.g., an aqueous solution, such as fresh water, underground water or seawater. In alternative embodiments, the invention provides compositions, e.g., nucleic acid probes, for use as sensors and/or identifying agents to detect the presence of a hydrocarbon in a sample (e.g., in fresh water, underground water or seawater, or a marine mud, sand or sediment), where the presence of the hydrocarbon indicates e.g., the presence of a subsurface oil, petroleum or gas accumulation or deposit. In alternative embodiments, the invention provides compositions and methods for use as tools for offshore oil exploration activities.

### BACKGROUND

Commercially relevant accumulations of oil and/or gas reside in geologic features that prevent their further migration, so-called trap structures. The seals of these traps are rarely perfect and leakage occurs. In cases where substantial amounts of petroleum escape, both liquid and gaseous components migrate upward through faults and fractures until they reach the surface. These type of seeps are referred to as ‘prolific’ or ‘macroseeps’ and often are laterally displaced significant distances from their source. Microseeps, in contrast, result from low molecular weight gases (e.g. methane, ethane, propane) escaping from petroleum reservoirs that migrate vertically with little or no lateral displacement creating a diffuse plume overlying the source.

The presence of surface hydrocarbon seeps has been used as an exploration tool for oil/gas reservoirs ever since wells have been drilled. Given the often significant lateral displacement of prolific seeps as a result of travelling through faults, these type of seeps are used as a general (basin-wide) indication of hydrocarbons and to gain clues as to the geochemical character (e.g. API gravity) and the source/age of the resource.

A number of challenges confront the scientist tasked with interpreting geochemical data from potential seep sites. Some of these challenges relate to the ephemeral nature of seeps (diurnal, seasonal variations) and to the effects of microbes actively metabolizing seeping hydrocarbons.

### SUMMARY

In alternative embodiments, the invention provides products of manufacture and compositions, e.g., nucleic acid probes and primers, for use as identifying agents or indicators to detect the presence of a hydrocarbon in a sample, e.g., an environmental sample, e.g., a marine sediment, sand or mud, or a solution, e.g., an aqueous solution, such as fresh water, underground water or seawater. In alternative embodiments, the invention provides compositions, e.g., nucleic acid probes, for use as a sensor, e.g., a bioindicator, to detect the presence (e.g., immediate or nearby) of a hydrocarbon in a sample, e.g., in fresh water, underground water or seawater, where the presence of the hydrocarbon indicates e.g., the presence of a subsurface oil, petroleum or gas accumulation, deposit or leak or spill. The identified or detected hydrocarbon can be a vertically migrating hydrocarbon, e.g., vertically migrating in fresh water, underground water or seawater or sand, shale or mud. In alternative embodiments, the invention provides compositions and methods for use as tools for offshore oil exploration activities.

In alternative embodiments, the invention provides isolated, synthetic or recombinant nucleic acids comprising or consisting of:

(a) a nucleic acid or a nucleic acid sequence as set forth in Table 1, Table 2, Table 3 or Table 4;

(b) a nucleic acid or a nucleic acid sequence having at least about 85%, 86%, 87%, 88%, 89%, 90%, 91%, 92%, 93%, 94%, 95%, 96%, 97%, 98% or 99% or complete (100%) sequence homology to a nucleic acid or a nucleic acid sequence as set forth in Table 1, Table 2, Table 3 or Table 4;

(c) a nucleic acid or a nucleic acid sequence having at least about 85%, 86%, 87%, 88%, 89%, 90%, 91%, 92%, 93%, 94%, 95%, 96%, 97%, 98% or 99% or complete (100%) sequence homology to a nucleic acid or a nucleic acid sequence: as set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165, SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192 SEQ ID NO:193, SEQ ID NO:194, SEQ ID NO:195, SEQ ID NO:196, SEQ ID NO:197, SEQ ID NO:198, SEQ ID NO:199 or SEQ ID NO:200 (hereinafter referenced as SEQ ID NO:1 to SEQ ID NO:200); or

(d) a nucleic acid or a nucleic acid sequence having at least about 85%, 86%, 87%, 88%, 89%, 90%, 91%, 92%, 93%, 94%, 95%, 96%, 97%, 98% or 99% or complete (100%) sequence homology to a nucleic acid or a nucleic acid sequence: as set forth in any one of SEQ ID NO:201 to SEQ ID NO:583,

and optionally the sequence identities are determined by analysis with a sequence comparison algorithm or by a visual inspection,

and optionally the sequence comparison algorithm is a BLAST version 2.2.2 algorithm where a filtering setting is set to blastall -p blastp -d “nr pataa”-F F, and all other options are set to default.

In alternative embodiments, the invention provides isolated, synthetic or recombinant nucleic acids comprising or consisting of a nucleic acid sequence capable of specifically (selectively) hybridizing (hybridizes under stringent conditions to) to a nucleic acid of the invention, or a nucleic acid sequence as set forth in Table 1, Table 2, Table 3 or Table 4, or a nucleic acid or nucleic acid sequence as set forth in any one of SEQ ID NO:1 to SEQ ID NO:200 or SEQ ID NO:201 to SEQ ID NO:583,

wherein optionally the stringent conditions include a wash step comprising a wash in 0.2×SSC at a temperature of about 65° C. for about 15 minutes.

In alternative embodiments, the nucleic acid sequence capable of specifically (selectively) hybridizing to (hybridizes under stringent conditions to) a nucleic acid of the invention, or a nucleic acid sequence as set forth in Table 1, Table 2, Table 3 or Table 4, comprises or consists of:

(a) a member of an amplification primer pair, a polymerase chain reaction (PCR) primer pair, ligase chain reaction (LCR) pair, or a qPCR primer pair capable of amplifying a nucleic acid sequence as set forth in Table 2; or,

(b) a hybridization probe sequence capable of specifically (selectively) hybridizing to a nucleic acid or nucleic acid sequence of the invention, or as set forth in Table 1, Table 2, Table 3 or Table 4, or a nucleic acid or nucleic acid sequence as set forth in any one of SEQ ID NO:1 to SEQ ID NO:200 or SEQ ID NO:201 to SEQ ID NO:583.

In alternative embodiments, a nucleic acid of the invention can further comprise a detectable moiety or an enzyme. In alternative embodiments, the detectable moiety comprises a radioactive probe, a fluorescent molecule (e.g., a fluorescent label or a fluorophore, e.g., a coumarin, resorufin, xanthene, benzoxanthene, cyanine or bodipy analog), a quantum dot or a colloidal quantum dot (QD) (e.g., a QDOT™ nanocrystal, Life Technologies, Carlsbad, Calif.), and/or an epitope or binding molecule (e.g. a ligand).

In alternative embodiments, a nucleic acid of the invention can further comprise, or can be immobilized or conjugated or bound to, a solid or semi-solid surface. The solid or semi-solid surface comprises or consists of an array, a biochip, a chip, a bead, a gel, a liposome, a fiber, a film, a membrane, a metal, a resin, a polymer, a ceramic, a glass, an electrode, a microelectrode, a graphitic particle, or a microparticle or a nanoparticle.

In alternative embodiments, the invention provides amplification primer pairs or amplification pairs, polymerase chain reaction (PCR) primer pairs, ligase chain reaction (LCR) pairs, or qPCR primer pairs, comprising or consisting of:

(a) a primer pair as set forth in Table 2, or one member of a primer pair as set forth in Table 2,

(b) a primer pair comprising or consisting of: SEQ ID NO:1 and SEQ ID NO:2; SEQ ID NO:3 and SEQ ID NO:4; SEQ ID NO:5 and SEQ ID NO:6; SEQ ID NO:7 and SEQ ID NO:8; SEQ ID NO:9 and SEQ ID NO:10; SEQ ID NO:11 and SEQ ID NO:12; SEQ ID NO:13 and SEQ ID NO:14; SEQ ID NO:15 and SEQ ID NO:16; SEQ ID NO:17 and SEQ ID NO:18; SEQ ID NO:19 and SEQ ID NO:20; SEQ ID NO:21 and SEQ ID NO:22; SEQ ID NO:23 and SEQ ID NO:24; SEQ ID NO:25 and SEQ ID NO:26; SEQ ID NO:27 and SEQ ID NO:28; SEQ ID NO:29 and SEQ ID NO:30; SEQ ID NO:31 and SEQ ID NO:32; SEQ ID NO:33 and SEQ ID NO:34; SEQ ID NO:35 and SEQ ID NO:36; SEQ ID NO:37 and SEQ ID NO:38; SEQ ID NO:39 and SEQ ID NO:40; SEQ ID NO:41 and SEQ ID NO:42; SEQ ID NO:43 and SEQ ID NO:44; SEQ ID NO:45 and SEQ ID NO:46; SEQ ID NO:47 and SEQ ID NO:48; SEQ ID NO:49 and SEQ ID NO:50; SEQ ID NO:51 and SEQ ID NO:52; SEQ ID NO:53 and SEQ ID NO:54; SEQ ID NO:55 and SEQ ID NO:56; SEQ ID NO:57 and SEQ ID NO:58; SEQ ID NO:59 and SEQ ID NO:60; SEQ ID NO:61 and SEQ ID NO:62, SEQ ID NO:63 and SEQ ID NO:64; SEQ ID NO:65 and SEQ ID NO:66; SEQ ID NO:67 and SEQ ID NO:68; SEQ ID NO:69 and SEQ ID NO:70; SEQ ID NO:71 and SEQ ID NO:72; SEQ ID NO:73 and SEQ ID NO:74; SEQ ID NO:75 and SEQ ID NO:76; SEQ ID NO:77 and SEQ ID NO:78; SEQ ID NO:79 and SEQ ID NO:80; SEQ ID NO:81 and SEQ ID NO:82; SEQ ID NO:83 and SEQ ID NO:84; SEQ ID NO:85 and SEQ ID NO:86; SEQ ID NO:87 and SEQ ID NO:88; SEQ ID NO:89 and SEQ ID NO:90; SEQ ID NO:91 and SEQ ID NO:92; SEQ ID NO:93 and SEQ ID NO:94; SEQ ID NO:95 and SEQ ID NO:96; SEQ ID NO:97 and SEQ ID NO:98; SEQ ID NO:99 and SEQ ID NO:100; SEQ ID NO:101 and SEQ ID NO:102; SEQ ID NO:103 and SEQ ID NO:104; SEQ ID NO:105 and SEQ ID NO:106; SEQ ID NO:107 and SEQ ID NO:108; SEQ ID NO:109 and SEQ ID NO:110; SEQ ID NO:111 and SEQ ID NO:112; SEQ ID NO:113 and SEQ ID NO:114; SEQ ID NO:115 and SEQ ID NO:116; SEQ ID NO:117 and SEQ ID NO:118; SEQ ID NO:119 and SEQ ID NO:120; SEQ ID NO:121 and SEQ ID NO:122; SEQ ID NO:123 and SEQ ID NO:124; SEQ ID NO:125 and SEQ ID NO:126; SEQ ID NO:127 and SEQ ID NO:128; SEQ ID NO:129 and SEQ ID NO:130; SEQ ID NO:131 and SEQ ID NO:132; SEQ ID NO:133 and SEQ ID NO:134; SEQ ID NO:135 and SEQ ID NO:136; SEQ ID NO:137 and SEQ ID NO:138; SEQ ID NO:139 and SEQ ID NO:140; SEQ ID NO:141 and SEQ ID NO:142; SEQ ID NO:143 and SEQ ID NO:144; SEQ ID NO:145 and SEQ ID NO:146; SEQ ID NO:147 and SEQ ID NO:148; SEQ ID NO:149 and SEQ ID NO:150; SEQ ID NO:151 and SEQ ID NO:152; SEQ ID NO:153 and SEQ ID NO:154; SEQ ID NO:155 and SEQ ID NO:156; SEQ ID NO:157 and SEQ ID NO:158; SEQ ID NO:159 and SEQ ID NO:160; SEQ ID NO:161 and SEQ ID NO:162; SEQ ID NO:163 and SEQ ID NO:164; SEQ ID NO:165 and SEQ ID NO:166; SEQ ID NO:167 and SEQ ID NO:168; SEQ ID NO:169 and SEQ ID NO:170; SEQ ID NO:171 and SEQ ID NO:172; SEQ ID NO:173 and SEQ ID NO:174; SEQ ID NO:175 and SEQ ID NO:176; SEQ ID NO:177 and SEQ ID NO:178; SEQ ID NO:179 and SEQ ID NO:180; SEQ ID NO:181 and SEQ ID NO:182; SEQ ID NO:183 and SEQ ID NO:184; SEQ ID NO:185 and SEQ ID NO:186; SEQ ID NO:187 and SEQ ID NO:188; SEQ ID NO:189 and SEQ ID NO:190; SEQ ID NO:191 and SEQ ID NO:192; SEQ ID NO:193 and SEQ ID NO:194; SEQ ID NO:195 and SEQ ID NO:196; SEQ ID NO:197 and SEQ ID NO:198; or, SEQ ID NO:199 and SEQ ID NO:200; (c) all of the primer pairs as set forth in Table 2; or (d) all of the primer pairs of (b).

In alternative embodiments, at least one member of the primer pair further comprises a detectable moiety. In alternative embodiments, the detectable moiety comprises a radioactive probe, a fluorescent molecule (e.g., a fluorescent label or a fluorophore, e.g., a coumarin, resorufin, xanthene, benzoxanthene, cyanine or bodipy analog), a quantum dot or a colloidal quantum dot (QD) (e.g., a QDOT™ nanocrystal, Life Technologies, Carlsbad, Calif.), and/or an epitope or binding molecule (e.g. a ligand).

In alternative embodiments, at least one member of the primer pair, or both members of the primer pair, further comprise, or are immobilized or conjugated or bound to, a solid or a semi-solid surface. The solid or semi-solid surface can comprise or consist of an array, a biochip, a chip, a bead, a gel, a liposome, a fiber, a film, a membrane, a metal, a resin, a polymer, a ceramic, a glass, an electrode, a microelectrode, a graphitic particle, or a microparticle or a nanoparticle.

In alternative embodiments, the invention provides products of manufacture, arrays, biochips, chips, beads, gels, liposomes, fibers, films, membranes, metals, resins, polymers, ceramics, glasses, electrodes, microelectrodes, graphitic particles, or microparticles or nanoparticles, comprising a nucleic acid of the invention, or a plurality of or all of the nucleic acids of the invention, or an amplification primer pair, polymerase chain reaction (PCR) primer pair, a ligase chain reaction (LCR) pair, or a qPCR primer pair of the invention, or all amplification primer pairs, polymerase chain reaction (PCR) primer pairs, a ligase chain reaction (LCR) pairs or qPCR primer pairs of the invention.

In alternative embodiments, the invention provides kits comprising a nucleic acid of the invention, or a plurality of or all of the nucleic acids of the invention, or an amplification primer pair, a polymerase chain reaction (PCR) primer pair, a ligase chain reaction (LCR) pair, or a qPCR primer pair of the invention, wherein optionally the kit comprises or is a PCR, LCR or qPCR kit, and optionally the nucleic acid, amplification primer pair, polymerase chain reaction (PCR) primer pair, ligase chain reaction (LCR) pair or qPCR primer pair is contained or stored in a solution, a test tube or a container.

In alternative embodiments, the invention provides methods of detecting, identifying, quantifying and/or indicating the presence of a hydrocarbon in a sample, comprising:

(a) obtaining or providing one sample or a set of samples,

wherein optionally the sample is an aqueous sample, a fresh water sample or a sea water sample, or a sediment, sand, shale or mud, or a marine sediment, sand, shale or mud, or a solution,

or optionally the samples comprise fresh water, underground water or seawater, or a production water, or an aqueous sample or a marine sediment, sand, shale or mud are taken from or prepared from a core sample;

(b) detecting, determining, quantifying and/or characterizing the presence of a nucleic acid in the sample or samples, wherein the detecting, determining, characterizing or quantifying (measuring) the presence of the nucleic acid in the sample or samples indicates the presence of, or quantifies or estimates the amount of, the hydrocarbon in the sample or solution,

and the nucleic acid detected, characterized or quantified comprises or consists of a nucleic acid of the invention, and/or

the nucleic acid is detected, characterized or quantified using:
a nucleic acid of the invention, or
an amplification primer pair, polymerase chain reaction (PCR) primer pair, ligase chain reaction (LCR) pair, or qPCR primer pair of the invention (for example, all of the primers pairs of the invention), or
an array, a biochip, a chip, a bead, a gel, a liposome, a fiber, a film, a membrane, a metal, a resin, a polymer, a ceramic, a glass, an electrode, a microelectrode, a graphitic particle, or a microparticle or a nanoparticle of the invention,
a product of manufacture, an array, a biochip, a chip, a bead, a gel, a liposome, a fiber, a film, a membrane, a metal, a resin, a polymer, a ceramic, a glass, an electrode, a microelectrode, a graphitic particle, or a microparticle or a nanoparticle of the invention;

wherein optionally the determining, quantifying and/or characterizing the presence of a nucleic acid in the sample or samples is by a method comprising an amplification, a polymerase chain reaction (PCR), a qPCR and/or a hybridization;

wherein optionally identifying, quantifying and/or characterizing a nucleic acid in the sample or samples also by correlation identifies, quantifies or indicates the presence of a hydrocarbon in the solution.

wherein detecting, quantifying, determining and/or characterizing the nucleic acid in the sample or samples quantifies, identifies or detects the presence of the hydrocarbon in the sample.

In alternative embodiments of the methods, each test sample is assayed for the presence of a plurality of, or many independent, bioindicators that are positively correlated with the presence of one or more hydrocarbons, wherein optionally the bioindicator comprises a nucleic acid of the invention.

In alternative embodiments of the methods, a test sample is assayed for the presence of one or more, or a plurality of, microbial bioindicator sequences or nucleic acids that are positively and negatively associated with the presence of a hydrocarbon, wherein optionally the microbial bioindicator sequence or nucleic acid comprises a nucleic acid of the invention.

In alternative embodiments of the methods, an RNA is extracted from the sample or samples, and the RNA converted to DNA prior to PCR amplification and/or hybridization, wherein optionally the RNA is ribosomal RNA, or optionally the RNA converted to DNA using a reverse transcriptase enzyme.

In alternative embodiments the methods further comprise characterizing and/or identifying one, all or substantially most of the microbes in the sample or samples, wherein optionally the microbial composition is determined by a chemical or analytical method, and optionally the chemical or analytical method comprises a fatty acid methyl ester analysis, a membrane lipid analysis and/or a cultivation-dependent method.

In alternative embodiments the invention provides methods of detecting the presence of a subsurface hydrocarbon, petroleum, oil or gas accumulation or deposit, or the presence of a petroleum or hydrocarbon seep, spill, pollutant or leak, comprising:

(a) obtaining or providing one samples or a set of samples,

wherein optionally the sample or samples are from, or comprise, a marine sediment, shale, sand or mud, or an aqueous source, or seawater, fresh water or production fluid,

and optionally the sample or samples comprise a fresh water, underground water or seawater source, or a production water, or the marine sediment, sand or mud, or aqueous sample is taken from or prepared from a core sample, and optionally the seep is a thermogenic hydrocarbon seep or a macroseep or a microseep;

(b) determining, detecting and/or characterizing the presence of a nucleic acid in the sample or samples, wherein the presence of a nucleic acid in the sample or samples indicates the presence of a subsurface hydrocarbon, petroleum, oil or gas accumulation or deposit, or a leak, pollutant, seep or spill,

and the nucleic acid detected, characterized or quantified comprises or consists of a nucleic acid of the invention, and/or

the nucleic acid is detected, characterized or quantified using:
a nucleic acid of the invention, or
an amplification primer pair, polymerase chain reaction (PCR) primer pair, ligase chain reaction (LCR) pair, or qPCR primer pair of the invention, or
an array, a biochip, a chip, a bead, a gel, a liposome, a fiber, a film, a membrane, a metal, a resin, a polymer, a ceramic, a glass, an electrode, a microelectrode, a graphitic particle, or a microparticle or a nanoparticle of the invention,
a product of manufacture, an array, a biochip, a chip, a bead, a gel, a liposome, a fiber, a film, a membrane, a metal, a resin, a polymer, a ceramic, a glass, an electrode, a microelectrode, a graphitic particle, or a microparticle or a nanoparticle of claim 14;

wherein optionally the detecting, quantifying, determining and/or characterizing the presence of a nucleic acid in the sample or samples is by a method comprising amplification, polymerase chain reaction (PCR), qPCR and/or hybridization;

wherein detecting, quantifying, determining and/or characterizing a nucleic acid in the sample or samples quantifies, identifies or detects the presence of a subsurface hydrocarbon, petroleum, oil or gas accumulation or deposit, or the presence of a petroleum or hydrocarbon seep, pollutant, spill or leak.

In alternative embodiments of the methods, each sample is assayed for the presence of a plurality of, or many independent, bioindicators that are positively correlated with the presence of one or more hydrocarbons. In alternative embodiments of the methods, the sample is assayed for the presence of one or more, or a plurality of, microbial bioindicator sequences that are positively and negatively associated with the presence of hydrocarbons.

In alternative embodiments of the methods, an RNA is extracted from samples and converted to DNA by methods well known in the art (e.g. using reverse transcriptase), prior to PCR amplification and/or hybridization, wherein optionally the RNA is ribosomal RNA.

In alternative embodiments the methods further comprise characterizing and/or identifying one, all or substantially most of the microbes in the sample or samples, wherein optionally the microbial composition is determined by a chemical or analytical method, and optionally the chemical or analytical method comprises a fatty acid methyl ester analysis, a membrane lipid analysis and/or a cultivation-dependent method.

In alternative embodiments, the invention provides kits comprising a kit of the invention and instructions comprising a method of the invention.

The details of one or more embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference for all purposes.

### BRIEF DESCRIPTION OF THE DRAWINGS

The drawings set forth herein are illustrative of embodiments of the invention and are not meant to limit the scope of the invention as encompassed by the claims.

FIG. 1 schematically illustrates a phylogenetic tree of 11,122 16S rRNA gene sequences from the Gulf of Mexico; branches have been collapsed to division taxonomic levels; as described in detail, below.

FIG. 2 illustrates a representation of Bacterial Divisions among 15 GOM sediment samples; as described in detail, below.

FIG. 3 illustrates a representation of Archaeal Divisions among 15 GOM sediment samples; as described in detail, below.

FIG. 4 illustrates SARD profiles of 15 GOM sediment samples; as described in detail, below.

FIG. 5 illustrates comparison of PTM-03 Consensus sequence with the Genbank Non-Redundant DNA sequence database using BLASTN; as described in detail, below.

FIG. 6 illustrates comparison of PTM-04_GOM2 Consensus sequence with the Genbank Non-Redundant DNA sequence database by BLASTN search; as described in detail, below.

FIG. 7 illustrates comparison of PTM-05_GOM3 Consensus sequence with the Genbank Non-Redundant DNA sequence database by BLASTN search; as described in detail, below.

FIG. 8 illustrates comparison of PTM-06_GOM1 Consensus sequence with the Genbank Non-Redundant DNA sequence database by BLASTN search; as described in detail, below.

FIG. 9 illustrates comparison of PTM-07 Consensus sequence with the Genbank Non-Redundant DNA sequence database by BLASTN search; as described in detail, below.

FIG. 10 illustrates comparison of PTM-08 Consensus sequence with the Genbank Non-Redundant DNA sequence database by BLASTN search; as described in detail, below.

FIG. 11 illustrates comparison of PTM-10 Consensus sequence with the Genbank Non-Redundant DNA sequence database by BLASTN search performed; as described in detail, below.

FIG. 12 illustrates comparison of PTM-11 Consensus sequence with the Genbank Non-Redundant DNA sequence database by BLASTN search; as described in detail, below.

FIG. 13 graphically illustrates comparison of the abundance and distribution of gasoline-range bioindicators (top panel) with the presence of gasoline-range hydrocarbons (lower panel) in GOM sediments; as described in detail, below.

FIG. 14 graphically illustrates a plot of gasoline-range hydrocarbon bioindicator composite values versus gasoline-range values from 93 GOM sediments comprising 16 samples with known hydrocarbon values (filled circles) and 77 samples that were geochemically blinded (filled triangles); as described in detail, below.

Like reference symbols in the various drawings indicate like elements.

Reference will now be made in detail to various exemplary embodiments of the invention, examples of which are illustrated in the accompanying drawings. The following detailed description is provided to give the reader a better understanding of certain details of aspects and embodiments of the invention, and should not be interpreted as a limitation on the scope of the invention.

### DETAILED DESCRIPTION

In one embodiment, the invention provides compositions and products of manufacture, e.g., nucleic acid primers and probes, for use as identifying agents or indicators to detect the presence of a hydrocarbon in a sample, e.g., a solution, e.g., an aqueous solution, or an environmental sample such as fresh water, underground water or seawater or sand, shale or mud. In alternative embodiments, the invention provides compositions and products of manufacture, e.g., nucleic acid primers and probes, for use as bioindicators and biodetectors to detect the presence of (e.g., immediate or nearby) vertically migrating (e.g., in fresh water, underground water or seawater) hydrocarbons that e.g., can indicate the presence of subsurface petroleum, oil or gas accumulations or deposits, or leaks or spills. In one embodiment, the invention provides methods for making and using the compositions of the invention.

In alternative embodiments, the invention provides compositions, e.g., nucleic acid probes, for use as indirect bioindicator assays to detect the presence of a hydrocarbon in a sample, e.g., an aqueous sample such as water or seawater (and methods for using them), e.g., to detect seep sites, e.g., seeping hydrocarbons, which can be a “prolific” or “macroseep” or a “microseep”, or to detect leaks or spills. In alternative embodiments, use of compositions and methods of the invention has advantages over direct chemical analysis. Thus, compositions and methods of the invention can be used to interpret geochemical data from potential seep sites. In alternative embodiments, compositions and methods of the invention are used to overcome challenges related to the ephemeral nature of seeps (e.g., which include diurnal, seasonal variations) and the effects of microbes actively metabolizing seeping hydrocarbons.

A study was conducted to characterize microbial communities associated with thermogenic hydrocarbon seeps in the Green Canyon block of the Gulf of Mexico (GOM). One of the goals of the project was to identify microbes that could themselves be used as bioindicators to detect the immediate, or nearby, presence of vertically migrating hydrocarbons that would indicate the presence of subsurface petroleum accumulations. A collection of 16S rRNA gene sequences was found comprising individual bioindicator sequences that each displayed significant statistical associations with certain hydrocarbons. The organisms these sequences identify also may possess value for chemical transformation (upgrading) of heavy oil or enhanced oil recovery.

### Generating and Manipulating Nucleic Acids

In alternative embodiments, the invention provides synthetic, recombinant and isolated nucleic acids, including amplification primer pairs and probes, e.g., hybridization probes, for detecting or quantifying a hydrocarbon in a sample such as water, fresh water, seawater, mud, shale or sand, or for detecting the presence of a subsurface petroleum, oil or gas accumulation or deposit, or for detecting the presence of a petroleum seep or leak or spill, and generally practicing methods of the invention.

The nucleic acids of the invention, or used to practice methods this invention, can be made, isolated and/or manipulated by, e.g., cloning and expression of cDNA libraries, amplification of message or genomic DNA by PCR, and the like. In practicing the methods of the invention, homologous genes can be modified by manipulating a template nucleic acid, as described herein. The invention can be practiced in conjunction with any method or protocol or device known in the art, which are well described in the scientific and patent literature.

General Techniques

The synthetic, recombinant and isolated nucleic acids of the invention, or used to practice methods this invention, whether RNA (e.g., rRNA), antisense nucleic acid, cDNA, genomic DNA, vectors, viruses and the like, may be isolated, or initially isolated, from a variety of sources, genetically engineered, amplified, and/or expressed/generated recombinantly. Recombinant polypeptides generated from these nucleic acids can be individually isolated or cloned and tested for a desired activity. Any recombinant expression system can be used, including bacterial, mammalian, yeast, insect or plant cell expression systems.

Alternatively, nucleic acids of the invention, or used to practice methods this invention, can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066. In alternative embodiments, nucleic acids used to practice this invention, or nucleic acids of this invention, can comprise entirely, or in part, any non-naturally-occurring oligonucleotide analogue, e.g., thioate-type oligonucleotides, or synthetic oligos comprising unsubstituted purin-9-yl, unsubstituted 2-oxo-pyrimidin-1-yl or a substituted purin-9-yl, e.g., as described in U.S. Pat. App. Pub. No. 20090149404. In alternative embodiments, a ribose sugar of one or more of a nucleotide used to practice this invention is replaced with another moiety, e.g., a non-carbohydrate, e.g., a cyclic carrier, e.g., as described in U.S. Pat. App. Pub. No. 20100069471. In alternative embodiments, nucleic acids used to practice this invention, or nucleic acids of this invention, can comprise entirely, or in part, any peptide nucleic acids (PNA), e.g., any polyamide nucleic acid (PNA) derivative, e.g., as described in U.S. Pat. App. Pub. No. 20100022016; PNA binds to complementary DNA and RNA even at low salt concentration.

In alternative embodiments, nucleic acids used to practice methods of this invention, or nucleic acids of this invention, can comprise (partially or entirely) peptide nucleic acids (PNAs) containing non-ionic backbones, such as N-(2-aminoethyl)glycine units; or can comprise phosphorothioate linkages, e.g., as described in WO 97/03211; WO 96/39154; Mata (1997) Toxicol. Appl. Pharmacol. 144:189-197; Antisense Therapeutics, ed. Agrawal (Humana Press, Totowa, N.J., 1996). In alternative embodiments, nucleic acids used to practice this invention, or nucleic acids of this invention, can comprise (partially or entirely) synthetic DNA backbone analogues comprising phosphoro-dithioate, methylphosphonate, phosphoramidate, alkyl phosphotriester, sulfamate, 3′-thioacetal, methylene(methylimino), 3′-N-carbamate, and morpholino carbamate nucleic acids.

Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

Amplification of Nucleic Acids

In alternative embodiments, nucleic acids of the invention, or used to practice methods this invention, are used in amplification reactions to detect nucleic acids in a sample, e.g., an aqueous sample, such as an environmental sample (such as fresh, sea or ground water, sand, mud, shale and the like) e.g., to detect and/or quantify the presence of a hydrocarbon in the sample, e.g., in a subsurface petroleum, oil or gas accumulation or deposit, or the presence of a petroleum seep, spill or leak. Alternatively, nucleic acids of the invention, or used to practice methods this invention, themselves can be made or reproduced by amplification. Amplification can also be used to clone or modify the nucleic acids of the invention, or used to practice methods this invention.

In alternative embodiments, amplification reactions are used to quantify the amount of nucleic acid in a sample (such as the amount of a specific rRNA sequence in a sample), to label the nucleic acid (e.g., to apply it to an array or a blot), detect the nucleic acid, or quantify the amount of a specific nucleic acid in a sample. In one aspect of the invention, RNA isolated from a sample is amplified, or reverse transcribed and then amplified.

In alternative embodiments, in addition to the amplification primers described herein, skilled artisan can select and design equivalent oligonucleotide amplification primers to practice the methods of this invention. Amplification methods are also well known in the art, and include, e.g., polymerase chain reaction, PCR (see, e.g., PCR PROTOCOLS, A GUIDE TO METHODS AND APPLICATIONS, ed. Innis, Academic Press, N.Y. (1990) and PCR STRATEGIES (1995), ed. Innis, Academic Press, Inc., N.Y., ligase chain reaction (LCR) (see, e.g., Wu (1989) Genomics 4:560; Landegren (1988) Science 241:1077; Barringer (1990) Gene 89:117); transcription amplification (see, e.g., Kwoh (1989) Proc. Natl. Acad. Sci. USA 86:1173); and, self-sustained sequence replication (see, e.g., Guatelli (1990) Proc. Natl. Acad. Sci. USA 87:1874); Q Beta replicase amplification (see, e.g., Smith (1997) J. Clin. Microbiol. 35:1477-1491), automated Q-beta replicase amplification assay (see, e.g., Burg (1996) Mol. Cell. Probes 10:257-271) and other RNA polymerase mediated techniques (e.g., NASBA, Cangene, Mississauga, Ontario); see also Berger (1987) Methods Enzymol. 152:307-316; Sambrook; Ausubel; U.S. Pat. Nos. 4,683,195 and 4,683,202; Sooknanan (1995) Biotechnology 13:563-564.

In practicing the invention, any apparatus for nucleic acid, e.g., DNA, amplification, e.g., for qualitative and/or quantitative measurements, can be used, e.g., as described in U.S. Pat. App. Pub. No. 20100075312. For example, practicing the invention can comprise methods or compositions as described in U.S. Pat. No. 5,994,056, which describes an approach to PCR in which there is simultaneous amplification and detection. Alternatively, practicing the invention can comprise using methods or compositions as described in U.S. Pat. No. 6,586,233, which describes an arrangement for convectively-driven thermal cycling to perform a polymerase chain reaction (PCR). Alternatively, practicing the invention can comprise using quantitative PCR (qPCR) arrays as described in e.g., U.S. Pat. App. Pub. No. 20090142759, describing qPCR assays.

Alternatively, practicing the invention can comprise using real-time polymerase chain reaction, also called quantitative real time polymerase chain reaction (Q-PCR/qPCR/qrt-PCR) or kinetic polymerase chain reaction (KPCR); or multiplex qPCR, real-time PCR, and/or reverse transcription quantitative PCR (RT-qPCR).

Hybridization of Nucleic Acids

In alternative embodiments, the invention provides nucleic acids that hybridize under stringent conditions (or selective, or highly selective) to polynucleotides whose presence in a sample detects or indicates the presence of a hydrocarbon, e.g., a subsurface petroleum, oil or gas accumulation or deposit, or the presence of a petroleum seep or leak or spill, or quantifies the presence of a hydrocarbon in the sample. The stringent conditions can be highly stringent conditions, medium stringent conditions, low stringent conditions. In one aspect, it is the stringency of the wash conditions that set forth the conditions which determine whether a nucleic acid binds to a desired target.

In alternative embodiments, nucleic acids of the invention are designed to hybridize under high stringency comprising conditions of about 50% formamide at about 37° C. to 42° C.; or designed to hybridize under reduced stringency comprising conditions in about 35% to 25% formamide at about 30° C. to 35° C.; or are designed to hybridize under high stringency comprising conditions at 42° C. in 50% formamide, 5×SSPE, 0.3% SDS, and a repetitive sequence blocking nucleic acid, such as cot-1 or salmon sperm DNA (e.g., 200 n/ml sheared and denatured salmon sperm DNA); or to hybridize under reduced stringency conditions comprising 35% formamide at a reduced temperature of 35° C.

In alternative embodiments, following hybridization, the hybridized nucleic acids are washed with 6×SSC, 0.5% SDS at 50° C. These conditions are considered to be “moderate” conditions above 25% formamide and “low” conditions below 25% formamide. In alternative embodiments, hybridization is conducted at 30% formamide; or hybridization is conducted at 10% formamide.

In alternative embodiments, hybridization is carried out in buffers, such as SSC, e.g., 6×SSC, e.g. containing formamide, e.g. at a temperature of 42° C. In alternative embodiments, the concentration of formamide in the hybridization buffer is reduced. In alternative embodiments, following hybridization, a filter may be washed with 6×SSC, 0.5% SDS at 50° C.

In alternative embodiments, selection of a hybridization format is not critical—it is the stringency of the wash conditions that set forth the conditions which determine whether a nucleic acid remains bound (hybridized) to a desired target. In alternative embodiments wash conditions include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. See Sambrook, Tijssen and Ausubel for a description of SSC buffer and equivalent conditions.

Determining the Degree of Sequence Identity

The invention provides isolated, synthetic or recombinant nucleic acids comprising sequences having at least about 85%, 86%, 87%, 88%, 89%, 90%, 91%, 92%, 93%, 94%, 95%, 96%, 97%, 98% or 99% or complete (100%) sequence identity (homology) to a nucleic acid or a nucleic acid sequence as set forth in Table 1, Table 2, Table 3 or Table 4, or SEQ ID NO:1 to SEQ ID NO:200, or SEQ ID NO:201 to SEQ ID NO:583.

The extent of sequence identity (homology) may be determined using any computer program and associated parameters, including those described herein, such as BLAST 2.2.2. or FASTA version 3.0t78, with the default parameters. In alternative embodiments, the sequence identify can be over a region of at least about 5, 10, 20, 30, 40, 50, 100, 150, 200, 250, 300, 350, 400 consecutive residues, or the full length of the nucleic acid. Algorithms and programs used to practice this invention include, but are not limited to, TBLASTN, BLASTP, FASTA, TFASTA, and CLUSTALW (Pearson and Lipman, Proc. Natl. Acad. Sci. USA 85(8):2444-2448, 1988; Altschul et al., J. Mol. Biol. 215(3):403-410, 1990; Thompson et al., Nucleic Acids Res. 22(2):4673-4680, 1994; Higgins et al., Methods Enzymol. 266:383-402, 1996; Altschul et al., J. Mol. Biol. 215(3):403-410, 1990; Altschul et al., Nature Genetics 3:266-272, 1993).

A “comparison window” includes reference to a segment of any one of the number of contiguous residues. For example, in alternative embodiments of the invention, contiguous residues ranging anywhere from 20 to the full length of an exemplary sequence of the invention are compared to a reference sequence of the same number of contiguous positions after the two sequences are optimally aligned. If the reference sequence has the requisite sequence identity to an exemplary sequence of the invention, e.g., 85%, 86%, 87%, 88%, 89%, 90%, 91%, 92%, 93%, 94%, 95%, 96%, 97%, 98%, 99%, or more sequence identity to a sequence of the invention, that sequence is within the scope of the invention. In alternative embodiments, subsequences ranging from about 20 to 600, about 50 to 200, and about 100 to 150 are compared to a reference sequence of the same number of contiguous positions after the two sequences are optimally aligned. Methods of alignment of sequence for comparison are well-known in the art. Optimal alignment of sequences for comparison can be conducted, e.g., by the local homology algorithm of Smith & Waterman, Adv. Appl. Math. 2:482, 1981, by the homology alignment algorithm of Needleman & Wunsch, J. Mol. Biol. 48:443, 1970, by the search for similarity method of person & Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection.

BLAST, BLAST 2.0 and BLAST 2.2.2 algorithms are also used to practice the invention. They are described, e.g., in Altschul (1977) Nuc. Acids Res. 25:3389-3402; Altschul (1990) J. Mol. Biol. 215:403-410. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul (1990) supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectations (E) of 10, and the BLOSUM62 scoring matrix (see Henikoff & Henikoff (1989) Proc. Natl. Acad. Sci. USA 89:10915) alignments (B) of 50, expectation (E) of 10, M=5, N=−4, and a comparison of both strands. The BLAST algorithm also performs a statistical analysis of the similarity between two sequences (see, e.g., Karlin & Altschul (1993) Proc. Natl. Acad. Sci. USA 90:5873). One measure of similarity provided by BLAST algorithm is the smallest sum probability (P(N)), which provides an indication of the probability by which a match between two nucleotide or amino acid sequences would occur by chance. For example, a nucleic acid is considered similar to a references sequence if the smallest sum probability in a comparison of the test nucleic acid to the reference nucleic acid is less than about 0.2, more preferably less than about 0.01, and most preferably less than about 0.001. In one aspect, protein and nucleic acid sequence homologies are evaluated using the Basic Local Alignment Search Tool (“BLAST”).

In one embodiment, to determine if a nucleic acid has the requisite sequence identity to be within the scope of the invention, the NCBI BLAST 2.2.2 programs is used. default options to blastp. There are about 38 setting options in the BLAST 2.2.2 program. In this exemplary aspect of the invention, all default values are used except for the default filtering setting (i.e., all parameters set to default except filtering which is set to OFF); in its place a “-F F” setting is used, which disables filtering. Use of default filtering often results in Karlin-Altschul violations due to short length of sequence. The default values used in this exemplary aspect of the invention, include:
“Filter for low complexity: ON
>Word Size: 3
>Matrix: Blosum62
>Gap Costs: Existence:11
>Extension:1”
   Other default settings are: filter for low complexity OFF, word size of 3 for protein, BLOSUM62 matrix, gap existence penalty of −11 and a gap extension penalty of −1. An exemplary NCBI BLAST 2.2.2 program setting is set forth in Example 1, below. Note that the “-W” option defaults to 0. This means that, if not set, the word size defaults to 3 for proteins and 11 for nucleotides.

### Arrays, or “BioChips”

Nucleic acids, e.g., the probes, of the invention can be immobilized to or applied to an array, chip, biochip and the like. Arrays, chips etc. can be used to screen for or monitor samples (e.g., environmental samples such as fresh water, sea water, mud, sand and the like) for practicing a method of the invention, e.g., identifying and/or indicating the presence of a hydrocarbon in a marine sediment, sand, mud or solution.

In alternative aspects, “arrays” or “microarrays” or “biochips” or “chips” of the invention comprise a plurality of target elements (e.g., positive controls or negative controls) in addition to a nucleic acid (e.g., probe) of the invention; each target element can comprises a defined amount of one or more nucleic acids immobilized onto a defined area of a substrate surface.

The present invention can be practiced with any known “array,” also referred to as a “microarray” or “nucleic acid array” or “bioarray” or “biochip,” or variation thereof. Arrays are generically a plurality of “spots” or “target elements,” each target element comprising a defined amount of one or more biological molecules, e.g., oligonucleotides, immobilized onto a defined area of a substrate surface for specific binding to a sample molecule, e.g., genomic nucleic acid or mRNA transcripts.

In practicing the methods of the invention, any known array and/or method of making and using arrays can be incorporated in whole or in part, or variations thereof, as described, for example, in U.S. Pat. Nos. 6,277,628; 6,277,489; 6,261,776; 6,258,606; 6,054,270; 6,048,695; 6,045,996; 6,022,963; 6,013,440; 5,965,452; 5,959,098; 5,856,174; 5,830,645; 5,770,456; 5,632,957; 5,556,752; 5,143,854; 5,807,522; 5,800,992; 5,744,305; 5,700,637; 5,556,752; 5,434,049; see also, e.g., WO 99/51773; WO 99/09217; WO 97/46313; WO 96/17958; see also, e.g., Johnston (1998) Curr. Biol. 8:R171-R174; Schummer (1997) Biotechniques 23:1087-1092; Kern (1997) Biotechniques 23:120-124; Solinas-Toldo (1997) Genes, Chromosomes & Cancer 20:399-407; Bowtell (1999) Nature Genetics Supp. 21:25-32. See also published U.S. patent applications Nos. 20010018642; 20010019827; 20010016322; 20010014449; 20010014448; 20010012537; 20010008765.

### Computer Systems and Computer Program Products

Nucleic acid sequences of the invention can be stored, recorded, and manipulated on any medium which can be read and accessed by a computer. In alternative embodiments, the invention provides computers, computer systems, computer readable mediums, computer programs products and the like recorded or stored thereon the nucleic acid sequences of the invention, e.g., an exemplary sequence of the invention. As used herein, the words “recorded” and “stored” refer to a process for storing information on a computer medium. A skilled artisan can readily adopt any known methods for recording information on a computer readable medium to generate manufactures comprising one or more of the nucleic acid and/or polypeptide sequences of the invention.

In alternative embodiments, the invention provides a computer readable medium having recorded thereon at least one nucleic acid sequence of the invention. Computer readable media include magnetically readable media, optically readable media, electronically readable media, magnetic/optical media, flash drives and flash memories. For example, the computer readable media may be a hard disk, a floppy disk, a magnetic tape, a flash memory, CD-ROM, Digital Versatile Disk (DVD), Random Access Memory (RAM), or Read Only Memory (ROM), or any type of media known to those skilled in the art.

### Kits and Instructions

The invention provides kits comprising compositions and methods of the invention, including instructions for use thereof. In alternative embodiments, the invention provides kits comprising a composition (e.g., a probe of the invention), a product of manufacture, or mixture (e.g., comprising a probe of the invention) or a culture of cells (e.g., expressing probes of the invention), of the invention; wherein optionally the kit further comprises instructions for practicing a method of the invention.

The invention will be further described with reference to the following examples; however, it is to be understood that the invention is not limited to such examples.

### EXAMPLES

### Example 1

### Characterization of Microbial Communities Associated with Thermogenic Hydrocarbon Seeps

This Example describes characterization of microbial communities associated with thermogenic hydrocarbon seeps in the Green Canyon block of the Gulf of Mexico (GOM). One of the goals of the project was to identify microbes that could themselves be used as bioindicators to detect the immediate, or nearby, presence of vertically migrating hydrocarbons that would indicate the presence of subsurface petroleum accumulations. A collection of 16S rRNA gene sequences was found comprising individual bioindicator sequences that each displayed significant statistical associations with certain hydrocarbons. The organisms these sequences identify also may possess value for chemical transformation (upgrading) of heavy oil or enhanced oil recovery.

In this study, piston core samples of marine sediment were collected over a number of well-defined seep features in the Gulf of Mexico (GOM). Many of the cores contained obvious oil staining and methane hydrates. A number of molecular biological and genomics tools were utilized to characterize the microbial communities present in these samples including serial analysis of ribosomal DNA (SARD), 454 pyrosequencing and Sanger sequencing of 16S rRNA gene libraries.

Analysis of the GOM SARD profile data identified about 20,000 unique types of microbes inhabiting offshore hydrocarbon seeps. About 600 of these were found to be associated with hydrocarbon seep components and represented a significant opportunity to develop new petroleum bioindicators. The detection of a given 16S rRNA gene sequence serves as a proxy for the presence of microbes that harbor that specific gene sequence. The DNA sequences from several of these microbes were utilized to develop quantitative polymerase chain reaction (qPCR) assays to detect their presence in marine sediments. A subset of these molecular bioindicator sequences were utilized in qPCR assays to detect the presence gasoline-range hydrocarbons in a geochemically blinded set of 77 marine sediments. The assays correctly predicted the presence of these hydrocarbons in 76/77 samples, thus demonstrating the accuracy and value of reagents of the invention as a new tool for offshore oil exploration activities.

A total of 33 piston cores (6 m) were collected across the seep field. Each core was sub-sampled at 3 intervals per core (i.e. top, middle, bottom). A total of 93 subsamples were collected from the piston cores. Some intervals were not obtained for samples with significant methane hydrates present. Expansion of methane hydrates as the piston cores were raised from high pressure of the seafloor resulted in sample loss in some cases. Samples from each interval were divided up to be sent to different labs for specific geochemical analysis. Subsamples for microbiological analysis were treated aseptically, transferred to sterile containers and immediately frozen at −20° C. These samples were kept frozen until they were processed for DNA extraction at Taxon's facility (Taxon Biosciences, Inc., Tiburon, Calif.).

A subset of 16 samples was chosen for a detailed microbial community profiling to comprise a gradient of the level of hydrocarbons present. Our laboratory was only provided the geochemical data for these 16 ‘unblinded’ samples. Geochemical data for the remaining 77 samples was withheld from our lab in order to create a geochemically ‘blinded’ set of samples. One objective the project was to test whether the bioindicators sequences identified by correlation to hydrocarbons in the 16 unblinded samples could be used to accurately predict the presence of hydrocarbons in the 77 unblinded samples.

All of the samples were from the lowest interval except for those from two cores where the top, middle and lower intervals were sampled from two complete piston cores. These two cores comprised a negative control core taken from outside the seep area and a highly positive core.

Genomic DNA was extracted from the samples by a bead beating procedure e.g., as described by Ashby, M. N.; J. Rine, et al. (2007). “Serial analysis of rRNA genes and the unexpected dominance of rare members of microbial communities.” Appl Environ Microbiol 73(14): 4532-42, and was utilized to construct three types of 16S rRNA gene profiles including Sanger sequencing of clone libraries, 454 pyrosequencing utilizing Roche's Titanium chemistry and SARD. All of these approaches began with PCR amplification of a portion of the 16S rRNA gene using the primers TX9 and 1391r that corresponds approximately to positions 800 to 1400 (E. coli numbering). This portion of the 16S rRNA gene includes four variable regions (V5-V8). Each of these approaches provides a different level of detail of microbial communities.

Clone libraries were constructed by ligating PCR products into the pUC19™ (Stratagene, San Diego, Calif.) vector. E. coli transformants were picked for plasmid preparation by blue/white screening on X-Gal-containing plates. 960 individual clones (10 plates of 96) were utilized for Sanger sequencing and further analysis. Low quality and short sequences were filtered out as were sequences that failed a chimera check program, e.g., using GREENGENES™, Center for Environmental Biotechnology Lawrence Berkeley National Laboratory, Berkeley, Calif. (Bellerophon, http://greengenes.lbl.gov). Phylogenetic trees were constructed either using the PHYLIP™ software package (Felsenstein, J. 2004. [phylogeny inference package], version 3.63. Department of Genome Sciences, University of Washington, Seattle, Wash.) utilizing neighbor-joining and the KIMURA 2™-parameter distance method or using the NAST aligner available from GREENGENES™, combined with the ARB™ software package (joint initiative of the Lehrstuhl für Mikrobiologie and the Lehrstuhl für Rechnertechnik and Rechnerorganisation/Parallelrechnerarchitektur of the Technische Universität, München, Germany).

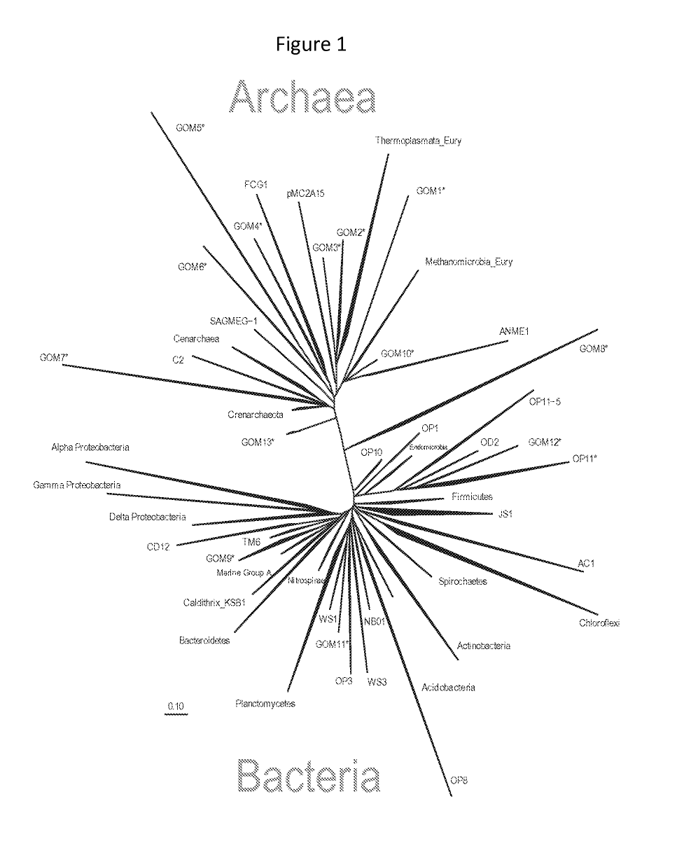

Analysis of more than 11,000 Sanger reads of 16S rRNA gene clone libraries revealed the 16 GOM sediment samples harbored significant biodiversity (FIG. 1). FIG. 1 illustrates a phylogenetic tree of 11,122 16S rRNA gene sequences from the Gulf of Mexico. Branches have been collapsed to division taxonomic levels. Divisions labeled as GOMxx are candidate divisions representing sequences that were not associated with known divisions. Eleven sets of sequences were not affiliated with known prokaryotic phylum-level divisions. These included 3 clades from the domain Bacteria and 8 clades from the domain Archaea. These clades were assigned the candidate division names GOM1-11.

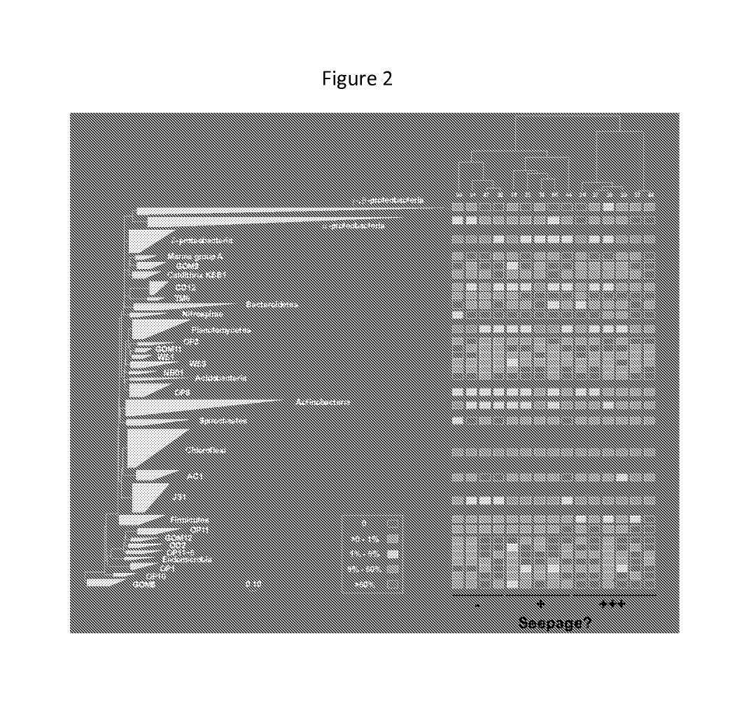

Comparison of the bacterial division representation among the 15 GOM sediment samples did not reveal any strong division-level bias toward samples that were located directly on seep features (strongly positive with visible oil staining of the sediment), adjacent to seep features (weakly positive) or outside the seepage area (negative) (FIG. 2). FIG. 2 illustrates a representation of Bacterial Divisions among 15 GOM sediment samples. Phylogenetic tree was constructed by neighbor-joining of 16S rRNA gene sequences and grouping at the division level. Samples (columns) were clustered according similarities in SARD tag composition (extracted from the longer clone library sequences).

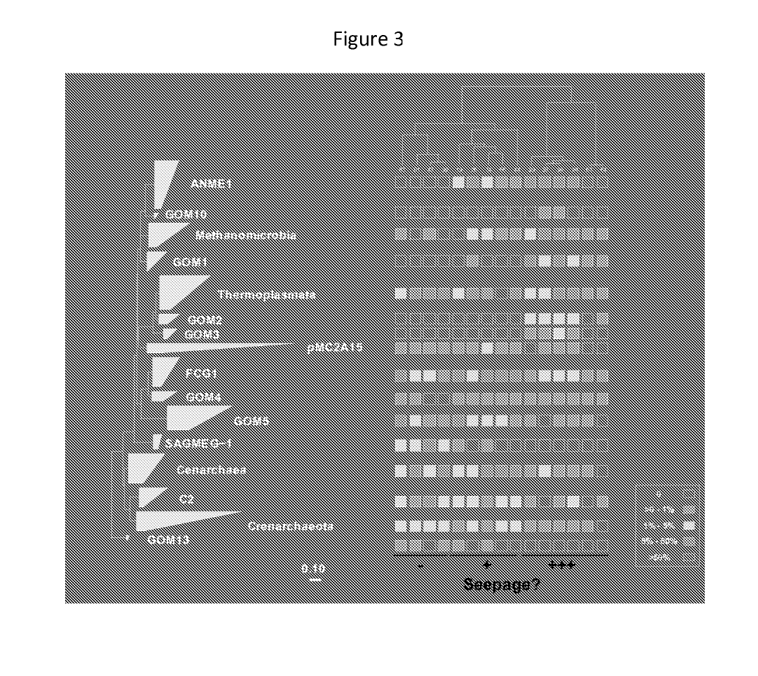

In contrast, Archaeal division representation revealed considerable bias toward and against the sample location relative to seep features (FIG. 3). FIG. 3 illustrates a representation of Archaeal Divisions among 15 GOM sediment samples. Phylogenetic tree was constructed by neighbor-joining of 16S rRNA gene sequences and grouping at the division level. Samples (columns) were clustered according similarities in SARD tag composition (extracted from the longer clone library sequences).

Representatives from the candidate Archaeal divisions GOM1, 2, 3, and 10 were seen exclusively on seep features with one exception. A single GOM1 sequence was identified from sample 16-25 that was adjacent to a seep feature and was weakly positive. Nevertheless, hundreds of sequences were observed from this candidate division among the ‘on feature’ strongly positive locations. ANME1 division sequences were only found in samples associated with the seep features (weakly or strongly positive). Representatives from the candidate division GOM13 and the division SAGMEG-1 were found with a strong bias against samples with oil and gas hydrates present.

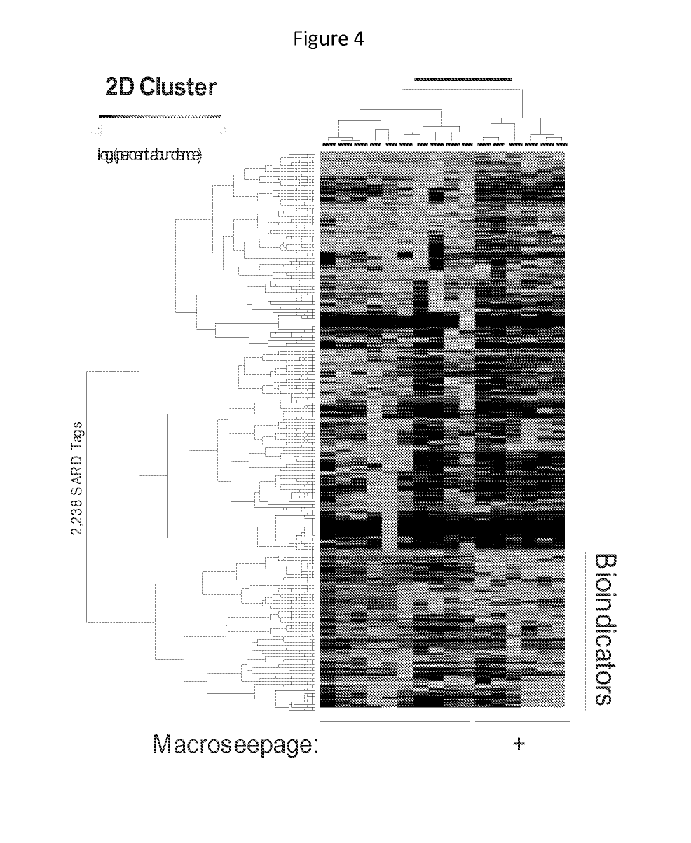

SARD libraries were also constructed from the 15 GOM samples as described previously (Ashby, Rine et al. 2007). A total of about 3.5 million V5 sequence tags were identified that comprised about 20,000 distinct or unique sequences. A 2-Dimensional dendrogram showing the distribution of SARD tags revealed non-random distribution among the sediment samples (FIG. 4). FIG. 4 illustrates SARD profiles of 15 GOM sediment samples. SARD tags (rows) were clustered with each other according to the degree of correlated distribution among the sediment samples. Samples (columns) were clustered with each other according to the correlated composition of SARD tags. The abundance of each SARD tag is denoted by color coding (see legend).

In FIG. 4, each SARD tag (rows) was clustered with that of other tags using correlation (Pearson, r) as the distance metric. Thus, SARD tags that tended to be found together in different samples were grouped together. The sediment samples (columns) were likewise clustered according to pairwise correlation of SARD tag composition between the samples. Approximately, 600 distinct SARD tag sequences were found to be strongly biased toward samples containing hydrocarbons. The microbes represented by these sequences are presumably involved in the metabolism of the petroleum and hydrocarbons present and possess value both as bioindicators and for their abilities to carry out specific chemical transformations.

16S rRNA gene sequences whose distribution correlated with specific hydrocarbons were identified by comparing their abundance in the set of GOM samples to the levels of hydrocarbons. Often clusters of related sequences (clades) were identified.

Quantitative PCR (qPCR) primers were designed by aligning the collection of 16S rRNA gene sequences that were correlated with a specific hydrocarbon type in the sediment samples. qPCR primers were chosen such that they were: 1) located within variable regions, 2) were of a sufficient length to confer an annealing temperature of approximately 63° C.; and 3), did not show any perfect matches to sequences present in GenBank using BLASTn (see e.g., Zheng Zhang et al. (2000), “A greedy algorithm for aligning DNA sequences”, J. Comput. Biol. 7(1-2):203-14). Primers were designed to 8 distinct composite 16S rRNA gene sequences that correlated with gasoline-range hydrocarbons.

Alignment of 16S rRNA gene sequences whose distributions among the samples were correlated with gasoline-range hydrocarbons. A consensus sequence is of each group is included in the alignment. The primers (oligonucleotides) designed to selectively amplify each group of sequences is indicated on the top line of the alignment, as indicated below in Table 1 (for ease of viewing, the reverse primer is shown as its reverse-complement).

In alternative embodiments, the invention provides nucleic acids comprising or consisting of the nucleic acids of Table 1, including the amplification probes (amplification primer pairs) described in Table 1, including substantially complementary probes which can amplify the same sequences as set forth in Table 1 as the described amplification primer pairs. In alternative embodiments, the invention provides nucleic acids comprising or consisting of the nucleic acids substantially complementary to the sequences of Table 1 such that they can be used as hybridization probes to identify, quantify, and/or isolate the sequences of Table 1 by sequence complementary hybridization.

For example, in one embodiment, an amplification primer pair of the invention comprises or consists of AG GGGATATCAA CTCCTCCGTG TCG (SEQ ID NO:1) and ATCACTCCGTGGCCACCCGTTG CAAC (SEQ ID NO:2), whose “reverse complement is: GGGTGGCCAC GGAGTGAT (SEQ ID NO:201), see the “PTM03” amplification primer pair; and Table 2).

In another embodiment, an amplification primer pair of the invention comprises or consists of GGGCGTAA ACGCTGTGGG CTTA (SEQ ID NO:3) and TGGATGGGTTTCGGGATTGCCTTCAC (SEQ ID NO:4), whose “reverse complement is: GTGAAGGCAA TCCCGAAACC CATCCA (SEQ ID NO:202) (see the “PTM04” amplification primer pair; and Table 2).

In an embodiment, an amplification primer pair of the invention comprises or consists of CGTAA ACGCTGCCCG CTTG (SEQ ID NO:5) and TCGAAGATAGCAACTAAGAGCGAG (SEQ ID NO:6), whose “reverse complement is: CTCG CTCTTAGTTG CTATCTTCGA (SEQ ID NO:203) (see the “PTM05” amplification primer pair; and Table 2).

In another embodiment, an amplification primer pair of the invention comprises or consists of G CTATGTGTCG GGAGATCCAC GT (SEQ ID NO:7) and TCGGGATCGGTACTCTTTGTTCCG (SEQ ID NO:8), whose “reverse complement is: CGGAA CAAAGAGTAC CGATCCCGA (SEQ ID NO:204) (see the “PTM06” amplification primer pair; and Table 2).

In one embodiment, an amplification primer pair of the invention comprises or consists of TGCTAG CTTGGTGTTG GATAACCTA (SEQ ID NO:9) and CGGACTTGAAAATAGCAACTGAAGATGG (SEQ ID NO:10); whose “reverse complement is: CCA TCTTCAGTTG CTATTTTCAA GTCCG (SEQ ID NO:205) (see the “PTM07” amplification primer pair; and Table 2).

In one embodiment, an amplification primer pair of the invention comprises or consists of CTCTGTG TCGAAGCTAA CGCCTTAA (SEQ ID NO:11) and CAGGATTTCTGGGCAGTTTCGTCAG (SEQ ID NO:12); whose “reverse complement is: CTGA CGAAACTGCC CAGAAATCCT G (SEQ ID NO:206) (see the “PTM08” amplification primer pair; and Table 2).

In one embodiment, an amplification primer pair of the invention comprises or consists of TCGA CCCCTTCTGT GCCGCA (SEQ ID NO:13) and ACCTTCCTCCGCATTATCTGCGA (SEQ ID NO:14); whose “reverse complement is: TCGCAGA TAATGCGGAG GAAGGT (SEQ ID NO:207) (see the “PTM10” amplification primer pair; and Table 2).

In one embodiment, an amplification primer pair of the invention comprises or consists of GATGTTCA CTTGGTGTCG GTCGCAC (SEQ ID NO:15) and TTGCAACTCTCTGTACCTTCCATTGTAG (SEQ ID NO:16); whose “reverse complement is: CT ACAATGGAAG GTACAGAGAG TTGCAA (SEQ ID NO:2xx) (see the “PTM11” amplification primer pair; and Table 2).

TABLE 1

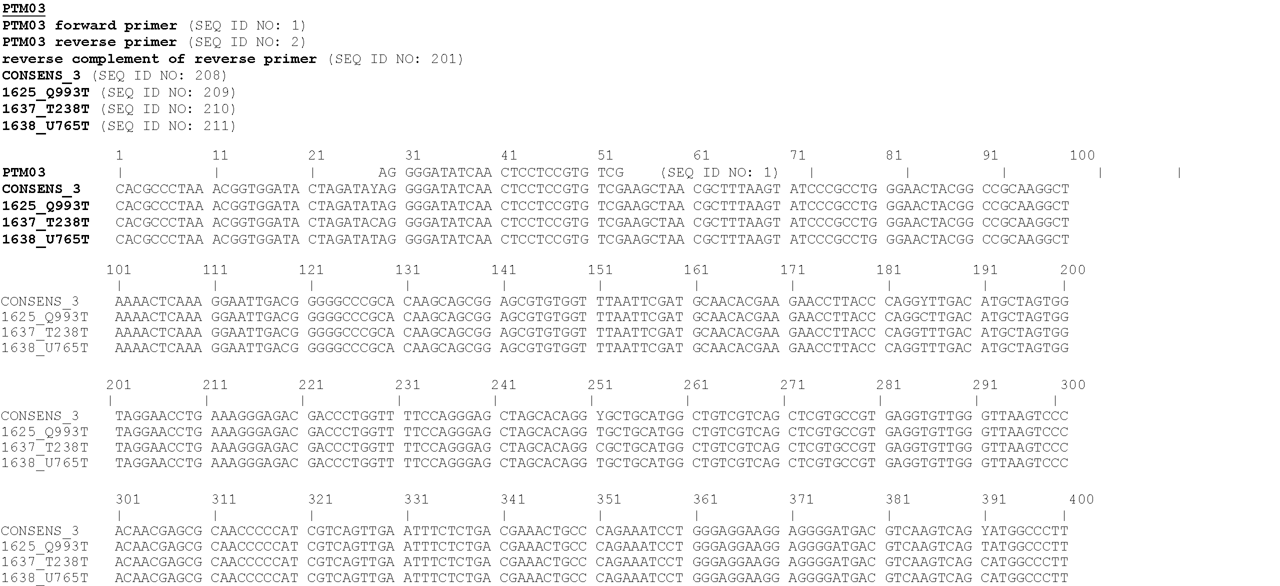

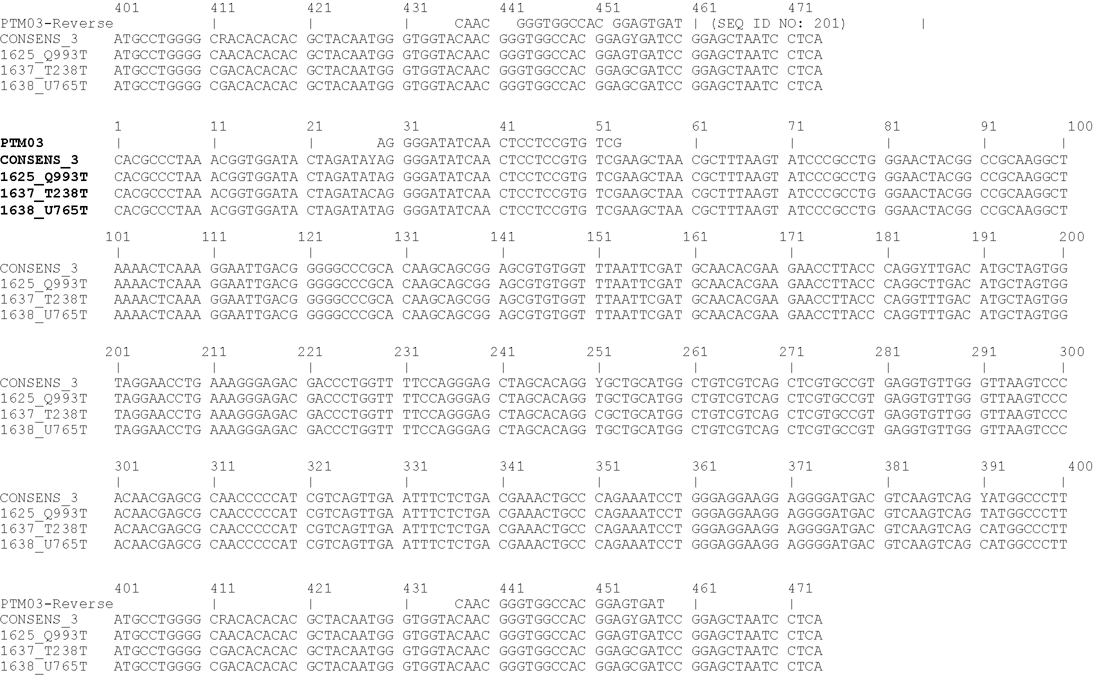

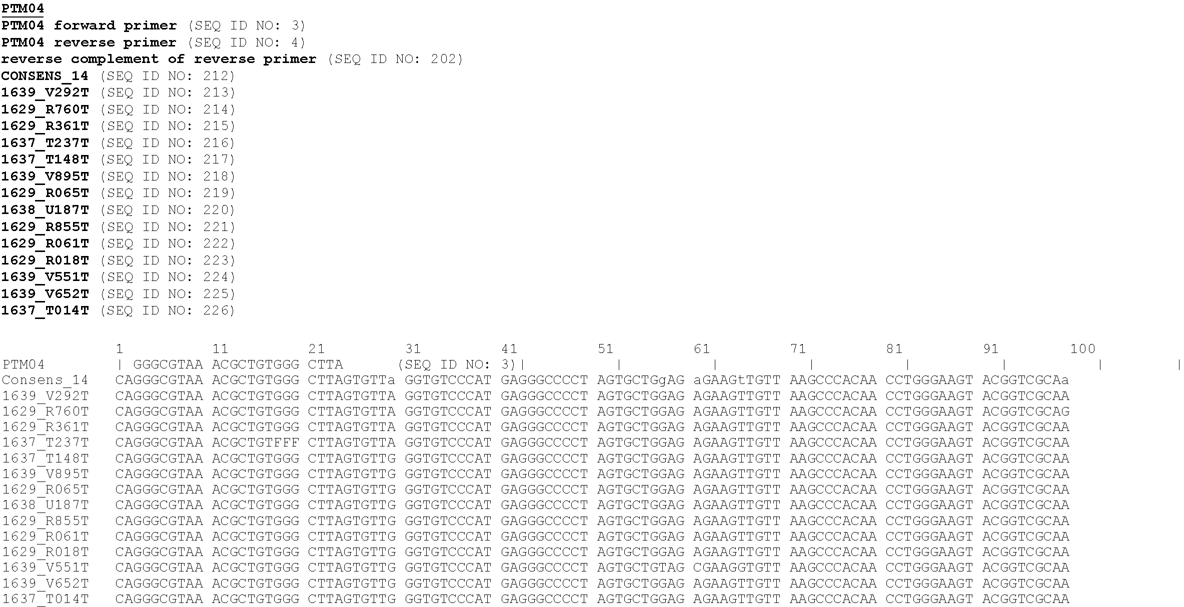

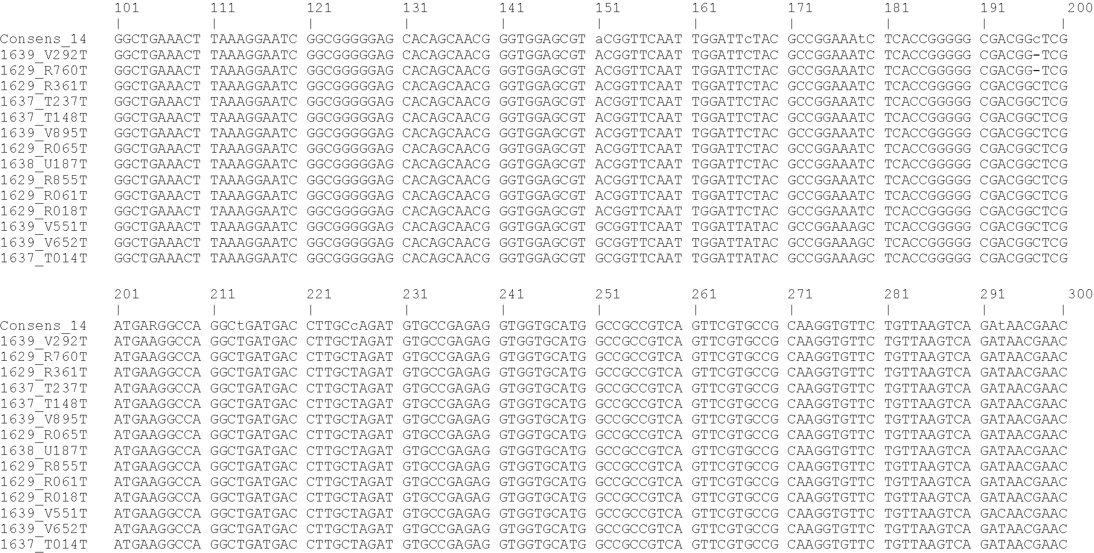

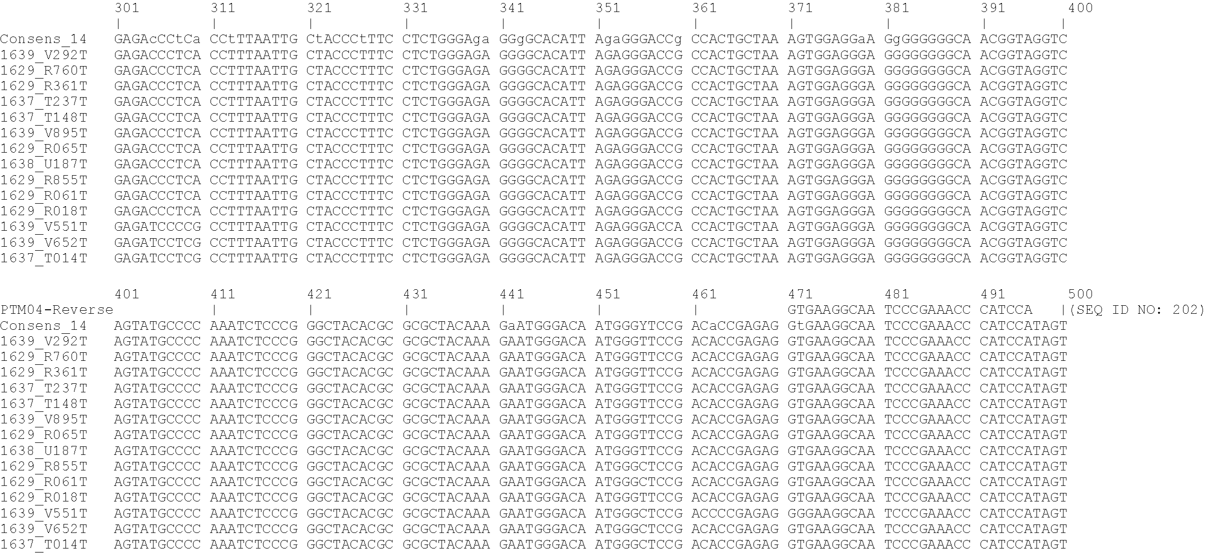

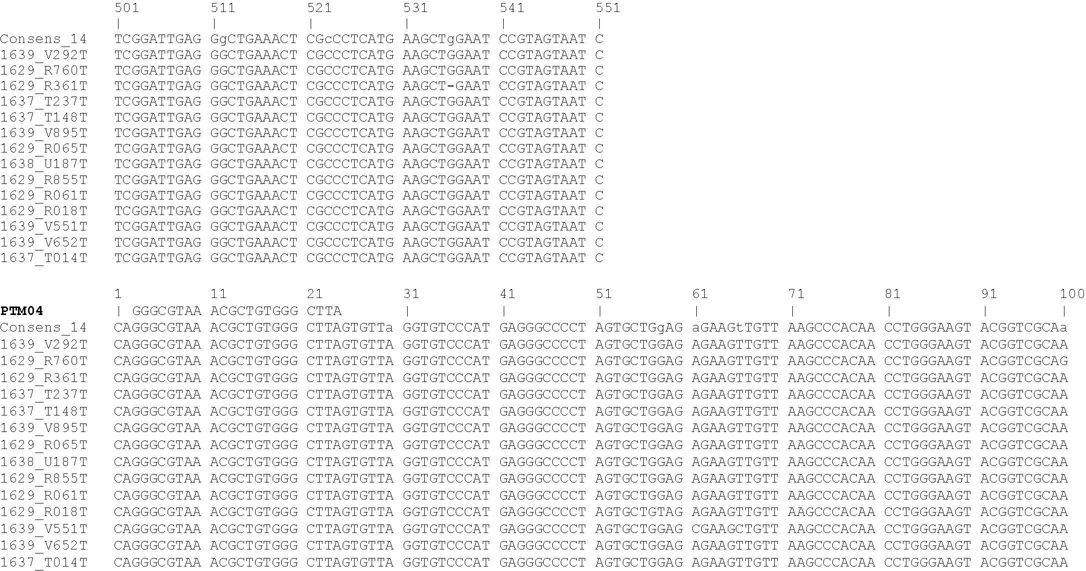

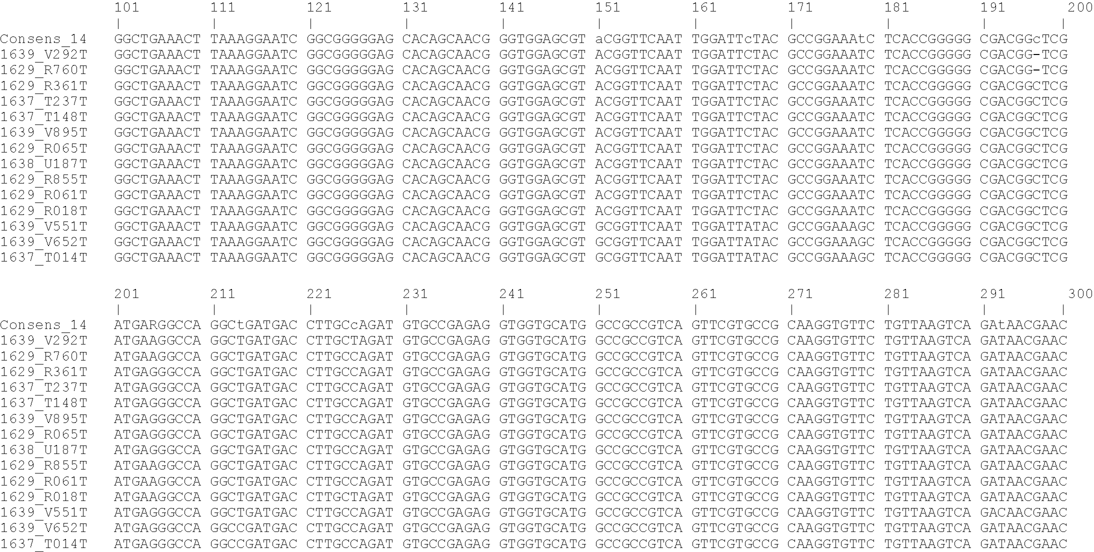

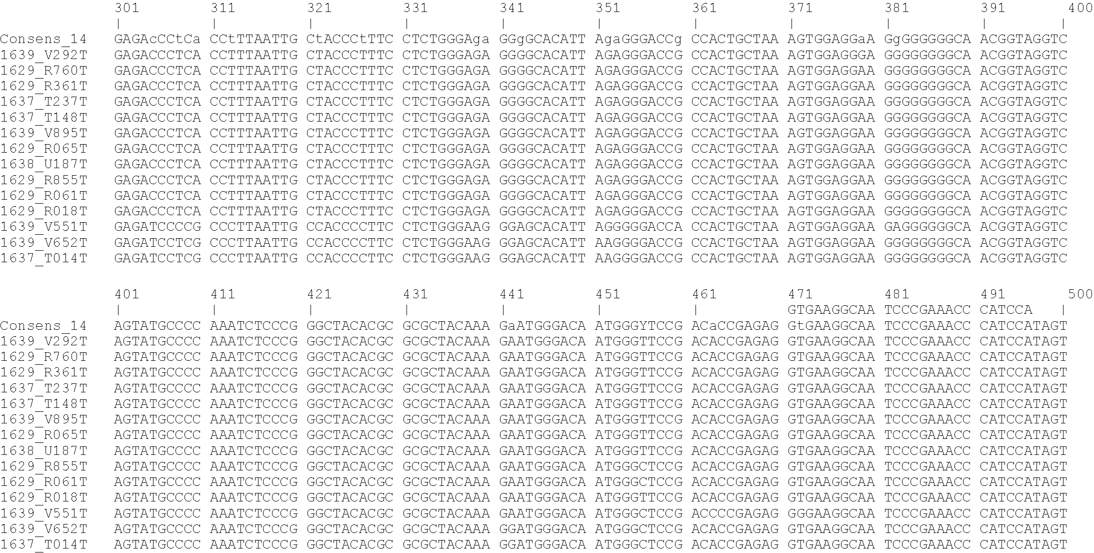

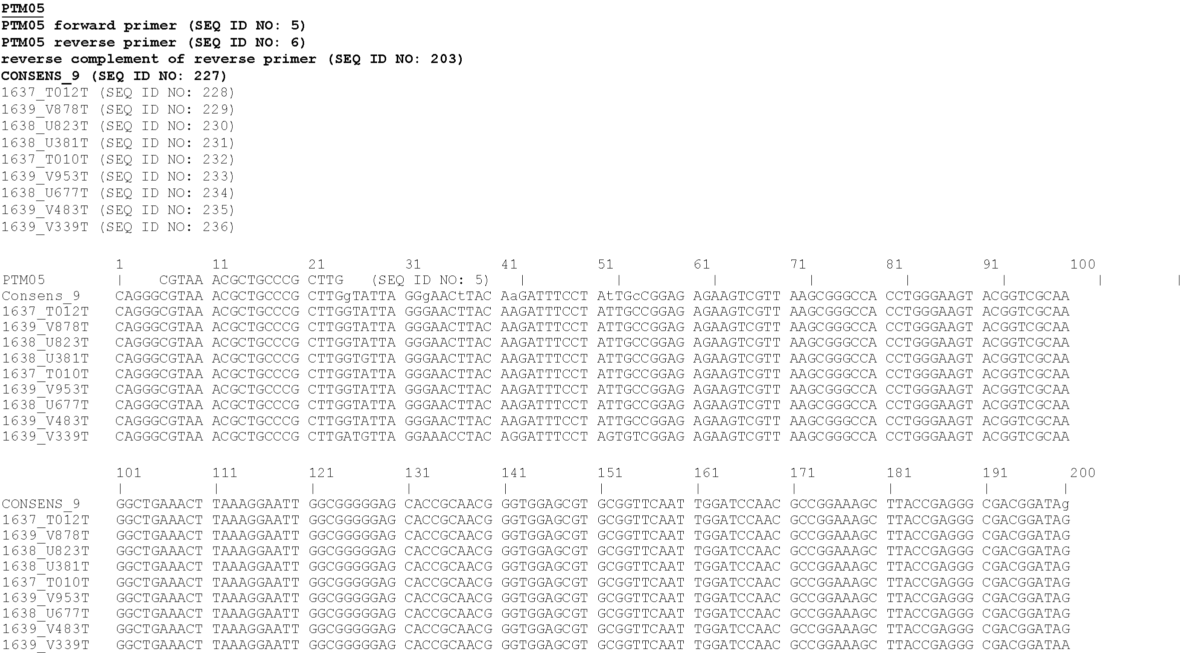

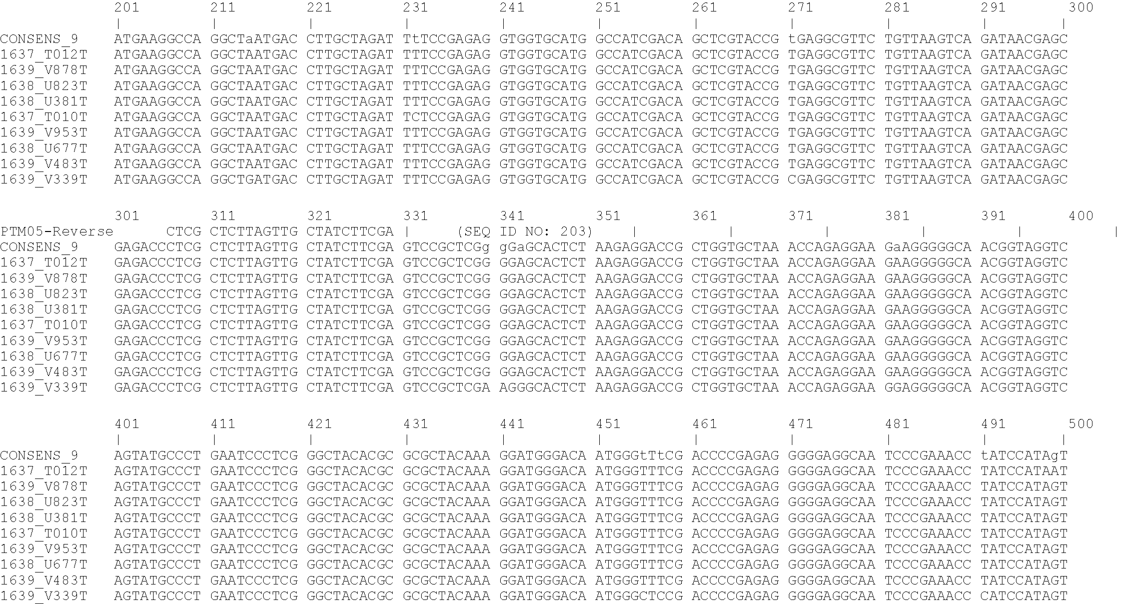

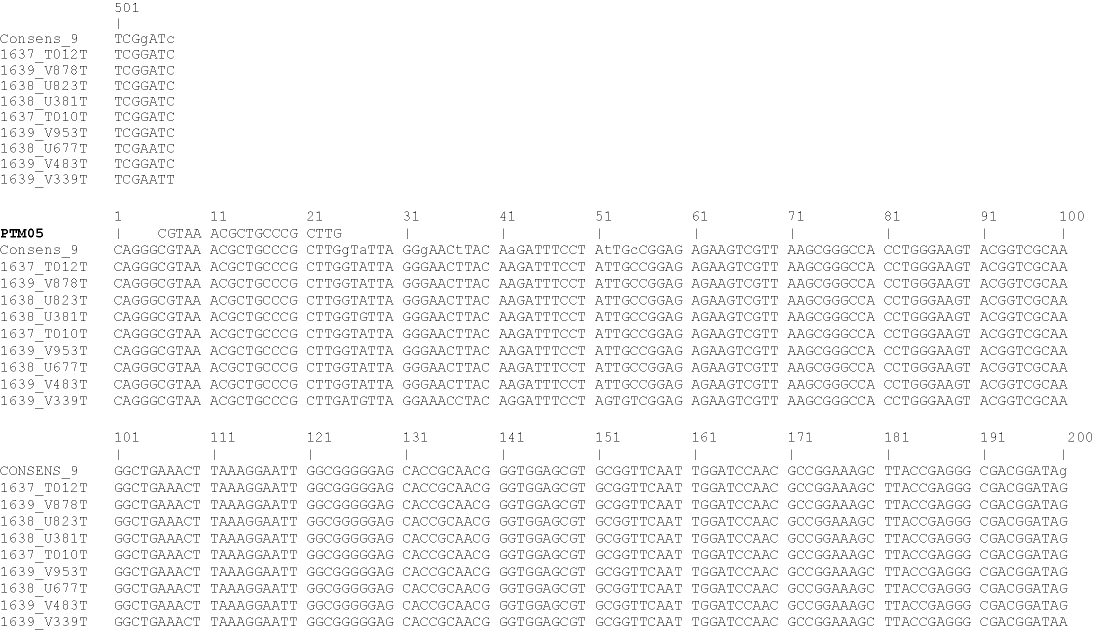

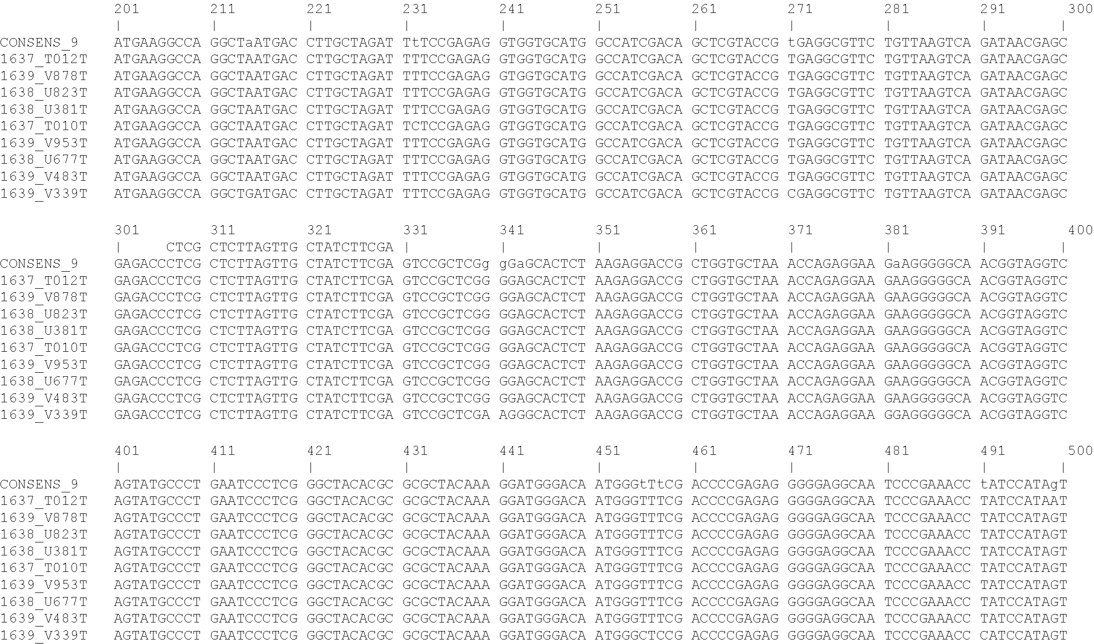

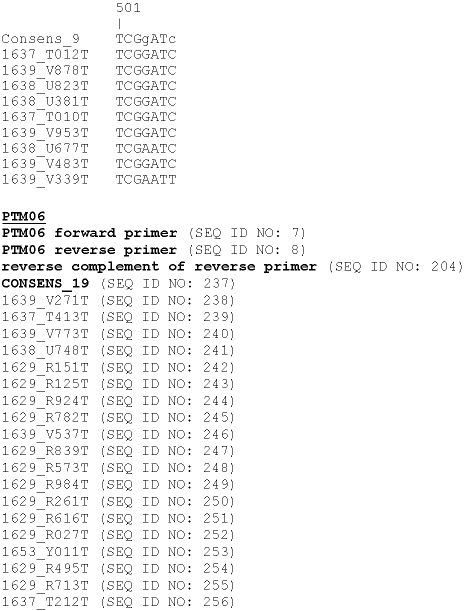

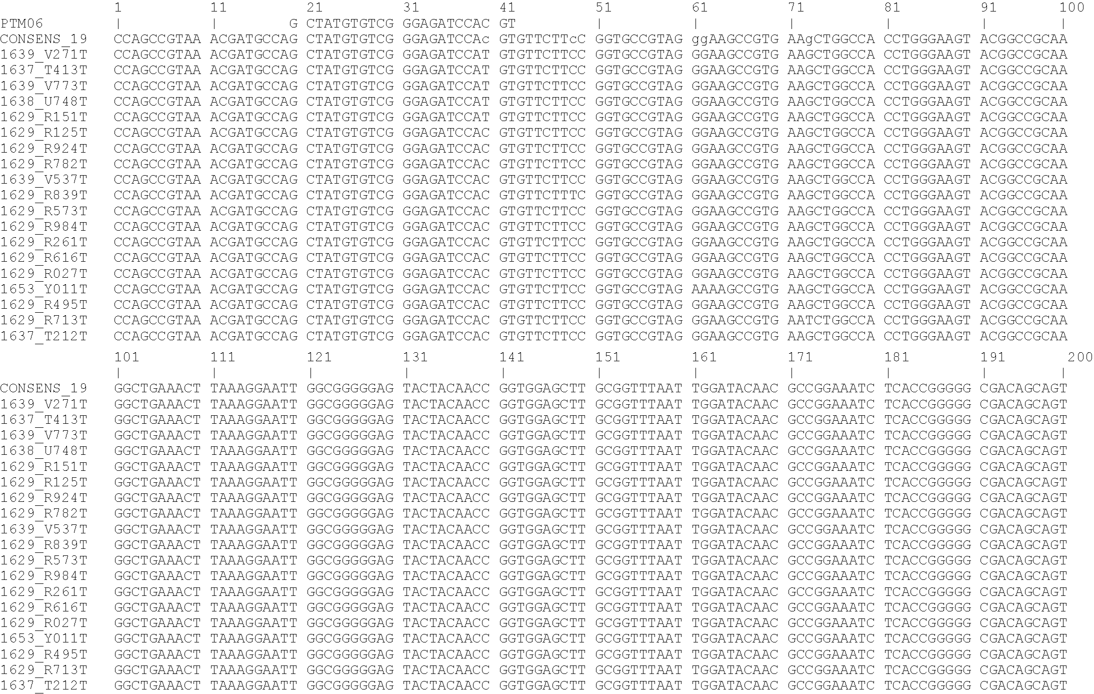

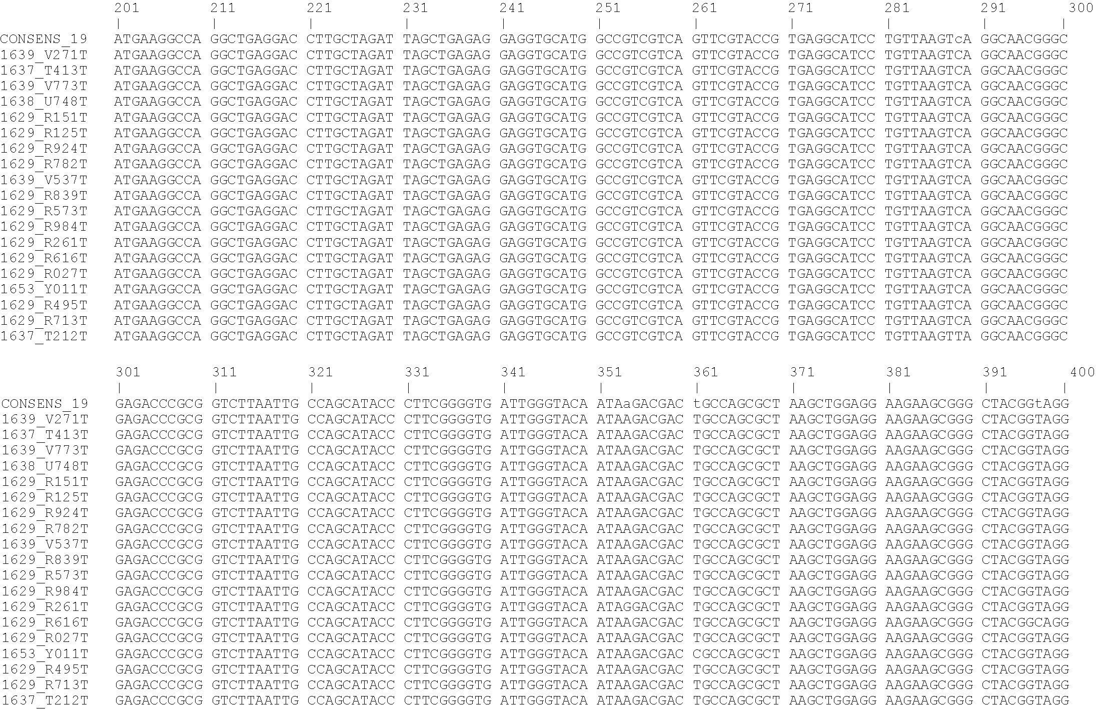

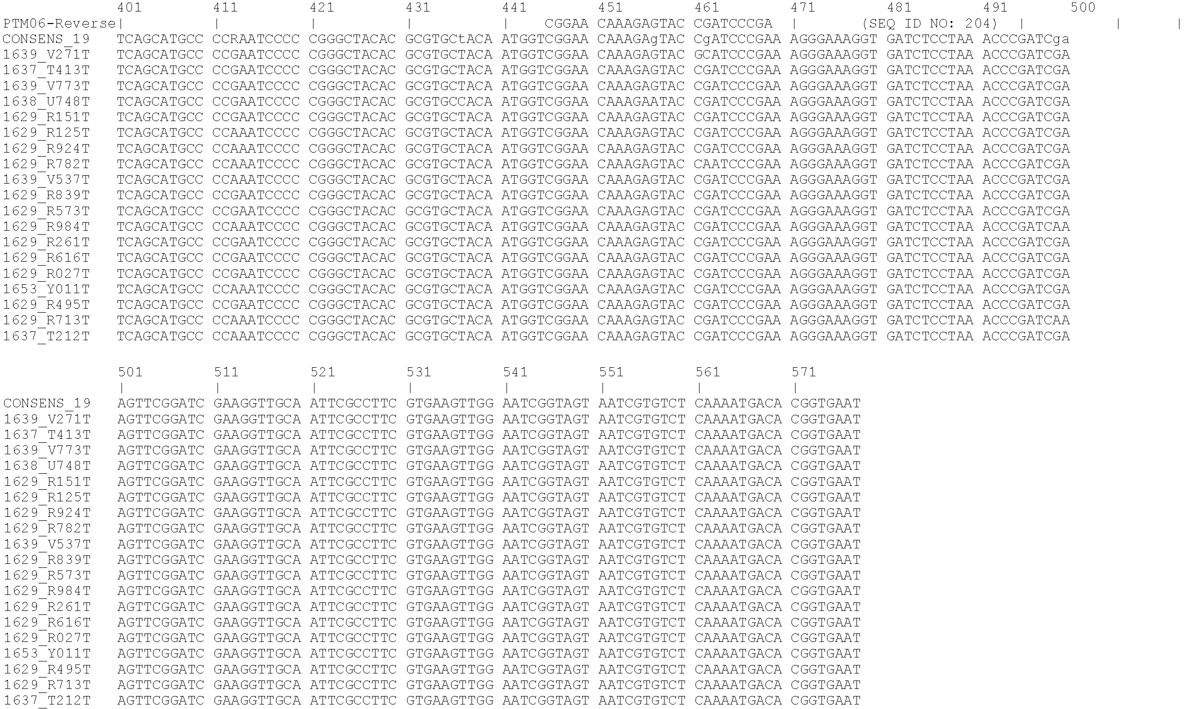

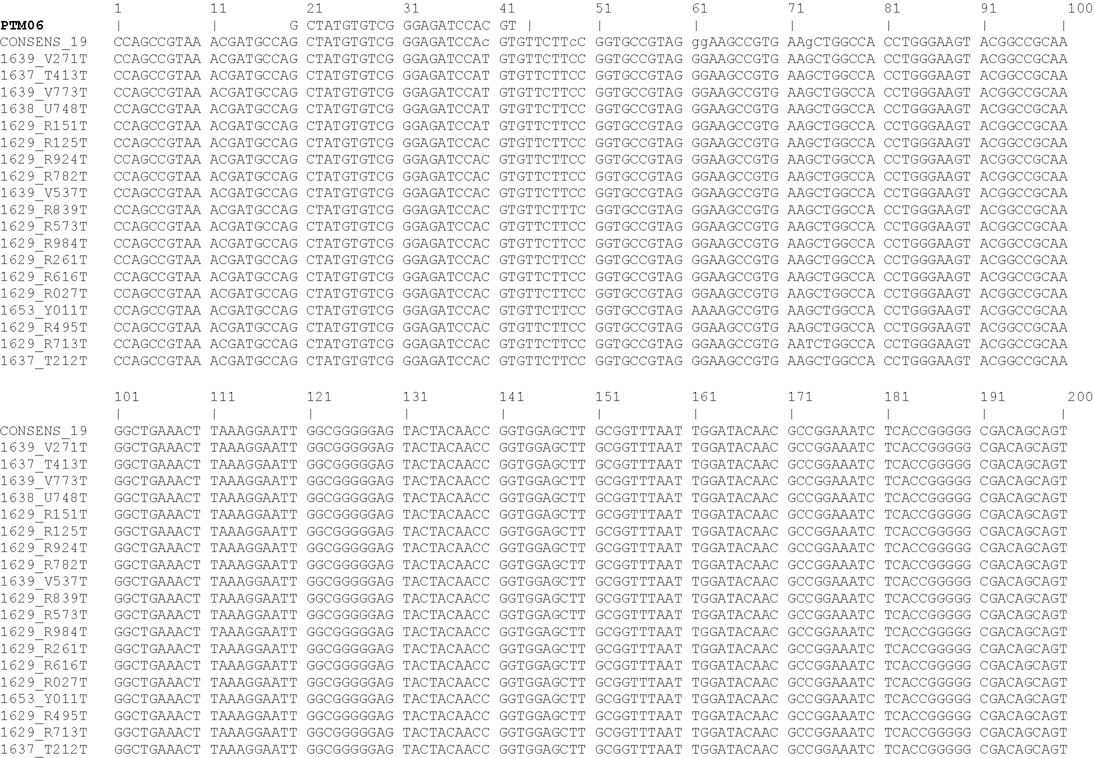

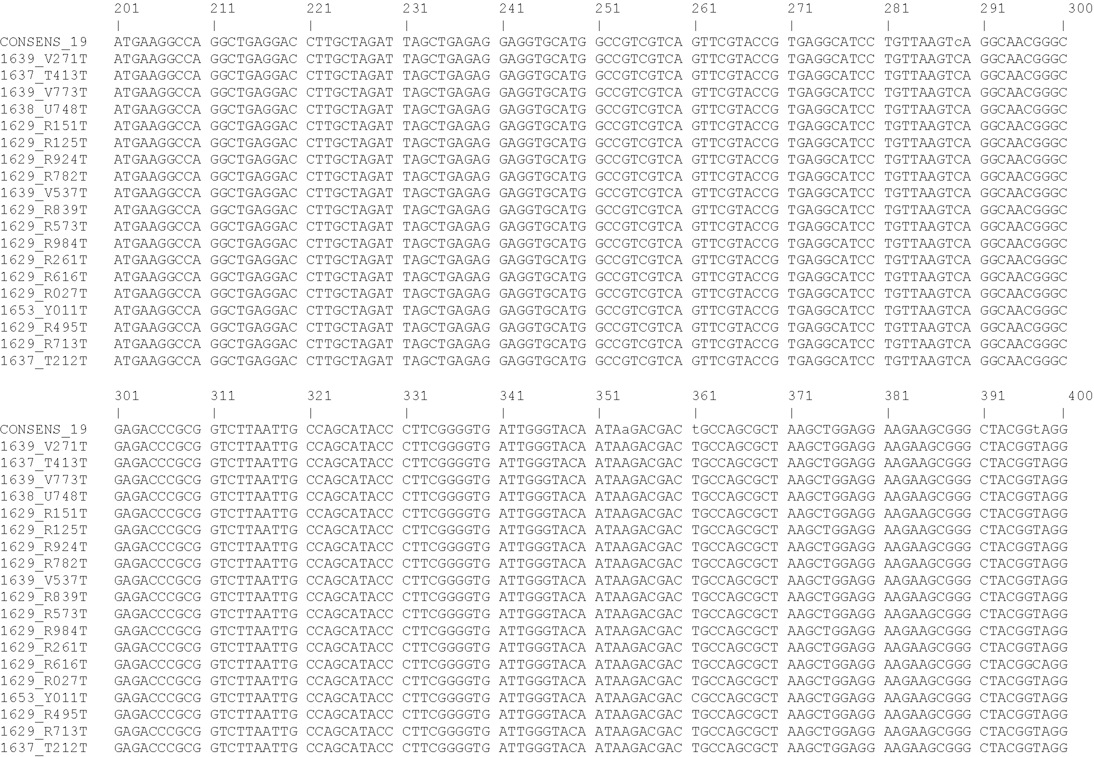

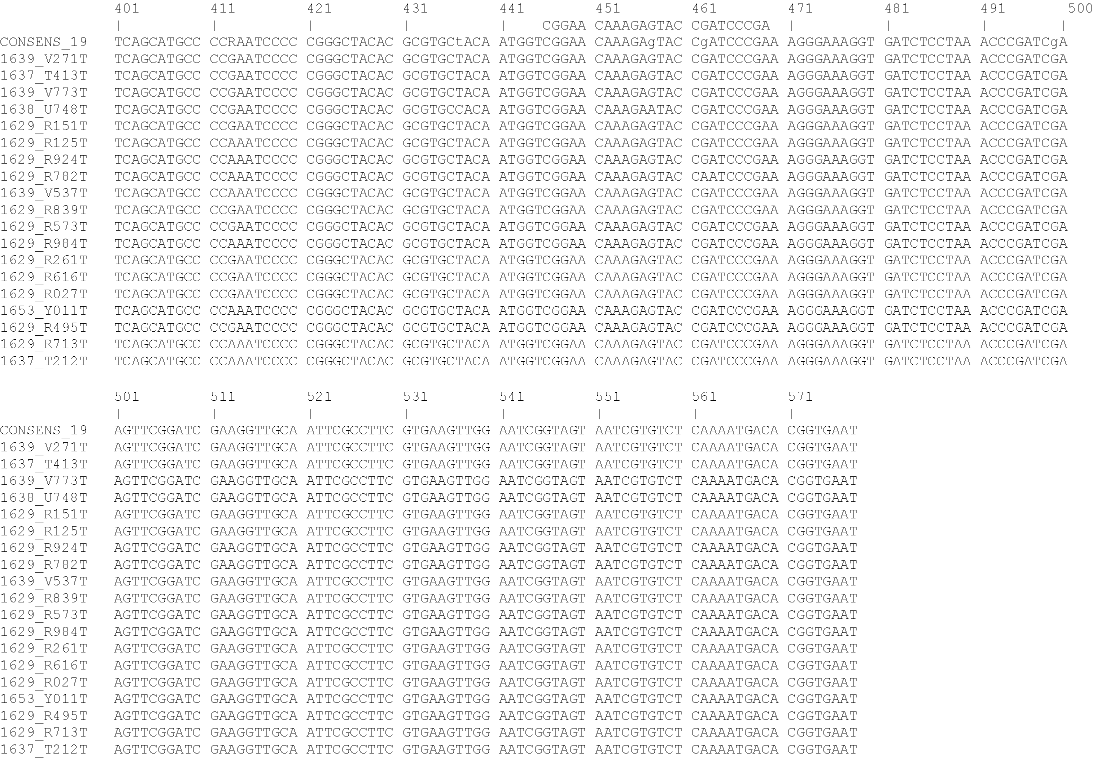

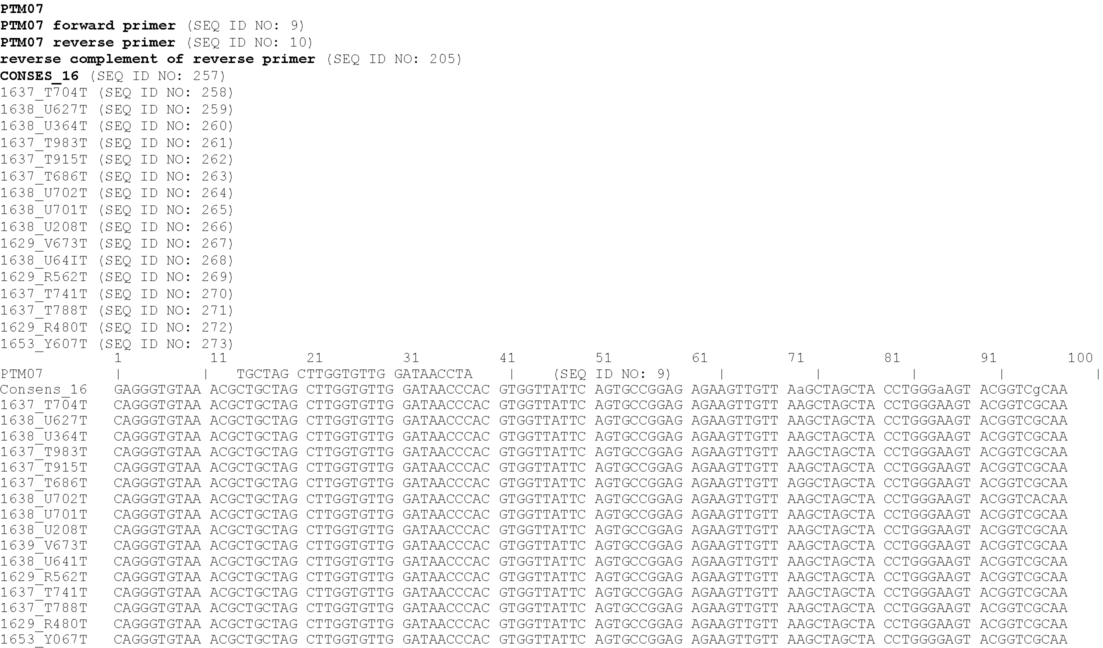

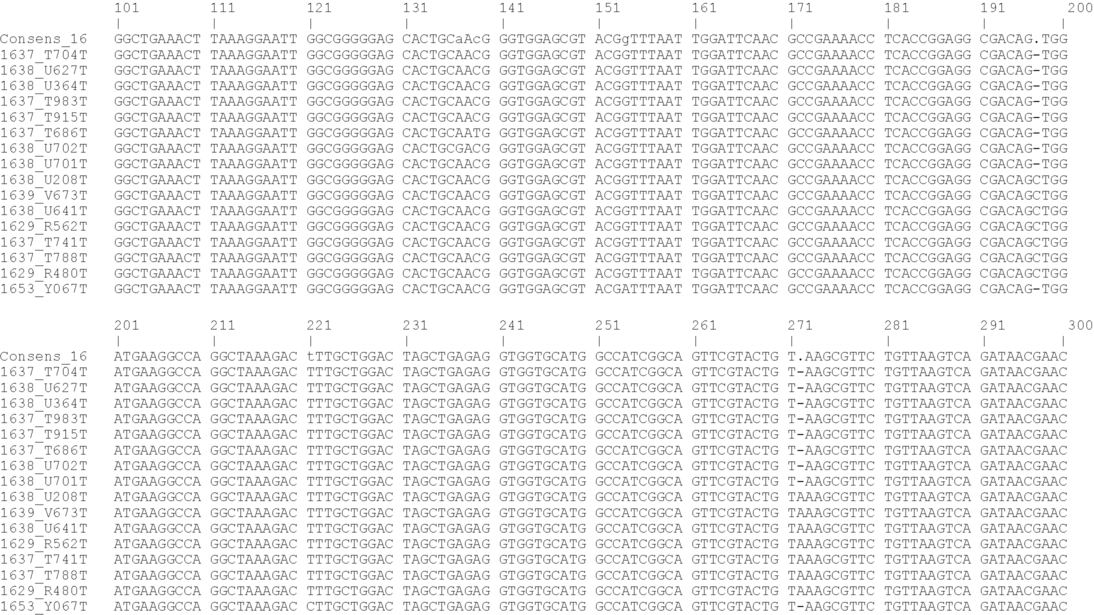

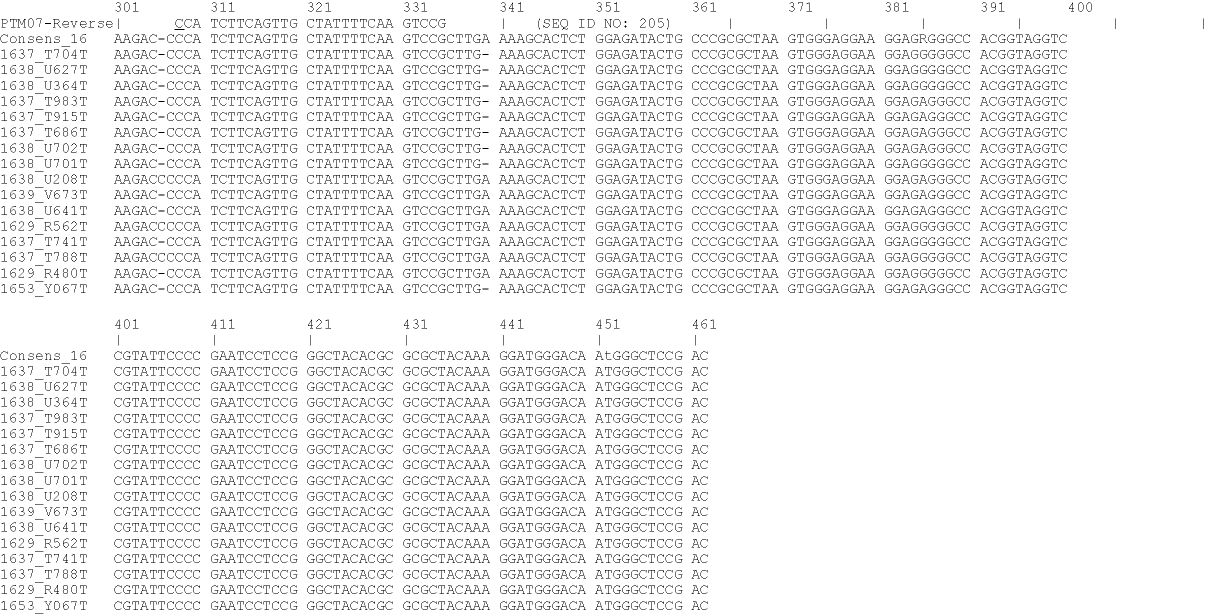

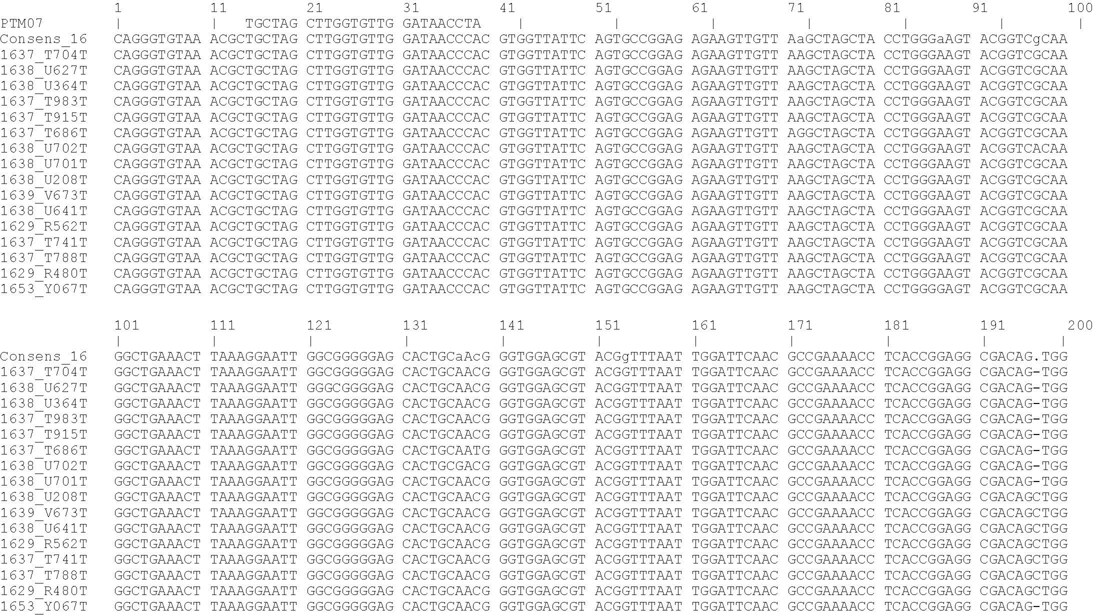

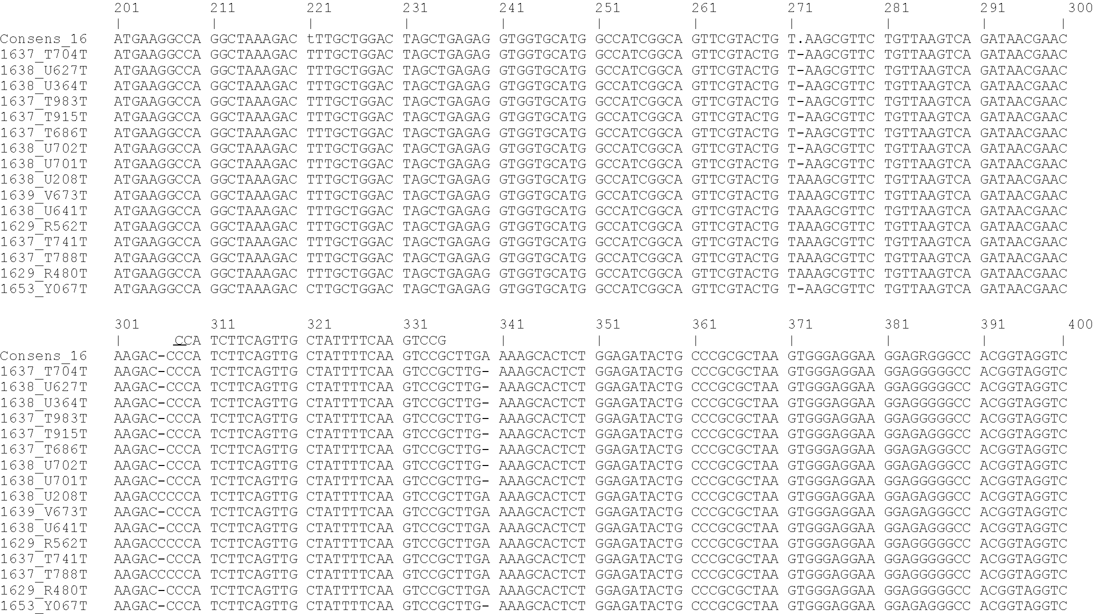

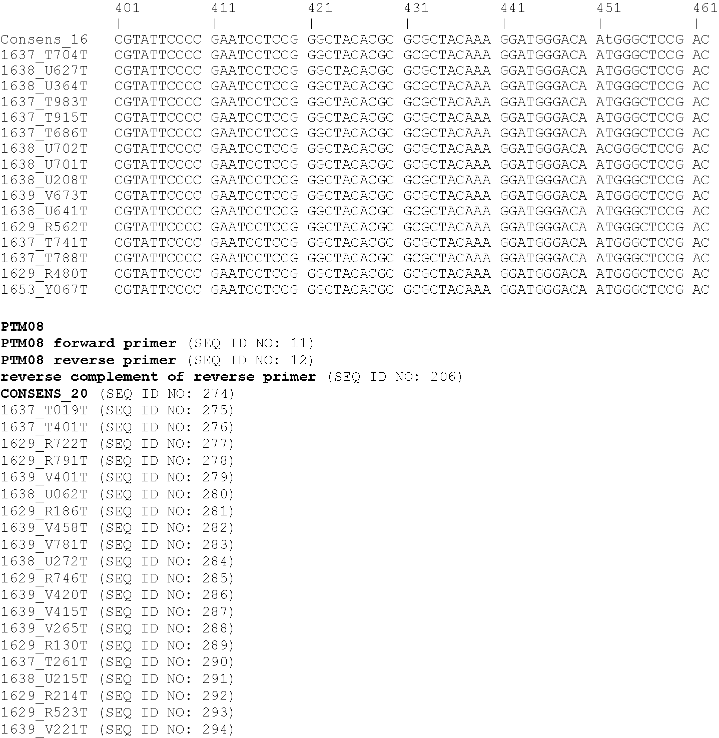

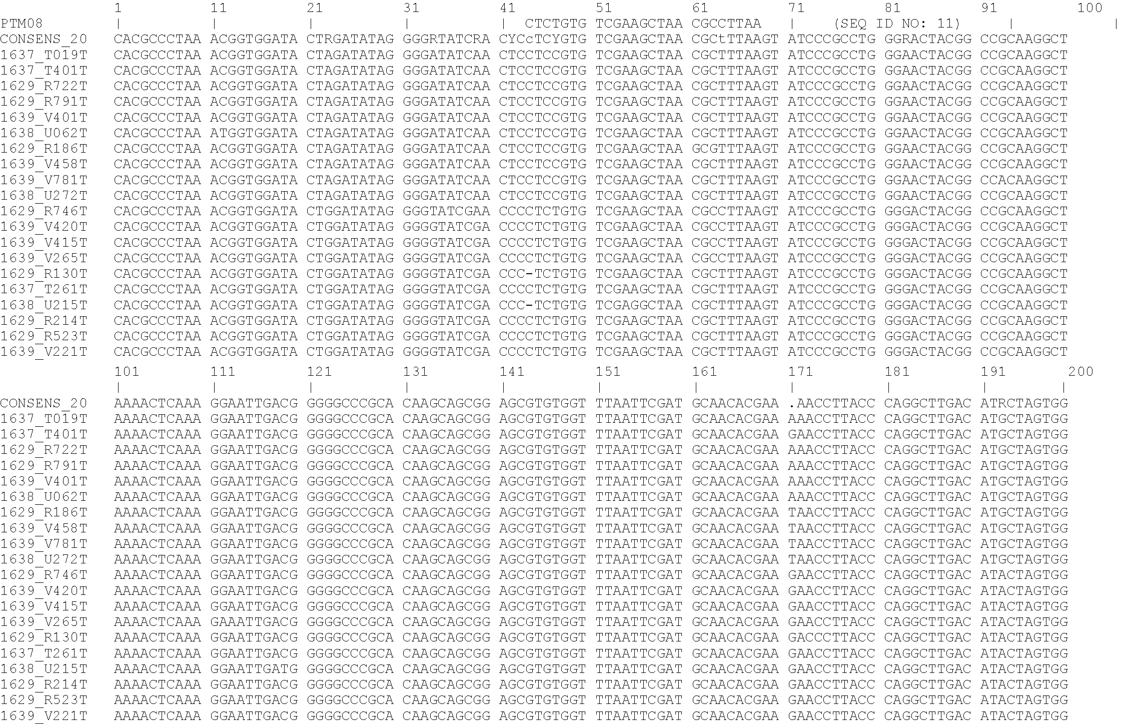

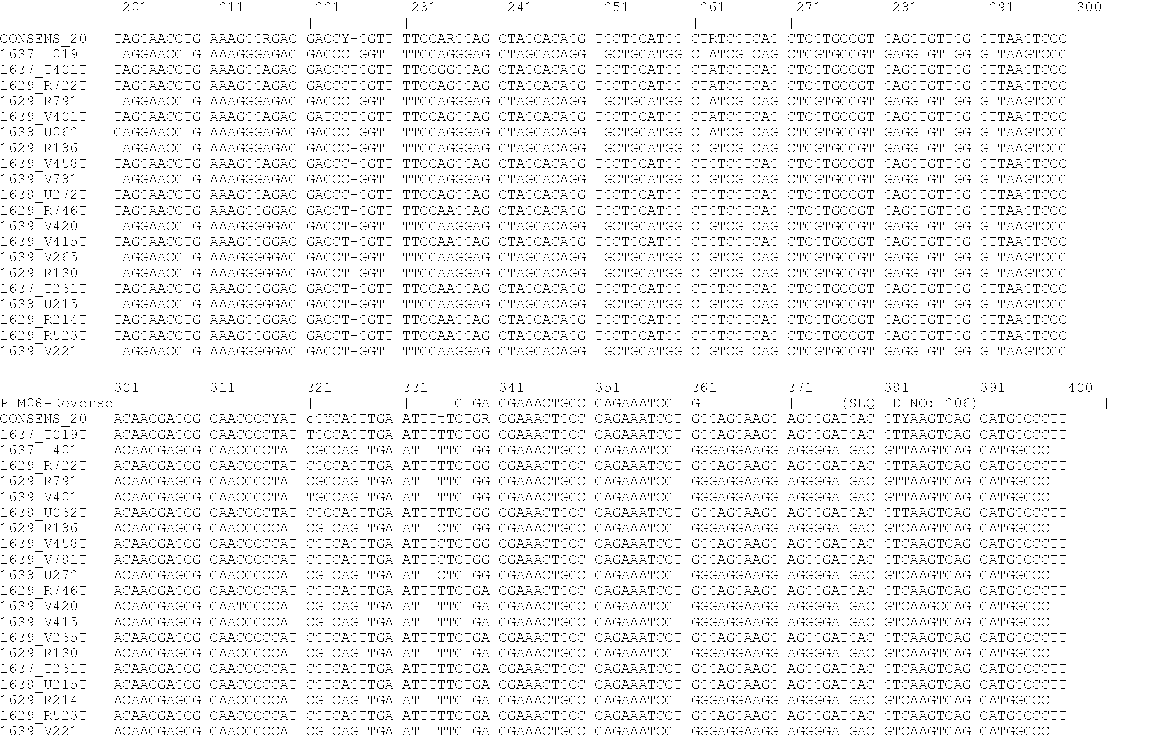

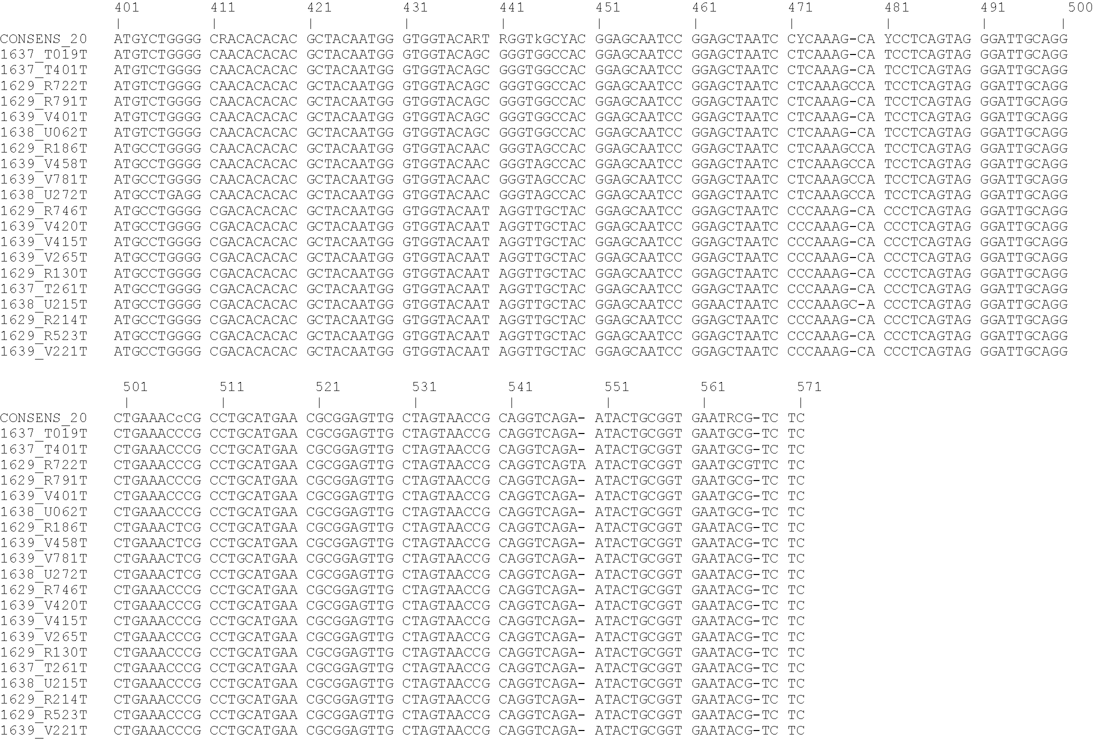

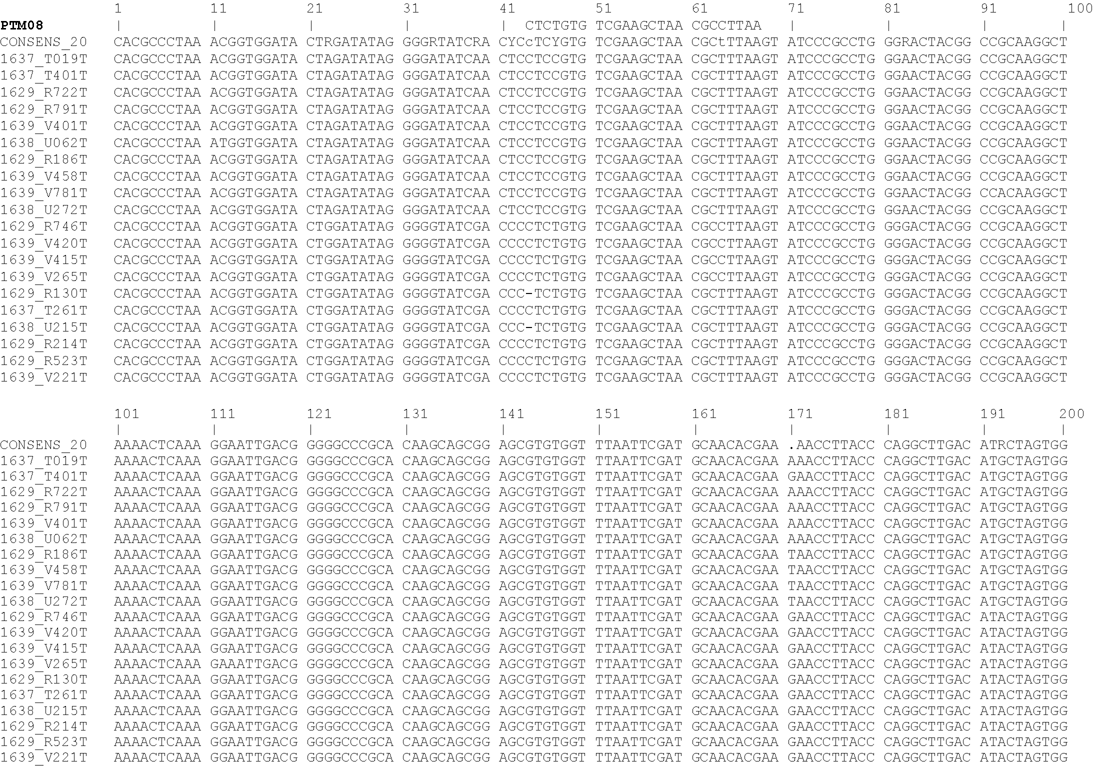

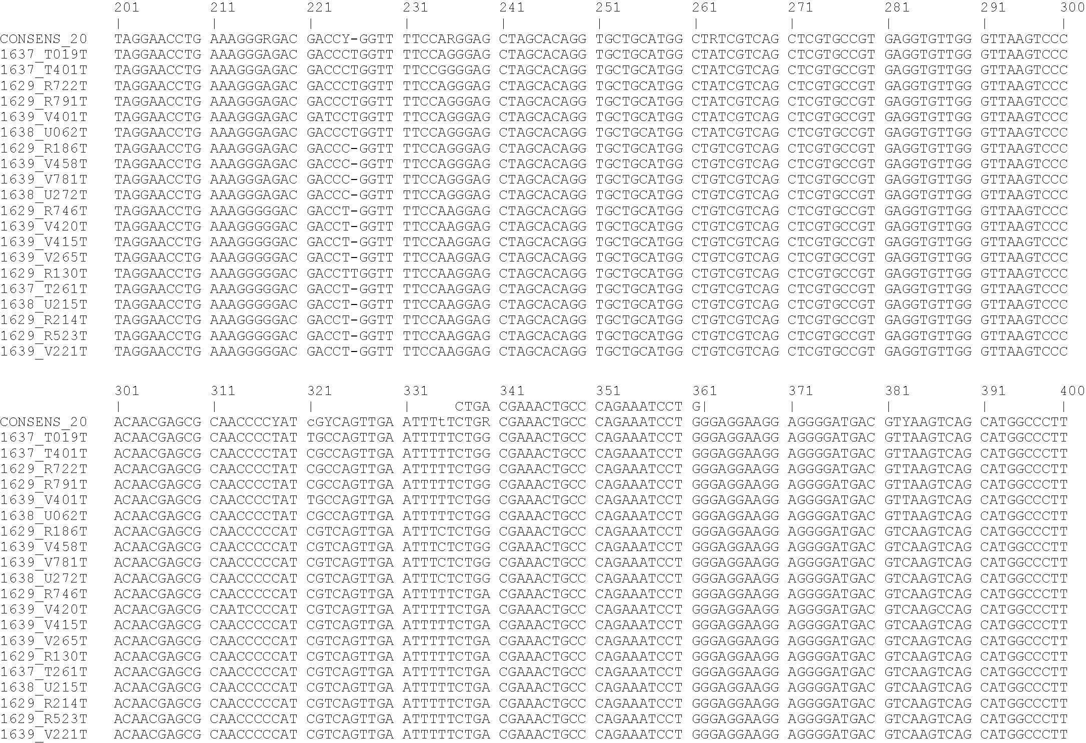

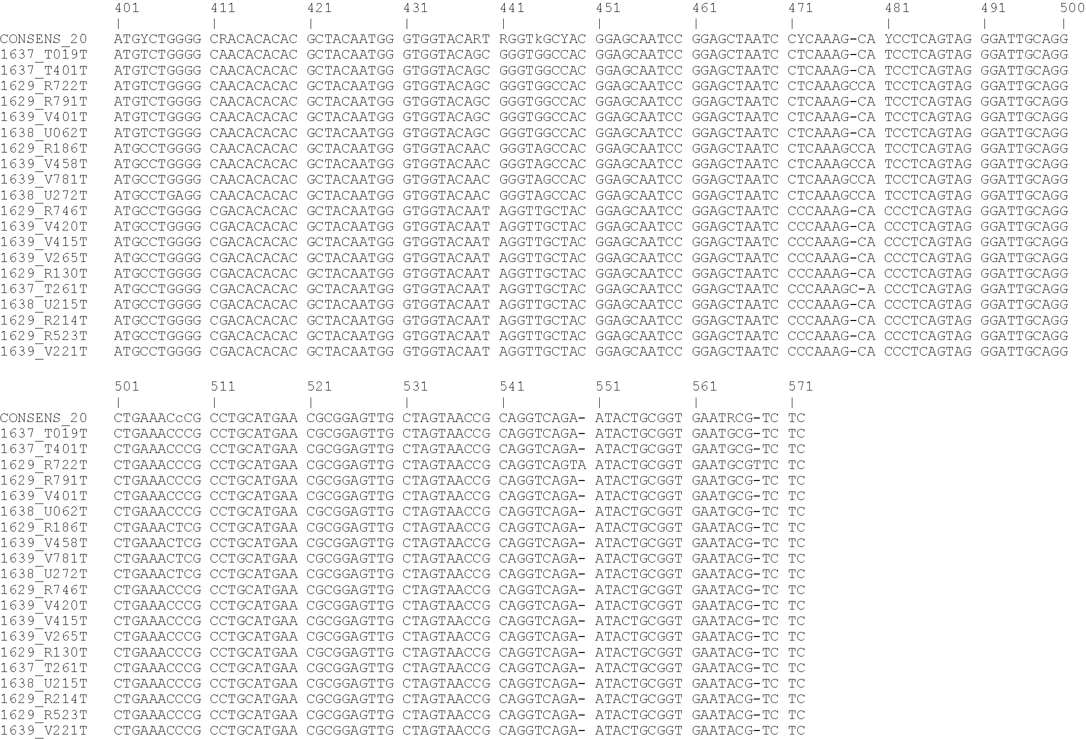

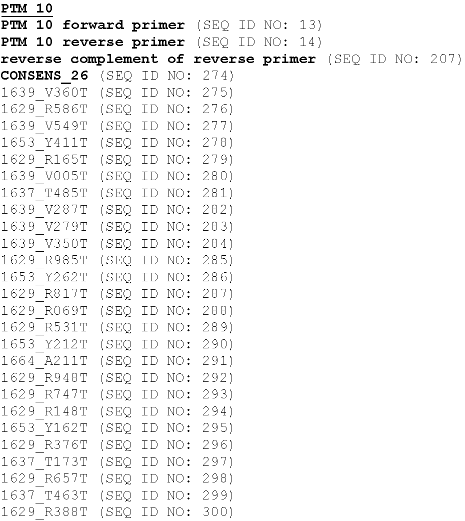

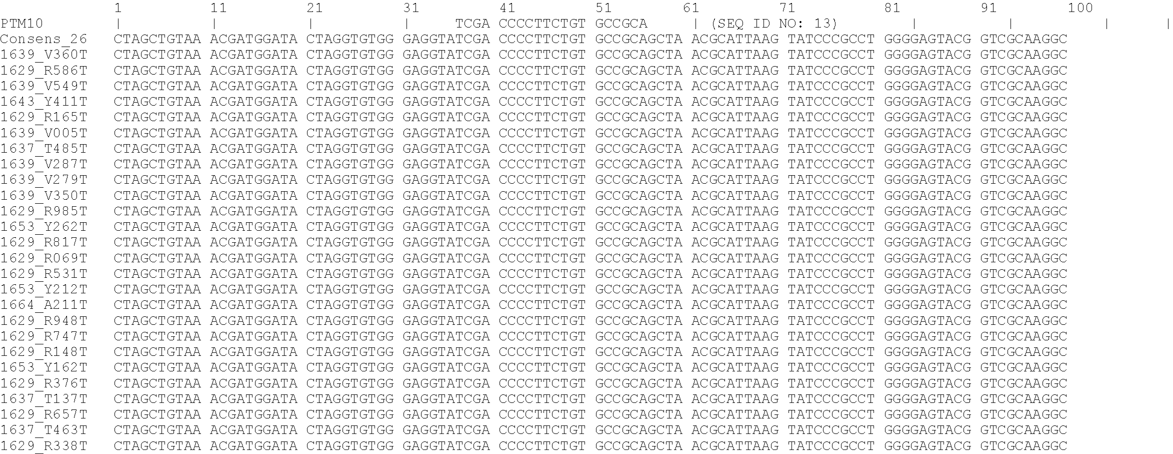

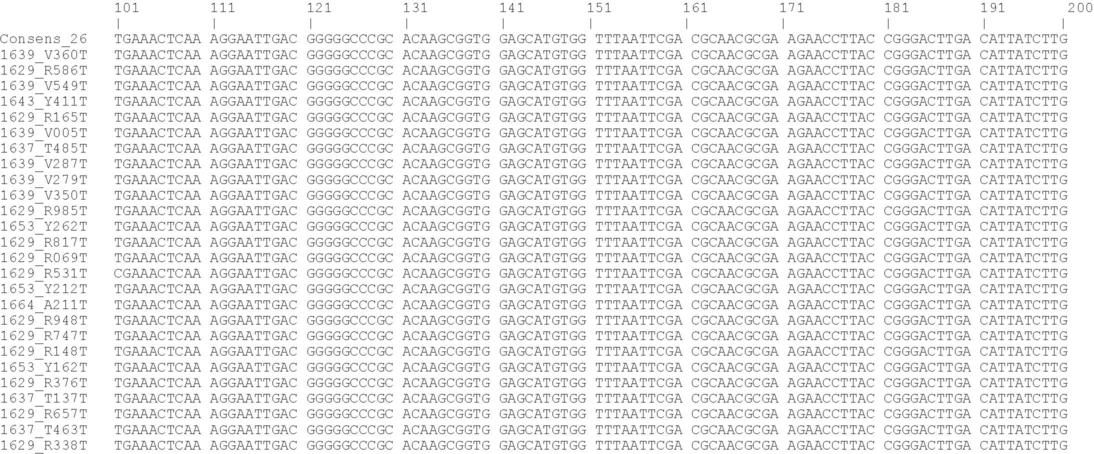

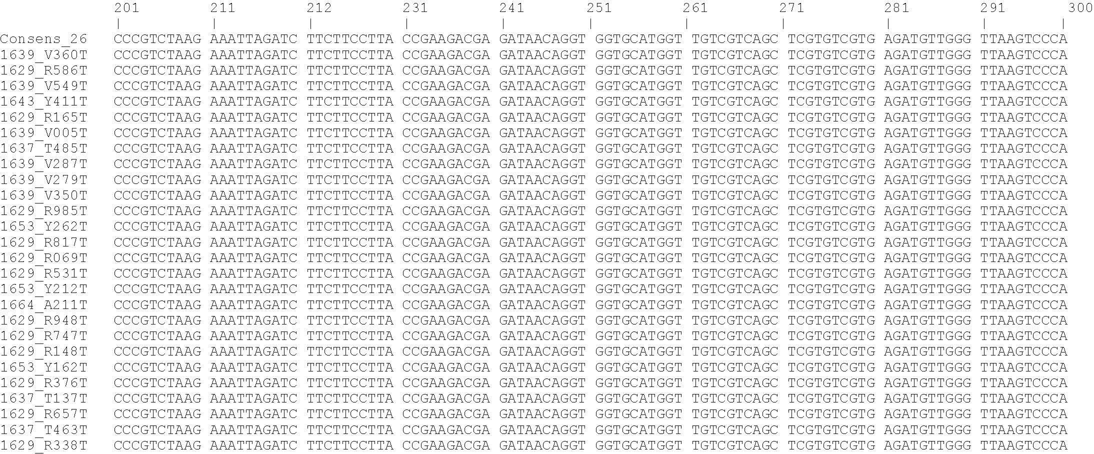

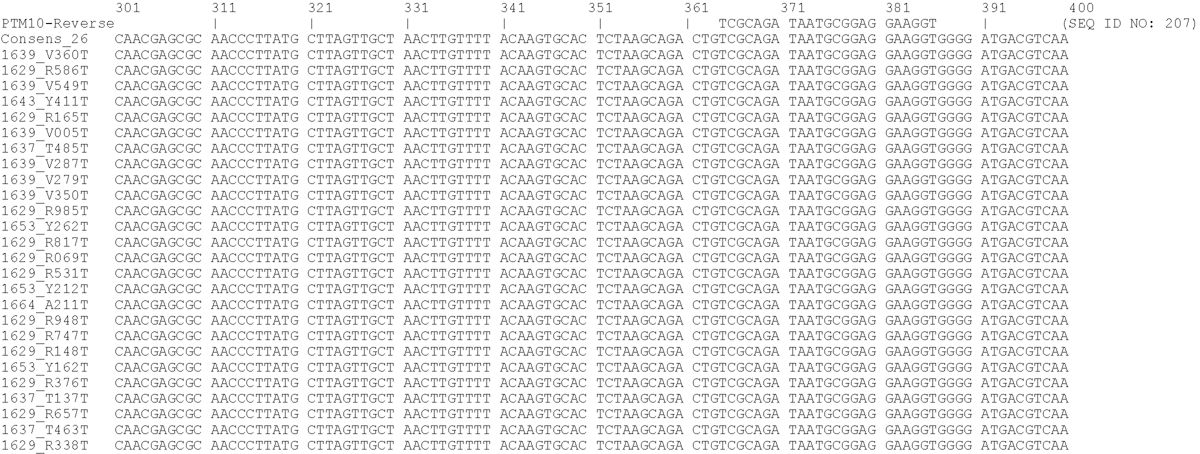

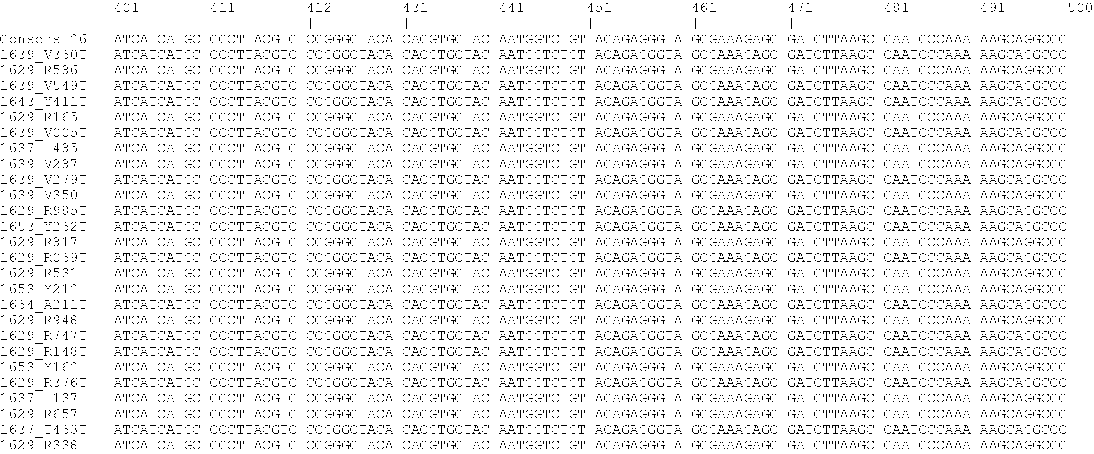

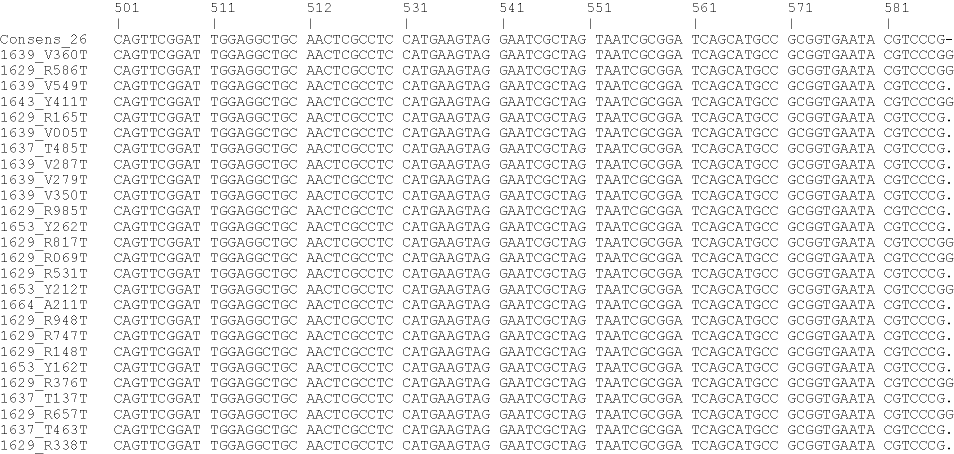

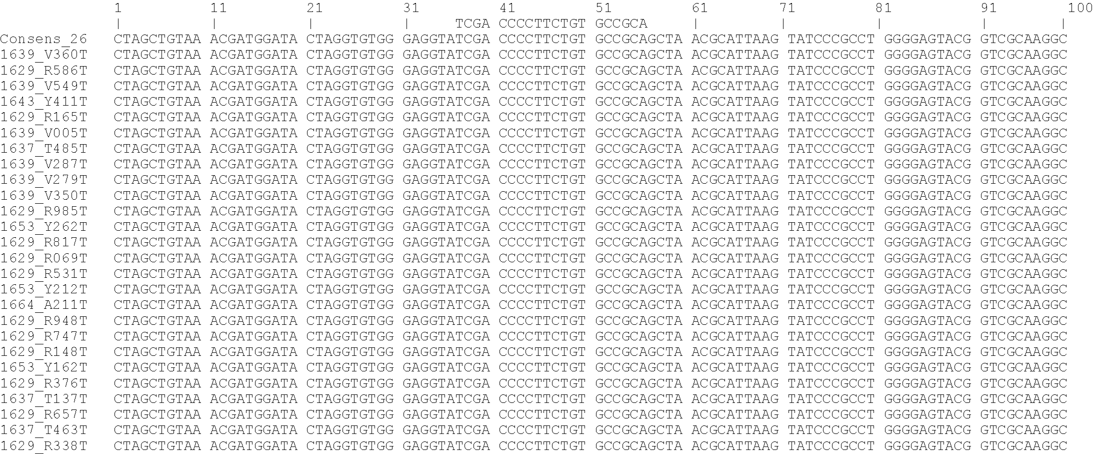

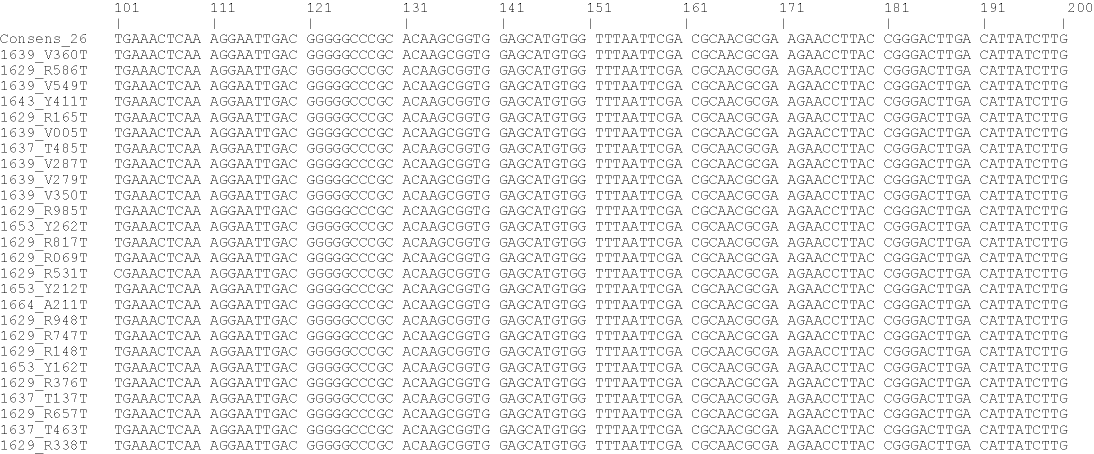

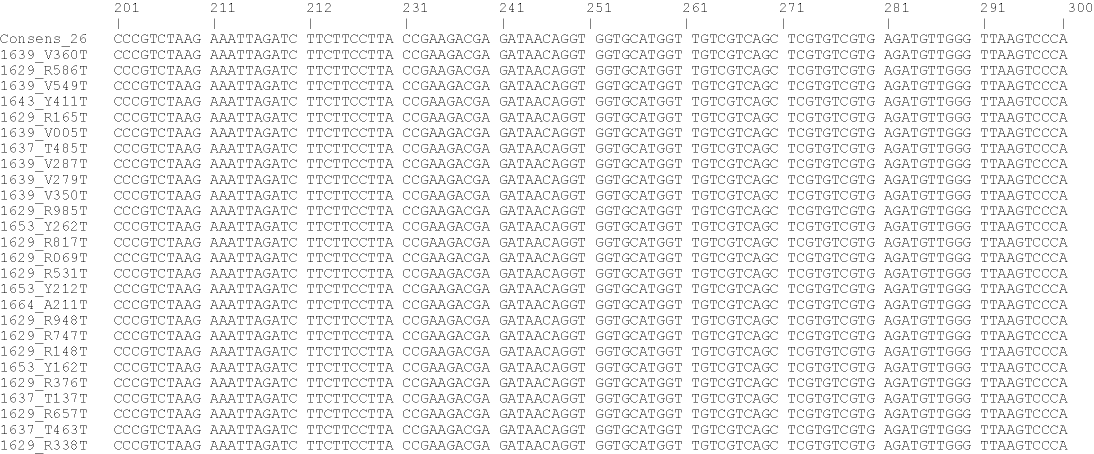

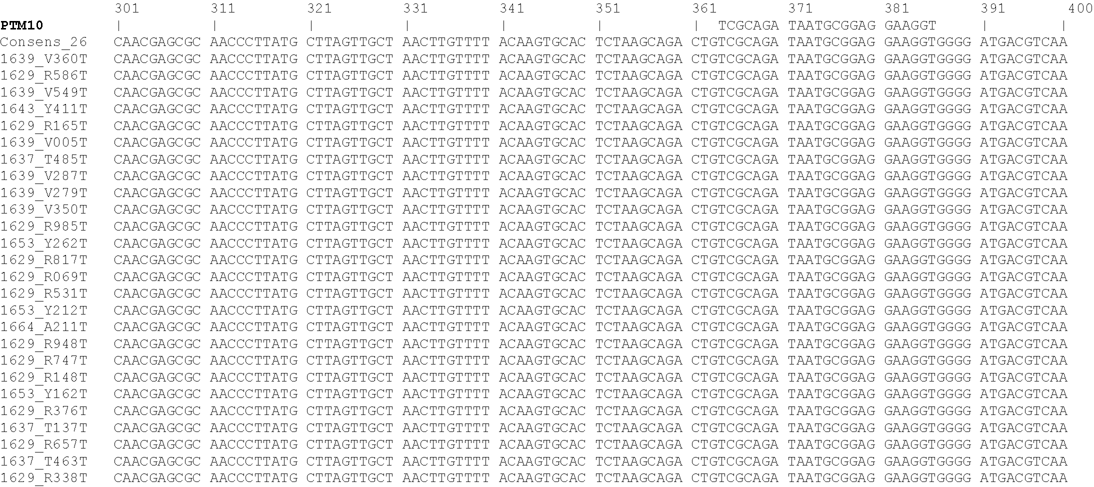

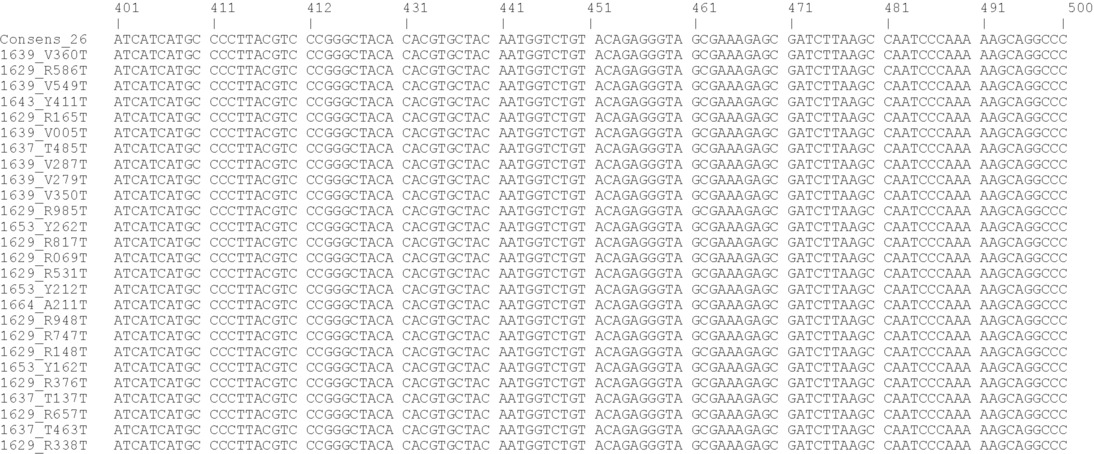

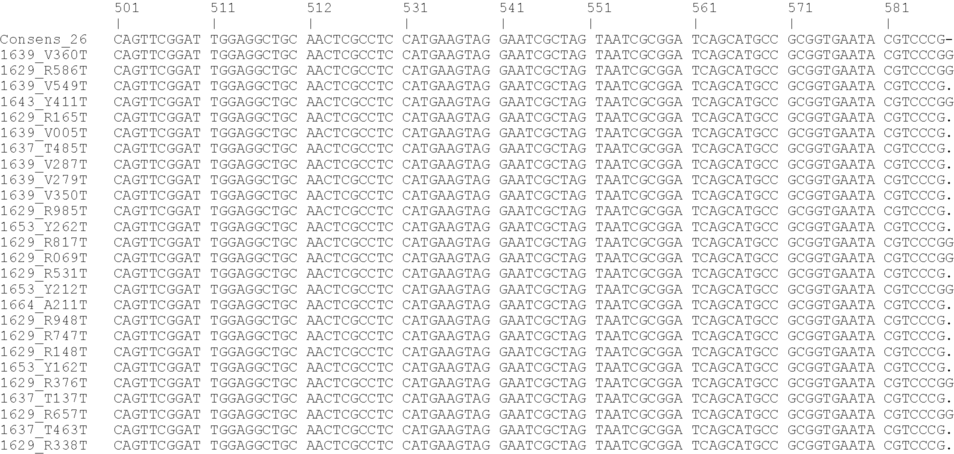

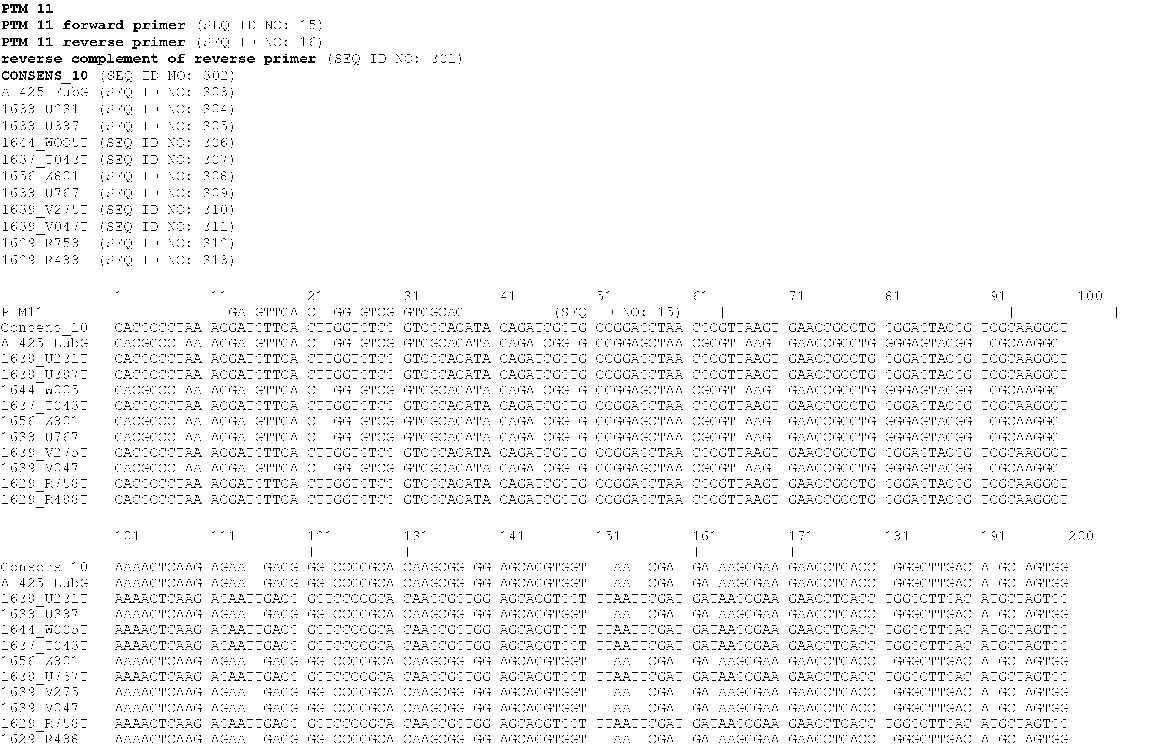

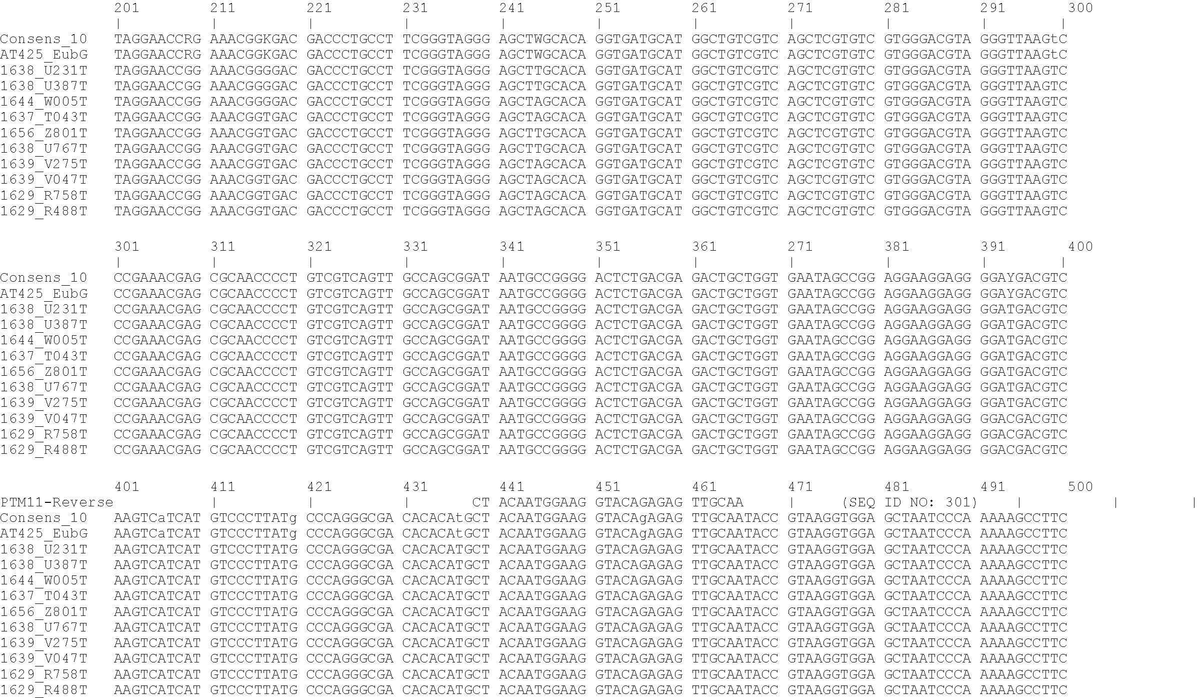

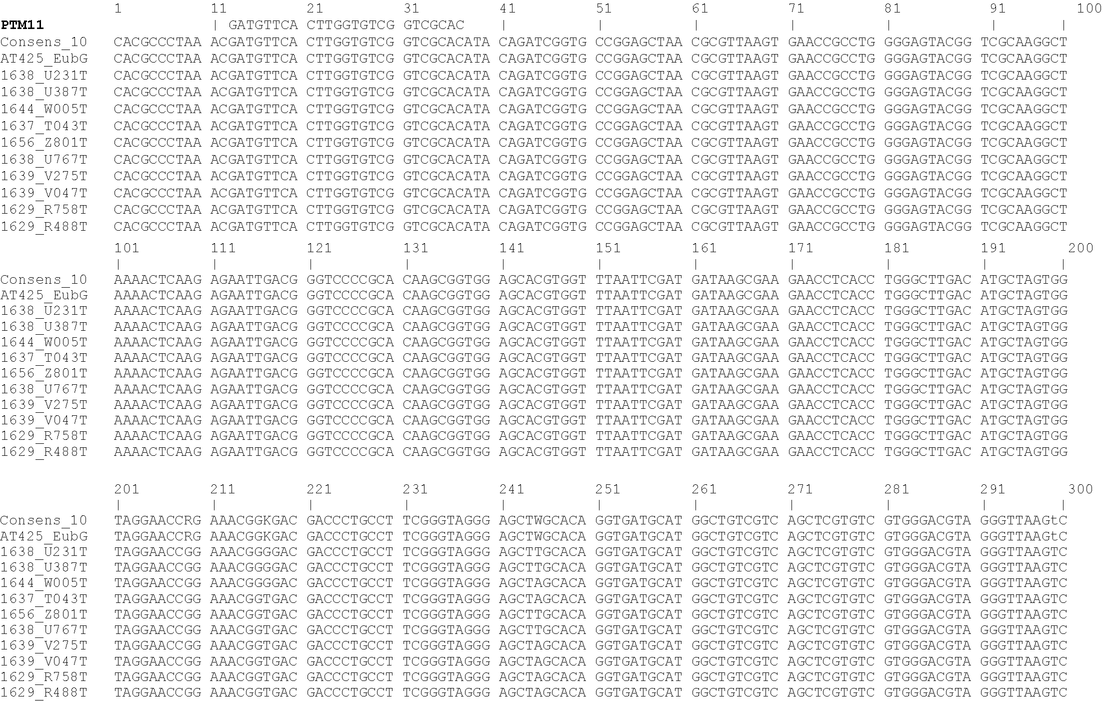

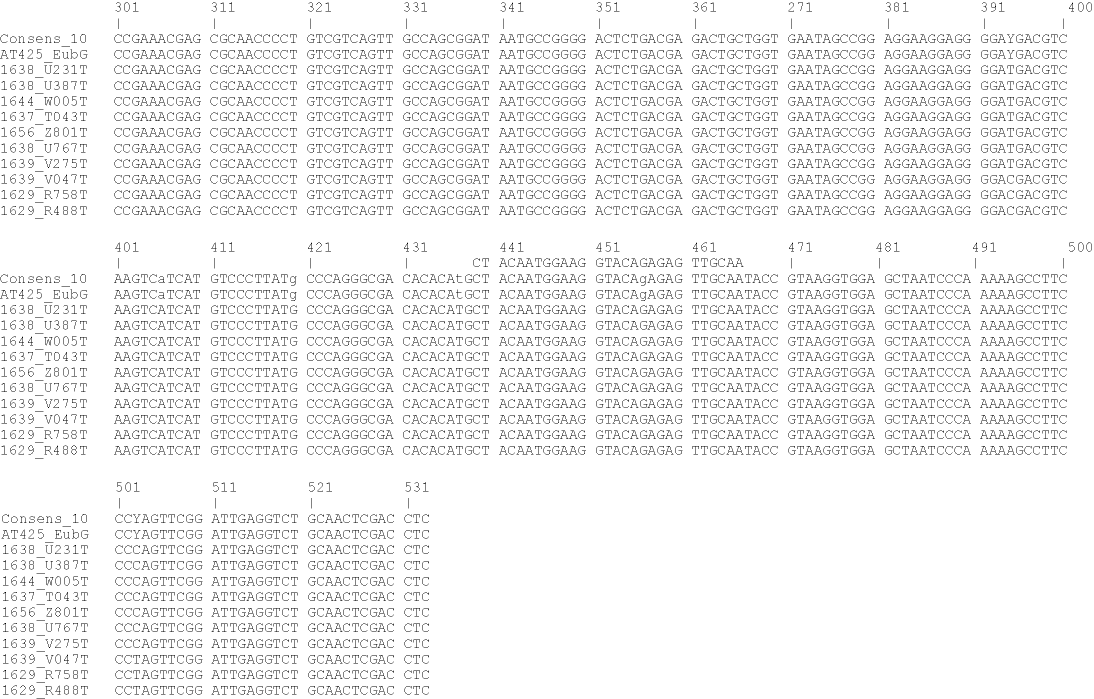

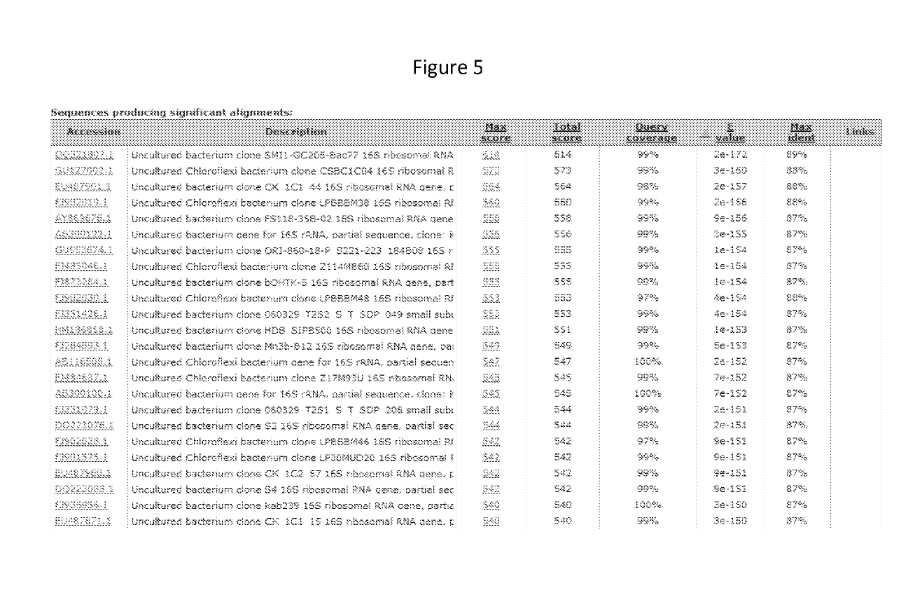

The composite (or consensus) gasoline-range bioindicator sequences were compared with sequences in the public database GenBank to identify known related sequences (FIGS. 5 to 12). In several cases either no related sequences (>90% identical) were found or a small number of sequences were found that had also only been identified in the Gulf of Mexico. These groups likely represent novel phylum-level divisions.

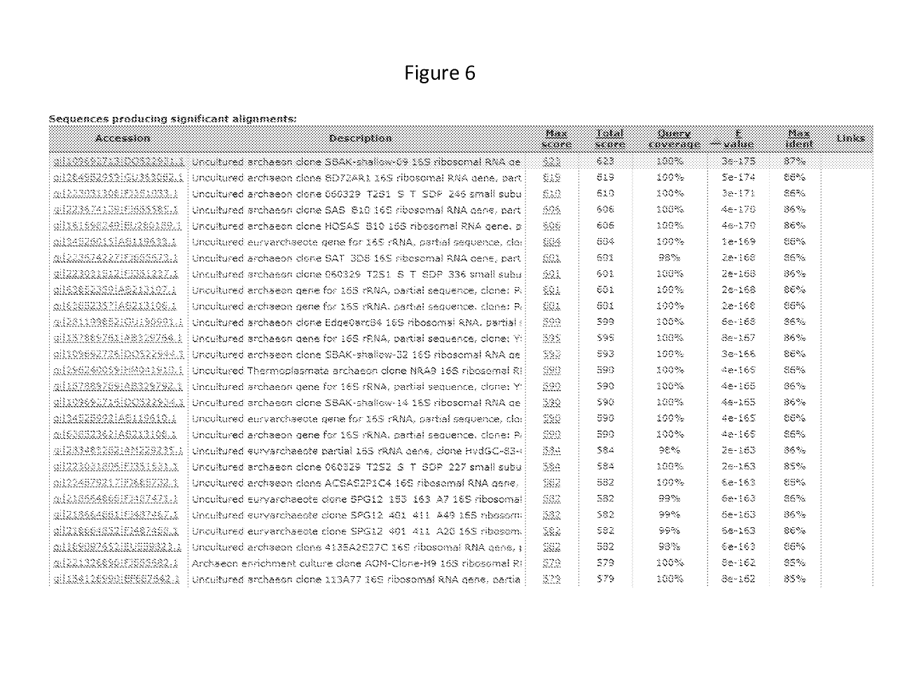

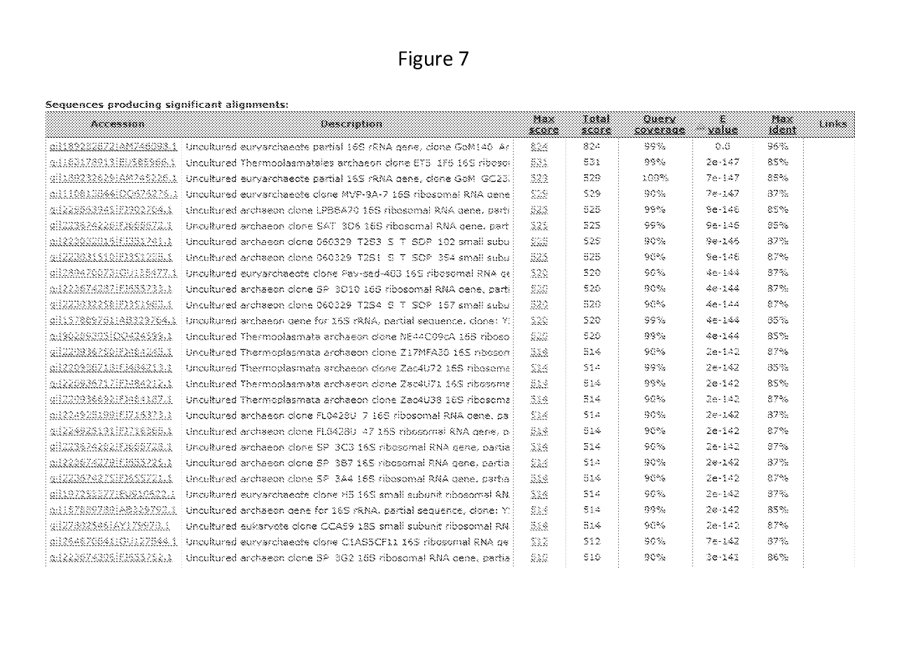

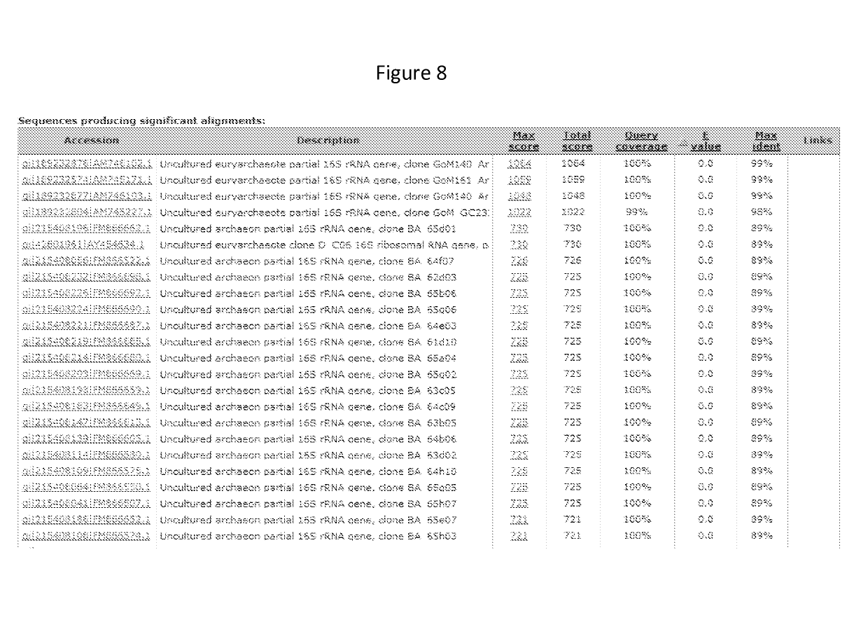

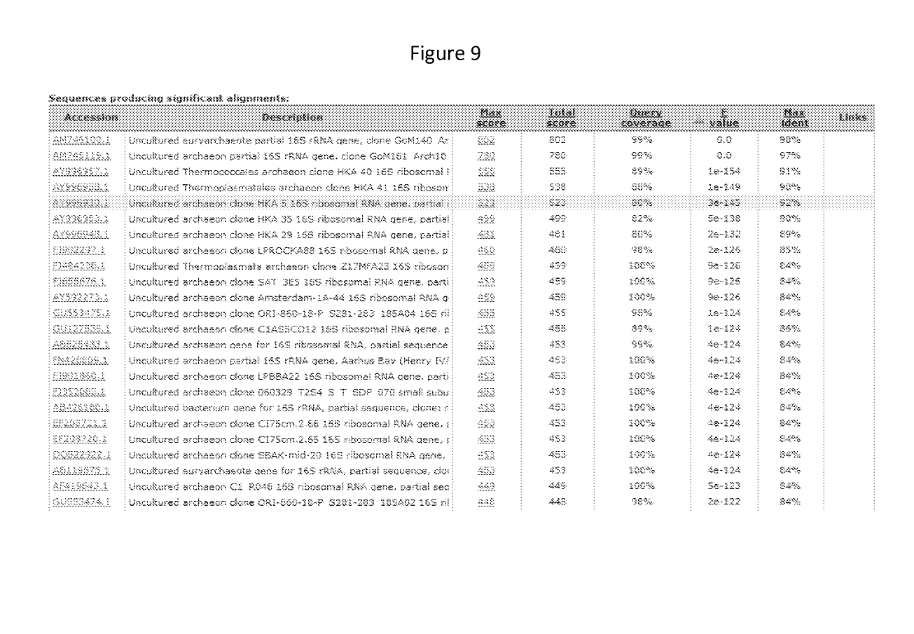

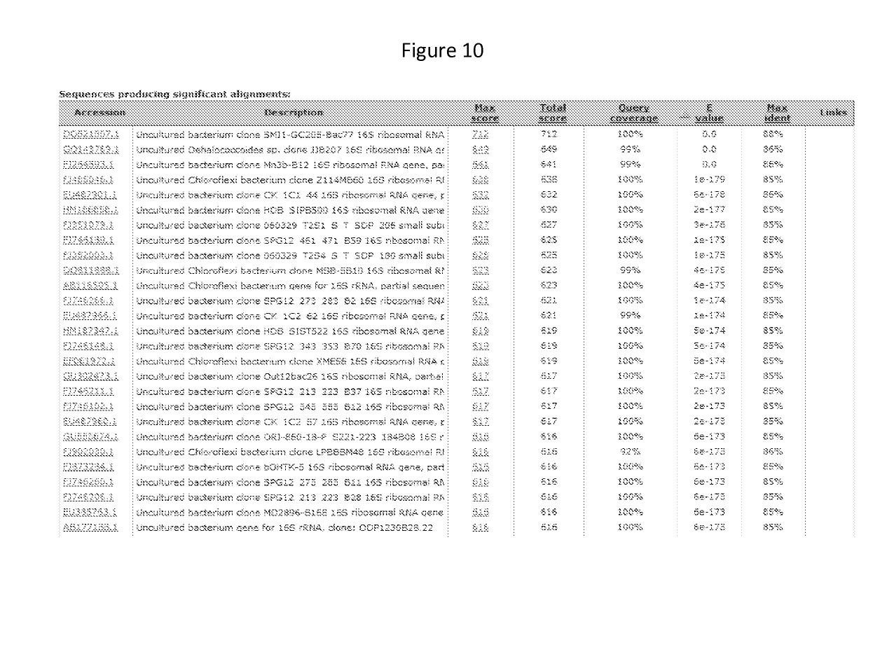

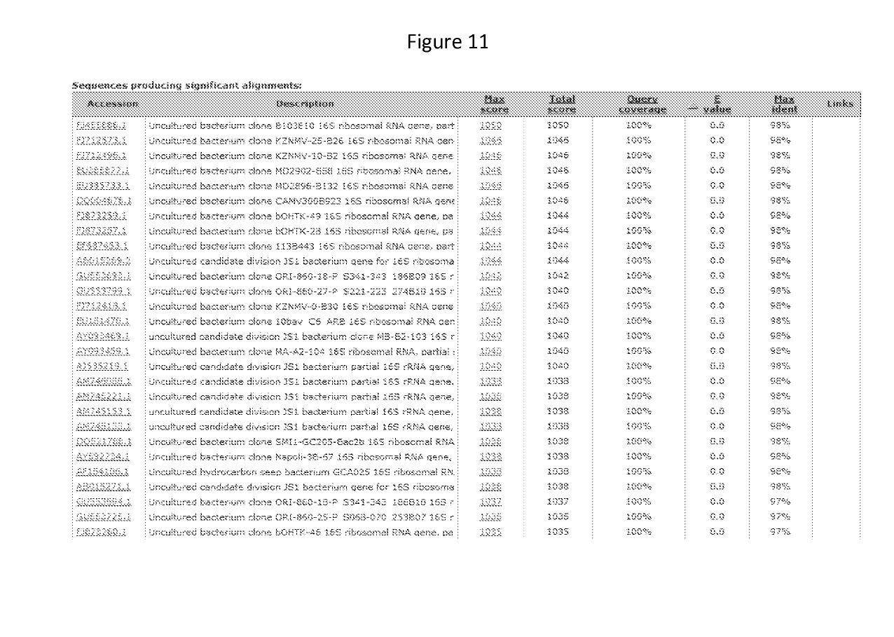

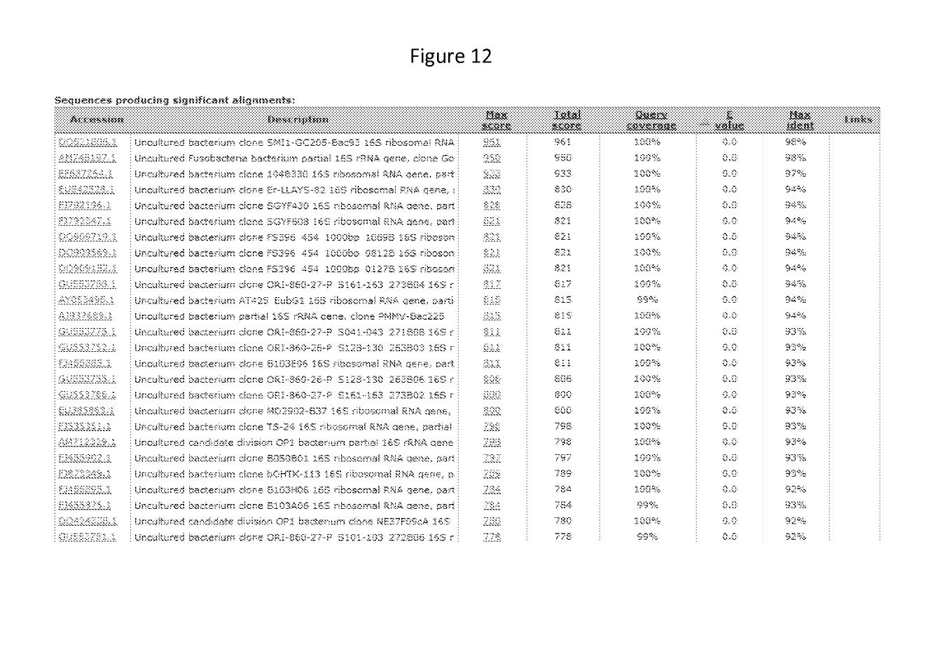

FIG. 5 illustrates comparison of PTM-03 Consensus sequence with the Genbank Non-Redundant DNA sequence database using BLASTN (ver. 2.2.24, see Zhang et al., 2000) search. FIG. 6 illustrates comparison of PTM-04_GOM2 Consensus sequence with the Genbank Non-Redundant DNA sequence database by BLASTN search. FIG. 7 illustrates comparison of PTM-05_GOM3 Consensus sequence with the Genbank Non-Redundant DNA sequence database by BLASTN search. FIG. 8 illustrates comparison of PTM-06_GOM1 Consensus sequence with the Genbank Non-Redundant DNA sequence database by BLASTN search. FIG. 9 illustrates comparison of PTM-07 Consensus sequence with the Genbank Non-Redundant DNA sequence database by BLASTN search. FIG. 10 illustrates comparison of PTM-08 Consensus sequence with the Genbank Non-Redundant DNA sequence database by BLASTN search. FIG. 11 illustrates comparison of PTM-10 Consensus sequence with the Genbank Non-Redundant DNA sequence database by BLASTN search performed. FIG. 12 illustrates comparison of PTM-11 Consensus sequence with the Genbank Non-Redundant DNA sequence database by BLASTN search.

qPCR assays were performed with SYBR™ Green (Invitrogen, Carlsbad, Calif.) in a ABI 7900HT™ instrument. Melt curves of the products were used to identify reactions with low Tm products. Cloned 16S rRNA genes from the bioindicator strains were used as copy control standards. The qPCR data, expressed as copies per gram of sediment, underwent further data transformation. This included adding a small value (e.g. 1/100^{th} lowest value in table) to each cell in the table, log transforming the data and convert to Z-scores. Z-scores were determined by subtracting the mean and dividing by the standard deviation. Z-score units are expressed as number of standard deviations above (positive) or below (negative) the mean. These units are intuitive and enable combining of Z-scores from different bioindicators (through averaging) to report a single consensus value.

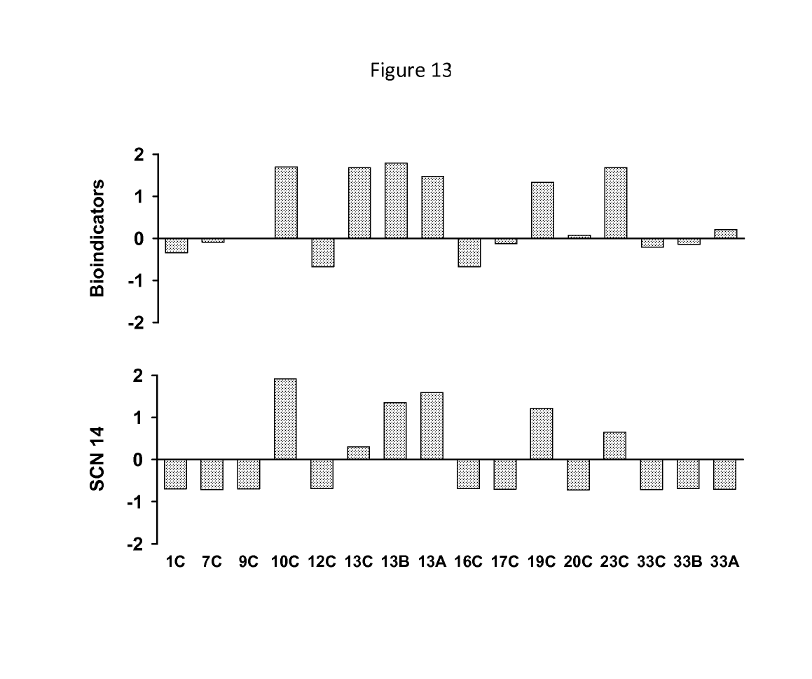

The qPCR assays were designed to detect specific 16S rRNA gene sequences whose sample distribution among the subset of 16 sediment samples was correlated with specific hydrocarbons (e.g. gasoline-range hydrocarbons) (FIG. 13). FIG. 13 graphically illustrates comparison of the abundance and distribution of gasoline-range bioindicators (top panel) with the presence of gasoline-range hydrocarbons (lower panel) in GOM sediments. All values are expressed as Z-scores (number of standard deviations above or below the mean).

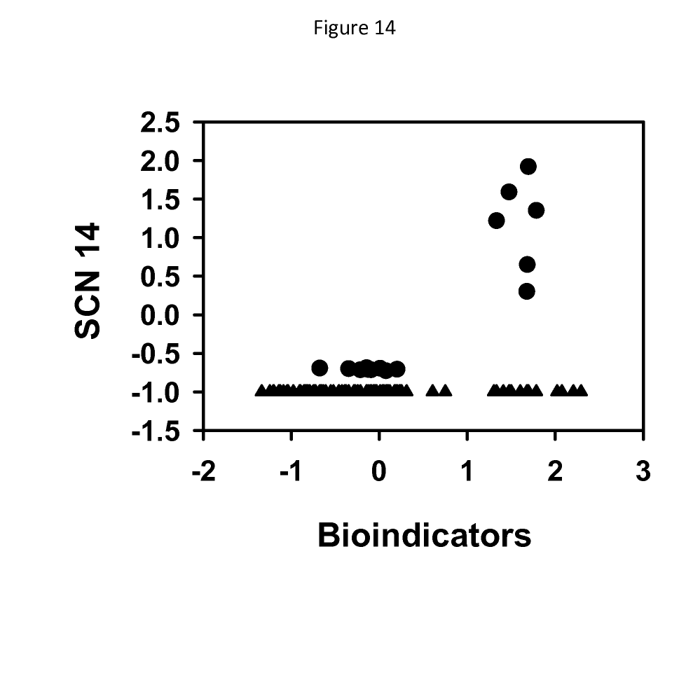

These assays were performed on both the set of 16 unblinded samples and the 77 blinded GOM samples to determine whether they could predict the presence of gasoline-range hydrocarbons (FIG. 14). FIG. 14 graphically illustrates a plot of gasoline-range hydrocarbon bioindicator composite values versus gasoline-range values from 93 GOM sediments comprising 16 samples with known hydrocarbon values (filled circles) and 77 samples that were geochemically blinded (filled triangles). The blinded samples were assigned an arbitrary hydrocarbon value of −1.0, for all values.

These assays revealed the relationship between the abundance of the bioindicators and the abundance of gasoline range hydrocarbons in the 16 unblinded GOM samples was binary in nature rather than linear. Thus, the bioindicators identified the presence of these hydrocarbons, but did not provide information as to the amounts. Examination of the bioindicator levels in the 77 blinded samples revealed, as was the case with the unblinded samples, two groups of samples with either high (Z-Score >1.25) or low (Z-Score <0.8) bioindicator values. The presence of gasoline range hydrocarbons in the unknown blinded samples were predicted based upon having bioindicator Z-score values above or below 1.0. This metric correctly predicted the predicted the presence of these hydrocarbons in 75/77 samples. One of the sample not predicted correctly had a bioindicator Z-score value that was borderline (Z-Score approximately 0.8). The other sample not correctly predicted may have been the result of incorrect geochemical determination of the presence of gasoline-range hydrocarbons. This possibility is supported by the observation that other gasoline-range hydrocarbon species (besides the 14-carbon molecules used in the test) were more consistent with the bioindicator value for this sample.

In one embodiment of the invention, each test sample is assayed for the presence of many independent bioindicators that are positively correlated with the presence of hydrocarbons. Microbes may exhibit different types of positive correlations to a geochemical parameter (e.g. linear, curvilinear, threshold, etc.) by virtue of the specific relationship. These are well known in the art and are described e.g., by Ashby, M. (2003). Methods for the survey and genetic analysis of populations, U.S. Pat. No. 6,613,520.

The sequence count data is expressed as absolute sequence counts per gram of sediment or per microgram of DNA recovered, as Z-scores (no. of standard deviations above/below the mean) with or without first log transforming the sequence count data.

Representative sequences from microbial divisions that were negatively correlated with the presence of hydrocarbons in sediment (e.g. GOM13 and SAGMEG-1 divisions) also have value as bioindicators for the presence of hydrocarbons. Demonstrating that a test (unknown) sediment sample BOTH harbors microbes that are positively correlated with the presence of hydrocarbons AND does not harbor microbes that are negatively correlated with hydrocarbons is a more robust association than the case of a sample only harboring microbes that are positively correlated with hydrocarbons.

In one embodiment of the invention, a test sample is assayed for the presence of microbial bioindicator sequences that are positively and negatively associated with the presence of hydrocarbons. The data could be expressed as absolute sequence counts per gram of sediment or per microgram of DNA recovered, as Z-scores (no. of standard deviations above/below the mean) or as ratios of these numbers derived from the positively correlated bioindicators divided by the negatively correlated bioindicators.

Alternative embodiments comprise methods of obtaining the bioindicator sequence data include qPCR, DNA sequencing technologies including, but not limited to, pyrosequencing (Roche), SOLEXA™ sequencing (Illumina), SOLiD™ (Applied Biosystems), Single Molecule Real Time (SMRT™) sequencing (Pacific Biosciences), Ion PGM™ (Ion Torrent), or hybridization-based methods of DNA detection such as gene chips. Any method that has the ability to capture and record greater than 100 variations in sequence and number of occurrences of 16S rRNA genes present in a sample is adequate to practice this invention.

In another embodiment, RNA is extracted from samples and converted to DNA by methods well known in the art (e.g. using reverse transcriptase), prior to PCR amplification of the 16S rRNA genes present in the sample. RNA is much less stable than DNA and will provide temporal information as to whether the microbes were active, or recently active, when the sample was collected. For example, microbes may persist in the environment in a dormant or dead state in some circumstances. Collection of 16S rRNA gene bioindicator data from both isolated DNA and from isolated RNA will provide both quantitative information (DNA) as well as whether the microbes were active (RNA). The combination of both RNA and DNA measurements will therefore allow one to distinguish active seep from dormant seep and dormant seep from recent organic matter (ROM) background.

### Example 2

### Characterization of Microbial Communities Associated with Thermogenic Hydrocarbon Seeps

This Example describes an alternative protocol for characterizing microbial communities associated with thermogenic hydrocarbon seeps.

Genomic DNA extracted as described in “Example 1: Characterization of microbial communities associated with thermogenic hydrocarbon seeps” were further prepared as follows. A portion of the 16S rRNA gene was amplified using the TX9/1391 primers as previously described (Ashby et al., 2007 AEM 73(14):4532-4542). Amplicons were agarose gel purified and quantitated using SYBR green (Invitrogen, Carlsbad, Calif.). A second round of PCR was performed using fusion primers that incorporated the ‘A’ and ‘B’ 454 pyrosequencing adapters onto the 5′ ends of the TX9/1391 primers, respectively. The forward fusion primer also included variable length barcodes that enabled multiplexing multiple samples into a single 454 sequencing run. These amplicons were PAGE purified and quantitated prior to combining into one composite library. The resulting library was sequenced using the standard 454 Life Sciences Lib-L emulsion PCR protocol and Titanium chemistry sequencing (Margulies, M., M. Egholm, et al. 2005 “Genome sequencing in microfabricated high-density picolitre reactors.” Nature 437(7057): 376-380). Sequences that passed the instrument QC filters were also subjected to additional filters that required all bases be Q20 or higher and the average of all bases in any read to be Q25 or greater. Furthermore, the TX9 primer was trimmed off of the 5′ end and the sequences were trimmed on the 3′ end at a conserved site distal to the V6 region (ca. position 1067, E. coli numbering). The final sequences were approximately 250 bp in length and included the V5 and V6 regions (V5V6 sequences). The term “V5V6” indicates sequences that include the fifth variable (V5) and sixth variable (V6) regions of the 16S rRNA gene.

The 93 samples profiled from the Green Canyon block of the Gulf of Mexico, resulted in 5,625,371 V5V6 sequences of which 552,568 were unique. The sequences were filtered to only include unique sequences with abundance greater than 0.5% in one of the 93 samples, and those 473 V5V6 sequences were correlated with geochemical data. A total of 198 V5V6 sequences were selected for bioindicator design based on strong correlation to gasoline-range hydrocarbons.

The 198 sequences were aligned with the NAST aligner available from GREENGENES™ and analyzed with the ARB™ software package (joint initiative of the Lehrstuhl für Mikrobiologie and the Lehrstuhl für Rechnertechnik and Rechnerorganisation/Parallelrechnerarchitektur of the Technische Universität, München, Germany). The analysis found 35 groups (clades) of sequences with similarity within a group greater than 97% and 57 sequences that did not cluster and were treated separately. Bioindicator primers were designed as previously described in Example 1 to the consensus sequence of the 35 groups (Table 3), and to each of the 57 unique un-grouped sequences (Table 4) resulting in 92 bioindicator probes (PTM12 through 103, Table 5).

Genomic DNA extracted as described in “Example 1: Characterization of microbial communities associated with thermogenic hydrocarbon seeps” were further prepared as follows. A portion of the 16S rRNA gene was amplified using the TX9/1391 primers as previously described (Ashby et al., 2007 AEM 73(14):4532-4542). Amplicons were agarose gel purified and quantitated using SYBR green (Invitrogen, Carlsbad, Calif.). A second round of PCR was performed using fusion primers that incorporated the ‘A’ and ‘B’ 454 pyrosequencing adapters onto the 5′ ends of the TX9/1391 primers, respectively. The forward fusion primer also included variable length barcodes that enabled multiplexing multiple samples into a single 454 sequencing run. These amplicons were PAGE purified and quantitated prior to combining into one composite library. The resulting library was sequenced using the standard 454 Life Sciences Lib-L emulsion PCR protocol and Titanium chemistry sequencing (Margulies, M., M. Egholm, et al. 2005 “Genome sequencing in microfabricated high-density picolitre reactors.” Nature 437(7057): 376-380). Sequences that passed the instrument QC filters were also subjected to additional filters that required all bases be Q20 or higher and the average of all bases in any read to be Q25 or greater. Furthermore, the TX9 primer was trimmed off of the 5′ end and the sequences were trimmed on the 3′ end at a conserved site distal to the V6 region (ca. position 1067, E. coli numbering). The final sequences were approximately 250 bp in length and included the V5 and V6 regions (V5V6 sequences).

Regarding discovery of the consensus sequences of PTM12 through PTM103, 93 samples were profiled from the Green Canyon block of the Gulf of Mexico, and this resulted in 5,625,371 V5V6 sequences, of which 552,568 were unique. The sequences were filtered to only include unique sequences with abundance greater than 0.5% in one of the 93 samples, and those 473 V5V6 sequences were correlated with geochemical data. A total of 198 V5V6 sequences were selected for bioindicator design based on strong correlation to gasoline-range hydrocarbons.

The 198 sequences were aligned with the NAST aligner available from GREENGENES™ and analyzed with the ARB™ software package (joint initiative of the Lehrstuhl für Mikrobiologie and the Lehrstuhl für Rechnertechnik and Rechnerorganisation/Parallelrechnerarchitektur of the Technische Universität, München, Germany). The analysis found 35 groups (clades) of sequences with similarity within a group greater than 97% and 57 sequences that did not cluster and were treated separately. Bioindicator primers were designed as previously described in Example 1 to the consensus sequence of the 35 groups (Table 3), and to each of the 57 unique un-grouped sequences (Table 4) resulting in 92 bioindicator probes (PTM12 through PTM103, Table 2).

### Table 2. Probes and Amplification Primer Pair Sequences of the Invention, e.g., for Hydrocarbon Detection, e.g., as Oil, Gasoline-Range Hydrocarbon or Pollution Bioindicators of the Invention

The exemplary sequences of the invention can be used individually or in groups as probes or detection molecules, or in pairs, e.g., as amplification pairs, e.g., as PCR primer pairs, to practice methods of the invention, e.g., methods of detecting the presence of a subsurface petroleum or gas accumulation or deposit, or the presence of a petroleum seep; or, methods of detecting the presence of a hydrocarbon, a petroleum or a gas accumulation, or the presence of a hydrocarbon, a petroleum or a gas pollutant.

In alternative embodiments, when sequences of the invention are used individually (or in groups), e.g., to practice methods of the invention, they can be used in hybridization reactions, e.g., in situ hybridizations, or as probes immobilized on a bead or a semisolid or solid surface, e.g., as probes immobilized on an array, a biochip, a chip, a bead, a gel, a liposome, a fiber, a film, a membrane, a metal, a resin, a polymer, a ceramic, a glass, an electrode, a microelectrode, a graphitic particle, or a microparticle or a nanoparticle. In alternative embodiments, sets of probes are used together in one detection reaction, e.g., one hybridization reaction, or immobilized individually on the same array, biochip, fiber, electrode and the like. For example, four probes, such as SEQ ID NO:1; SEQ ID NO:2; SEQ ID NO:3; SEQ ID NO:4 can be used in one detection reaction, or can be immobilized on the same array, biochip, fiber, electrode and the like. In alternative embodiments, all of the sequences (e.g., probes) of the invention are immobilized on the same product of manufacture of the invention, e.g., all can be immobilized on the same array, biochip, chip, bead, gel, liposome, fiber, film, membrane, metal, resin, polymer, ceramic, glass, electrode, microelectrode, graphitic particle, or microparticle or nanoparticle.

In alternative embodiments, sequences of the invention are used as amplification pairs, e.g., as PCR primer pairs, e.g., to practice methods of the invention. In alternative embodiments, sets of amplification (e.g., PCR) primer pairs are used together in one amplification (e.g., PCR) reaction. For example, two amplification pairs, such as SEQ ID NO:1/2 and SEQ ID NO:3/4 can be used in one detection reaction.

In Table 2, the “PT” number references the consensus sequence from which the primer pair was derived; thus, for example, the exemplary embodiments SEQ ID NO:1 and SEQ ID NO:2, are a sense and antisense (respectively) nucleic acid primer pair (amplification pair; primer pair sequence) that can be used to amplify, detect and/or quantify a genus of sequences based on the same consensus sequence, in this example, PTOM-03. The number after the “PTOM” designation (for example, for SEQ ID NO:1 and SEQ ID NO:2, is 834F and 1270R) indicates the residue number of the consensus sequence the forward, or “F” amplification primer, begins (the 5′-most residue) on the sense strand (e.g., 834 for SEQ ID NO:1), and the residue number of the consensus sequence the reverse amplification primer, or “R”, sequence begins (the 5′-most residue) on the antisense strand (e.g., 1270 for SEQ ID NO:2). To further illustrate, in Table 2: for SEQ ID NO:1, the 834F residue is in bold (it's a “A” nucleotide) and for SEQ ID NO:2 the 1270R residue is in bold (it's a “G” nucleotide).

In practicing the methods of the invention (e.g., methods of detecting the presence of a subsurface petroleum or gas accumulation or deposit, or the presence of a petroleum seep; or, methods of detecting the presence of a hydrocarbon, a petroleum or a gas accumulation, or the presence of a hydrocarbon, a petroleum or a gas pollutant), or when using the compositions, e.g., the amplification primer pairs of the invention, in polymerase chain reaction (PCR), exemplary (alternative) conditions for PCR include: 20 sec at 94° C.; 25 sec at 63° C. and 30 sec at 72° C. In Table 2, the “TM” is the melting temperature (Tₘ). In alternative embodiments, Tₘ melting temperatures are important for determining the appropriate temperatures to use in a protocol such as an amplification reaction (e.g., PCR), or Tₘ melting temperatures can also be used as a proxy for equalizing the hybridization strengths of a set of molecules, e.g. the oligonucleotide probes of arrays or microarrays of the invention.

TABLE 2

| PRIMER | SEQUENCE (5′-3′) | TM | SEQ ID NO: |
|---|---|---|---|
| PTM03-834F | AGGGGATATCAACTCCTCCGTGTCG | 63 | SEQ ID NO: 1 |
| PTM03-1270R | ATCACTCCGTGGCCACCCGTTG | 63 | SEQ ID NO: 2 |
| PTM04-808F | GGGCGTAAACGCTGTGGGCTTA | 63 | SEQ ID NO: 3 |
| PTM04-1301R | TGGATGGGTTTCGGGATTGCCTTCAC | 63 | SEQ ID NO: 4 |
| PTM05-811F | CGTAAACGCTGCCCGCTTG | 62 | SEQ ID NO: 5 |
| PTM05-1135R | TCGAAGATAGCAACTAAGAGCGAG | 62 | SEQ ID NO: 6 |
| PTM06-820F | GCTATGTGTCGGGAGATCCACGT | 62 | SEQ ID NO: 7 |
| PTM06-1267R | TCGGGATCGGTACTCTTTGTTCCG | 62 | SEQ ID NO: 8 |
| PTM07-820E-ALT | TGCTAGCTTGGTGTTGGATAACCTA | 63 | SEQ ID NO: 9 |
| PTM07-1115R-ALT | CGGACTTGAAAATAGCAACTGAAGATGG | 62 | SEQ ID NO: 10 |
| PTM08-849F | CTCTGTGTCGAAGCTAACGCTTTAA | 62 | SEQ ID NO: 11 |
| PTM08-1142R | CAGGATTTCTGGGCAGTTTCGTCAG | 63 | SEQ ID NO: 12 |
| PTM10-840F | TCGACCCCTTCTGTGCCGCA | 63 | SEQ ID NO: 13 |
| PTM10-1190R | ACCTTCCTCCGCATTATCTGCGA | 63 | SEQ ID NO: 14 |
| PTM11-818F | GATGTTCACTTGGTGTCGGTCGCAC | 63 | SEQ ID NO: 15 |
| PTM11-1244R | TTGCAACTCTCTGTACCTTCCATTGTAG | 62 | SEQ ID NO: 16 |
| PTM12-851F | GCCCCAGTGCCGCAGGGAA | 63 | SEQ ID NO: 17 |
| PTM12-1045R | CTCTCAGCTTGTCTGGCAAGGTC | 63 | SEQ ID NO: 18 |
| PTM13-844F | ACGTGGTTATTCAGTGCCGGAGAG | 63 | SEQ ID NO: 19 |
| PTM13-1046R | CCTCTCAGCTAGTCCAGCAAAGTC | 63 | SEQ ID NO: 20 |
| PTM14-819F | CTGCTTGCTTGATGTTAGTTGGCT | 63.5 | SEQ ID NO: 21 |
| PTM14-1042R | CTCTCGGAAAATCAGGCAAGGTCATCAG | 62 | SEQ ID NO: 22 |
| PTM15-817F | CGATGCCAGCTATGTGTCGGAAG | 64 | SEQ ID NO: 23 |
| PTM15-1046R | CTCTCAGCTAATCTGGCAAGGTCC | 63 | SEQ ID NO: 24 |
| PTM16-810F | CCGTAAACGATGCAGGCTAGGTGT | 63 | SEQ ID NO: 25 |
| PTM16-1045R | CTCTCAGCTCGTCCAGCAAGAC | 63 | SEQ ID NO: 26 |
| PTM17-828F | CCAGCTGTAAACGATGCAGGCTA | 63 | SEQ ID NO: 27 |
| PTM17-1050R | ACCTCCTCTCAGCTTGTCTGGTAAG | 63 | SEQ ID NO: 28 |
| PTM18-851F | CTAAACATCAGTACCTCCTCGAGAGG | 62 | SEQ ID NO: 29 |
| PTM18-1049R | ACTCCTCTCAGCGTGTCAGGTAAG | 63 | SEQ ID NO: 30 |
| PTM19-809F | GCAGTAAACGATGCGGGCYAGG | 62-63 | SEQ ID NO: 31 |
| PTM19-1048R | CACCTCTCAGCTAATTCAGCAAAGTC | 62.5 | SEQ ID NO: 32 |
| PTM20-844F | GATGCTCGCTAGGTGTTAAATACCCTG | 63 | SEQ ID NO: 33 |
| PTM20-1049R | CTTCCTCTCAGCGAATTTGGTAAGGTC | 63 | SEQ ID NO: 34 |
| PTM21-833F | GGCCGTAAACGATGCATACTAGGTGA | 62.5 | SEQ ID NO: 35 |
| PTM21-1051R | CACCTCCTCTCAGCTCGTCGG | 63.5 | SEQ ID NO: 36 |
| PTM22-849F | TACTAGGTGATGGTACGGCTATGAGC | 63 | SEQ ID NO: 37 |
| PTM22-1050R | ACCTCCTCTCAGCTCGTTGGGTAA | 63 | SEQ ID NO: 38 |
| PTM23-838F | GTAAATGATGTGGGCTAGGTGCAAAGC | 63 | SEQ ID NO: 39 |
| PTM23-1038R | CTTGTCTGGTAAGGTCATCAGCCTG | 62 | SEQ ID NO: 40 |
| PTM24-852F | AGGTGTGGCATTACTGCGAGTGAT | 63 | SEQ ID NO: 41 |
| PTM24-1051R | CGCCACCTCTCAGCTAATCTGG | 63 | SEQ ID NO: 42 |
| PTM25-809F | GGCGTAAACGATGTGGGCTTCG | 62.5 | SEQ ID NO: 43 |
| PTM25-1053R | GCACCACCTCTCTGCCTATTATTCG | 63 | SEQ ID NO: 44 |
| PTM26-809F | GCTGTAAACGATGCGGGCCAG | 63 | SEQ ID NO: 45 |
| PTM26-1052R | CGCCACCTCTCAGCTAATCCAG | 63 | SEQ ID NO: 46 |
| PTM27-812F | GTAACGATGCGGGCCAGGTGTTG | 64 | SEQ ID NO: 47 |
| PTM27-1052R | CGCCACCTCTCAGCTAATCCG | 63 | SEQ ID NO: 48 |
| PTM28-829F | GGTGTAGCGGGTATTGATCCCTGC | 62 | SEQ ID NO: 49 |
| PTM28-1058R | CAGCACCTGTCACTTTGTCCCGA | 62 | SEQ ID NO: 50 |
| PTM29-841F | GGGCACTAGGTGCAGGGGGTG | 63 | SEQ ID NO: 51 |
| PTM29-1051R | TGTCACCAGGTTCCCCCGAAGGG | 63 | SEQ ID NO: 52 |
| PTM30-832F | CACGCCSTAAACAGTGGACACTAGATA | 62-63 | SEQ ID NO: 53 |
| PTM30-1061R | CAGCACCTGTGACAGTTCCTGACT | 63 | SEQ ID NO: 54 |
| PTM31-806F | CTAGCTGTAAACGATGCGGGCT | 63 | SEQ ID NO: 55 |
| PTM31-1040R | AGCTAATCCGGTAAGGTCTTCAGCC | 63 | SEQ ID NO: 56 |
| PTM32-807F | TAGCCGTAAACGATGGGCACTAGAT | 63 | SEQ ID NO: 57 |
| PTM32-1054R | ACCTCTGCTGGCTTCCTGGC | 63 | SEQ ID NO: 58 |
| PTM33-818F | GATGGGCACTTGACGTAGGCGAT | 63 | SEQ ID NO: 59 |
| PTM33-1053R | CACCTGTACAGGCTCCGGATTGG | 63 | SEQ ID NO: 60 |
| PTM34-839F | CGATGTGGACTTGGCGTTGGTGG | 63 | SEQ ID NO: 61 |
| PTM34-1056R | GCAGCACCTGTCCAGGCTCC | 63 | SEQ ID NO: 62 |
| PTM35-838F | GCTGTAAACGATGGATACTAGATTTTGCAA | 62 | SEQ ID NO: 63 |
| PTM35-1032R | CGAAGAGGATAACCAACCCTTTCAGG | 62 | SEQ ID NO: 64 |
| PTM36-808F | AGCTGTAAACGATGGATACTAGGTGTGG | 63 | SEQ ID NO: 65 |
| PTM36-1063R | GCACCACCTGTTATYTCGTCTTCCCTAA | 63 | SEQ ID NO: 66 |
| PTM37-829F | CACGCCCTAAACGGTGGACACTAG | 63 | SEQ ID NO: 67 |
| PTM37-1059R | GCACCTGTGGCAGCTCCTGAC | 63 | SEQ ID NO: 68 |
| PTM38-808F | AGCCGTAAACGATGGACACTTGACG | 64 | SEQ ID NO: 69 |
| PTM38-1031R | GTTACCGGTTGTCACCCTTTCGGGC | 63 | SEQ ID NO: 70 |
| PTM39-838F | ACGATGCTCGCTATGTGTCAGGT | 63 | SEQ ID NO: 71 |
| PTM39-1045R | CTCTCAGCGGATCTGGTAAGGTCT | 63 | SEQ ID NO: 72 |
| PTM40-834F | GCCCTAAACGATGTACACTTGGCATG | 63 | SEQ ID NO: 73 |
| PTM40-1051R | CCTGTGCTGACTTTCCACCAGAGG | 63 | SEQ ID NO: 74 |
| PTM41-838F | GGTATTGACCCCTGCTGTGCCG | 63 | SEQ ID NO: 75 |
| PTM41-1042R | GGGTTCCCCGAAGGGCACATCCC | 63 | SEQ ID NO: 76 |
| PTM42-837F | AGGTATCGACCCCTTCTGTGCCG | 63.5 | SEQ ID NO: 77 |
| PTM42-1060R | GCACCACCTGTTATCTCGTCTTCCG | 64 | SEQ ID NO: 78 |
| PTM43-824F | CGCTAGGTGTCAGACACGGTGC | 64 | SEQ ID NO: 79 |
| PTM43-1048R | TCCTCTCAGCGATTCAGGTAAGACC | 63 | SEQ ID NO: 80 |
| PTM44-809F | GCTGTAAACGATGTGGACTTGGCG | 63 | SEQ ID NO: 81 |
| PTM44-1045R | CCAGGCTCCCCGAAGGGTCG | 64 | SEQ ID NO: 82 |
| PTM45-842F | AGGTATCGACCCCTTCTGTGCCG | 63.5 | SEQ ID NO: 83 |
| PTM45-1063R | GCACCACCTGTTATCCTGTCTTCCCT | 63 | SEQ ID NO: 84 |
| PTM46-837F | CGACCCCTTCTGTGCCGTAGC | 63 | SEQ ID NO: 85 |
| PTM46-1060R | GCACCACCTGTTATCCTGTCTTCGG | 63 | SEQ ID NO: 86 |
| PTM47-816F | ACGATGCGTGCTAGGTGTTGGTAG | 63 | SEQ ID NO: 87 |
| PTM47-1037R | TTGTCTGGTAAGGTCGTCAGCCTGA | 63 | SEQ ID NO: 88 |
| PTM48-817F | CGATGCGGGCTAGGTGTTGGG | 63 | SEQ ID NO: 89 |
| PTM48-1045R | CTCTCAGCTTGTCCAGCAAGACC | 63 | SEQ ID NO: 90 |
| PTM49-818F | GCTGTGGGCTTAGTGTTGGGTGTCT | 63 | SEQ ID NO: 91 |
| PTM49-1046R | ACCTCTCGGCAATCCAGCAAGG | 63 | SEQ ID NO: 92 |
| PTM50-811F | CGTAAACGATGCATACTAGGTGATGGC | 63 | SEQ ID NO: 93 |
| PTM50-1041R | CAGCTCGTCAGGTAAGGTCGTCAA | 63 | SEQ ID NO: 94 |
| PTM51-835F | TGCATACTAGGTGATGGTACGGCCAT | 63 | SEQ ID NO: 95 |
| PTM51-1045R | CCTCTCAGCTCGTCGGGTAAGG | 63 | SEQ ID NO: 96 |
| PTM52-817F | CGATGCGGGCTAGGTGTTAGGG | 63 | SEQ ID NO: 97 |
| PTM52-1041R | CAGCTTGTCTGGCAAGATCGTCA | 63 | SEQ ID NO: 98 |
| PTM53-811F | TGTAAACGCTGCCTGCTTAGTGTTAG | 63 | SEQ ID NO: 99 |
| PTM53-1049R | CTCTCTACCTATTGATCGAGCAAGGTC | 63 | SEQ ID NO: 100 |
| PTM54-817F | CGCTGCCCGCTTGGTATTAGG | 63 | SEQ ID NO: 101 |
| PTM54-1043R | CTCGGAGAATTCAGCAAGGTCTTCA | 63 | SEQ ID NO: 102 |
| PTM55-817F | CGCTGCTTGCTTGATGTTAGTTGG | 63 | SEQ ID NO: 103 |
| PTM55-1044R | TCTCGGAAAATCAGGCAAGGTCATCA | 63 | SEQ ID NO: 104 |
| PTM56-817F | CGCTGCAGGCTTGGTGTTGG | 63 | SEQ ID NO: 105 |
| PTM56-1044R | TCTCGGAAAATCAGGCAAAGTCATCAG | 63 | SEQ ID NO: 106 |
| PTM57-816F | ACGCTGCAGACTTGGTGTCGG | 63 | SEQ ID NO: 107 |
| PTM57-1045R | CTCTCGGAAAATCGGGCAAAGTCATC | 63 | SEQ ID NO: 108 |
| PTM58-817F | CGCTGCAGGCTTGGTGTTGG | 63 | SEQ ID NO: 109 |
| PTM58-1046R | CCTCTCGAAAAATCAGGTAAGGTCATCAG | 63 | SEQ ID NO: 110 |
| PTM59-816F | ACGATGCGAGCTAGGTGGTAGTC | 63 | SEQ ID NO: 111 |
| PTM59-1044R | TCTCAGCTAATCTGACAAGGTCTTCAG | 63 | SEQ ID NO: 112 |
| PTM60-820F | TGCGGGCTAGGTGTTGGCATTAC | 63 | SEQ ID NO: 113 |
| PTM60-1041R | CAGCTAATTTGGTAAGGTCTTCAGCCT | 63 | SEQ ID NO: 114 |
| PTM61-817F | CGATGCGGGCCAGGTGTTGG | 63 | SEQ ID NO: 115 |
| PTM61-1047R | ACCTCTCAGCTAATCCGGTAAGGTCT | 63 | SEQ ID NO: 116 |
| PTM62-817F | CGATGCGCGTTAGGTGTGCC | 63 | SEQ ID NO: 117 |
| PTM62-1039R | GCTGGTCAAGCAAGGTCTTCAGC | 63 | SEQ ID NO: 118 |
| PTM63-811F | CGTAAACGATGTGAGCTAGGTGTCAG | 63 | SEQ ID NO: 119 |
| PTM63-1046R | CCTCTCAGCGAATCGGGTAAGGTC | 63 | SEQ ID NO: 120 |
| PTM64-817F | CGATGTGAGCTAGGTGTCAGTCATG | 63 | SEQ ID NO: 121 |
| PTM64-1047R | ACCTCTCAGCGAATTTGGTAAGGTCTT | 63 | SEQ ID NO: 122 |
| PTM65-811F | CGTAAACGATGCGAGCTAGGTGT | 63 | SEQ ID NO: 123 |
| PTM65-1043R | CTCAGCAAGTCTGGCAAGGTCTTC | 63 | SEQ ID NO: 124 |
| PTM66-817F | CGATGCTTGCTAGGTGTCAGCC | 63 | SEQ ID NO: 125 |
| PTM66-1047R | ACCTCTCAGCTAATCGGGTAAGGTCT | 63 | SEQ ID NO: 126 |
| PTM67-814F | AAACGATGCTCGCTAGGTGTCAG | 63 | SEQ ID NO: 127 |
| PTM67-1046R | CCTCTCAGCGAATCAGGTAAGGTCTTC | 63 | SEQ ID NO: 128 |
| PTM68-821F | GGGTACTAGGTGTAGGAGGTATCGACCC | 63 | SEQ ID NO: 129 |
| PTM68-1057R | ACCACCTGTCTCCCTGTTCTTCCG | 63 | SEQ ID NO: 130 |
| PTM69-819F | GTAAACGATGGGCACTAGGTGTTGGAG | 63 | SEQ ID NO: 131 |
| PTM69-1052R | TCTCCCTGTCTCAAGAAAATCTTAAGAGGA | 63 | SEQ ID NO: 132 |
| PTM70-815F | AACGATGGATACTAGGTGTAGGGGGTTTAG | 63 | SEQ ID NO: 133 |
| PTM70-1053R | CCACCTGTATACCTGTCCCCGAAAGG | 63 | SEQ ID NO: 134 |
| PTM71-809F | GCTGTAAACGATGGATACTAGGTGTAGGG | 63 | SEQ ID NO: 135 |
| PTM71-1055R | CACCACCTGTTTACCTGTCCCCTAAAGG | 63 | SEQ ID NO: 136 |
| PTM72-815F | AACGATGGATACTAGGTGTGGGAGGTATC | 63 | SEQ ID NO: 137 |
| PTM72-1058R | CACCTGTTATCTCGTCTTCCCCAAAGG | 63 | SEQ ID NO: 138 |
| PTM73-816F | ACGATGTGCACTTGGCATGCG | 63 | SEQ ID NO: 139 |
| PTM73-1050R | TGCTGACTTTTCACCAGAGGCGA | 63 | SEQ ID NO: 140 |
| PTM74-812F | GCAAACGATGTTCACTGGGTGTCGG | 63 | SEQ ID NO: 141 |
| PTM74-1037R | CTGTGCTAGCTCCTCTACCCGA | 63 | SEQ ID NO: 142 |
| PTM75-809F | GCCGTAAACGATGGATGCTTGGTG | 63 | SEQ ID NO: 143 |
| PTM75-1055R | GCACGGGTAACAGAGATTACTCTCTGA | 63 | SEQ ID NO: 144 |
| PTM76-821F | GGCTACTAGCTGTTTGAAGTATCGACC | 63 | SEQ ID NO: 145 |
| PTM76-1050R | CTGCTCTAGTGTCCTTGTAGGTAGACA | 63 | SEQ ID NO: 146 |
| PTM77-815F | AACGATGGACACTGGCTATTTGAAGTGT | 63 | SEQ ID NO: 147 |
| PTM77-1049R | TGGGCTAGTGTCCTTGTGGGTAGACT | 63 | SEQ ID NO: 148 |
| PTM78-817F | CTTTGGACACTAGGTATGGAGGGTATCG | 63 | SEQ ID NO: 149 |
| PTM78-1052R | TGTGCCGGCTCCTGGCTTTAC | 63 | SEQ ID NO: 150 |
| PTM79-813F | CAAACGATGGACACTAGGTATGGGGGGT | 63 | SEQ ID NO: 151 |
| PTM79-1048R | TGTGCACCCGTCCTGCGAAG | 63 | SEQ ID NO: 152 |
| PTM80-817F | CGGTGGATACTGGATATAGGGGGTATCG | 63 | SEQ ID NO: 153 |
| PTM80-1052R | GTGCTAGCTCCTTGGAAAACCAAGGT | 63 | SEQ ID NO: 154 |
| PTM81-814F | AAACGGTGGACATTAGGTATGGGGAGTATC | 63 | SEQ ID NO: 155 |
| PTM81-1056R | CCTGTGCCAGCTCCTGACTGG | 63 | SEQ ID NO: 156 |
| PTM82-816F | ACGGTGGACACTAGACATGGGAGGTAT | 63 | SEQ ID NO: 157 |
| PTM82-1055R | CTGTGACAGCTCCTGACTGGATACA | 63 | SEQ ID NO: 158 |
| PTM83-812F | CTAAACGGTGGACACTAGATATGGGGAG | 63 | SEQ ID NO: 159 |
| PTM83-1048R | AGTTCCTGACTGGATACAGGTCGTCC | 63 | SEQ ID NO: 160 |
| PTM84-817F | CGATGGACACTAGGTATAGGGAGTATCG | 63 | SEQ ID NO: 161 |
| PTM84-1055R | ACCTGTGACGGCTCCTGATTTAACAG | 63 | SEQ ID NO: 162 |
| PTM85-807F | ACGCCCTAAACGTTGGACACTAGGTAT | 63 | SEQ ID NO: 163 |
| PTM85-1048R | AGCTCCTGACTGGATACAGGTCGT | 63 | SEQ ID NO: 164 |
| PTM86-811F | CGTAAACTATGGACACTAGGTATGGGGAG | 63 | SEQ ID NO: 165 |
| PTM86-1052R | TGTGCCGGCTCCTGACTCAACA | 63 | SEQ ID NO: 166 |
| PTM87-817F | CGATGGATACTAGGTGTGGGTGGCA | 63 | SEQ ID NO: 167 |
| PTM87-1049R | CTGTGCTGGCTCCCTTGCG | 63 | SEQ ID NO: 168 |
| PTM88-815F | AACGATGGATGCTGGGTGTGGGG | 63 | SEQ ID NO: 169 |
| PTM88-1046R | TGCAGGCTCCCCGAAGGGTC | 63 | SEQ ID NO: 170 |
| PTM89-818F | GATGCAGACTTGGTGTTGGTGGTTTAATAG | 63 | SEQ ID NO: 171 |
| PTM89-1055R | CAGCACCTGTGCGCGCT | 63 | SEQ ID NO: 172 |
| PTM90-817F | CGATGCCTACTAGGTTGTGGTGGTTC | 63 | SEQ ID NO: 173 |
| PTM90-1054R | CCTGTGCAAGTTTCACCCGAAGGTAA | 63 | SEQ ID NO: 174 |
| PTM91-810F | CCGTAAACGATGGGCACTTGACGTA | 63 | SEQ ID NO: 175 |
| PTM91-1293R | CACCTGTCAGATTCCGGACTGATTACC | 63 | SEQ ID NO: 176 |
| PTM92-808F | AGCTGTAAACGATGGATACTAGATTTTGCA | 63 | SEQ ID NO: 177 |
| PTM92-1043R | ATAGGTTCCTCCGAAGAGGATAGCCA | 63 | SEQ ID NO: 178 |
| PTM93-816F | ACGATGGGCACTAGATGTTTCTGCT | 63 | SEQ ID NO: 179 |
| PTM93-1053R | ACCTCTGCTGGCTTCCTGCAA | 63 | SEQ ID NO: 180 |
| PTM94-817F | CGATGGGCACTAGATGTTTCTGCTT | 63 | SEQ ID NO: 181 |
| PTM94-1053R | CCTCTGCTGGCTTCCTGGCA | 63 | SEQ ID NO: 182 |
| PTM95-812F | GTAAACGATGATCACTCGTTGTTGGCG | 63 | SEQ ID NO: 183 |
| PTM95-1042R | GATTCCCTTCGGGGCAGATTGCAA | 63 | SEQ ID NO: 184 |
| PTM96-818F | GATGAGTGCTAGGTGTTGGGGGGTTTC | 63 | SEQ ID NO: 185 |
| PTM96-1051R | CCTGTCACCATTGTCCCCGAAGGG | 63 | SEQ ID NO: 186 |
| PTM97-817F | CGATGTTCACTAGGTGTTGGGAGTATTGAC | 63 | SEQ ID NO: 187 |
| PTM97-1053R | ACCTGTCACCGAGTTCCCCGAAG | 63 | SEQ ID NO: 188 |
| PTM98-820F | TGTTCACTAGGTGTTGGGAGTATTGACCCT | 63 | SEQ ID NO: 189 |
| PTM98-1051R | CCTGTCACCAAGTTCCCCGAAGGG | 63 | SEQ ID NO: 190 |
| PTM99-813F | TAAACGATGAGAACTAGGTGTAGCGGG | 63 | SEQ ID NO: 191 |
| PTM99-1046R | TCTGTTCCGACAAAGTCGGAAAGATCC | 63 | SEQ ID NO: 192 |
| PTM100-817F | CGATGAACACTAGGTGTAGCGGGTATT | 63 | SEQ ID NO: 193 |
| PTM100-1044R | CCGAGTTCCCCGAAGGGCACA | 63 | SEQ ID NO: 194 |
| PTM101-813F | TAAACTATGGGTGCTAGCCGTCGG | 63 | SEQ ID NO: 195 |
| PTM101-1046R | CACCTGTCACCGGCCAATTGAAGA | 63 | SEQ ID NO: 196 |
| PTM102-821F | GGGTATTAGACATCGGCCGAAATTCG | 63 | SEQ ID NO: 197 |
| PTM102-1040R | CAGGTTCTCTTACGAGCACTCCG | 63 | SEQ ID NO: 198 |
| PTM103-812F | GTAAACGATGTCAACTAACTGTTGGGCG | 63 | SEQ ID NO: 199 |
| PTM103-1046R | CTGTATCAGAGTTCCCGAAGGCACC | 63 | SEQ ID NO: 200 |

Consensus sequences 16S rRNA genes whose distribution among the 16 GOM sediment samples were found to be significantly negatively associated with the presence of hydrocarbons. The two consensus sequences were derived from the Archaeal candidate division GOM13 and the division SAGMEG-1.

>Consensus_GOM13

(SEQ ID NO: 582)

CCGGATTAGA WACCCBGGTA GTCCTATGCY GTAAACGATG
CTCAcTAAGT GTTAGGtAAT GCAAGACRTT rTCTAGTGCC
GAAGcGAAAg CGTTAAgTGA GCCGCCTGGG AAGTACGTTC
GCAAGAATGA AACTTAAAGG AATTGGCGGG GGCCTACTAC
AAGAAGTGGA GCCTGCGGTT TAATTGGACT CAACTCCGGG
AARCTCACCT GGGCCGYAAC RtGRATGATT GTCCTGcTGA
AGACACTRCT TGAYGYGTTA CTGGAGGTGC ATGGCCATCG
TCAGTTCGTG CCGTGAGGTG TCCTGTTAAG TCAGGCAACG
AACGAGATCC CYRCCGctAa TTGCCAGCGa gaMcW...gK
tcGTCGGGGA CATTaGCGGG ACTGCTCGCG AAAAAGTGAG
AGGAAGGAAG GGCCAACGGT AGGTCAGTAT GCCCCGATAT
GCCCAGGGCT ACACGCGGGC TACAATGGCK RGTACAgAGG
GTTCCwACaC CGAaAGGtGA cGGYAATCTC c.AAAmYCGT
CTCAGTTGGg ATTGYGGGCT GCAACTCGCC CRCATGAACT
TGGAATTTCT AGTA
>Consensus_SAGMEG

(SEQ ID NO: 583)

GATTAGAWAC CCgGGTAGTC CTAGCTGTAA AGCATGCGGG
CCAGGTGTCT AGCGCTCCTT GAGGGCGCTA KGTGCCGGAG
GGAAGCCGTT AAGCCCGCCG CCTGGGAAGT ACGG.CGCAA
GGCTGAAACT TAAAGAAATT GGCGGGGGAG CACCACAAGR
GGTGGRACCT GCGGTTCAAT TGGATTCAAC GCCGGAMAAC
TCACCAGGGG CGACAGYTGG TTGAMGGCCA GRTTGACGAY
YTTGCYsGAC TAGCTGAGAG GTGGTGCATG GCCATCGTCA
GCTCGTACCG TGAGGCGTCC TGTTAAGTCA GGCAACGAGC
GAGATCCTCG cCCYTAGTTG CCATCGGTGG RAAGCCGGGC
ACTCTAGGGG GACCGCTGGC GCTAAGTCAG AGGAAGGAGA
GGGCGACGGT AGGTCAGTAt GCCCCGAATC CCCTGGGCTA
CACGCGGGTY ACAATGCGCA GGACAATGaG ATGCAACCCC
GTAAGGGGRA GCCAARCCCM TAAACCTGCG CTCGGTTCGG
ATCGAGGGCT GTAACTCGCC CTCGTGAAGC TGGAATCYCT
AGTAATCGCG TGCCAACACC GCGCGg

In summary, the following are consensus sequences of eight (8) bioindicator sequences, e.g, gasoline-range hydrocarbon bioindicator sequences, of the invention:

>Consensus_PTM03
(SEQ ID NO: 208)
CACGCCCTAAACGGTGGATACTAGATAYAGGGGATATCAACTCCTCCGTGTCGAAGCTAACGCTTTAAGTATCCCGCCTGGGAACT
ACGGCCGCAAGGCTAAAACTCAAAGGAATTGACGGGGGCCCGCACAAGCAGCGGAGCGTGTGGTTTAATTCGATGCAACACGAAGA
ACCTTACCCAGGYTTGACATGCTAGTGGTAGGAACCTGAAAGGGAGACGACCCTGGTTTTCCAGGGAGCTAGCACAGGYGCTGCAT
GGCTGTCGTCAGCTCGTGCCGTGAGGTGTTGGGTTAAGTCCCACAACGAGCGCAACCCCCATCGTCAGTTGAATTTCTCTGACGAA
ACTGCCCAGAAATCCTGGGAGGAAGGAGGGGATGACGTCAAGTCAGYATGGCCCTTATGCCTGGGGCRACACACACGCTACAATGG
GTGGTACAACGGGTGGCCACGGAGYGATCCGGAGCTAATCCTCA
>Consensus_PTM04
(SEQ ID NO: 212)
CAGGGCGTAAACGCTGTGGGCTTAGTGTTaGGTGTCCCATGAGGGCCCCTAGTGCTGgAGaGAAGtTGTTAAGCCCACAACCTGGG
AAGTACGGTCGCAaGGCTGAAACTTAAAGGAATCGGCGGGGGAGCACAGCAACGGGTGGAGCGTaCGGTTCAATTGGATTcTACGC
CGGAAAtCTCACCGGGGGCGACGGcTCGATGARGGCCAGGCtGATGACCTTGCcAGATGTGCCGAGAGGTGGTGCATGGCCGCCGT
CAGTTCGTGCCGCAAGGTGTTCTGTTAAGTCAGAtAACGAACGAGAcCCtCaCCtTTAATTGCtACCCtTTCCTCTGGGAgaGGgG
CACATTAgaGGGACCgCCACTGCTAAAGTGGAGGaAGgGGGGGGCAACGGTAGGTCAGTATGCCCCAAATCTCCCGGGCTACACGC
GCGCTACAAAGaATGGGACAATGGGYTCCGACaCCGAGAGGtGAAGGCAATCCCGAAACCCATCCATAGTTCGGATTGAGGgCTGA
AACTCGcCCTCATGAAGCTgGAATCCGTAGTAATC
>Consensus_PTM05
(SEQ ID NO: 227)
CAGGGCGTAAACGCTGCCCGCTTGgTaTTAGGgAACtTACAaGATTTCCTAtTGcCGGAGAGAAGTCGTTAAGCGGGCCACCTGGG
AAGTACGGTCGCAAGGCTGAAACTTAAAGGAATTGGCGGGGGAGCACCGCAACGGGTGGAGCGTGCGGTTCAATTGGATCCAACGC
CGGAAAGCTTACCGAGGGCGACGGATAgATGAAGGCCAGGCTaATGACCTTGCTAGATTtTCCGAGAGGTGGTGCATGGCCATCGA
CAGCTCGTACCGtGAGGCGTTCTGTTAAGTCAGATAACGAGCGAGACCCTCGCTCTTAGTTGCTATCTTCGAGTCCGCTCGggGaG
CACTCTAAGAGGACCGCTGGTGCTAAACCAGAGGAAGaAGGGGGCAACGGTAGGTCAGTATGCCCTGAATCCCTCGGGCTACACGC
GCGCTACAAAGGATGGGACAATGGGtTtCGACCCCGAGAGGGGGAGGCAATCCCGAAACCtATCCATAgTTCGgATc
>Consensus_PTM06
(SEQ ID NO: 237)
CCAGCCGTAAACGATGCCAGCTATGTGTCGGGAGATCCAcGTGTTCTTcCGGTGCCGTAGggAAGCCGTGAAgCTGGCCACCTGGG
AAGTACGGCCGCAAGGCTGAAACTTAAAGGAATTGGCGGGGGAGTACTACAACCGGTGGAGCTTGCGGTTTAATTGGATACAACGC
CGGAAATCTCACCGGGGGCGACAGCAGTATGAAGGCCAGGCTGAGGACCTTGCTAGATTAGCTGAGAGGAGGTGCATGGCCGTCGT
CAGTTCGTACCGTGAGGCATCCTGTTAAGTcAGGCAACGGGCGAGACCCGCGGTCTTAATTGCCAGCATACCCTTCGGGGTGATTG
GGTACAATAaGACGACtGCCAGCGCTAAGCTGGAGGAAGAAGCGGGCTACGGtAGGTCAGCATGCCCCRAATCCCCCGGGCTACAC
GCGTGCtACAATGGTCGGAACAAAGAgTACCgATCCCGAAAGGGAAAGGTGATCTCCTAAACCCGATCgAAGTTCGGATCGAAGGT
TGCAATTCGCCTTCGTGAAGTTGGAATCGGTAGTAATCGTGTCTCAAAATGACACGGTGAAT
>Consensus_PTM07
(SEQ ID NO: 257)
CAGGGTGTAAACGCTGCTAGCTTGGTGTTGGATAACCCACGTGGTTATTCAGTGCCGGAGAGAAGTTGTTAaGCTAGCTACCTGGG
aAGTACGGTCgCAAGGCTGAAACTTAAAGGAATTGGCGGGGGAGCACTGCaAcGGGTGGAGCGTACGgTTTAATTGGATTCAACGC
CGAAAACCTCACCGGAGGCGACAG.TGGATGAAGGCCAGGCTAAAGACtTTGCTGGACTAGCTGAGAGGTGGTGCATGGCCATCGG
CAGTTCGTACTGT.AAGCGTTCTGTTAAGTCAGATAACGAACAAGAC-
CCCATCTTCAGTTGCTATTTTCAAGTCCGCTTGAAAAGCACTCTGGAGATACTGCCCGCGCTAAGTGGGAGGAAGGAGRGGGCCAC
GGTAGGTCCGTATTCCCCGAATCCTCCGGGCTACACGCGCGCTACAAAGGATGGGACAAtGGGCTCCGAC
>Consensus_PTM08
(SEQ ID NO: 274)
CACGCCCTAAACGGTGGATACTRGATATAGGGGRTATCRACYCcTCYGTGTCGAAGCTAACGCtTTAAGTATCCCGCCTGGGRACT
ACGGCCGCAAGGCTAAAACTCAAAGGAATTGACGGGGGCCCGCACAAGCAGCGGAGCGTGTGGTTTAATTCGATGCAACACGAA.A
ACCTTACCCAGGCTTGACATRCTAGTGGTAGGAACCTGAAAGGGRGACGACCYGGTTTTCCARGGAGCTAGCACAGGTGCTGCATG
GCTRTCGTCAGCTCGTGCCGTGAGGTGTTGGGTTAAGTCCCACAACGAGCGCAACCCCYATcGYCAGTTGAATTTtTCTGRCGAAA
CTGCCCAGAAATCCTGGGAGGAAGGAGGGGATGACGTYAAGTCAGCATGGCCCTTATGYCTGGGGCRACACACACGCTACAATGGG
TGGTACARYRGGTkGCYACGGAGCAATCCGGAGCTAATCCYCAAAG-
CAYCCTCAGTAGGGATTGCAGGCTGAAACcCGCCTGCATGAACGCGGAGTTGCTAGTAACCGCAGGTCAGA-
ATACTGCGGTGAATRCG-TCTC
>Consensus_PTM10
(SEQ ID NO: 295)
CTAGCTGTAAACGATGGATACTAGGTGTGGGAGGtaTCGAccCCTTCTGTGCCGcAGCTAACGCATTAAGTATCCCGCCTGGGGAG
TACGGTCGCAAGGCTGAAACTCAAAGGAATTGACGGGGGCCCGCACAAGCGGTGGAGCATGTGGTTTAATTCGACGCAACGCGAAG
AACCTTACCgGGacTTGACATTatctTGCCCGTCTAAGAAATtagaTcTTcttcctTtcgGaagacRRgATaaCAGGTGGTGCATG
GTTGTCGTCAGCTCGTGTCGTgAGATGTTGGGTTAAGTCCCACAACGAGCGCAACCCTTRTGCYTAGTTGCTAActTgtTTtacAA
GTGCACTCTARGCAGACTGTCGCAGATAATGCGGAGGAAGGTGGGGATGACGTCAAATCATCATGCCCCTTACGTCCCGGGCTACA
CACGTGCTaCAATGGYCTGTACAgAGGGTAGCGAAAGAGCGATCTTAaGCCAATCcCAAAAAGCAGGCCcCAGTTCGGATTgGAGG
CtGcAACTCGCCTCCATGAAGTAGGAATCGCTAGTAATCGCGGAtcagCATGCCGCGGTGAAtACGTCCCGGG
>Consensus_PTM11
(SEQ ID NO: 302)
CACGCCCTAAACGATGTTCACTTGGTGTCGGTCGCACATACAGATCGGTGCCGGAGCTAACGCGTTAAGTGAACCGCCTGGGGAGT
ACGGTCGCAAGGCTAAAACTCAAGAGAATTGACGGGTCCCCGCACAAGCGGTGGAGCACGTGGTTTAATTCGATGATAAGCGAAGA
ACCTCACCTGGGCTTGACATGCTAGTGGTAGGAACCRGAAACGGKGACGACCCTGCCTTCGGGTAGGGAGCTWGCACAGGTGATGC
ATGGCTGTCGTCAGCTCGTGTCGTGGGACGTAGGGTTAAGtCCCGAAACGAGCGCAACCCCTGTCGTCAGTTGCCAGCGGATAATG
CCGGGGACTCTGACGAGACTGCTGGTGAATAGCCGGAGGAAGGAGGGGAYGACGTCAAGTCaTCATGTCCCTTATgCCCAGGGCGA
CACACAtGCTACAATGGAAGGTACAgAGAGTTGCAATACCGTAAGGTGGAGCTAATCCCAAAAAGCCTTCCCYAGTTCGGATTGAG
GTCTGCAACTCGACCTC

Rules for Consensus Sequence:

dash (-)=>60% of sequences have gap there

Other letters (used when a few letters are each seen in >30% of sequences):

M=A or C

R=A or G

W=A or T

S=C or G

Y=C or T

K=G or T

V=A, C, or G

H=A, C, or T

D=A, G, or T

B=C, G, or T

N=G, A, T, or C

UPPER CASE=>95% of sequences are same letter

lower case=>70% of sequences are same letter

dot (.)=<50% of sequences are same letter (note: this applies to “other letters” also)

In Table 3, below, alignment of partial 16S rRNA gene sequences (V5V6 sequences), whose distributions among the samples were correlated with gasoline-range hydrocarbons. The consensus sequence of each group is included in the alignment and primers (oligonucleotides) designed to selectively amplify each group of sequences is indicated on the top line of the alignment. For ease of viewing, the reverse primer is shown as its reverse-complement.

```
TABLE 3
PTM12
PTM12 forward primer (SEQ ID NO: 17)
PTM12 reverse primer (SEQ ID NO: 18)
reverse complement of reverse primer (SEQ ID NO: 314)
CONSENS_3 (SEQ ID NO: 315)
TXv5v6-0593770 (SEQ ID NO: 316)
TXv5v6-0219684 (SEQ ID NO: 317)

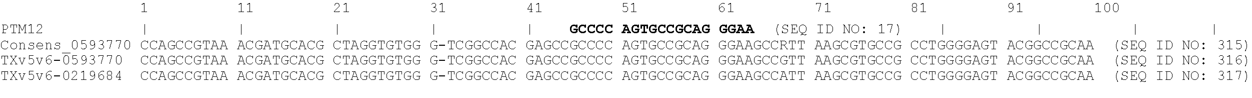

                                101 111 121 131 141 151 161 171 181 191 200
                                |  |  |  |  |  |  |  |  |  |  |
Consens_0593770 GGCTGAAACT TAAAGGAATT GGCGGGGGAG CACCACCAGG CGTGAAGCCT GCGGTTTAAT TGGAGTCAAC GCCGGGAACC TTACCGGGAG CGACAGCAGA
TXv5v6-0593770 GGCTGAAACT TAAAGGAATT GGCGGGGGAG CACCACCAGG CGTGAAGCCT GCGGTTTAAT TGGAGTCAAC GCCGGGAACC TTACCGGGAG CGACAGCAGA
TXv5v6-0219684 GGCTGAAACT TAAAGGAATT GGCGGGGGAG CACCACCAGG CGTGAAGCCT GCGGTTTAAT TGGAGTCAAC GCCGGGAACC TTACCGGGAG CGACAGCAGA

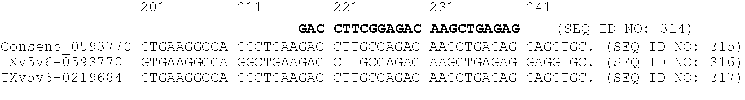

PTM13
PTM13 forward primer (SEQ ID NO: 19)
PTM13 reverse primer (SEQ ID NO: 20)
reverse complement of reverse primer (SEQ ID NO: 318)
CONSENS_0208415 (SEQ ID NO: 319)
TXv5v6-0208415 (SEQ ID NO: 320)
TXv5v6-0208460 (SEQ ID NO: 321)

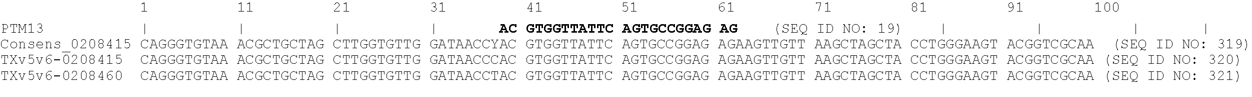

                                101 111 121 131 141 151 161 171 181 191 200
                                |  |  |  |  |  |  |  |  |  |  |
Consens_0208415 GGCTGAAACT TAAAGGAATT GGCGGGGGAG CACTGCAACG GGTGGAGCGT ACGGTTTAAT TGGATTCAAC GCCGAAAACC TCACCGGAGG CGACAGCTGR
TXv5v6-0208415 GGCTGAAACT TAAAGGAATT GGCGGGGGAG CACTGCAACG GGTGGAGCGT ACGGTTTAAT TGGATTCAAC GCCGAAAACC TCACCGGAGG CGACAGCTGA
TXv5v6-0208460 GGCTGAAACT TAAAGGAATT GGCGGGGGAG CACTGCAACG GGTGGAGCGT ACGGTTTAAT TGGATTCAAC GCCGAAAACC TCACCGGAGG CGACAGCTGG

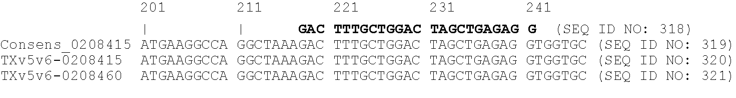

PTM14
PTM14 forward primer (SEQ ID NO: 21)
PTM14 reverse primer (SEQ ID NO: 22)
reverse complement of reverse primer (SEQ ID NO: 322)
CONSENS_0208552 (SEQ ID NO: 323)
TXv5v6-0208552 (SEQ ID NO: 324)
TXv5v6-0208531 (SEQ ID NO: 325)

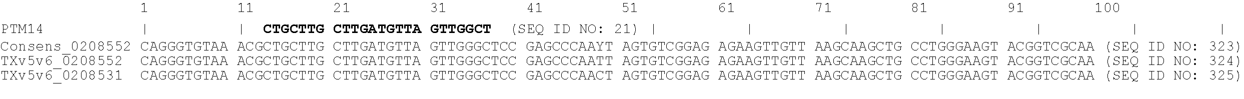

                                101 111 121 131 141 151 161 171 181 191 200
                                |  |  |  |  |  |  |  |  |  |  |
Consens_0208552 GRCTGAAACT TAAAGGAATT GGCGGGGGAG CACAGCAACG GGTGGAGCGT GCGGTTTAAT TGGATTCAAC GCCGGAAAAC TCACCGGAGG CGACGGTTAC
TXv5v6-0208552 GACTGAAACT TAAAGGAATT GGCGGGGGAG CACAGCAACG GGTGGAGCGT GCGGTTTAAT TGGATTCAAC GCCGGAAAAC TCACCGGAGG CGACGGTTAC
TXv5v6-0208531 GGCTGAAACT TAAAGGAATT GGCGGGGGAG CACAGCAACG GGTGGAGCGT GCGGTTTAAT TGGATTCAAC GCCGGAAAAC TCACCGGAGG CGACGGTTAC

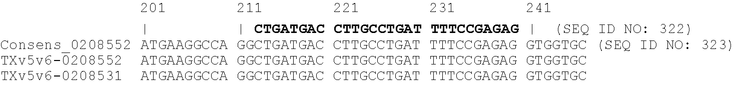

PTM15
PTM15 forward primer (SEQ ID NO: 23)
PTM15 reverse primer (SEQ ID NO: 24)
reverse complement of reverse primer (SEQ ID NO: 326)
CONSENS_0217476 (SEQ ID NO: 327)
TXv5v6-0217476 (SEQ ID NO: 328)
TXv5v6-0219822 (SEQ ID NO: 329)
TXv5v6-0219861 (SEQ ID NO: 330)
TXv5v6-0219863 (SEQ ID NO: 331)
TXv5v6-0219845 (SEQ ID NO: 332)

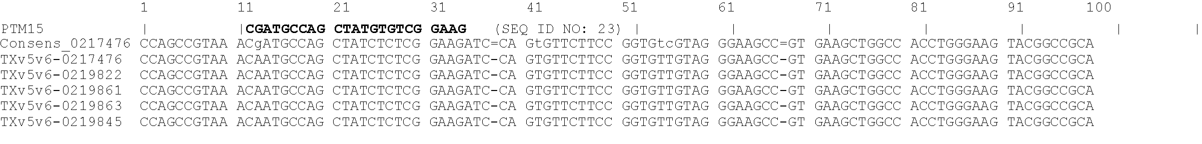

                                101 111 121 131 141 151 161 171 181 191 200
                                |  |  |  |  |  |  |  |  |  |  |
Consens_0217476 AGGCTGAAAC TTAAAGGAAT TGGCGGGGGA GTACTACAAC CGGTGGAGCT TGCGGTTTAA TTGGATACAA CGCCGGAAAT CT=ACCGGGG GCGACAGCAG
TXv5v6-0217476 AGGCTGAAAC TTAAAGGAAT TGGCGGGGGA GTACTACAAC CGGTGGAGCT TGCGGTTTAA TTGGATACAA CGCCGGAAAT CT-ACCGGGG GCGACAGCAG
TXv5v6-0219822 AGGCTGAAAC TTAAAGGAAT TGGCGGGGGA GTACTACAAC CGGTGGAGCT TGCGGTTTAA TTGGATACAA CGCCGGAAAT CT-ACCGGGG GCGACAGCAG
TXv5v6-0219861 AGGCTGAAAC TTAAAGGAAT TGGCGGGGGA GTACTACAAC CGGTGGAGCT TGCGGTTTAA TTGGATACAA CGCCGGAAAT CT-ACCGGGG GCGACAGCAG
TXv5v6-0219863 AGGCTGAAAC TTAAAGGAAT TGGCGGGGGA GTACTACAAC CGGTGGAGCT TGCGGTTTAA TTGGATACAA CGCCGGAAAT CT-ACCGGGG GCGACAGCAG
TXv5v6-0219845 AGGCTGAAAC TTAAAGGAAT TGGCGGGGGA GTACTACAAC CGGTGGAGCT TGCGGTTTAA TTGGATACAA CGCCGGAAAT CT-ACCGGGG GCGACAGCAG

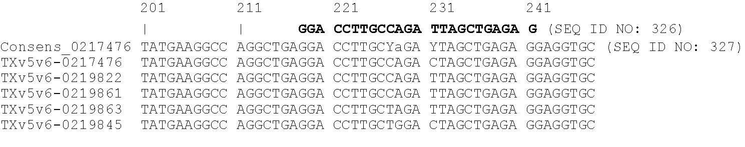

PTM16
PTM16 forward primer (SEQ ID NO: 25)
PTM16 reverse primer (SEQ ID NO: 26)
reverse complement of reverse primer (SEQ ID NO: 333)
CONSENS_0219799 (SEQ ID NO: 334)
TXv5v6-0219799 (SEQ ID NO: 335)
TXv5v6-0219794 (SEQ ID NO: 336)
TXv5v6-0596935 (SEQ ID NO: 337)
TXv5v6-0219795 (SEQ ID NO: 338)

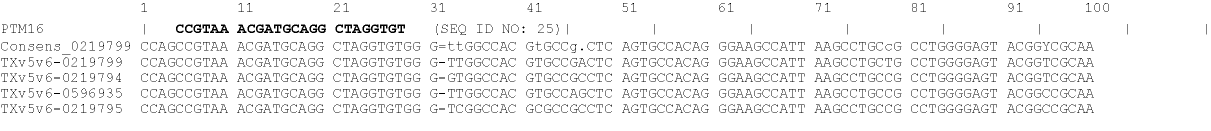

                                101 111 121 131 141 151 161 171 181 191 200
                                |  |  |  |  |  |  |  |  |  |  |
Consens_0219799 GGCTGAAACT TAAAGGAATT GGCGGGgGAG CACCACCAGG CGTGAAGCCT GCGGTTTAAT TGGAGTCAAC GCCGGGAAcC TTACCGGGAG CGACAGCAGA
TXv5v6-0219799 GGCTGAAACT TAAAGGAATT GGCGGGAGAG CACCACCAGG CGTGAAGCCT GCGGTTTAAT TGGAGTCAAC GCCGGGAACC TTACCGGGAG CGACAGCAGA
TXv5v6-0219794 GGCTGAAACT TAAAGGAATT GGCGGGGGAG CACCACCAGG CGTGAAGCCT GCGGTTTAAT TGGAGTCAAC GCCGGGAACC TTACCGGGAG CGACAGCAGA
TXv5v6-0596935 GGCTGAAACT TAAAGGAATT GGCGGGGGAG CACCACCAGG CGTGAAGCCT GCGGTTTAAT TGGAGTCAAC GCCGGGAATC TTACCGGGAG CGACAGCAGA
TXv5v6-0219795 GGCTGAAACT TAAAGGAATT GGCGGGGGAG CACCACCAGG CGTGAAGCCT GCGGTTTAAT TGGAGTCAAC GCCGGGAACC TTACCGGGAG CGACAGCAGA

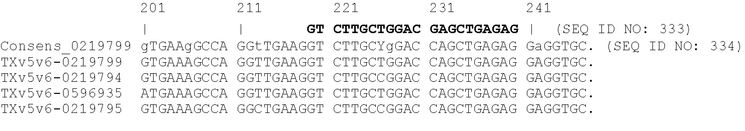

PTM17
PTM17 forward primer (SEQ ID NO: 27)
PTM17 reverse primer (SEQ ID NO: 28)
reverse complement of reverse primer (SEQ ID NO: 339)
CONSENS_0235530 (SEQ ID NO: 340)
TXv5v6-0235530 (SEQ ID NO: 341)
TXv5v6-0235545 (SEQ ID NO: 342)

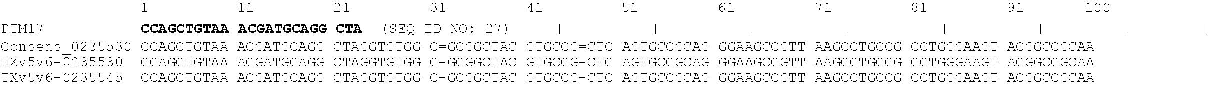

                                101 111 121 131 141 151 161 171 181 191 200
                                |  |  |  |  |  |  |  |  |  |  |
Consens_0235530 GGCTGAAACT TAAAGGAATT GGCGGGGGAG CACCACAAGG KGTGAAGCTT GCGGTTTAAT TGGAGTCAAC GCCGGAAATC TCACCGGGGG CGACAGCAGA
TXv5v6-0235530 GGCTGAAACT TAAAGGAATT GGCGGGGGAG CACCACAAGG GGTGAAGCTT GCGGTTTAAT TGGAGTCAAC GCCGGAAATC TCACCGGGGG CGACAGCAGA
TXv5v6-0235545 GGCTGAAACT TAAAGGAATT GGCGGGGGAG CACCACAAGG TGTGAAGCTT GCGGTTTAAT TGGAGTCAAC GCCGGAAATC TCACCGGGGG CGACAGCAGA

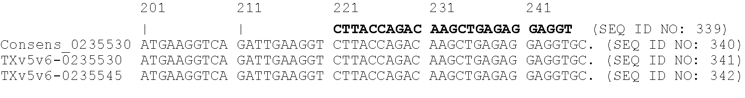

PTM18
PTM18 forward primer (SEQ ID NO: 29)
PTM18 reverse primer (SEQ ID NO: 30)
reverse complement of reverse primer (SEQ ID NO: 343)
CONSENS_0242586 (SEQ ID NO: 344)
TXv5v6-0242586 (SEQ ID NO: 345)
TXv5v6-0242630 (SEQ ID NO: 346)
TXv5v6-0647404 (SEQ ID NO: 347)
TXv5v6-0242596 (SEQ ID NO: 348)
TXv5v6-0242606 (SEQ ID NO: 349)
TXv5v6-0642293 (SEQ ID NO: 350)
TXv5v6-0651560 (SEQ ID NO: 351)
TXv5v6-0644101 (SEQ ID NO: 352)
TXv5v6-0242619 (SEQ ID NO: 353)
TXv5v6-0646437 (SEQ ID NO: 354)
TXv5v6-0641596 (SEQ ID NO: 355)
TXv5v6-0644254 (SEQ ID NO: 356)
TXv5v6-0643665 (SEQ ID NO: 357)
TXv5v6-0647677 (SEQ ID NO: 358)

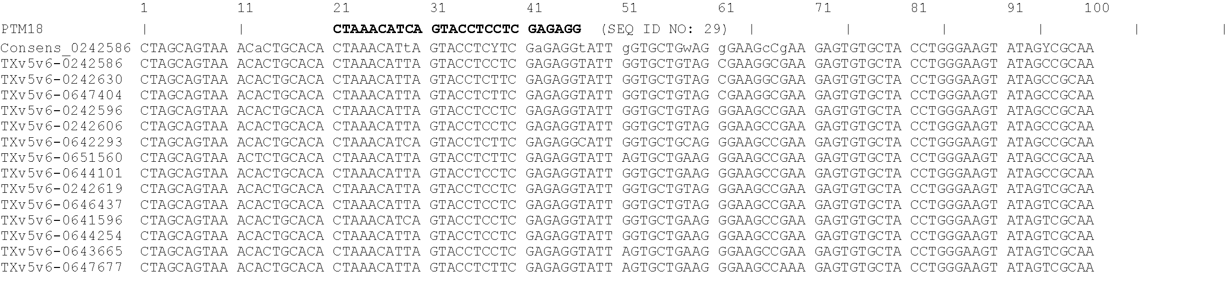

                                101 111 121 131 141 151 161 171 181 191 200
                                |  |  |  |  |  |  |  |  |  |  |
Consens_0242586 GGCcGAAACT TAAAGGAATW GGCGGGGAGa CACTACAACR GGTGACGCGT GCGGTTCAAT TAGATTaTAC ACCGTGAAcC TcACCAGGag CGAcAGCAGa
TXv5v6-0242586 GGCCGAAACT TAAAGGAATA GGCGGGGAGG CACTACAACG GGTGACGCGT GCGGTTCAAT TAGATTATAC ACCGTGAACC TCACCAGGAG CGATAGCAGA
TXv5v6-0242630 GGCCGAAACT TAAAGGAATA GGCGGGGAGG CACTACAACG GGTGACGCGT GCGGTTCAAT TAGATTATAC ACCGTGAACC TCACCAGGAG CGATAGCAGA
TXv5v6-0647404 GGCCGAAACT TAAAGGAATA GGCGGGGAGA CACTACAACG GGTGACGCGT GCGGTTCAAT TAGATTATAC ACCGTGAACC TCACCAGGAG CGATAGCAGA
TXv5v6-0242596 GGCCGAAACT TAAAGGAATA GGCGGGGAGA CACTACAACG GGTGACGCGT GCGGTTCAAT TAGATTATAC ACCGTGAACC TCACCAGGAG CGATAGCAGA
TXv5v6-0242606 GGCCGAAACT TAAAGGAATA GGCGGGGAGA CACTACAACG GGTGACGCGT GCGGTTCAAT TAGATTCTAC ACCGTGAACC TCACCAGGAG CGACAGCAGG
TXv5v6-0642293 GGCCGAAACT TAAAGGAATA GGCGGGGAGG CACTACAACG GGTGACGCGT GCGGTTCAAT TAGATTATAC ACCGTGAACC TCACCAGGAG CGACAGCAGA
TXv5v6-0651560 GGCCGAAACT TAAAGGAATT GGCGGGGAGA CACTACAACA GGTGACGCGT GCGGTTCAAT TAGATTATAC ACCGTGAACC TCACCAGGAG CGACAGCAGA
TXv5v6-0644101 GGCCGAAACT TAAAGGAATT GGCGGGGAGA CACTACAACA GGTGACGCGT GCGGTTCAAT TAGATTATAC ACCGTGAACC TCACCAGGAG CGACAGCAGG
TXv5v6-0242619 GGCCGAAACT TAAAGGAATT GGCGGGGAGA CACTACAACG GGTGACGCGT GCGGTTCAAT TAGATTATAC ACCGTGAACC TCACCAGGAG CGACAGCAGA
TXv5v6-0646437 GGCCGAAACT TAAAGGAATT GGCGGGGAGA CACTACAACG GGTGACGCGT GCGGTTCAAT TAGATTATAC ACCGTGAACC TCACCAGGGG CGACAGCAGA
TXv5v6-0641596 GGCCGAAACT TAAAGGAATT GGCGGGGAGA CACTACAACA GGTGACGCGT GCGGTTCAAT TAGATTATAC ACCGTGAACC TCACCAGGAG CGACAGCAGA
TXv5v6-0644254 GGCCGAAACT TAAAGGAATT GGCGGGGAGA CACTACAACA GGTGACGCGT GCGGTTCAAT TAGATTATAC ACCGTGAACC TTACCAGGAC CGACAGCAGA
TXv5v6-0643665 GGCCGAAACT TAAAGGAATT GGCGGGGAGG CACTACAACG GGTGACGCGT GCGGTTCAAT TAGATTATAC ACCGTGAACC TCACCAGGAG CGACAGCAGA
TXv5v6-0647677 GGCTGAAACT TAAAGGAATT GGCGGGGAGA CACTACAACA GGTGACGCGT GCGGTTCAAT TAGATTATAC ACCGTGAATC TCACCAGGAC CGACAGCAGA

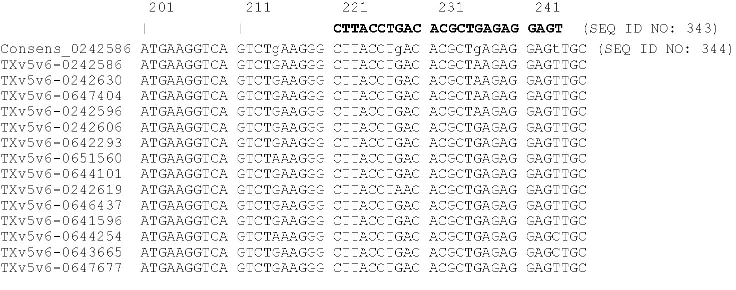

PTM19
PTM19 forward primer (SEQ ID NO: 31)
PTM19 reverse primer (SEQ ID NO: 32)
reverse complement of reverse primer (SEQ ID NO: 359)
CONSENS_0242690 (SEQ ID NO: 360)
TXv5v6-0242690 (SEQ ID NO: 361)
TXv5v6-0242726 (SEQ ID NO: 362)

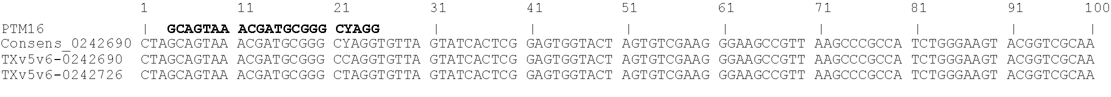

                                101 111 121 131 141 151 161 171 181 191 200
                                |  |  |  |  |  |  |  |  |  |  |

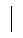

Consens_0242690 GGCTGAAACT TAAAGGAATT GGCGGGGGAG CACTACAACG GGTGGAGCTT GCGGTTTAAT TGGATTCAAC GCCGTGAATC TTACCGGGGA AGACAGCAAG
TXv5v6-0242690 GGCTGAAACT TAAAGGAATT GGCGGGGGAG CACTACAACG GGTGGAGCTT GCGGTTTAAT TGGATTCAAC GCCGTGAATC TTACCGGGGA AGACAGCAAG
TXv5v6-0242726 GGCTGAAACT TAAAGGAATT GGCGGGGGAG CACTACAACG GGTGGAGCTT GCGGTTTAAT TGGATTCAAC GCCGTGAATC TTACCGGGGA AGACAGCAAG

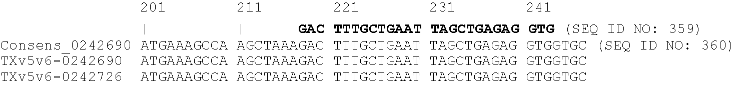

PTM20
PTM20 forward primer (SEQ ID NO: 33)
PTM20 reverse primer (SEQ ID NO: 34)
reverse complement of reverse primer (SEQ ID NO: 363)
CONSENS_0248376 (SEQ ID NO: 364)
TXv5v6-0248376 (SEQ ID NO: 365)
TXv5v6-0671483 (SEQ ID NO: 366)

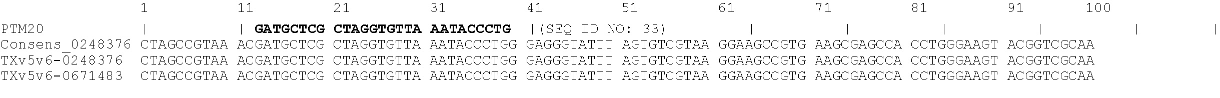

                                101 111 121 131 141 151 161 171 181 191 200
                                |  |  |  |  |  |  |  |  |  |  |
Consens_0248376 GGCTGAAACT TAAAGGAATT GGCGGGGGAG CACAACAACG GGTGGATGCT GCGGTTTAAT TGGATTCAAC GCCGGAAATC TTACCGGAGG CGACAG=AAT
TXv5v6-0248376 GGCTGAAACT TAAAGGAATT GGCGGGGGAG CACAACAACG GGTGGATGCT GCGGTTTAAT TGGATTCAAC GCCGGAAATC TTACCGGAGG CGACAG-AAT
TXv5v6-0671483 GGCTGAAACT TAAAGGAATT GGCGGGGGAG CACAACAACG GGTGGATGCT GCGGTTTAAT TGGATTCAAC GCCGGAAATC TTACCGGAGG CGACAG-AAT

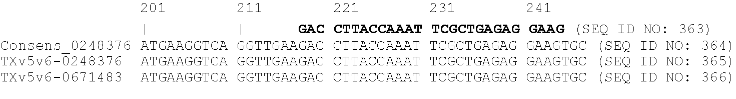

PTM21
PTM21 forward primer (SEQ ID NO: 35)
PTM21 reverse primer (SEQ ID NO: 36)
reverse complement of reverse primer (SEQ ID NO: 367)
CONSENS_0266750 (SEQ ID NO: 368)
TXv5v6-0266750 (SEQ ID NO: 369)
TXv5v6-0771140 (SEQ ID NO: 370)
TXv5v6-0770570 (SEQ ID NO: 371)

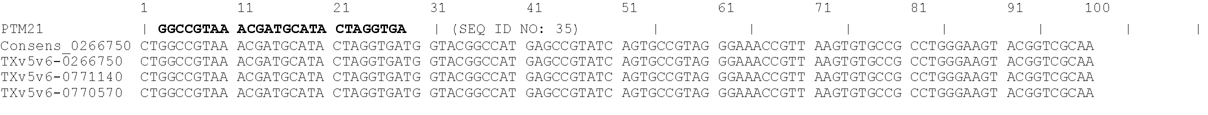

                                101 111 121 131 141 151 161 171 181 191 200
                                |  |  |  |  |  |  |  |  |  |  |
Consens_0266750 GGCTAAAACT TAAAGGAATT GGC.GGGGAG CACCACAAGG GGTGAAGCCT GCGGTTCAAT TGGACTCAAC GCCGGGAAAC TTACCAGGGG AGACAGCAGT
TXv5v6-0266750 GGCTAAAACT TAAAGGAATT GGCGGGGGAG CACCACAAGG GGTGAAGCCT GCGGTTCAAT TGGACTCAAC GCCGGGAAAC TTACCAGGGG AGACAGCAGT
TXv5v6-0771140 GGCTAAAACT TAAAGGAATT GGCGGGGGAG CACCACAAGG GGTGAAGCCT GCGGTTCAAT TGGACTCAAC GCCGGGAAAC TTACCAGGGG AGACAGCAGT
TXv5v6-0770570 GGCTAAAACT TAAAGGAATT GGC-GGGGAG CACCACAAGG GGTGAAGCCT GCGGTTCAAT TGGACTCAAC GCCGGGAAAC TTACCAGGGG AGACAGCAGT

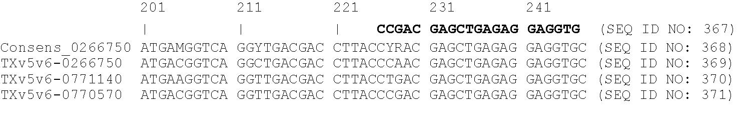

PTM22
PTM22 forward primer (SEQ ID NO: 37)
PTM22 reverse primer (SEQ ID NO: 38)
reverse complement of reverse primer (SEQ ID NO: 372)
CONSENS_0266796 (SEQ ID NO: 373)
TXv5v6-0266796 (SEQ ID NO: 374)
TXv5v6-0772899 (SEQ ID NO: 375)

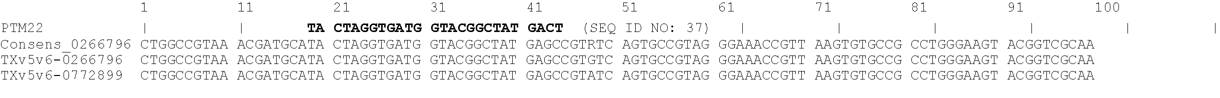

                                101 111 121 131 141 151 161 171 181 191 200
                                |  |  |  |  |  |  |  |  |  |  |
Consens_0266796 GGCTAAAACT TAAAGGAATT GGCGGGGGAG CACCACAAGG GGTGAAGCCT GCGGTTCAAT TGGACTCAAC GCCGGGAAAC TTACCAGGGG AGACAGCAGW
TXv5v6-0266796 GGCTAAAACT TAAAGGAATT GGCGGGGGAG CACCACAAGG GGTGAAGCCT GCGGTTCAAT TGGACTCAAC GCCGGGAAAC TTACCAGGGG AGACAGCAGA
TXv5v6-0772899 GGCTAAAACT TAAAGGAATT GGCGGGGGAG CACCACAAGG GGTGAAGCCT GCGGTTCAAT TGGACTCAAC GCCGGGAAAC TTACCAGGGG AGACAGCAGT

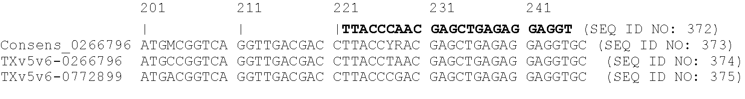

PTM23
PTM23 forward primer (SEQ ID NO: 39)
PTM23 reverse primer (SEQ ID NO: 40)
reverse complement of reverse primer (SEQ ID NO: 376)
CONSENS_0283719 (SEQ ID NO: 377)
TXv5v6-0283719 (SEQ ID NO: 378)
TXv5v6-0283712 (SEQ ID NO: 379)
TXv5v6-0788889 (SEQ ID NO: 380)

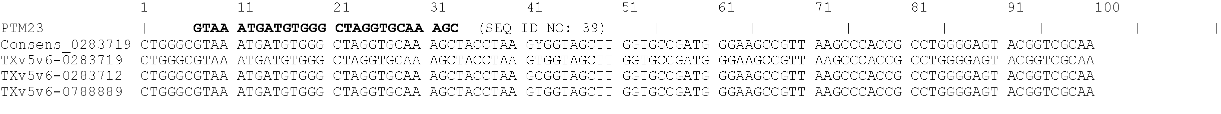

                                101 111 121 131 141 151 161 171 181 191 200
                                |  |  |  |  |  |  |  |  |  |  |
Consens_0283719 GGCTGAAACT TAAAGGAATT GGCGGGKGAG CACCACAAGG GGTGGAGGCT GCGGTTTAAT TGGATTCAAC GCCGGGAAAC TCACCGGGGG CGACAGCAGT
TXv5v6-0283719 GGCTGAAACT TAAAGGAATT GGCGGGGGAG CACCACAAGG GGTGGAGGCT GCGGTTTAAT TGGATTCAAC GCCGGGAAAC TCACCGGGGG CGACAGCAGT
TXv5v6-0283712 GGCTGAAACT TAAAGGAATT GGCGGGTGAG CACCACAAGG GGTGGAGGCT GCGGTTTAAT TGGATTCAAC GCCGGGAAAC TCACCGGGGG CGACAGCAGT
TXv5v6-0788889 GGCTGAAACT TAAAGGAATT GGCGGGTGAG CACCACAAGG GGTGGAGGCT GCGGTTTAAT TGGATTCAAC GCCGGGAAAC TCACCGGGGG CGACAGCAGT

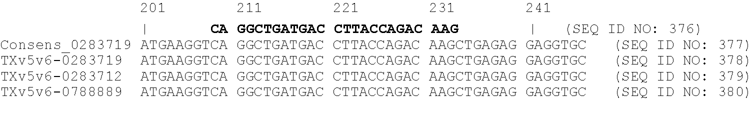

PTM24
PTM24 forward primer (SEQ ID NO: 41)
PTM24 reverse primer (SEQ ID NO: 42)
reverse complement of reverse primer (SEQ ID NO: 381)
CONSENS_0714814 (SEQ ID NO: 382)
TXv5v6-0714814 (SEQ ID NO: 383)
TXv5v6-0257743 (SEQ ID NO: 384)

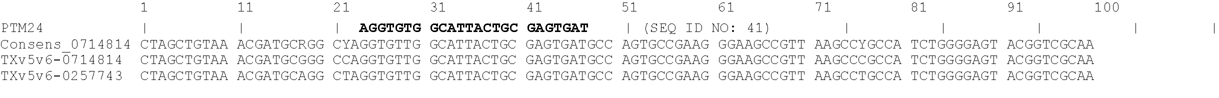

                                101 111 121 131 141 151 161 171 181 191 200
                                |  |  |  |  |  |  |  |  |  |  |
Consens_0714814 GGCTGAAACT TAAAGGAATT GGCGGGGGAG CACYACAACG GGTGGAGCYT GCGGTTCAAT TGGATTCAAC GCCGGAAAMC TCACCGGRGG MGACAGCGAK
TXv5v6-0714814 GGCTGAAACT TAAAGGAATT GGCGGGGGAG CACCACAACG GGTGGAGCTT GCGGTTCAAT TGGATTCAAC GCCGGAAAAC TCACCGGGGG AGACAGCGAG
TXv5v6-0257743 GGCTGAAACT TAAAGGAATT GGCGGGGGAG CACTACAACG GGTGGAGCCT GCGGTTCAAT TGGATTCAAC GCCGGAAACC TCACCGGAGG CGACAGCGAT

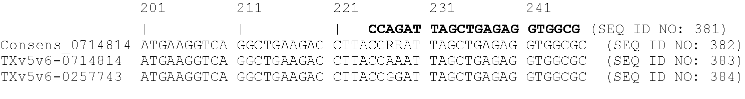

PTM25
PTM25 forward primer (SEQ ID NO: 43)
PTM25 reverse primer (SEQ ID NO: 44)
reverse complement of reverse primer (SEQ ID NO: 385)
CONSENS_1349302 (SEQ ID NO: 386)
TXv5v6-1349302 (SEQ ID NO: 387)
TXv5v6-1349224 (SEQ ID NO: 388)

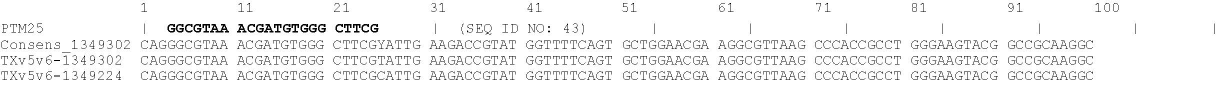

                                101 111 121 131 141 151 161 171 181 191 200
                                |  |  |  |  |  |  |  |  |  |  |
Consens_1349302 TGAAACTTAA AGGAATTGAC GGGGGAGCAC AGCAACGGGA GGAGCGTGCG GTTCAATTGG ATTCAACGCC GGAAAACTCA CCGGAGGAGA CTGCCAGATG
TXv5v6-1349302 TGAAACTTAA AGGAATTGAC GGGGGAGCAC AGCAACGGGA GGAGCGTGCG GTTCAATTGG ATTCAACGCC GGAAAACTCA CCGGAGGAGA CTGCCAGATG
TXv5v6-1349224 TGAAACTTAA AGGAATTGAC GGGGGAGCAC AGCAACGGGA GGAGCGTGCG GTTCAATTGG ATTCAACGCC GGAAAACTCA CCGGAGGAGA CTGCCAGATG

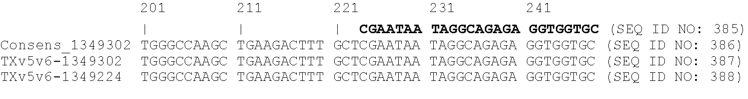

PTM26
PTM26 forward primer (SEQ ID NO: 45)
PTM26 reverse primer (SEQ ID NO: 46)
reverse complement of reverse primer (SEQ ID NO: 389)
CONSENS_1689428 (SEQ ID NO: 390)
TXv5v6-1689428 (SEQ ID NO: 391)
TXv5v6-1425443 (SEQ ID NO: 392)
TXv5v6-1688200 (SEQ ID NO: 393)
TXv5v6-0257863 (SEQ ID NO: 394)
TXv5v6-0716397 (SEQ ID NO: 395)
TXv5v6-0258422 (SEQ ID NO: 396)
TXv5v6-0258367 (SEQ ID NO: 397)
TXv5v6-0258396 (SEQ ID NO: 398)
TXv5v6-1689332 (SEQ ID NO: 399)
TXv5v6-0715252 (SEQ ID NO: 400)
TXv5v6-0258423 (SEQ ID NO: 401)
TXv5v6-0258384 (SEQ ID NO: 402)
TXv5v6-0258379 (SEQ ID NO: 403)
TXv5v6-1425442 (SEQ ID NO: 404)
TXv5v6-0258269 (SEQ ID NO: 405)
TXv5v6-1689136 (SEQ ID NO: 406)
TXv5v6-0258307 (SEQ ID NO: 407)
TXv5v6-1689106 (SEQ ID NO: 408)
TXv5v6-0258247 (SEQ ID NO: 409)
TXv5v6-0258276 (SEQ ID NO: 410)
TXv5v6-0258315 (SEQ ID NO: 411)

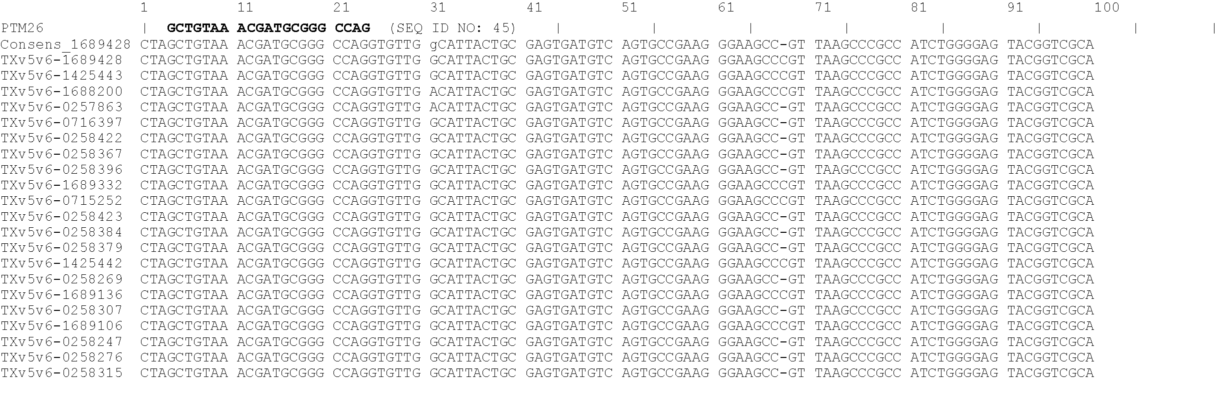

                                101 111 121 131 141 151 161 171 181 191 200
                                |  |  |  |  |  |  |  |  |  |  |
Consens_1689428 AGGCTGAAAC TTAAAGGAAT TGGCGGGGGA GCACCACAAC GGGTGGAGCC TGCGGTTCAA TTGGATTCAA CGCCGGRAAA CTCACCGGAG GCGACAGCAA
TXv5v6-1689428 AGGCTGAAAC TTAAAGGAAT TGGCGGGGGA GCACCACAAC GGGTGGAGCC TGCGGTTCAA TTGGATTCAA CGCCGGGAAA CTCACCGGAG GCGACAGCAA
TXv5v6-1425443 AGGCTGAAAC TTAAAGGAAT TGGCGGGGGA GCACCACAAC GGGTGGAGCC TGCGGTTCAA TTGGATTCAA CGCCGGGAAA CTCACCGGAG GCGACAGCAA
TXv5v6-1688200 AGGCTGAAAC TTAAAGGAAT TGGCGGGGGA GCACCACAAC GGGTGGAGCC TGCGGTTCAA TTGGATTCAA CGCCGGGAAA CTCACCGGAG GCGACAGCAA
TXv5v6-0257863 AGGCTGAAAC TTAAAGGAAT TGGCGGGGGA GCACCACAAC GGGTGGAGCC TGCGGTTCAA TTGGATTCAA CGCCGGGAAA CTCACCGGAG GCGACAGCAA
TXv5v6-0716397 AGGCTGAAAC TTAAAGGAAT TGGCGGGGGA GCACCACAAC GGGTGGAGCC TGCGGTTCAA TTGGATTCAA CGCCGGGAAA CTCACCGGAG GCGACAGCAA
TXv5v6-0258422 AGGCTGAAAC TTAAAGGAAT TGGCGGGGGA GCACCACAAC GGGTGGAGCC TGCGGTTCAA TTGGATTCAA CGCCGGGAAA CTCACCGGAG GCGACAGCAA
TXv5v6-0258367 AGGCTGAAAC TTAAAGGAAT TGGCGGGGGA GCACCACAAC GGGTGGAGCC TGCGGTTCAA TTGGATTCAA CGCCGGGAAA CTCACCGGAG GCGACAGCAA
TXv5v6-0258396 AGGCTGAAAC TTAAAGGAAT TGGCGGGGGA GCACCACAAC GGGTGGAGCC TGCGGTTCAA TTGGATTCAA CGCCGGGAAA CTCACCGGAG GCGACAGCAA
TXv5v6-1689332 AGGCTGAAAC TTAAAGGAAT TGGCGGGGGA GCACCACAAC GGGTGGAGCC TGCGGTTCAA TTGGATTCAA CGCCGGGAAA CTCACCGGAG GCGACAGCAA
TXv5v6-0715252 AGGCTGAAAC TTAAAGGAAT TGGCGGGGGA GCACCACAAC GGGTGGAGCC TGCGGTTCAA TTGGATTCAA CGCCGGGAAA CTCACCGGAG GCGACAGCAA
TXv5v6-0258423 AGGCTGAAAC TTAAAGGAAT TGGCGGGGGA GCACCACAAC GGGTGGAGCC TGCGGTTCAA TTGGATTCAA CGCCGGGAAA CTCACCGGAG GCGACAGCAA
TXv5v6-0258384 AGGCTGAAAC TTAAAGGAAT TGGCGGGGGA GCACCACAAC GGGTGGAGCC TGCGGTTCAA TTGGATTCAA CGCCGGGAAA CTCACCGGAG GCGACAGCAA
TXv5v6-0258379 AGGCTGAAAC TTAAAGGAAT TGGCGGGGGA GCACCACAAC GGGTGGAGCC TGCGGTTCAA TTGGATTCAA CGCCGGGAAA CTCACCGGAG GCGACAGCAA
TXv5v6-1425442 AGGCTGAAAC TTAAAGGAAT TGGCGGGGGA GCACCACAAC GGGTGGAGCC TGCGGTTCAA TTGGATTCAA CGCCGGGAAA CTCACCGGAG GCGACAGCAA
TXv5v6-0258269 AGGCTGAAAC TTAAAGGAAT TGGCGGGGGA GCACCACAAC GGGTGGAGCC TGCGGTTCAA TTGGATTCAA CGCCGGAAAA CTCACCGGAG GCGACAGCAA
TXv5v6-1689136 AGGCTGAAAC TTAAAGGAAT TGGCGGGGGA GCACCACAAC GGGTGGAGCC TGCGGTTCAA TTGGATTCAA CGCCGGAAAA CTCACCGGAG GCGACAGCAA
TXv5v6-0258307 AGGCTGAAAC TTAAAGGAAT TGGCGGGGGA GCACCACAAC GGGTGGAGCC TGCGGTTCAA TTGGATTCAA CGCCGGAAAA CTCACCGGAG GCGACAGCAA
TXv5v6-1689106 AGGCTGAAAC TTAAAGGAAT TGGCGGGGGA GCACCACAAC GGGTGGAGCC TGCGGTTCAA TTGGATTCAA CGCCGGAAAA CTCACCGGAG GCGACAGCAA
TXv5v6-0258247 AGGCTGAAAC TTAAAGGAAT TGGCGGGGGA GCACCACAAC GGGTGGAGCC TGCGGTTCAA TTGGATTCAA CGCCGGAAAA CTCACCGGAG GCGACAGCAA
TXv5v6-0258276 AGGCTGAAAC TTAAAGGAAT TGGCGGGGGA GCACCACAAC GGGTGGAGCC TGCGGTTCAA TTGGATTCAA CGCCGGAAAA CTCACCGGAG GCGACAGCAA
TXv5v6-0258315 AGGCTGAAAC TTAAAGGAAT TGGCGGGGGA GCACCACAAC GGGTGGAGCC TGCGGTTCAA TTGGATTCAA CGCCGGAAAA CTCACCGGAG GCGACAGCGA

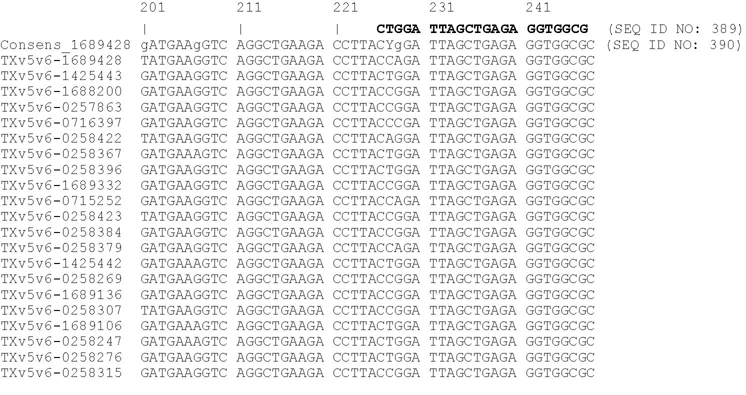

PTM27
PTM27 forward primer (SEQ ID NO: 47)
PTM27 reverse primer (SEQ ID NO: 48)
reverse complement of reverse primer (SEQ ID NO: 412)
CONSENS_1671056 (SEQ ID NO: 413)
TXv5v6-1671056 (SEQ ID NO: 414)
TXv5v6-0237067 (SEQ ID NO: 415)
TXv5v6-1672136 (SEQ ID NO: 416)
TXv5v6-0237299 (SEQ ID NO: 417)
TXv5v6-0237037 (SEQ ID NO: 418)
TXv5v6-1376733 (SEQ ID NO: 419)
TXv5v6-0237185 (SEQ ID NO: 420)
TXv5v6-0237083 (SEQ ID NO: 421)
TXv5v6-1377062 (SEQ ID NO: 422)
TXv5v6-0236558 (SEQ ID NO: 423)
TXv5v6-0237291 (SEQ ID NO: 424)
TXv5v6-0236906 (SEQ ID NO: 425)
TXv5v6-0236917 (SEQ ID NO: 426)
TXv5v6-0624771 (SEQ ID NO: 427)
TXv5v6-0236386 (SEQ ID NO: 428)
TXv5v6-0236838 (SEQ ID NO: 429)
TXv5v6-0236818 (SEQ ID NO: 430)
TXv5v6-0236985 (SEQ ID NO: 431)
TXv5v6-0621787 (SEQ ID NO: 432)

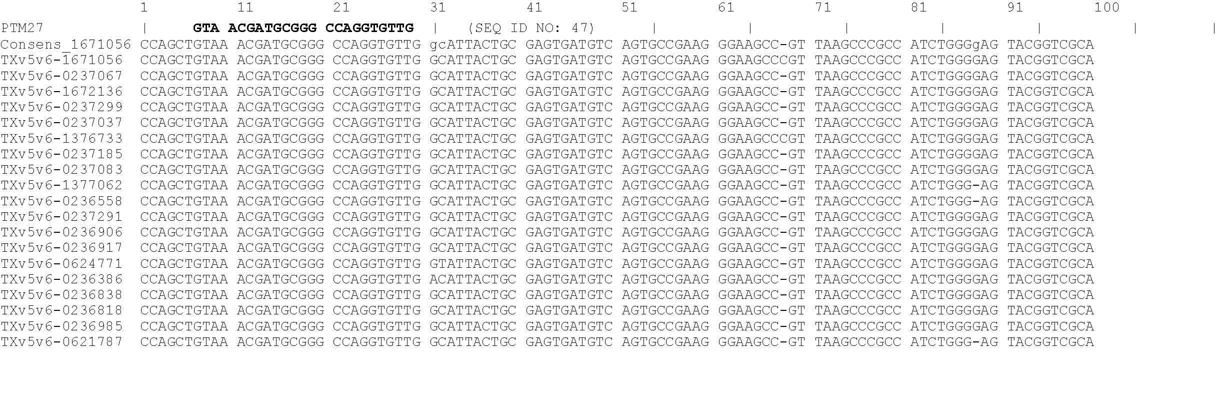

                                101 111 121 131 141 151 161 171 181 191 200
                                |  |  |  |  |  |  |  |  |  |  |
Consens_1671056 AGGCTGAAAC TTAAAGaAAT TGGCGGGGGA GCACCACAAC GGGTGGAGCc TGCGGTTCAA TYGGATTCAA CGCCGGAAAa CTCACCGGAG GCgACAGCgA
TXv5v6-1671056 AGGCTGAAAC TTAAAGAAAT TGGCGGGGGA GCACCACAAC GGGTGGAGCC TGCGGTTCAA TTGGATTCAA CGCCGGAAAA CTCACCGGAG GCGACAGCGA
TXv5v6-0237067 AGGCTGAAAC TTAAAGAAAT TGGCGGGGGA GCACCACAAC GGGTGGAGCC TGCGGTTCAA TTGGATTCAA CGCCGGAAAA CTCACCGGAG GCGACAGCGA
TXv5v6-1672136 AGGCTGAAAC TTAAAGAAAT TGGCGGGGGA GCACCACAAC GGGTGGAGCC TGCGGTTCAA TTGGATTCAA CGCCGGAAAA CTCACCGGAG GCGACAGCGA
TXv5v6-0237299 AGGCTGAAAC TTAAAGGAAT TGGCGGGGGA GCACCACAAC GGGTGGAGCC TGCGGTTCAA TTGGATTCAA CGCCGGAAAA CTCACCGGAG GCGACAGCGA
TXv5v6-0237037 AGGCTGAAAC TTAAAGAAAT TGGCGGGGGA GCACCACAAC GGGTGGAGCC TGCGGTTCAA TTGGATTCAA CGCCGGAAAA CTCACCGGAG GCAACAGCGA
TXv5v6-1376733 AGGCTGAAAC TTAAAGAAAT TGGCGGGGGA GCACCACAAC GGGTGGAGCC TGCGGTTCAA TTGGATTCAA CGCCGGAAAA CTCACCGGAG GCGACAGCGA
TXv5v6-0237185 AGGCTGAAAC TTAAAGAAAT TGGCGGGGGA GCACCACAAC GGGTGGAGCT TGCGGTTCAA TTGGATTCAA CGCCGGAAAA CTCACCGGAG GCGACAGCGA
TXv5v6-0237083 AGGCTGAAAC TTAAAGAAAT TGGCGGGGGA GCACCACAAC GGGTGGAGCC TGCGGTTCAA TTGGATTCAA CGCCGGAAAA CTCACCGGAG GCGACAGCGA
TXv5v6-1377062 AGGCTGAAAC TTAAAGAAAT TGGCGGGGGA GCACCACAAC GGGTGGAGCT TGCGGTTCAA TTGGATTCAA CGCCGGAAAA CTCACCGGAG GCGACAGCGA
TXv5v6-0236558 AGGCTGAAAC TTAAAGAAAT TGGCGGGGGA GCACCACAAC GGGTGGAGCC TGCGGTTCAA TTGGATTCAA CGCCGGAAAA CTCACCGGAG GCGACAGCGA
TXv5v6-0237291 AGGCTGAAAC TTAAAGGAAT TGGCGGGGGA GCACCACAAC GGGTGGAGCC TGCGGTTCAA TCGGATTCAA CGCCGGAAAA CTCACCGGAG GCGACAGCAA
TXv5v6-0236906 AGGCTGAAAC TTAAAGAAAT TGGCGGGGGA GCACCACAAC GGGTGGAGCC TGCGGTTCAA TCGGATTCAA CGCCGGAAAA CTCACCGGAG GCGACAGCGA
TXv5v6-0236917 AGGCTGAAAC TTAAAGAAAT TGGCGGGGGA GCACCACAAC GGGTGGAGCC TGCGGTTCAA TCGGATTCAA CGCCGGAAAA CTCACCGGAG GCGACAGCGA
TXv5v6-0624771 AGGCTGAAAC TTAAAGAAAT TGGCGGGGGA GCACCACAAC GGGTGGAGCC TGCGGTTCAA TCGGATTCAA CGCCGGAAAA CTCACCGGAG GCGACAGCGA
TXv5v6-0236386 AGGCTGAAAC TTAAAGAAAT TGGCGGGGGA GCACCACAAC GGGTGGAGCC TGCGGTTCAA TCGGATTCAA CGCCGGAAAA CTCACCGGAG GCGACAGCGA
TXv5v6-0236838 AGGCTGAAAC TTAAAGAAAT TGGCGGGGGA GCACCACAAC GGGTGGAGCC TGCGGTTCAA TCGGATTCAA CGCCGGAAAA CTCACCGGAG GCGACAGCAA
TXv5v6-0236818 AGGCTGAAAC TTAAAGAAAT TGGCGGGGGA GCACCACAAC GGGTGGAGCC TGCGGTTCAA TCGGATTCAA CGCCGGAAAA CTCACCGGAG GCGACAGCAA
TXv5v6-0236985 AGGCTGAAAC TTAAAGAAAT TGGCGGGGGA GCACCACAAC GGGTGGAGCC TGCGGTTCAA TCGGATTCAA CGCCGGAAAT CTCACCGGAG GCGACAGCGA
TXv5v6-0621787 AGGCTGAAAC TTAAAGAAAT TGGCGGGGGA GCACCACAAC GGGTGGAGCC TGCGGTTCAA TCGGATTCAA CGCCGGAAAA CTCACCGGAG GCGACAGCGA

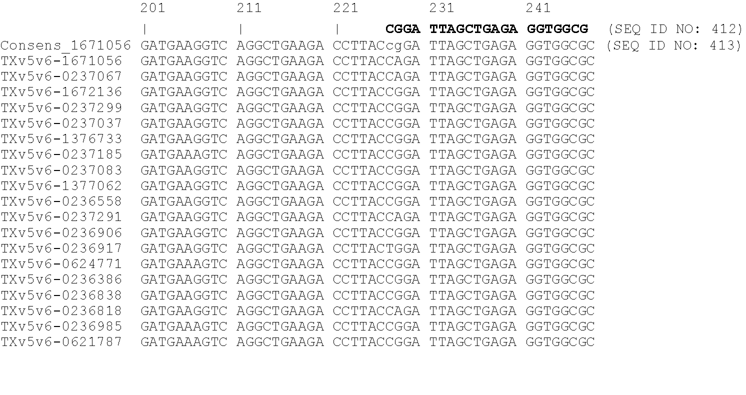

PTM28
PTM28 forward primer (SEQ ID NO: 49)
PTM28 reverse primer (SEQ ID NO: 50)
reverse complement of reverse primer (SEQ ID NO: 433)
CONSENS_0545759 (SEQ ID NO: 434)
TXv5v6-0545759 (SEQ ID NO: 435)
TXv5v6-0194637 (SEQ ID NO: 436)

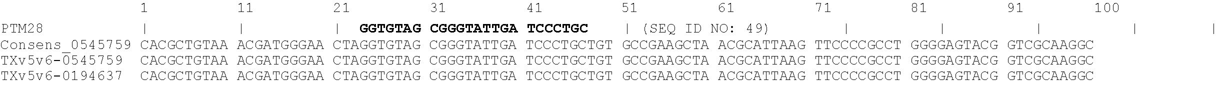

                                101 111 121 131 141 151 161 171 181 191 200
                                |  |  |  |  |  |  |  |  |  |  |
Consens_0545759 TAAAACTCAA AGGAATTGAC GGGGGCCCGC ACAAGCGGTG GAGCATGTGG TTCAATTCGA TGCAACGCGA AAAACCTTAC CTGGGTTTGA CATCCTTTGA
TXv5v6-0545759 TAAAACTCAA AGGAATTGAC GGGGGCCCGC ACAAGCGGTG GAGCATGTGG TTCAATTCGA TGCAACGCGA AAAACCTTAC CTGGGTTTGA CATCCTTTGA
TXv5v6-0194637 TAAAACTCAA AGGAATTGAC GGGGGCCCGC ACAAGCGGTG GAGCATGTGG TTCAATTCGA TGCAACGCGA AAAACCTTAC CTGGGTTTGA CATCCTTTGA

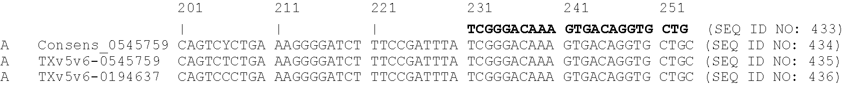

PTM29
PTM29 forward primer (SEQ ID NO: 51)
PTM29 reverse primer (SEQ ID NO: 52)
reverse complement of reverse primer (SEQ ID NO: 437)
CONSENS_0045163 (SEQ ID NO: 438)
TXv5v6-0045163 (SEQ ID NO: 439)
TXv5v6-0045206 (SEQ ID NO: 440)

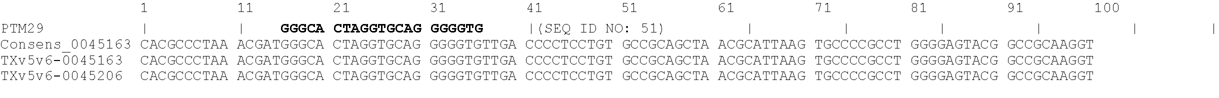

                                101 111 121 131 141 151 161 171 181 191 200
                                |  |  |  |  |  |  |  |  |  |  |
Consens_0045163 TGAAACTCAA AGGAATTGAC GGGGGCCCGC ACAAGCGGTG GAGCATGTGG TTYAATTCGA CGCAACGCGA AGAACCTTAC CTGGGCTTGA CATCCCGGGA
TXv5v6-0045163 TGAAACTCAA AGGAATTGAC GGGGGCCCGC ACAAGCGGTG GAGCATGTGG TTCAATTCGA CGCAACGCGA AGAACCTTAC CTGGGCTTGA CATCCCGGGA
TXv5v6-0045206 TGAAACTCAA AGGAATTGAC GGGGGCCCGC ACAAGCGGTG GAGCATGTGG TTTAATTCGA CGCAACGCGA AGAACCTTAC CTGGGCTTGA CATCCCGGGA

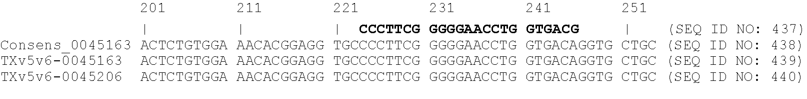

PTM30
PTM30 forward primer (SEQ ID NO: 53)
PTM30 reverse primer (SEQ ID NO: 54)
reverse complement of reverse primer (SEQ ID NO: 441)
CONSENS_0063016 (SEQ ID NO: 442)
TXv5v6-0063016 (SEQ ID NO: 443)
TXv5v6-1284822 (SEQ ID NO: 444)

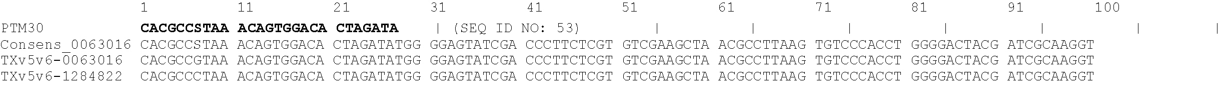

                                101 111 121 131 141 151 161 171 181 191 200
                                |  |  |  |  |  |  |  |  |  |  |
Consens_0063016 TAAAACTCAA AGGAATTGAC GGGGGCCCGC ACAAGCAGCG GAGCGTGTGG TTTAATTCGA TGCTACGCGA AGAACCTTAC CAGGGCTTGA CATGTCAGTA
TXv5v6-0063016 TAAAACTCAA AGGAATTGAC GGGGGCCCGC ACAAGCAGCG GAGCGTGTGG TTTAATTCGA TGCTACGCGA AGAACCTTAC CAGGGCTTGA CATGTCAGTA
TXv5v6-1284822 TAAAACTCAA AGGAATTGAC GGGGGCCCGC ACAAGCAGCG GAGCGTGTGG TTTAATTCGA TGCTACGCGA AGAACCTTAC CAGGGCTTGA CATGTCAGTA

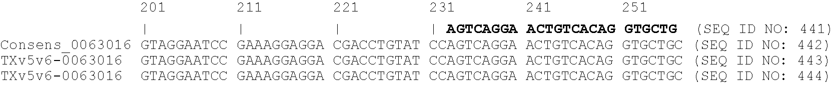

PTM31
PTM31 forward primer (SEQ ID NO: 55)
PTM31 reverse primer (SEQ ID NO: 56)
reverse complement of reverse primer (SEQ ID NO: 445)
CONSENS_0258790 (SEQ ID NO: 446)
TXv5v6-0258790 (SEQ ID NO: 447)
TXv5v6-0717922 (SEQ ID NO: 448)
TXv5v6-0258776 (SEQ ID NO: 449)
TXv5v6-0258773 (SEQ ID NO: 450)
TXv5v6-1691264 (SEQ ID NO: 451)
TXv5v6-0718915 (SEQ ID NO: 452)
TXv5v6-0258774 (SEQ ID NO: 453)

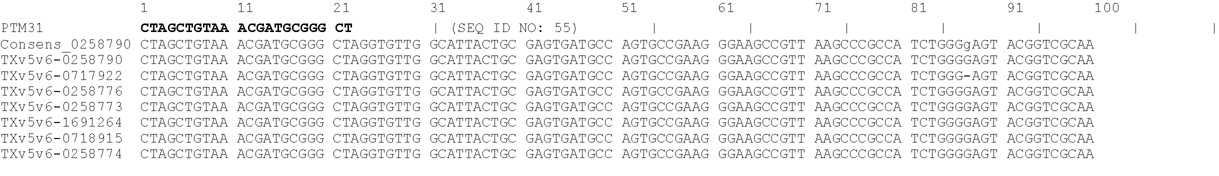

                                101 111 121 131 141 151 161 171 181 191 200
                                |  |  |  |  |  |  |  |  |  |  |
Consens_0258790 GGCTGAAACT TAAAGGAATT GGCGGGGGAG CACTACAACG GGTGGAGCCT GCGGTTCAAT TGGATTCAAC GCCGGaAAAC TCACCGGAGG CGACAGCGAg
TXv5v6-0258790 GGCTGAAACT TAAAGGAATT GGCGGGGGAG CACTACAACG GGTGGAGCCT GCGGTTCAAT TGGATTCAAC GCCGGGAAAC TCACCGGAGG CGACAGCGAG
TXv5v6-0717922 GGCTGAAACT TAAAGGAATT GGCGGGGGAG CACTACAACG GGTGGAGCCT GCGGTTCAAT TGGATTCAAC GCCGGGAAAC TCACCGGAGG CGACAGCGAG
TXv5v6-0258776 GGCTGAAACT TAAAGGAATT GGCGGGGGAG CACTACAACG GGTGGAGCCT GCGGTTCAAT TGGATTCAAC GCCGGAAAAC TCACCGGAGG CGACAGCGAT
TXv5v6-0258773 GGCTGAAACT TAAAGGAATT GGCGGGGGAG CACTACAACG GGTGGAGCCT GCGGTTCAAT TGGATTCAAC GCCGGAAAAC TCACCGGAGG CGACAGCGAG
TXv5v6-1691264 GGCTGAAACT TAAAGGAATT GGCGGGGGAG CACTACAACG GGTGGAGCCT GCGGTTCAAT TGGATTCAAC GCCGGAAAAC TCACCGGAGG CGACAGCGAG
TXv5v6-0718915 GGCTGAAACT TAAAGGAATT GGCGGGGGAG CACTACAACG GGTGGAGCCT GCGGTTCAAT TGGATTCAAC GCCGGAAAAC TCACCGGAGG CGACAGCGAG
TXv5v6-0258774 GGCTGAAACT TAAAGGAATT GGCGGGGGAG CACTACAACG GGTGGAGCCT GCGGTTCAAT TGGATTCAAC GCCGGAAAAC TCACCGGAGG CGACAGCGAG

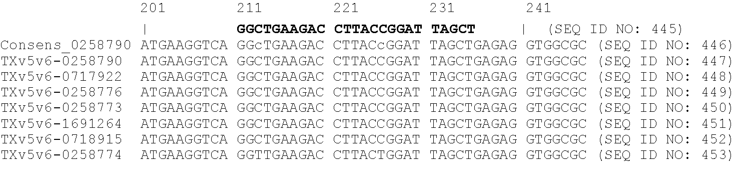

PTM32
PTM32 primer (SEQ ID NO: 57)
PTM32 reverse primer (SEQ ID NO: 58)
reverse complement of reverse primer (SEQ ID NO: 454)
CONSENS_0252248 (SEQ ID NO: 455)
TXv5v6-0252248 (SEQ ID NO: 456)
TXv5v6-0689158 (SEQ ID NO: 457)
TXv5v6-0252247 (SEQ ID NO: 458)

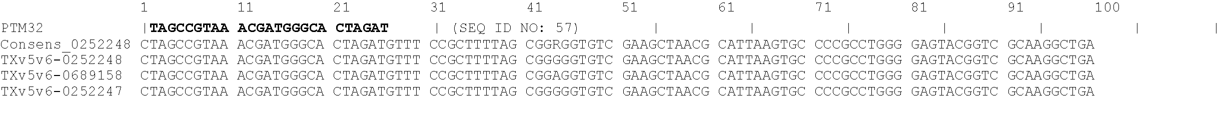

                                101 111 121 131 141 151 161 171 181 191 200
                                |  |  |  |  |  |  |  |  |  |  |
Consens_0252248 AACTCAAAGG AATTGACGGG GGCCCGCACA AGCGGTGGAG CATGTGGTTC AATTCGACGC AACGCGAAGA ACCTTACCTG GGTTTGAACT GCTGGTGGTA
TXv5v6-0252248 AACTCAAAGG AATTGACGGG GGCCCGCACA AGCGGTGGAG CATGTGGTTC AATTCGACGC AACGCGAAGA ACCTTACCTG GGTTTGAACT GCTGGTGGTA
TXv5v6-0689158 AACTCAAAGG AATTGACGGG GGCCCGCACA AGCGGTGGAG CATGTGGTTC AATTCGACGC AACGCGAAGA ACCTTACCTG GGTTTGAACT GCTGGTGGTA
TXv5v6-0252247 AACTCAAAGG AATTGACGGG GGCCCGCACA AGCGGTGGAG CATGTGGTTC AATTCGACGC AACGCGAAGA ACCTTACCTG GGTTTGAACT GCTGGTGGTA

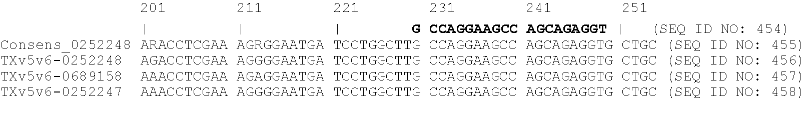

PTM33
PTM33 forward primer (SEQ ID NO: 59)
PTM33 reverse primer (SEQ ID NO: 60)
reverse complement of reverse primer (SEQ ID NO: 459)
CONSENS_0254691 (SEQ ID NO: 460)
TXv5v6-0254691 (SEQ ID NO: 461)
TXv5v6-0254679 (SEQ ID NO: 462)

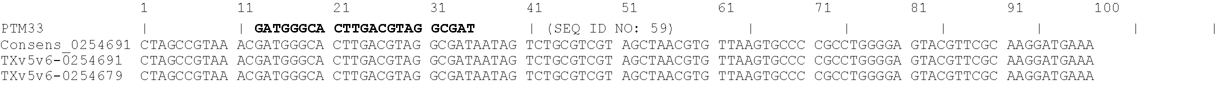

                                101 111 121 131 141 151 161 171 181 191 200
                                |  |  |  |  |  |  |  |  |  |  |
Consens_0254691 CTCAAAGGAA TTGACGGGGA CCCGCACAAG CGGTGGAGGA TGTGGTTTAA TTCGAGGCAA CGCGAAGAAC CTTACCTGGG CTTGACATAC AGGAAGTAGG
TXv5v6-0254691 CTCAAAGGAA TTGACGGGGA CCCGCACAAG CGGTGGAGGA TGTGGTTTAA TTCGAGGCAA CGCGAAGAAC CTTACCTGGG CTTGACATAC AGGAAGTAGG
TXv5v6-0254679 CTCAAAGGAA TTGACGGGGA CCCGCACAAG CGGTGGAGGA TGTGGTTTAA TTCGAGGCAA CGCGAAGAAC CTTACCTGGG CTTGACATAC AGGAAGTAGG

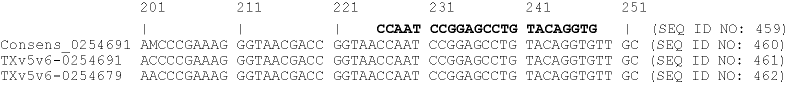

PTM34
PTM34 forward primer (SEQ ID NO: 61)
PTM34 reverse primer (SEQ ID NO: 62)
reverse complement of reverse primer (SEQ ID NO: 463)
CONSENS_0262828 (SEQ ID NO: 464)
TXv5v6-0262828 (SEQ ID NO: 465)
TXv5v6-0262852 (SEQ ID NO: 466)

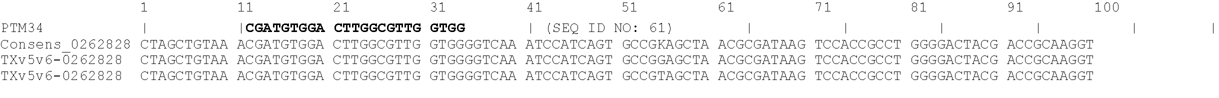

                                101 111 121 131 141 151 161 171 181 191 200
                                |  |  |  |  |  |  |  |  |  |  |
Consens_0262828 TAAAACTCAA AGGAATTGGC GGGGGCCCGC ACAAGCAGCG GAGCGTGTGG TTTAATTCGA TGCTACACGA AGAACCTTAC CCGGGTTTGA CATCCAGGTG
TXv5v6-0262828 TAAAACTCAA AGGAATTGGC GGGGGCCCGC ACAAGCAGCG GAGCGTGTGG TTTAATTCGA TGCTACACGA AGAACCTTAC CCGGGTTTGA CATCCAGGTG
TXv5v6-0262852 TAAAACTCAA AGGAATTGGC GGGGGCCCGC ACAAGCAGCG GAGCGTGTGG TTTAATTCGA TGCTACACGA AGAACCTTAC CCGGGTTTGA CATCCAGGTG

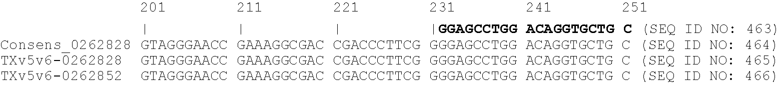

PTM35
PTM35 forward primer (SEQ ID NO: 63)
PTM35 reverse primer (SEQ ID NO: 64)
reverse complement of reverse primer (SEQ ID NO: 467)
CONSENS_1434138 (SEQ ID NO: 468)
TXv5v6-1434138 (SEQ ID NO: 469)
TXv5v6-0259077 (SEQ ID NO: 470)
TXv5v6-0722828 (SEQ ID NO: 471)

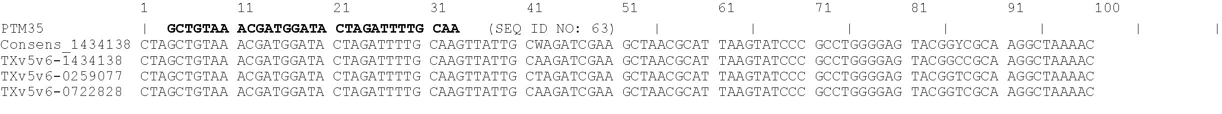

                                101 111 121 131 141 151 161 171 181 191 200
                                |  |  |  |  |  |  |  |  |  |  |
Consens_1434138 TCAAAGGAAT TGACGGGGAC CCGCACAAGC AGTGGAGCAT GTGGTTTAAT TCGATGCAAC GCGAAGAACC TTACCTGGGC TTGAACTGTA GGCATTAGCC
TXv5v6-1434138 TCAAAGGAAT TGACGGGGAC CCGCACAAGC AGTGGAGCAT GTGGTTTAAT TCGATGCAAC GCGAAGAACC TTACCTGGGC TTGAACTGTA GGCATTAGCC
TXv5v6-0259077 TCAAAGGAAT TGACGGGGAC CCGCACAAGC AGTGGAGCAT GTGGTTTAAT TCGATGCAAC GCGAAGAACC TTACCTGGGC TTGAACTGTA GGCATTAGCC
TXv5v6-0722828 TCAAAGGAAT TGACGGGGAC CCGCACAAGC AGTGGAGCAT GTGGTTTAAT TCGATGCAAC GCGAAGAACC TTACCTGGGC TTGAACTGTA GGCATTAGCC

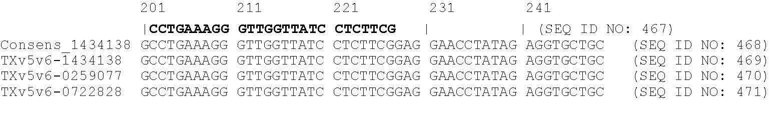

PTM36
PTM36 forward primer (SEQ ID NO: 65)
PTM36 reverse primer (SEQ ID NO: 66)
reverse complement of reverse primer (SEQ ID NO: 472)
CONSENS_1437489 (SEQ ID NO: 473)
TXv5v6-1437489 (SEQ ID NO: 474)
TXv5v6-0726865 (SEQ ID NO: 475)

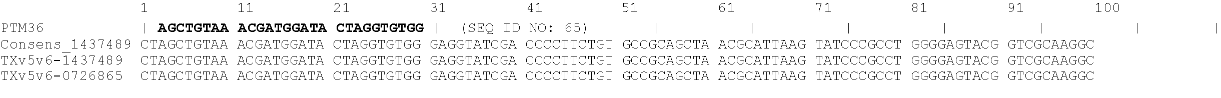

                                101 111 121 131 141 151 161 171 181 191 200
                                |  |  |  |  |  |  |  |  |  |  |
Consens_1437489 TGAAACTCAA AGGAATTGAC GGGGGCCCGC ACAAGCGGTG GAGCATGTGG TTTAATTCGA CGCAACGCGA AGAACCTTAC CGGGACTTGA CATTATYTTG
TXv5v6-1437489 TGAAACTCAA AGGAATTGAC GGGGGCCCGC ACAAGCGGTG GAGCATGTGG TTTAATTCGA CGCAACGCGA AGAACCTTAC CGGGACTTGA CATTATTTTG
TXv5v6-0726865 TGAAACTCAA AGGAATTGAC GGGGGCCCGC ACAAGCGGTG GAGCATGTGG TTTAATTCGA CGCAACGCGA AGAACCTTAC CGGGACTTGA CATTATCTTG

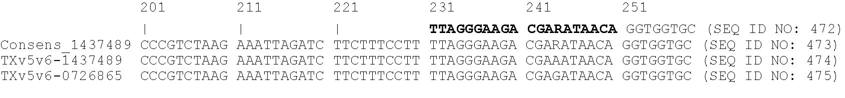

PTM37
PTM37 forward primer (SEQ ID NO: 67)
PTM37 reverse primer (SEQ ID NO: 68)
reverse complement of reverse primer (SEQ ID NO: 476)
CONSENS_0489473 (SEQ ID NO: 477)
TXv5v6-0489473 (SEQ ID NO: 478)
TXv5v6-0059568 (SEQ ID NO: 479)

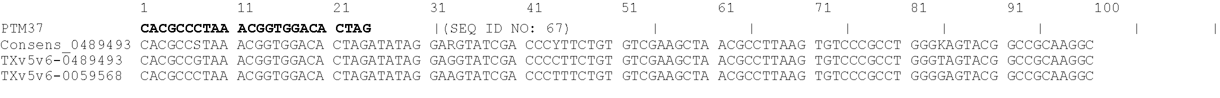

                                101 111 121 131 141 151 161 171 181 191 200
                                |  |  |  |  |  |  |  |  |  |  |
Consens_0489473 TGAAACTCAA AGGAATTGAC GGGGGCCCGC ACAAGCAGCG GAGCGTGTGG TTTAATTCGA TGCTACRCGA AGAACCTTAC CAGGGCTTGA CATGRCAGAA
TXv5v6-0489473 TGAAACTCAA AGGAATTGAC GGGGGCCCGC ACAAGCAGCG GAGCGTGTGG TTTAATTCGA TGCTACACGA AGAACCTTAC CAGGGCTTGA CATGGCAGAA
TXv5v6-0059568 TGAAACTCAA AGGAATTGAC GGGGGCCCGC ACAAGCAGCG GAGCGTGTGG TTTAATTCGA TGCTACGCGA AGAACCTTAC CAGGGCTTGA CATGACAGAA

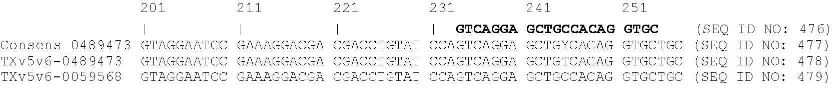

PTM38
PTM38 forward primer (SEQ ID NO: 69)
PTM38 reverse primer (SEQ ID NO: 70)
reverse complement of reverse primer (SEQ ID NO: 480)
CONSENS_0678112 (SEQ ID NO: 481)
TXv5v6-0678112 (SEQ ID NO: 482)
TXv5v6-0249051 (SEQ ID NO: 483)
TXv5v6-0249046 (SEQ ID NO: 484)

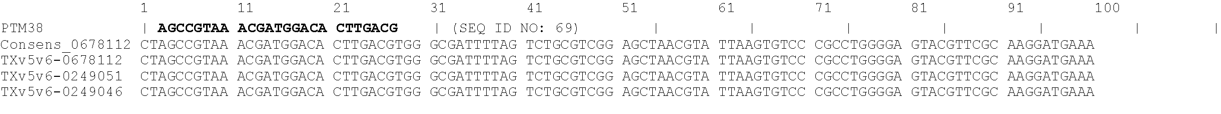

                                101 111 121 131 141 151 161 171 181 191 200
                                |  |  |  |  |  |  |  |  |  |  |
Consens_0678112 CTCAAAGGAA TTGACGGGGA CCCGCACAAG CGGTGGAGGA TGTGGTTTAA TTCGAGGCAA CGCGAAGAAC CTTACCTGGG TTTGACATGC AGAAAGTAGG
TXv5v6-0678112 CTCAAAGGAA TTGACGGGGA CCCGCACAAG CGGTGGAGGA TGTGGTTTAA TTCGAGGCAA CGCGAAGAAC CTTACCTGGG TTTGACATGC AGAAAGTAGG
TXv5v6-0249051 CTCAAAGGAA TTGACGGGGA CCCGCACAAG CGGTGGAGGA TGTGGTTTAA TTCGAGGCAA CGCGAAGAAC CTTACCTGGG TTTGACATGC AGAAAGTAGG
TXv5v6-0249046 CTCAAAGGAA TTGACGGGGA CCCGCACAAG CGGTGGAGGA TGTGGTTTAA TTCGAGGCAA CGCGAAGAAC CTTACCTGGG TTTGACATGC AGAAAGTAGG

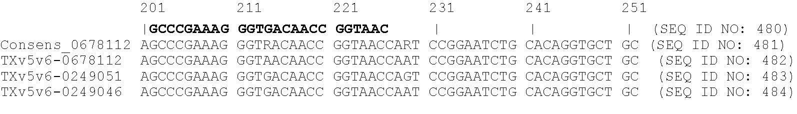

PTM39
PTM39 forward primer (SEQ ID NO: 71)
PTM39 reverse primer (SEQ ID NO: 72)
reverse complement of reverse primer (SEQ ID NO: 485)
CONSENS_0231931 (SEQ ID NO: 486)
TXv5v6-0231931 (SEQ ID NO: 487)
TXv5v6-0232006 (SEQ ID NO: 488)
TXv5v6-0231898 (SEQ ID NO: 489)

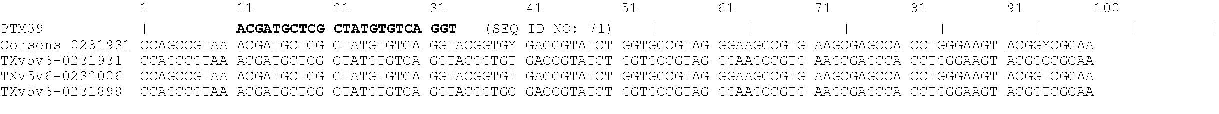

                                101 111 121 131 141 151 161 171 181 191 200
                                |  |  |  |  |  |  |  |  |  |  |
Consens_0231931 GRCTGAAACT TAAAGGAATT GGCGGGGGAG CACTACAAC- GGTGGAGCCT GCGGTTTAAT TGGATTCAAC GCCGGAAATC TTACCGGGKG AGACAGCARY
TXv5v6-0231931 GGCTGAAACT TAAAGGAATT GGCGGGGGAG CACTACAACG GGTGGAGCCT GCGGTTTAAT TGGATTCAAC GCCGGAAATC TTACCGGGGG AGACAGCAGC
TXv5v6-0232006 GACTGAAACT TAAAGGAATT GGCGGGGGAG CACTACAAC- GGTGGAGCCT GCGGTTTAAT TGGATTCAAC GCCGGAAATC TTACCGGGGG AGACAGCAGC
TXv5v6-0231898 GACTGAAACT TAAAGGAATT GGCGGGGGAG CACTACAAC- GGTGGAGCCT GCGGTTTAAT TGGATTCAAC GCCGGAAATC TTACCGGGTG AGACAGCAAT

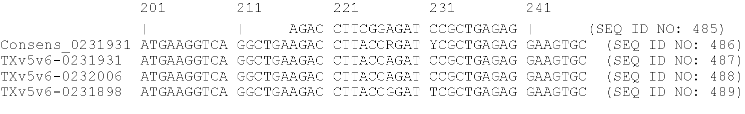

PTM40
PTM40 forward primer (SEQ ID NO: 73)
PTM40 reverse primer (SEQ ID NO: 74)
reverse complement of reverse primer (SEQ ID NO: 490)
CONSENS_0217253 (SEQ ID NO: 491)
TXv5v6-0217253 (SEQ ID NO: 492)
TXv5v6-0217292 (SEQ ID NO: 493)

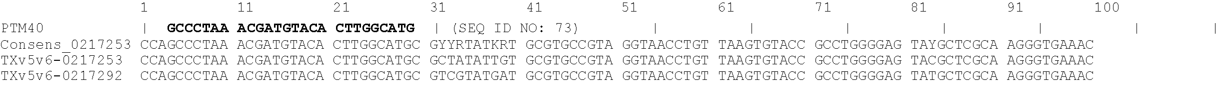

                                101 111 121 131 141 151 161 171 181 191 200
                                |  |  |  |  |  |  |  |  |  |  |
Consens_0217253 TCAAAGGAAT TGACGGGGAC CCGCACAAGC GGTGGAGGAT GTGGTTCAAT TCGAGGCAAC GCGAAGAACC TTACCTGGGC TTGACATGCT GATAGTACTR
TXv5v6-0217253 TCAAAGGAAT TGACGGGGAC CCGCACAAGC GGTGGAGGAT GTGGTTCAAT TCGAGGCAAC GCGAAGAACC TTACCTGGGC TTGACATGCT GATAGTACTG
TXv5v6-0217292 TCAAAGGAAT TGACGGGGAC CCGCACAAGC GGTGGAGGAT GTGGTTCAAT TCGAGGCAAC GCGAAGAACC TTACCTGGGC TTGACATGCT GATAGTACTA

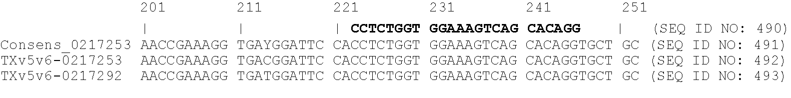

PTM41
PTM41 forward primer (SEQ ID NO: 75)
PTM41 reverse primer (SEQ ID NO: 76)
reverse complement of reverse primer (SEQ ID NO: 494)
CONSENS_0025886 (SEQ ID NO: 495)
TXv5v6-0025886 (SEQ ID NO: 496)
TXv5v6-0025873 (SEQ ID NO: 497)
TXv5v6-0025863 (SEQ ID NO: 498)
TXv5v6-0025876 (SEQ ID NO: 499)

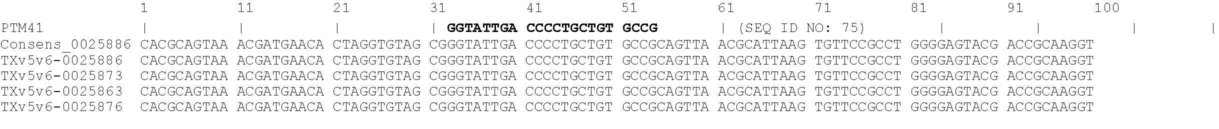

                                101 111 121 131 141 151 161 171 181 191 200
                                |  |  |  |  |  |  |  |  |  |  |
Consens_0025886 TAAAACTCAA AGGAATTGAC GGGGGCCCGC ACAAGCGGTG GAGCATGTGG TTTAATTCGA CGCAACGCGA AGAACCTTAC CTGGATTTGA CATCCcGGGA
TXv5v6-0025886 TAAAACTCAA AGGAATTGAC GGGGGCCCGC ACAAGCGGTG GAGCATGTGG TTTAATTCGA CGCAACGCGA AGAACCTTAC CTGGATTTGA CATCCTGGGA
TXv5v6-0025873 TAAAACTCAA AGGAATTGAC GGGGGCCCGC ACAAGCGGTG GAGCATGTGG TTTAATTCGA CGCAACGCGA AGAACCTTAC CTGGATTTGA CATCCCGGGA
TXv5v6-0025863 TAAAACTCAA AGGAATTGAC GGGGGCCCGC ACAAGCGGTG GAGCATGTGG TTTAATTCGA CGCAACGCGA AGAACCTTAC CTGGATTTGA CATCCCGGGA
TXv5v6-0025876 TAAAACTCAA AGGAATTGAC GGGGGCCCGC ACAAGCGGTG GAGCATGTGG TTTAATTCGA CGCAACGCGA AGAACCTTAC CTGGATTTGA CATCCCGGGA

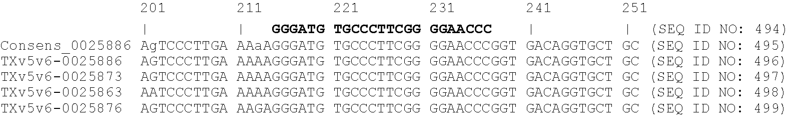

PTM42
PTM42 forward primer (SEQ ID NO: 77)
PTM42 reverse primer (SEQ ID NO: 78)
reverse complement of reverse primer (SEQ ID NO: 500)
CONSENS_0726759 (SEQ ID NO: 501)
TXv5v6-0726759 (SEQ ID NO: 502)
TXv5v6-0260150 (SEQ ID NO: 503)
TXv5v6-0259561 (SEQ ID NO: 504)
TXv5v6-0259703 (SEQ ID NO: 505)

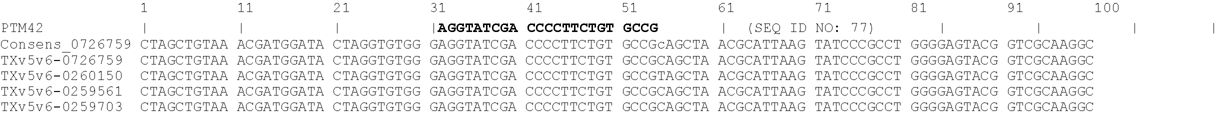

                                101 111 121 131 141 151 161 171 181 191 200
                                |  |  |  |  |  |  |  |  |  |  |
Consens_0726759 TGAAACTCAA AGGAATTGAC GGGGGCCCGC ACAAGCGGTG GAGCATGTGG TTTAATTCGA CGCAACGCGA AGAACCTTAC CGGGRCTTGA CATTaTCTTG
TXv5v6-0726759 TGAAACTCAA AGGAATTGAC GGGGGCCCGC ACAAGCGGTG GAGCATGTGG TTTAATTCGA CGCAACGCGA AGAACCTTAC CGGGACTTGA CATTATCTTG
TXv5v6-0260150 TGAAACTCAA AGGAATTGAC GGGGGCCCGC ACAAGCGGTG GAGCATGTGG TTTAATTCGA CGCAACGCGA AGAACCTTAC CGGGGCTTGA CATTATCTTG
TXv5v6-0259561 TGAAACTCAA AGGAATTGAC GGGGGCCCGC ACAAGCGGTG GAGCATGTGG TTTAATTCGA CGCAACGCGA AGAACCTTAC CGGGACTTGA CATTATCTTG
TXv5v6-0259703 TGAAACTCAA AGGAATTGAC GGGGGCCCGC ACAAGCGGTG GAGCATGTGG TTTAATTCGA CGCAACGCGA AGAACCTTAC CGGGGCTTGA CATTGTCTTG

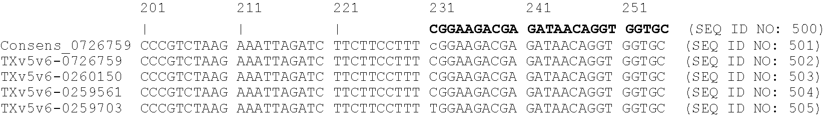

PTM43
PTM43 forward primer (SEQ ID NO: 79)
PTM43 reverse primer (SEQ ID NO: 80)
reverse complement of reverse primer (SEQ ID NO: 506)
CONSENS_0258903 (SEQ ID NO: 507)
TXv5v6-0258903 (SEQ ID NO: 508)
TXv5v6-1692076 (SEQ ID NO: 509)
TXv5v6-0258906 (SEQ ID NO: 510)
TXv5v6-0719836 (SEQ ID NO: 511)

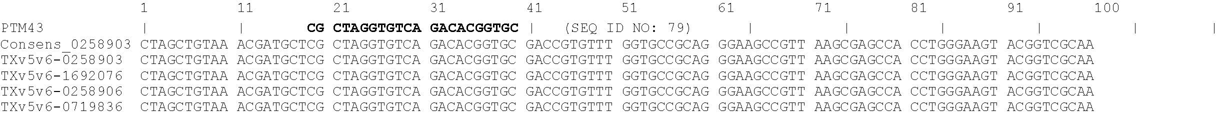

                                101 111 121 131 141 151 161 171 181 191 200
                                |  |  |  |  |  |  |  |  |  |  |
Consens_0258903 GGCTGAAACT TAAAGGAATT GGCGGGGGAG CACTACAACG GGTGGAGCCT GCGGTTTAAT TGGATTCAAC GCCGGAAAAC TCACCGGGTG CGACAGCAAt
TXv5v6-0258903 GGCTGAAACT TAAAGGAATT GGCGGGGGAG CACTACAACG GGTGGAGCCT GCGGTTTAAT TGGATTCAAC GCCGGAAAAC TCACCGGGTG CGACAGCAAC
TXv5v6-1692076 GGCTGAAACT TAAAGGAATT GGCGGGGGAG CACTACAACG GGTGGAGCCT GCGGTTTAAT TGGATTCAAC GCCGGAAAAC TCACCGGGTG CGACAGCAAT
TXv5v6-0258906 GGCTGAAACT TAAAGGAATT GGCGGGGGAG CACTACAACG GGTGGAGCCT GCGGTTTAAT TGGATTCAAC GCCGGAAAAC TCACCGGGTG CGACAGCAAT
TXv5v6-0719836 GGCTGAAACT TAAAGGAATT GGCGGGGGAG CACTACAACG GGTGGAGCCT GCGGTTTAAT TGGATTCAAC GCCGGAAAAC TCACCGGGTG CGACAGCAAT

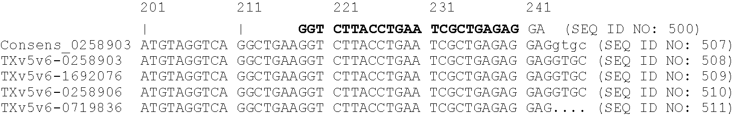

PTM44
PTM44 forward primer (SEQ ID NO: 81)
PTM44 reverse primer (SEQ ID NO: 82)
reverse complement of reverse primer (SEQ ID NO: 512)
CONSENS_0262835 (SEQ ID NO: 513)
TXv5v6-026283 (SEQ ID NO: 514)
TXv5v6-0262867 (SEQ ID NO: 515)

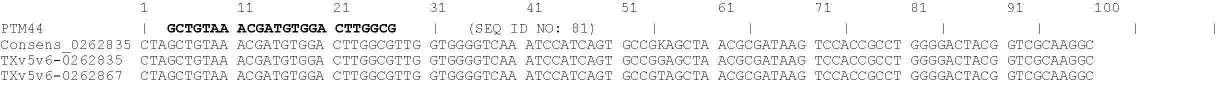

                                101 111 121 131 141 151 161 171 181 191 200
                                |  |  |  |  |  |  |  |  |  |  |
Consens_0262835 TAAAACTCAA AGGAATTGGC GGGGGCCCGC ACAAGCAGCG GAGCGTGTGG TTTAATTCGA TGCTACACGA AGAACCTTAC CCGGGTTTGA CATCCAGGTG
TXv5v6-0262835 TAAAACTCAA AGGAATTGGC GGGGGCCCGC ACAAGCAGCG GAGCGTGTGG TTTAATTCGA TGCTACACGA AGAACCTTAC CCGGGTTTGA CATCCAGGTG
TXv5v6-0262867 TAAAACTCAA AGGAATTGGC GGGGGCCCGC ACAAGCAGCG GAGCGTGTGG TTTAATTCGA TGCTACACGA AGAACCTTAC CCGGGTTTGA CATCCAGGTG

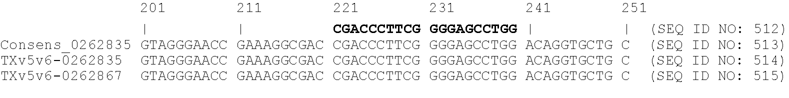

PTM45
PTM45 forward primer (SEQ ID NO: 83)
PTM45 reverse primer (SEQ ID NO: 84)
reverse complement of reverse primer (SEQ ID NO: 516)
CONSENS_0260001 (SEQ ID NO: 517)
TXv5v6-0260001 (SEQ ID NO: 518)
TXv5v6-1439641 (SEQ ID NO: 519)
TXv5v6-0725610 (SEQ ID NO: 520)

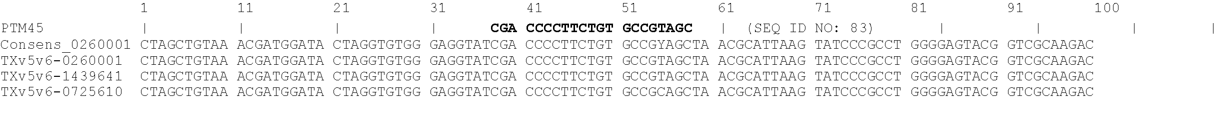

                                101 111 121 131 141 151 161 171 181 191 200
                                |  |  |  |  |  |  |  |  |  |  |
Consens_0260001 TGAAACTCAA AGGAATTGAC GGGGGCCCGC ACAAGCGGTG GAGCATGTGG TTTAATTCGA CGCAACGCGA AGAACCTTAC CGGGGCTTGA CATTGTCTTG
TXv5v6-0260001 TGAAACTCAA AGGAATTGAC GGGGGCCCGC ACAAGCGGTG GAGCATGTGG TTTAATTCGA CGCAACGCGA AGAACCTTAC CGGGGCTTGA CATTGTCTTG
TXv5v6-1439641 TGAAACTCAA AGGAATTGAC GGGGGCCCGC ACAAGCGGTG GAGCATGTGG TTTAATTCGA CGCAACGCGA AGAACCTTAC CGGGGCTTGA CATTGTCTTG
TXv5v6-0725610 TGAAACTCAA AGGAATTGAC GGGGGCCCGC ACAAGCGGTG GAGCATGTGG TTTAATTCGA CGCAACGCGA AGAACCTTAC CGGGGCTTGA CATTGTCTTG

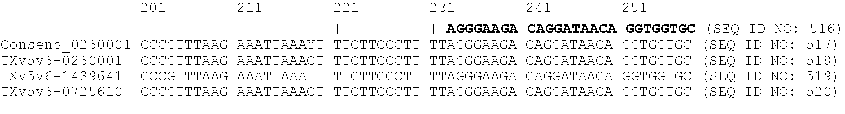

PTM46
PTM46 forward primer (SEQ ID NO: 85)
PTM46 reverse primer (SEQ ID NO: 86)
reverse complement of reverse primer (SEQ ID NO: 521)
CONSENS_0259164 (SEQ ID NO: 522)
TXv5v6-0259164 (SEQ ID NO: 523)
TXv5v6-0729803 (SEQ ID NO: 524)

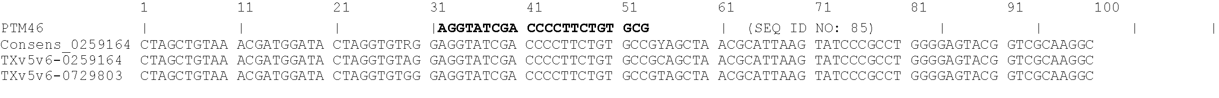

                                101 111 121 131 141 151 161 171 181 191 200
                                |  |  |  |  |  |  |  |  |  |  |
Consens_0259164 TGAAACTCAA AGGAATTGAC GGGGGCCCGC ACAAGCGGTG GAGCATGTGG TTTAATTCGA CGCAACGCGA AGAACCTTAC CGGGACTTGA CATTATCTTG
TXv5v6-0259164 TGAAACTCAA AGGAATTGAC GGGGGCCCGC ACAAGCGGTG GAGCATGTGG TTTAATTCGA CGCAACGCGA AGAACCTTAC CGGGACTTGA CATTATCTTG
TXv5v6-0729803 TGAAACTCAA AGGAATTGAC GGGGGCCCGC ACAAGCGGTG GAGCATGTGG TTTAATTCGA CGCAACGCGA AGAACCTTAC CGGGACTTGA CATTATCTTG

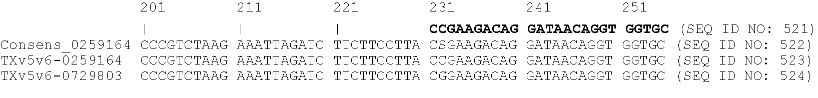

Summary Table 3 sequences:
PTM12_CONSENSUS(SEQ ID NO: 315)
CCAGCCGTAAACGATGCACGCTAGGTGTGGGTCGGCCACGAGCCGCCCCAGTGCCGCAGGGAAGCCRTTAAGCGTGCCGCCTGGGGAGTACGGCCGCAAGGCTGAAACTTAAAGGAATTGGCGGGGGAGCACCACCAGGCG
TGAAGCCTGCGGTTTAATTGGAGTCAACGCCGGGAACCTTACCGGGAGCGACAGCAGAGTGAAGGCCAGGCTGAAGACCTTGCCAGACAAGCTGAGAGGAGGTGC
TXv5v60593770(SEQ ID NO: 316)
CCAGCCGTAAACGATGCACGCTAGGTGTGGGTCGGCCACGAGCCGCCCCAGTGCCGCAGGGAAGCCGTTAAGCGTGCCGCCTGGGGAGTACGGCCGCAAGGCTGAAACTTAAAGGAATTGGCGGGGGAGCACCACCAGGCG
TGAAGCCTGCGGTTTAATTGGAGTCAACGCCGGGAACCTTACCGGGAGCGACAGCAGAGTGAAGGCCAGGCTGAAGACCTTGCCAGACAAGCTGAGAGGAGGTGC
TXv5v60219684(SEQ ID NO: 317)
CCAGCCGTAAACGATGCACGCTAGGTGTGGGTCGGCCACGAGCCGCCCCAGTGCCGCAGGGAAGCCATTAAGCGTGCCGCCTGGGGAGTACGGCCGCAAGGCTGAAACTTAAAGGAATTGGCGGGGGAGCACCACCAGGCG
TGAAGCCTGCGGTTTAATTGGAGTCAACGCCGGGAACCTTACCGGGAGCGACAGCAGAGGCTGAAACTTAAAGGAATTGGCGGGGGAGCACCACCAGGCGTGAAGCCTGCGGTTTAATTGGAGTCAACGCCGGGAACCTTAC
CGGGAGCGACAGCAGA
PTM13_CONSENSUS
CAGGGTGTAAACGCTGCTAGCTTGGTGTTGGATAACCYACGTGGTTATTCAGTGCCGGAGAGAAGTTGTTAAGCTAGCTACCTGGGAAGTACGGTCGCAAGGCTGAAACTTAAAGGAATTGGCGGGGGAGCACTGCAACGGG
TGGAGCGTACGGTTTAATTGGATTCAACGCCGAAAACCTCACCGGAGGCGACAGCTGRATGAAGGCCAGGCTAAAGACTTTGCTGGACTAGCTGAGAGGTGGTGC
TXv5v60208415
CAGGGTGTAAACGCTGCTAGCTTGGTGTTGGATAACCCACGTGGTTATTCAGTGCCGGAGAGAAGTTGTTAAGCTAGCTACCTGGGAAGTACGGTCGCAAGGCTGAAACTTAAAGGAATTGGCGGGGGAGCACTGCAACGGG
TGGAGCGTACGGTTTAATTGGATTCAACGCCGAAAACCTCACCGGAGGCGACAGCTGAATGAAGGCCAGGCTAAAGACTTTGCTGGACTAGCTGAGAGGTGGTGC
TXv5v60208460
CAGGGTGTAAACGCTGCTAGCTTGGTGTTGGATAACCTACGTGGTTATTCAGTGCCGGAGAGAAGTTGTTAAGCTAGCTACCTGGGAAGTACGGTCGCAAGGCTGAAACTTAAAGGAATTGGCGGGGGAGCACTGCAACGGG
TGGAGCGTACGGTTTAATTGGATTCAACGCCGAAAACCTCACCGGAGGCGACAGCTGGATGAAGGCCAGGCTAAAGACTTTGCTGGACTAGCTGAGAGGTGGTGC
>PTM14_CONSENSUS
CAGGGTGTAAACGCTGCTTGCTTGATGTTAGTTGGGCTCCGAGCCCAAYTAGTGTCGGAGAGAAGTTGTTAAGCAAGCTGCCTGGGAAGTACGGTCGCAAGRCTGAAACTTAAAGGAATTGGCGGGGGAGCACAGCAACGGG
TGGAGCGTGCGGTTTAATTGGATTCAACGCCGGAAAACTCACCGGAGGCGACGGTTACATGAAGGCCAGGCTGATGACCTTGCCTGATTTTCCGAGAGGTGGTGC
TXv5v60208552
CAGGGTGTAAACGCTGCTTGCTTGATGTTAGTTGGGCTCCGAGCCCAATTAGTGTCGGAGAGAAGTTGTTAAGCAAGCTGCCTGGGAAGTACGGTCGCAAGACTGAAACTTAAAGGAATTGGCGGGGGAGCACAGCAACGGG
TGGAGCGTGCGGTTTAATTGGATTCAACGCCGGAAAACTCACCGGAGGCGACGGTTACATGAAGGCCAGGCTGATGACCTTGCCTGATTTTCCGAGAGGTGGTGC
TXv5v60208531
CAGGGTGTAAACGCTGCTTGCTTGATGTTAGTTGGGCTCCGAGCCCAACTAGTGTCGGAGAGAAGTTGTTAAGCAAGCTGCCTGGGAAGTACGGTCGCAAGGCTGAAACTTAAAGGAATTGGCGGGGGAGCACAGCAACGGG
TGGAGCGTGCGGTTTAATTGGATTCAACGCCGGAAAACTCACCGGAGGCGACGGTTACATGAAGGCCAGGCTGATGACCTTGCCTGATTTTCCGAGAGGTGGTGC
>PTM15_CONSENSUS
CCAGCCGTAAACgATGCCAGCTATGTGTCGGAAGATCCAGtGTTCTTCCGGTGtcGTAGGGAAGCCGTGAAGCTGGCCACCTGGGAAGTACGGCCGCAAGGCTGAAACTTAAAGGAATTGGCGGGGGAGTACTACAACCGGTG
GAGCTTGCGGTTTAATTGGATACAACGCCGGAAATCTACCGGGGGCGACAGCAGTATGAAGGCCAGGCTGAGGACCTTGCYaGAYTAGCTGAGAGGAGGTGC
TXv5v60217476
CCAGCCGTAAACAATGCCAGCTATGTGTCGGAAGATCCAGTGTTCTTCCGGTGTTGTAGGGAAGCCGTGAAGCTGGCCACCTGGGAAGTACGGCCGCAAGGCTGAAACTTAAAGGAATTGGCGGGGGAGTACTACAACCGGT
GGAGCTTGCGGTTTAATTGGATACAACGCCGGAAATCTACCGGGGGCGACAGCAGTATGAAGGCCAGGCTGAGGACCTTGCCAGACTAGCTGAGAGGAGGTGC
TXv5v60219822
CCAGCCGTAAACGATGCCAGCTATGTGTCGGAAGATCCAGCGTTCTTCCGGTGTCGTAGGGAAGCCGTGAAGCTGGCCACCTGGGAAGTACGGCCGCAAGGCTGAAACTTAAAGGAATTGGCGGGGGAGTACTACAACCGGT
GGAGCTTGCGGTTTAATTGGATACAACGCCGGAAATCTACCGGGGGCGACAGCAGTATGAAGGCCAGGCTGAGGACCTTGCCAGATTAGCTGAGAGGAGGTGC
TXv5v60219861
CCAGCCGTAAACGATGCCAGCTATGTGTCGGAAGATCCAGTGTTCTTCCGGTGTCGTAGGGAAGCCGTGAAGCTGGCCACCTGGGAAGTACGGCCGCAAGGCTGAAACTTAAAGGAATTGGCGGGGGAGTACTACAACCGGT
GGAGCTTGCGGTTTAATTGGATACAACGCCGGAAATCTACCGGGGGCGACAGCAGTATGAAGGCCAGGCTGAGGACCTTGCCAGATTAGCTGAGAGGAGGTGC
TXv5v60219863
CCAGCCGTAAACGATGCCAGCTATGTGTCGGAAGATCCAGTGTTCTTCCGGTGTCGTAGGGAAGCCGTGAAGCTGGCCACCTGGGAAGTACGGCCGCAAGGCTGAAACTTAAAGGAATTGGCGGGGGAGTACTACAACCGGT
GGAGCTTGCGGTTTAATTGGATACAACGCCGGAAATCTACCGGGGGCGACAGCAGTATGAAGGCCAGGCTGAGGACCTTGCTAGATTAGCTGAGAGGAGGTGC
TXv5v60219845
CCAGCCGTAAACGATGCCAGCTATGTGTCGGAAGATCCAGTGTTCTTCCGGTGCCGTAGGGAAGCCGTGAAGCTGGCCACCTGGGAAGTACGGCCGCAAGGCTGAAACTTAAAGGAATTGGCGGGGGAGTACTACAACCGGT
GGAGCTTGCGGTTTAATTGGATACAACGCCGGAAATCTACCGGGGGCGACAGCAGTATGAAGGCCAGGCTGAGGACCTTGCTGGACTAGCTGAGAGGAGGTGC
>PTM16_CONSENSUS
CCAGCCGTAAACGATGCAGGCTAGGTGTGGGttGGCCACGtGCCgCTCAGTGCCACAGGGAAGCCATTAAGCCTGCcGCCTGGGGAGTACGGYCGCAAGGCTGAAACTTAAAGGAATTGGCGGGgGAGCACCACCAGGCGTGA
AGCCTGCGGTTTAATTGGAGTCAACGCCGGGAAcCTTACCGGGAGCGACAGCAGAgTGAAgGCCAGGtTGAAGGTCTTGCYgGACGAGCTGAGAGGaGGTGC
TXv5v60219799
CCAGCCGTAAACGATGCAGGCTAGGTGTGGGTTGGCCACGTGCCGACTCAGTGCCACAGGGAAGCCATTAAGCCTGCTGCCTGGGGAGTACGGTCGCAAGGCTGAAACTTAAAGGAATTGGCGGGAGAGCACCACCAGGCGT
GAAGCCTGCGGTTTAATTGGAGTCAACGCCGGGAACCTTACCGGGAGCGACAGCAGAGTGAAAGCCAGGTTGAAGGTCTTGCTGGACGAGCTGAGAGGAGGTGC
TXv5v60219794
CCAGCCGTAAACGATGCAGGCTAGGTGTGGGGTGGCCACGTGCCGCCTCAGTGCCACAGGGAAGCCATTAAGCCTGCCGCCTGGGGAGTACGGTCGCAAGGCTGAAACTTAAAGGAATTGGCGGGGGAGCACCACCAGGCG
TGAAGCCTGCGGTTTAATTGGAGTCAACGCCGGGAACCTTACCGGGAGCGACAGCAGAGTGAAGGCCAGGTTGAAGGTCTTGCCGGACGAGCTGAGAGGAGGTGC
TXv5v60596935
CCAGCCGTAAACGATGCAGGCTAGGTGTGGGTTGGCCACGTGCCAGCTCAGTGCCACAGGGAAGCCATTAAGCCTGCCGCCTGGGGAGTACGGCCGCAAGGCTGAAACTTAAAGGAATTGGCGGGGGAGCACCACCAGGCG
TGAAGCCTGCGGTTTAATTGGAGTCAACGCCGGGAATCTTACCGGGAGCGACAGCAGAATGAAGGCCAGGTTGAAGGTCTTGCTGGACGAGCTGAGAGGTGGTGC
TXv5v60219795
CCAGCCGTAAACGATGCAGGCTAGGTGTGGGTCGGCCACGCGCCGCCTCAGTGCCACAGGGAAGCCATTAAGCCTGCCGCCTGGGGAGTACGGCCGCAAGGCTGAAACTTAAAGGAATTGGCGGGGGAGCACCACCAGGCG
TGAAGCCTGCGGTTTAATTGGAGTCAACGCCGGGAACCTTACCGGGAGCGACAGCAGAGTGAAGGCCAGGCTGAAGGTCTTGCCAGACGAGCTGAGAGGAGGTGC
>PTM17_CONSENSUS
CCAGCTGTAAACGATGCAGGCTAGGTGTGGCGCGGCTACGTGCCGCTCAGTGCCGCAGGGAAGCCGTTAAGCCTGCCGCCTGGGAAGTACGGCCGCAAGGCTGAAACTTAAAGGAATTGGCGGGGGAGCACCACAAGGKGT
GAAGCTTGCGGTTTAATTGGAGTCAACGCCGGAAATCTCACCGGGGGCGACAGCAGAATGAAGGTCAGATTGAAGGTCTTACCAGACAAGCTGAGAGGAGGTGC
TXv5v60235530
CCAGCTGTAAACGATGCAGGCTAGGTGTGGCGCGGCTACGTGCCGCTCAGTGCCGCAGGGAAGCCGTTAAGCCTGCCGCCTGGGAAGTACGGCCGCAAGGCTGAAACTTAAAGGAATTGGCGGGGGAGCACCACAAGGGGT
GAAGCTTGCGGTTTAATTGGAGTCAACGCCGGAAATCTCACCGGGGGCGACAGCAGAATGAAGGTCAGATTGAAGGTCTTACCAGACAAGCTGAGAGGAGGTGC
TXv5v60235545
CCAGCTGTAAACGATGCAGGCTAGGTGTGGCGCGGCTACGTGCCGCTCAGTGCCGCAGGGAAGCCGTTAAGCCTGCCGCCTGGGAAGTACGGCCGCAAGGCTGAAACTTAAAGGAATTGGCGGGGGAGCACCACAAGGTGT
GAAGCTTGCGGTTTAATTGGAGTCAACGCCGGAAATCTCACCGGGGGCGACAGCAGAATGAAGGTCAGATTGAAGGTCTTACCAGACAAGCTGAGAGGAGGTGC
>PTM18_CONSENSUS
CTAGCAGTAAACaCTGCACACTAAACATtAGTACCTCYTCGaGAGGtATTgGTGCTGwAGgGAAGcCgAAGAGTGTGCTACCTGGGAAGTATAGYCGCAAGGCcGAAACTTAAAGGAATWGGCGGGGAGaCACTACAACRGGTG
ACGCGTGCGGTTCAATTAGATTaTACACCGTGAAcCTcACCAGGagCGAcAGCAGaATGAAGGTCAGTCTgAAGGGCTTACCTgACACGCTgAGAGGAGtTGC
TXv5v60242586
CTAGCAGTAAACACTGCACACTAAACATTAGTACCTCCTCGAGAGGTATTGGTGCTGTAGCGAAGGCGAAGAGTGTGCTACCTGGGAAGTATAGCCGCAAGGCCGAAACTTAAAGGAATAGGCGGGGAGGCACTACAACGGG
TGACGCGTGCGGTTCAATTAGATTATACACCGTGAACCTCACCAGGAGCGATAGCAGAATGAAGGTCAGTCTGAAGGGCTTACCTGACACGCTAAGAGGAGTTGC
TXv5v60242630
CTAGCAGTAAACACTGCACACTAAACATTAGTACCTCTTCGAGAGGTATTGGTGCTGTAGCGAAGGCGAAGAGTGTGCTACCTGGGAAGTATAGCCGCAAGGCCGAAACTTAAAGGAATAGGCGGGGAGGCACTACAACGGG
TGACGCGTGCGGTTCAATTAGATTATACACCGTGAACCTCACCAGGAGCGATAGCAGAATGAAGGTCAGTCTGAAGGGCTTACCTGACACGCTAAGAGGAGTTGC
TXv5v60647404
CTAGCAGTAAACACTGCACACTAAACATTAGTACCTCTTCGAGAGGTATTGGTGCTGTAGCGAAGGCGAAGAGTGTGCTACCTGGGAAGTATAGCCGCAAGGCCGAAACTTAAAGGAATAGGCGGGGAGACACTACAACGGG
TGACGCGTGCGGTTCAATTAGATTATACACCGTGAACCTCACCAGGAGCGATAGCAGAATGAAGGTCAGTCTGAAGGGCTTACCTGACACGCTAAGAGGAGTTGC
TXv5v60242596
CTAGCAGTAAACACTGCACACTAAACATTAGTACCTCCTCGAGAGGTATTGGTGCTGTAGGGAAGCCGAAGAGTGTGCTACCTGGGAAGTATAGCCGCAAGGCCGAAACTTAAAGGAATAGGCGGGGAGACACTACAACGGG
TGACGCGTGCGGTTCAATTAGATTATACACCGTGAACCTCACCAGGAGCGATAGCAGAATGAAGGTCAGTCTGAAGGGCTTACCTGACACGCTAAGAGGAGTTGC
TXv5v60242606
CTAGCAGTAAACACTGCACACTAAACATTAGTACCTCCTCGAGAGGTATTGGTGCTGTAGGGAAGCCGAAGAGTGTGCTACCTGGGAAGTATAGCCGCAAGGCCGAAACTTAAAGGAATAGGCGGGGAGACACTACAACGGG
TGACGCGTGCGGTTCAATTAGATTCTACACCGTGAACCTCACCAGGAGCGACAGCAGGATGAAGGTCAGTCTGAAGGGCTTACCTGACACGCTGAGAGGAGTTGC
TXv5v60642293
CTAGCAGTAAACACTGCACACTAAACATCAGTACCTCTTCGAGAGGCATTGGTGCTGCAGGGAAGCCGAAGAGTGTGCTACCTGGGAAGTATAGCCGCAAGGCCGAAACTTAAAGGAATAGGCGGGGAGGCACTACAACGGG
TGACGCGTGCGGTTCAATTAGATTATACACCGTGAACCTCACCAGGAGCGACAGCAGAATGAAGGTCAGTCTGAAGGGCTTACCTGACACGCTGAGAGGAGTTGC
TXv5v60651560
CTAGCAGTAAACTCTGCACACTAAACATTAGTACCTCTTCGAGAGGTATTAGTGCTGAAGGGAAGCCGAAGAGTGTGCTACCTGGGAAGTATAGCCGCAAGGCCGAAACTTAAAGGAATTGGCGGGGAGACACTACAACAGGT
GACGCGTGCGGTTCAATTAGATTATACACCGTGAACCTCACCAGGAGCGACAGCAGAATGAAGGTCAGTCTAAAGGGCTTACCTGACACGCTGAGAGGAGTTGC
TXv5v60644101
CTAGCAGTAAACACTGCACACTAAACATTAGTACCTCCTCGAGAGGTATTGGTGCTGAAGGGAAGCCGAAGAGTGTGCTACCTGGGAAGTATAGCCGCAGGCCGAAACTTAAAGGAATTGGCGGGGAGACACTACAACAGGT
GACGCGTGCGGTTCAATTAGATTATACACCGTGAACCTCACCAGGAGCGACAGCAGGATGAAGGTCAGTCTGAAGGGCTTACCTGACACGCTGAGAGGAGTTGC
TXv5v60242619
CTAGCAGTAAACACTGCACACTAAACATTAGTACCTCCTCGAGAGGTATTGGTGCTGTAGGGAAGCCGAAGAGTGTGCTACCTGGGAAGTATAGTCGCAAGGCCGAAACTTAAAGGAATTGGCGGGGAGACACTACAACGGG
TGACGCGTGCGGTTCAATTAGATTATACACCGTGAACCTCACCAGGAGCGACAGCAGAATGAAGGTCAGTCTGAAGGGCTTACCTAACACGCTGAGAGGAGTTGC
TXv5v60646437
CTAGCAGTAAACACTGCACACTAAACATTAGTACCTCCTCGAGAGGTATTGGTGCTGTAGGGAAGCCGAAGAGTGTGCTACCTGGGAAGTATAGTCGCAAGGCCGAAACTTAAAGGAATTGGCGGGGAGACACTACAACGGG
TGACGCGTGCGGTTCAATTAGATTATACACCGTGAACCTCACCAGGGGCGACAGCAGAATGAAGGTCAGTCTGAAGGGCTTACCTGACACGCTGAGAGGAGTTGC
TXv5v60641596
CTAGCAGTAAACACTGCACACTAAACATCAGTACCTCCTCGAGAGGTATTGGTGCTGAAGGGAAGCCGAAGAGTGTGCTACCTGGGAAGTATAGTCGCAAGGCCGAAACTTAAAGGAATTGGCGGGGAGACACTACAACAGG
TGACGCGTGCGGTTCAATTAGATTATACACCGTGAACCTCACCAGGAGCGACAGCAGAATGAAGGTCAGTCTGAAGGGCTTACCTGACACGCTGAGAGGAGTTGC
TXv5v60644254
CTAGCAGTAAACACTGCACACTAAACATTAGTACCTCCTCGAGAGGTATTGGTGCTGAAGGGAAGCCGAAGAGTGTGCTACCTGGGAAGTATAGTCGCAAGGCCGAAACTTAAAGGAATTGGCGGGGAGACACTACAACAGG
TGACGCGTGCGGTTCAATTAGATTATACACCGTGAACCTTACCAGGACCGACAGCAGAATGAAGGTCAGTCTAAAGGGCTTACCTGACACGCTGAGAGGAGCTGC
TXv5v60643665
CTAGCAGTAAACACTGCACACTAAACATTAGTACCTCCTCGAGAGGTATTAGTGCTGAAGGGAAGCCGAAGAGTGTGCTACCTGGGAAGTATAGTCGCAAGGCCGAAACTTAAAGGAATTGGCGGGGAGGCACTACAACGGG
TGACGCGTGCGGTTCAATTAGATTATACACCGTGAACCTCACCAGGAGCGACAGCAGAATGAAGGTCAGTCTGAAGGGCTTACCTGACACGCTGAGAGGAGCTGC
TXv5v60647677
CTAGCAGTAAACACTGCACACTAAACATTAGTACCTCTTCGGGAGGTATTAGTGCTGAAGGGAAGCCAAAGAGTGTGCTACCTGGGAAGTATAGTCGCAAGGCTGAAACTTAAAGGAATTGGCGGGGAGACACTACAACAGGT
GACGCGTGCGGTTCAATTAGATTATACACCGTGAATCTCACCAGGACCGACAGCAGAATGAAGGTCAGTCTGAAGGGCTTACCTGACACGCTGAGAGGAGTTGC
>PTM19_CONSENSUS
CTAGCAGTAAACGATGCGGGCYAGGTGTTAGTATCACTGCGAGTGGTACTAGTGTCGAAGGGAAGCCGTTAAGCCCGCCATCTGGGAAGTACGGTCGCAAGGCTGAAACTTAAAGGAATTGGCGGGGGAGCACTACAACGGG
TGGAGCTTGCGGTTTAATTGGATTCAACGCCGTGAATCTTACCGGGGAAGACAGCAAGATGAAAGCCAAGCTAAAGACTTTGCTGAATTAGCTGAGAGGTGGTGC
TXv5v60242690
CTAGCAGTAAACGATGCGGGCCAGGTGTTAGTATCACTGCGAGTGGTACTAGTGTCGAAGGGAAGCCGTTAAGCCCGCCATCTGGGAAGTACGGTCGCAAGGCTGAAACTTAAAGGAATTGGCGGGGGAGCACTACAACGG
GTGGAGCTTGCGGTTTAATTGGATTCAACGCCGTGAATCTTACCGGGGAAGACAGCAAGATGAAAGCCAAGCTAAAGACTTTGCTGAATTAGCTGAGAGGTGGTGC
TXv5v60242726
CTAGCAGTAAACGATGCGGGCTAGGTGTTAGTATCACTGCGAGTGGTACTAGTGTCGAAGGGAAGCCGTTAAGCCCGCCATCTGGGAAGTACGGTCGCAAGGCTGAAACTTAAAGGAATTGGCGGGGGAGCACTACAACGGG
TGGAGCTTGCGGTTTAATTGGATTCAACGCCGTGAATCTTACCGGGGAAGACAGCAAGATGAAAGCCAAGCTAAAGACTTTGCTGAATTAGCTGAGAGGTGGTGC
>PTM20_CONSENSUS
CTAGCCGTAAACGATGCTCGCTAGGTGTTAAATACCCTGGGAGGGTATTTAGTGTCGTAAGGAAGCCGTGAAGCGAGCCACCTGGGAAGTACGGTCGCAAGGCTGAAACTTAAAGGAATTGGCGGGGGAGCACAACAACGG
GTGGATGCTGCGGTTTAATTGGATTCAACGCCGGAAATCTTACCGGAGGCGACAGAATATGAAGGTCAGGTTGAAGACCTTACCAAATTCGCTGAGAGGAAGTGC
TXv5v60248376
CTAGCCGTAAACGATGCTCGCTAGGTGTTAAATACCCTGGGAGGGTATTTAGTGTCGTAAGGAAGCCGTGAAGCGAGCCACCTGGGAAGTACGGTCGCAAGGCTGAAACTTAAAGGAATTGGCGGGGGAGCACAACAACGG
GTGGATGCTGCGGTTTAATTGGATTCAACGCCGGAAATCTTACCGGAGGCGACAGCAATATGAAGGTCAGGTTGAAGACCTTACCAAATTCGCTGAGAGGAAGTGC
TXv5v60671483
CTAGCCGTAAACGATGCTCGCTAGGTGTTAAATACCCTGGGAGGGTATTTAGTGTCGTAAGGAAGCCGTGAAGCGAGCCACCTGGGAAGTACGGTCGCAAGGCTGAAACTTAAAGGGAATTGGCGGGGGAGCACAACAACG
GGTGGATGCTGCGGTTTAATTGGATTCAACGCCGGAAATCTTACCGGAGGCGACAGCAATATGAAGGTCAGGTTGAAGACCTTACCAAATTCGCTGAGAGGAAGTGC
>PTM21_CONSENSUS
CTGGCCGTAAACGATGCATACTAGGTGATGGTACGGCCATGAGCCGTATCAGTGCCGTAGGGAAACCGTTAAGTGTGCCGCCTGGGAAGTACGGTCGCAAGGCTAAAACTTAAAGGAATTGGCGGGGAGCACCACAAGGGGT
GAAGCCTGCGGTTCAATTGGACTCAACGCCGGGAAACTTACCAGGGGAGACAGCAGTATGAMGGTCAGGYTGACGACCTTACCYRACGAGCTGAGAGGAGGTGC
TXv5v60266750
CTGGCCGTAAACGATGCATACTAGGTGATGGTACGGCCATGAGCCGTATCAGTGCCGTAGGGAAACCGTTAAGTGTGCCGCCTGGGAAGTACGGTCGCAAGGCTAAAACTTAAAGGAATTGGCGGGGGAGCACCACAAGGG
GTGAAGCCTGCGGTTCAATTGGACTCAACGCCGGGAAACTTACCAGGGGAGACAGCAGTATGACGGTCAGGCTGACGACCTTACCCAACGAGCTGAGAGGAGGTGC
TXv5v60771140
CTGGCCGTAAACGATGCATACTAGGTGATGGTACGGCCATGAGCCGTATCAGTGCCGTAGGGAAACCGTTAAGTGTGCCGCCTGGGAAGTACGGTCGCAAGGCTAAAACTTAAAGGAATTGGCGGGGGAGCACCACAAGGG
GTGAAGCCTGCGGTTCAATTGGACTCAACGCCGGGAAACTTACCAGGGGAGACAGCAGTATGAAGGTCAGGTTGACGACCTTACCTGACGAGCTGAGAGGAGGTGC
TXv5v60770570
CTGGCCGTAAACGATGCATACTAGGTGATGGTACGGCCATGAGCCGTATCAGTGCCGTAGGGAAACCGTTAAGTGTGCCGCCTGGGAAGTACGGTCGCAAGGCTAAAACTTAAAGGAATTGGCGGGGAGCACCACAAGGGGT
GAAGCCTGCGGTTCAATTGGACTCAACGCCGGGAAACTTACCAGGGGAGACAGCAGTATGACGGTCAGGTTGACGACCTTACCCGACGAGCTGAGAGGAGGTGC
>PTM22_CONSENSUS
CTGGCCGTAAACGATGCATACTAGGTGATGGTACGGCTATGAGCCGTRTCAGTGCCGTAGGGAAACCGTTAAGTGTGCCGCCTGGGAAGTACGGTCGCAAGGCTAAAACTTAAAGGAATTGGCGGGGGAGCACCACAAGGGG
TGAAGCCTGCGGTTCAATTGGACTCAACGCCGGGAAACTTACCAGGGGAGACAGCAGWATGMCGGTCAGGTTGACGACCTTACCYRACGAGCTGAGAGGAGGTGC
TXv5v60266796
CTGGCCGTAAACGATGCATACTAGGTGATGGTACGGCTATGAGCCGTGTCAGTGCCGTAGGGAAACCGTTAAGTGTGCCGCCTGGGAAGTACGGTCGCAAGGCTAAAACTTAAAGGAATTGGCGGGGGAGCACCACAAGGG
GTGAAGCCTGCGGTTCAATTGGACTCAACGCCGGGAAACTTACCAGGGGAGACAGCAGAATGCCGGTCAGGTTGACGACCTTACCTAACGAGCTGAGAGGAGGTGC
TXv5v60772899
CTGGCCGTAAACGATGCATACTAGGTGATGGTACGGCTATGAGCCGTATCAGTGCCGTAGGGAAACCGTTAAGTGTGCCGCCTGGGAAGTACGGTCGCAAGGCTAAAACTTAAAGGAATTGGCGGGGGAGCACCACAAGGG
GTGAAGCCTGCGGTTCAATTGGACTCAACGCCGGGAAACTTACCAGGGGAGACAGCAGTATGACGGTCAGGTTGACGACCTTACCCGACGAGCTGAGAGGAGGTGC
>PTM23_CONSENSUS
CTGGGCGTAAATGATGTGGGCTAGGTGCAAAGCTACCTAAGYGGTAGCTTGGTGCCGATGGGAAGCCGTTAAGCCCACCGCCTGGGGAGTACGGTCGCAAGGCTGAAACTTAAAGGAATTGGCGGGKGAGCACCACAAGGG
GTGGAGGCTGCGGTTTAATTGGATTCAACGCCGGGAAACTCACCGGGGGCGACAGCAGTATGAAGGTCAGGCTGATGACCTTACCAGACAAGCTGAGAGGAGGTGC
TXv5v60283719
CTGGGCGTAAATGATGTGGGCTAGGTGCAAAGCTACCTAAGTGGTAGCTTGGTGCCGATGGGAAGCCGTTAAGCCCACCGCCTGGGGAGTACGGTCGCAAGGCTGAAACTTAAAGGAATTGGCGGGGGAGCACCACAAGGG
GTGGAGGCTGCGGTTTAATTGGATTCAACGCCGGGAAACTCACCGGGGGCGACAGCAGTATGAAGGTCAGGCTGATGACCTTACCAGACAAGCTGAGAGGAGGTGC
TXv5v60283712
CTGGGCGTAAATGATGTGGGCTAGGTGCAAAGCTACCTAAGCGGTAGCTTGGTGCCGATGGGAAGCCGTTAAGCCCACCGCCTGGGGAGTACGGTCGCAAGGCTGAAACTTAAAGGAATTGGCGGGTGAGCACCACAAGGG
GTGGAGGCTGCGGTTTAATTGGATTCAACGCCGGGAAACTCACCGGGGGCGACAGCAGTATGAAGGTCAGGCTGATGACCTTACCAGACAAGCTGAGAGGAGGTGC
TXv5v60788889
CTGGGCGTAAATGATGTGGGCTAGGTGCAAAGCTACCTAAGTGGTAGCTTGGTGCCGATGGGAAGCCGTTAAGCCCACCGCCTGGGGAGTACGGTCGCAAGGCTGAAACTTAAAGGAATTGGCGGGTGAGCACCACAAGGG
GTGGAGGCTGCGGTTTAATTGGATTCAACGCCGGGAAACTCACCGGGGGCGACAGCAGTATGAAGGTCAGGCTGATGACCTTACCAGACAAGCTGAGAGGAGGTGC
>PTM24_CONSENSUS
CTAGCTGTAAACGATGCRGGCYAGGTGTTGGCATTACTGCGAGTGATGCCAGTGCCGAAGGGAAGCCGTTAAGCCYGCCATCTGGGGAGTACGGTCGCAAGGCTGAAACTTAAAGGAATTGGCGGGGGAGCACYACAACGGG
TGGAGCYTGCGGTTCAATTGGATTCAACGCCGGAAAMCTCACCGGRGGMGACAGCGAKATGAAGGTCAGGCTGAAGACCTTACCRRATTAGCTGAGAGGTGGCGC
TXv5v60714814
CTAGCTGTAAACGATGCGGGCCAGGTGTTGGCATTACTGCGAGTGATGCCAGTGCCGAAGGGAAGCCGTTAAGCCCGCCATCTGGGGAGTACGGTCGCAAGGCTGAAACTTAAAGGAATTGGCGGGGGAGCACCACAACGG
GTGGAGCTTGCGGTTCAATTGGATTCAACGCCGGAAAACTCACCGGGGGAGACAGCGAGATGAAGGTCAGGCTGAAGACCTTACCAAATTAGCTGAGAGGTGGCGC
TXv5v60257743
CTAGCTGTAAACGATGCAGGCTAGGTGTTGGCATTACTGCGAGTGATGCCAGTGCCGAAGGGAAGCCGTTAAGCCTGCCATCTGGGGAGTACGGTCGCAAGGCTGAAACTTAAAGGAATTGGCGGGGGAGCACTACAACGGG
TGGAGCCTGCGGTTCAATTGGATTCAACGCCGGAAACCTCACCGGAGGCGACAGCGATATGAAGGTCAGGCTGAAGACCTTACCGGATTAGCTGAGAGGTGGCGC
>PTM25_CONSENSUS
CAGGGCGTAAACGATGTGGGCTTCGYATTGAAGACCGTATGGTTTTCAGTGCTGGAACGAAGGCGTTAAGCCCACCGCCTGGGAAGTACGGCCGCAAGGCTGAAACTTAAAGGAATTGACGGGGGAGCACAGCAACGGGAG
GAGCGTGCGGTTCAATTGGATTCAACGCCGGAAAACTCACCGGAGGAGACTGCCAGATGTGGGCCAAGCTGAAGACTTTGCTCGAATAATAGGCAGAGAGGTGGTGC
TXv5v61349302
CAGGGCGTAAACGATGTGGGCTTCGTATTGAAGACCGTATGGTTTTCAGTGCTGGAACGAAGGCGTTAAGCCCACCGCCTGGGAAGTACGGCCGCAAGGCTGAAACTTAAAGGAATTGACGGGGGAGCACAGCAACGGGAG
GAGCGTGCGGTTCAATTGGATTCAACGCCGGAAAACTCACCGGAGGAGACTGCCAGATGTGGGCCAAGCTGAAGACTTTGCTCGAATAATAGGCAGAGAGGTGGTGC
TXv5v61349224
CAGGGCGTAAACGATGTGGGCTTCGCATTGAAGACCGTATGGTTTTCAGTGCTGGAACGAAGGCGTTAAGCCCACCGCCTGGGAAGTACGGCCGCAAGGCTGAAACTTAAAGGAATTGACGGGGGAGCACAGCAACGGGAG
GAGCGTGCGGTTCAATTGGATTCAACGCCGGAAAACTCACCGGAGGAGACTGCCAGATGTGGGCCAAGCTGAAGACTTTGCTCGAATAATAGGCAGAGAGGTGGTGC
>PTM26_CONSENSUS
CTAGCTGTAAACGATGCGGGCCAGGTGTTGgCATTACTGCGAGTGATGTCAGTGCCGAAGGGAAGCCGTTAAGCCCGCCATCTGGGGAGTACGGTCGCAAGGCTGAAACTTAAAGGAATTGGCGGGGGAGCACCACAACGGG
TGGAGCCTGCGGTTCAATTGGATTCAACGCCGGRAAACTCACCGGAGGCGACAGCAAgATGAAgGTCAGGCTGAAGACCTTACYgGATTAGCTGAGAGGTGGCGC
TXv5v61689428
CTAGCTGTAAACGATGCGGGCCAGGTGTTGGCATTACTGCGAGTGATGTCAGTGCCGAAGGGAAGCCCGTTAAGCCCGCCATCTGGGGAGTACGGTCGCAAGGCTGAAACTTAAAGGAATTGGCGGGGGAGCACCACAACG
GGTGGAGCCTGCGGTTCAATTGGATTCAACGCCGGGAAACTCACCGGAGGCGACAGCAATATGAAGGTCAGGCTGAAGACCTTACCAGATTAGCTGAGAGGTGGCGC
TXv5v61425443
CTAGCTGTAAACGATGCGGGCCAGGTGTTGGCATTACTGCGAGTGATGTCAGTGCCGAAGGGAAGCCCGTTAAGCCCGCCATCTGGGGAGTACGGTCGCAAGGCTGAAACTTAAAGGAATTGGCGGGGGAGCACCACAACG
GGTGGAGCCTGCGGTTCAATTGGATTCAACGCCGGGAAACTCACCGGAGGCGACAGCAAGATGAAGGTCAGGCTGAAGACCTTACTGGATTAGCTGAGAGGTGGCGC
TXv5v61688200
CTAGCTGTAAACGATGCGGGCCAGGTGTTGACATTACTGCGAGTGATGTCAGTGCCGAAGGGAAGCCCGTTAAGCCCGCCATCTGGGGAGTACGGTCGCAAGGCTGAAACTTAAAGGAATTGGCGGGGGAGCACCACAACGG
GTGGAGCCTGCGGTTCAATTGGATTCAACGCCGGGAAACTCACCGGAGGCGACAGCAAGATGAAGGTCAGGCTGAAGACCTTACCGGATTAGCTGAGAGGTGGCGC
TXv5v60257863
CTAGCTGTAAACGATGCGGGCCAGGTGTTGACATTACTGCGAGTGATGTCAGTGCCGAAGGGAAGCCGTTAAGCCCGCCATCTGGGGAGTACGGTCGCAAGGCTGAAACTTAAAGGAATTGGCGGGGGAGCACCACAACGG
GTGGAGCCTGCGGTTCAATTGGATTCAACGCCGGGAAACTCACCGGAGGCGACAGCAAGATGAAGGTCAGGCTGAAGACCTTACCGGATTAGCTGAGAGGTGGCGC
TXv5v60716397
CTAGCTGTAAACGATGCGGGCCAGGTGTTGGCATTACTGCGAGTGATGTCAGTGCCGAAGGGAAGCCGTTAAGCCCGCCATCTGGGGAGTACGGTCGCAAGGCTGAAACTTAAAGGAATTGGCGGGGGAGCACCACAACGG
GTGGAGCCTGCGGTTCAATTGGATTCAACGCCGGGAAACTCACCGGAGGCGACAGCAAGATGAAGGTCAGGCTGAAGACCTTACCCGATTAGCTGAGAGGTGGCGC
TXv5v60258422
CTAGCTGTAAACGATGCGGGCCAGGTGTTGGCATTACTGCGAGTGATGTCAGTGCCGAAGGGAAGCCGTTAAGCCCGCCATCTGGGGAGTACGGTCGCAAGGCTGAAACTTAAAGGAATTGGCGGGGGAGCACCACAACGG
GTGGAGCCTGCGGTTCAATTGGATTCAACGCCGGGAAACTCACCGGAGGCGACAGCAATATGAAGGTCAGGCTGAAGACCTTACCAGATTAGCTGAGAGGTGGCGC
TXv5v60258367
CTAGCTGTAAACGATGCGGGCCAGGTGTTGGCATTACTGCGAGTGATGTCAGTGCCGAAGGGAAGCCGTTAAGCCCGCCATCTGGGGAGTACGGTCGCAAGGCTGAAACTTAAAGGAATTGGCGGGGGAGCACCACAACGG
GTGGAGCCTGCGGTTCAATTGGATTCAACGCCGGGAAACTCACCGGAGGCGACAGCAAGATGAAAGTCAGGCTGAAGACCTTACTGGATTAGCTGAGAGGTGGCGC
TXv5v60258396
CTAGCTGTAAACGATGCGGGCCAGGTGTTGGCATTACTGCGAGTGATGTCAGTGCCGAAGGGAAGCCGTTAAGCCCGCCATCTGGGGAGTACGGTCGCAAGGCTGAAACTTAAAGGAATTGGCGGGGGAGCACCACAACGG
GTGGAGCCTGCGGTTCAATTGGATTCAACGCCGGGAAACTCACCGGAGGCGACAGCAAGATGAAGGTCAGGCTGAAGACCTTACTGGATTAGCTGAGAGGTGGCGC
TXv5v61689332
CTAGCTGTAAACGATGCGGGCCAGGTGTTGGCATTACTGCGAGTGATGTCAGTGCCGAAGGGAAGCCCGTTAAGCCCGCCATCTGGGGAGTACGGTCGCAAGGCTGAAACTTAAAGGAATTGGCGGGGGAGCACCACAACG
GGTGGAGCCTGCGGTTCAATTGGATTCAACGCCGGGAAACTCACCGGAGGCGACAGCAAGATGAAGGTCAGGCTGAAGACCTTACCGGATTAGCTGAGAGGTGGCGC
TXv5v60715252
CTAGCTGTAAACGATGCGGGCCAGGTGTTGGCATTACTGCGAGTGATGTCAGTGCCGAAGGGAAGCCCGTTAAGCCCGCCATCTGGGGAGTACGGTCGCAAGGCTGAAACTTAAAGGAATTGGCGGGGGAGCACCACAACG
GGTGGAGCCTGCGGTTCAATTGGATTCAACGCCGGGAAACTCACCGGAGGCGACAGCAAGATGAAGGTCAGGCTGAAGACCTTACCAGATTAGCTGAGAGGTGGCGC
TXv5v60258423
CTAGCTGTAAACGATGCGGGCCAGGTGTTGGCATTACTGCGAGTGATGTCAGTGCCGAAGGGAAGCCGTTAAGCCCGCCATCTGGGGAGTACGGTCGCAAGGCTGAAACTTAAAGGAATTGGC
GGGGGAGCACCACAACGGGTGGAGCCTGCGGTTCAATTGGATTCAACGCCGGGAAACTCACCGGAGGCGACAGCAATATGAAGGTCAGGCTGAAGACCTTACCGGATTAGCTGAGAGGTGGCGC
TXv5v60258384
CTAGCTGTAAACGATGCGGGCCAGGTGTTGGCATTACTGCGAGTGATGTCAGTGCCGAAGGGAAGCCGTTAAGCCCGCCATCTGGGGAGTACGGTCGCAAGGCTGAAACTTAAAGGAATTGGCGGGGGAGCACCACAACGG
GTGGAGCCTGCGGTTCAATTGGATTCAACGCCGGGAAACTCACCGGAGGCGACAGCAAGATGAAGGTCAGGCTGAAGACCTTACCGGATTAGCTGAGAGGTGGCGC
TXv5v60258379
CTAGCTGTAAACGATGCGGGCCAGGTGTTGGCATTACTGCGAGTGATGTCAGTGCCGAAGGGAAGCCGTTAAGCCCGCCATCTGGGGAGTACGGTCGCAAGGCTGAAACTTAAAGGAATTGGCGGGGGAGCACCACAACGG
GTGGAGCCTGCGGTTCAATTGGATTCAACGCCGGGAAACTCACCGGAGGCGACAGCAAGATGAAGGTCAGGCTGAAGACCTTACCAGATTAGCTGAGAGGTGGCGC
TXv5v61425442
CTAGCTGTAAACGATGCGGGCCAGGTGTTGGCATTACTGCGAGTGATGTCAGTGCCGAAGGGAAGCCCGTTAAGCCCGCCATCTGGGGAGTACGGTCGCAAGGCTGAAACTTAAAGGAATTGGCGGGGGAGCACCACAACG
GGTGGAGCCTGCGGTTCAATTGGATTCAACGCCGGGAAACTCACCGGAGGCGACAGCAAGATGAAAGTCAGGCTGAAGACCTTACTGGATTAGCTGAGAGGTGGCGC
TXv5v60258269
CTAGCTGTAAACGATGCGGGCCAGGTGTTGGCATTACTGCGAGTGATGTCAGTGCCGAAGGGAAGCCGTTAAGCCCGCCATCTGGGGAGTACGGTCGCAAGGCTGAAACTTAAAGGAATTGGCGGGGGAGCACCACAACGG
GTGGAGCCTGCGGTTCAATTGGATTCAACGCCGGAAAACTCACCGGAGGCGACAGCAAGATGAAGGTCAGGCTGAAGACCTTACCGGATTAGCTGAGAGGTGGCGC
TXv5v61689136
CTAGCTGTAAACGATGCGGGCCAGGTGTTGGCATTACTGCGAGTGATGTCAGTGCCGAAGGGAAGCCCGTTAAGCCCGCCATCTGGGGAGTACGGTCGCAAGGCTGAAACTTAAAGGAATTGGCGGGGGAGCACCACAACG
GGTGGAGCCTGCGGTTCAATTGGATTCAACGCCGGAAAACTCACCGGAGGCGACAGCAAGATGAAGGTCAGGCTGAAGACCTTACCGGATTAGCTGAGAGGTGGCGC
TXv5v60258307
CTAGCTGTAAACGATGCGGGCCAGGTGTTGGCATTACTGCGAGTGATGTCAGTGCCGAAGGGAAGCCGTTAAGCCCGCCATCTGGGGAGTACGGTCGCAAGGCTGAAACTTAAAGGAATTGGCGGGGGAGCACCACAACGG
GTGGAGCCTGCGGTTCAATTGGATTCAACGCCGGAAAACTCACCGGAGGCGACAGCAATATGAAGGTCAGGCTGAAGACCTTACCGGATTAGCTGAGAGGTGGCGC
TXv5v61689106
CTAGCTGTAAACGATGCGGGCCAGGTGTTGGCATTACTGCGAGTGATGTCAGTGCCGAAGGGAAGCCCGTTAAGCCCGCCATCTGGGGAGTACGGTCGCAAGGCTGAAACTTAAAGGAATTGGCGGGGGAGCACCACAACG
GGTGGAGCCTGCGGTTCAATTGGATTCAACGCCGGAAAACTCACCGGAGGCGACAGCAAGATGAAAGTCAGGCTGAAGACCTTACTGGATTAGCTGAGAGGTGGCGC
TXv5v60258247
CTAGCTGTAAACGATGCGGGCCAGGTGTTGGCATTACTGCGAGTGATGTCAGTGCCGAAGGGAAGCCGTTAAGCCCGCCATCTGGGGAGTACGGTCGCAAGGCTGAAACTTAAAGGAATTGGCGGGGGAGCACCACAACGG
GTGGAGCCTGCGGTTCAATTGGATTCAACGCCGGAAAACTCACCGGAGGCGACAGCAAGATGAAAGTCAGGCTGAAGACCTTACTGGATTAGCTGAGAGGTGGCGC
TXv5v60258276
CTAGCTGTAAACGATGCGGGCCAGGTGTTGGCATTACTGCGAGTGATGTCAGTGCCGAAGGGAAGCCGTTAAGCCCGCCATCTGGGGAGTACGGTCGCAAGGCTGAAACTTAAAGGAATTGGCGGGGGAGCACCACAACGG
GTGGAGCCTGCGGTTCAATTGGATTCAACGCCGGAAAACTCACCGGAGGCGACAGCAAGATGAAGGTCAGGCTGAAGACCTTACTGGATTAGCTGAGAGGTGGCGC
TXv5v60258315
CTAGCTGTAAACGATGCGGGCCAGGTGTTGGCATTACTGCGAGTGATGTCAGTGCCGAAGGGAAGCCGTTAAGCCCGCCATCTGGGGAGTACGGTCGCAAGGCTGAAACTTAAAGGAATTGGCGGGGGAGCACCACAACGG
GTGGAGCCTGCGGTTCAATTGGATTCAACGCCGGAAAACTCACCGGAGGCGACAGCGAGATGAAGGTCAGGCTGAAGACCTTACCGGATTAGCTGAGAGGTGGCGC
>PTM27_CONSENSUS
CCAGCTGTAAACGATGCGGGCCAGGTGTTGgcATTACTGCGAGTGATGTCAGTGCCAAAGGGAAGCCGTTAAGCCCGCCATCTGGGgAGTACGGTCGCAAGGCTGAAACTTAAAGaAATTGGCGGGGGAGCACCACAACGGG
TGGAGCcTGCGGTTCAATYGGATTCAACGCCGGAAAaCTCACCGGAGGCgACAGCgAGATGAAGGTCAGGCTGAAGACCTTACcgGATTAGCTGAGAGGTGGCGC
TXv5v6-1671056
CCAGCTGTAAACGATGCGGGCCAGGTGTTGGCATTACTGCGAGTGATGTCAGTGCCAAAGGGAAGCCCGTTAAGCCCGCCATCTGGGGAGTACGGTCGCAAGGCTGAAACTTAAAGAAATTGGCGGGGGAGCACCACAACGG
GTGGAGCCTGCGGTTCAATTGGATTCAACGCCGGAAAACTCACCGGAGGCGACAGCGAGATGAAGGTCAGGCTGAAGACCTTACCAGATTAGCTGAGAGGTGGCGC
TXv5v6-0237067
CCAGCTGTAAACGATGCGGGCCAGGTGTTGGCATTACTGCGAGTGATGTCAGTGCCAAAGGGAAGCCGTTAAGCCCGCCATCTGGGGAGTACGGTCGCAAGGCTGAAACTTAAAGAAATTGGCGGGGGAGCACCACAACGG
GTGGAGCCTGCGGTTCAATTGGATTCAACGCCGGAAAACTCACCGGAGGCGACAGCGAGATGAAGGTCAGGCTGAAGACCTTACCAGATTAGCTGAGAGGTGGCGC
TXv5v6-1672136
CCAGCTGTAAACGATGCGGGCCAGGTGTTGGCATTACTGCGAGTGATGTCAGTGCCAAAGGGGAAGCCGTTAAGCCCGCCATCTGGGGAGTACGGTCGCAAGGCTGAAACTTAAAGAAATTGGCGGGGGAGCACCACAACG
GGTGGAGCCTGCGGTTCAATTGGATTCAACGCCGGAAAACTCACCGGAGGCGACAGCGAGATGAAGGTCAGGCTGAAGACCTTACCGGATTAGCTGAGAGGTGGCGC
TXv5v6-0237299
CCAGCTGTAAACGATGCGGGCCAGGTGTTGGCATTACTGCGAGTGATGTCAGTGCCAAAGGGAAGCCGTTAAGCCCGCCATCTGGGGAGTACGGTCGCAAGGCTGAAACTTAAAGGAAATTGGCGGGGGAGCACCACAACG
GGTGGAGCCTGCGGTTCAATTGGATTCAACGCCGGAAAACTCACCGGAGGCGACAGCGAGATGAAGGTCAGGCTGAAGACCTTACCGGATTAGCTGAGAGGTGGCGC
TXv5v6-0237037
CCAGCTGTAAACGATGCGGGCCAGGTGTTGGCATTACTGCGAGTGATGTCAGTGCCAAAGGGAAGCCGTTAAGCCCGCCATCTGGGGAGTACGGTCGCAAGGCTGAAACTTAAAGAAATTGGCGGGGGAGCACCACAACGG
GTGGAGCCTGCGGTTCAATTGGATTCAACGCCGGAAAACTCACCGGAGGCAACAGCGAGATGAAGGTCAGGCTGAAGACCTTACCGGATTAGCTGAGAGGTGGCGC
TXv5v6-1376733
CCAGCTGTAAACGATGCGGGCCAGGTGTTGGCATTACTGCGAGTGATGTCAGTGCCAAAGGGAAGCCCGTTAAGCCCGCCATCTGGGGAGTACGGTCGCAAGGCTGAAACTTAAAGAAATTGGCGGGGGAGCACCACAACGG
GTGGAGCCTGCGGTTCAATTGGATTCAACGCCGGAAAACTCACCGGAGGCGACAGCGAGATGAAGGTCAGGCTGAAGACCTTACCGGATTAGCTGAGAGGTGGCGC
TXv5v6-0237185
CCAGCTGTAAACGATGCGGGCCAGGTGTTGGCATTACTGCGAGTGATGTCAGTGCCAAAGGGAAGCCGTTAAGCCCGCCATCTGGGGAGTACGGTCGCAAGGCTGAAACTTAAAGAAATTGGCGGGGGAGCACCACAACGG
GTGGAGCTTGCGGTTCAATTGGATTCAACGCCGGAAAACTCACCGGAGGCGACAGCGAGATGAAGGTCAGGCTGAAGACCTTACCGGATTAGCTGAGAGGTGGCGC
TXv5v6-0237083
CCAGCTGTAAACGATGCGGGCCAGGTGTTGGCATTACTGCGAGTGATGTCAGTGCCAAAGGGAAGCCGTTAAGCCCGCCATCTGGGGAGTACGGTCGCAAGGCTGAAACTTAAAGAAATTGGCGGGGGAGCACCACAACGG
GTGGAGCCTGCGGTTCAATTGGATTCAACGCCGGAAAACTCACCGGAGGCGACAGCGAGATGAAGGTCAGGCTGAAGACCTTACCGGATTAGCTGAGAGGTGGCGC
TXv5v6-1377062
CCAGCTGTAAACGATGCGGGCCAGGTGTTGGCATTACTGCGAGTGATGTCAGTGCCAAAGGGAAGCCGTTAAGCCCGCCATCTGGGAGTACGGTCGCAAGGCTGAAACTTAAAGAAATTGGCGGGGGAGCACCACAACGGGT
GGAGCTTGCGGTTCAATTGGATTCAACGCCGGAAAACTCACCGGAGGCGACAGCGAGATGAAGGTCAGGCTGAAGACCTTACCGGATTAGCTGAGAGGTGGCGC
TXv5v6-0236558
CCAGCTGTAAACGATGCGGGCCAGGTGTTGGCATTACTGCGAGTGATGTCAGTGCCAAAGGGAAGCCGTTAAGCCCGCCATCTGGGAGTACGGTCGCAAGGCTGAAACTTAAAGAAATTGGCGGGGGAGCACCACAACGGGT
GGAGCCTGCGGTTCAATTGGATTCAACGCCGGAAAACTCACCGGAGGCGACAGCGAGATGAAGGTCAGGCTGAAGACCTTACCGGATTAGCTGAGAGGTGGCGC
TXv5v6-0237291
CCAGCTGTAAACGATGCGGGCCAGGTGTTGGCATTACTGCGAGTGATGTCAGTGCCAAAGGGAAGCCGTTAAGCCCGCCATCTGGGGAGTACGGTCGCAAGGCTGAAACTTAAAGGAAATTGGCGGGGGAGCACCACAACG
GGTGGAGCCTGCGGTTCAATCGGATTCAACGCCGGAAAACTCACCGGAGGCGACAGCAAGATGAAGGTCAGGCTGAAGACCTTACCAGATTAGCTGAGAGGTGGCGC
TXv5v6-0236906
CCAGCTGTAAACGATGCGGGCCAGGTGTTGGCATTACTGCGAGTGATGTCAGTGCCAAAGGGAAGCCGTTAAGCCCGCCATCTGGGGAGTACGGTCGCAAGGCTGAAACTTAAAGAAATTGGCGGGGGAGCACCACAACGG
GTGGAGCCTGCGGTTCAATCGGATTCAACGCCGGAAAACTCACCGGAGGCGACAGCGAGATGAAGGTCAGGCTGAAGACCTTACCGGATTAGCTGAGAGGTGGCGC
TXv5v6-0236917
CCAGCTGTAAACGATGCGGGCCAGGTGTTGGCATTACTGCGAGTGATGTCAGTGCCAAAGGGAAGCCGTTAAGCCCGCCATCTGGGGAGTACGGTCGCAAGGCTGAAACTTAAAGAAATTGGCGGGGGAGCACCACAACGG
GTGGAGCCTGCGGTTCAATCGGATTCAACGCCGGAAAACTCACCGGAGGCGACAGCGAGATGAAGGTCAGGCTGAAGACCTTACTGGATTAGCTGAGAGGTGGCGC
TXv5v6-0624771
CCAGCTGTAAACGATGCGGGCCAGGTGTTGGTATTACTGCGAGTGATGTCAGTGCCAAAGGGAAGCCGTTAAGCCCGCCATCTGGGGAGTACGGTCGCAAGGCTGAAACTTAAAGAAATTGGCGGGGGAGCACCACAACGG
GTGGAGCCTGCGGTTCAATCGGATTCAACGCCGGAAAACTCACCGGAGGCGACAGCGAGATGAAGGTCAGGCTGAAGACCTTACCGGATTAGCTGAGAGGTGGCGC
TXv5v6-0236386
CCAGCTGTAAACGATGCGGGCCAGGTGTTGACATTACTGCGAGTGATGTCAGTGCCAAAGGGAAGCCGTTAAGCCCGCCATCTGGGGAGTACGGTCGCAAGGCTGAAACTTAAAGAAATTGGCGGGGGAGCACCACAACGG
GTGGAGCCTGCGGTTCAATCGGATTCAACGCCGGAAAACTCACCGGAGGCGACAGCGAGATGAAGGTCAGGCTGAAGACCTTACCGGATTAGCTGAGAGGTGGCGC
TXv5v6-0236838
CCAGCTGTAAACGATGCGGGCCAGGTGTTGGCATTACTGCGAGTGATGTCAGTGCCAAAGGGAAGCCGTTAAGCCCGCCATCTGGGGAGTACGGTCGCAAGGCTGAAACTTAAAGAAATTGGCGGGGGAGCACCACAACGG
GTGGAGCCTGCGGTTCAATCGGATTCAACGCCGGAAAACTCACCGGAGGCGACAGCAAGATGAAGGTCAGGCTGAAGACCTTACCGGATTAGCTGAGAGGTGGCGC
TXv5v6-0236818
CCAGCTGTAAACGATGCGGGCCAGGTGTTGGCATTACTGCGAGTGATGTCAGTGCCAAAGGGAAGCCGTTAAGCCCGCCATCTGGGGAGTACGGTCGCAAGGCTGAAACTTAAAGAAATTGGCGGGGGAGCACCACAACGG
GTGGAGCCTGCGGTTCAATCGGATTCAACGCCGGAAAACTCACCGGAGGCGACAGCAAGATGAAGGTCAGGCTGAAGACCTTACCAGATTAGCTGAGAGGTGGCGC
TXv5v6-0236985
CCAGCTGTAAACGATGCGGGCCAGGTGTTGGCATTACTGCGAGTGATGTCAGTGCCAAAGGGAAGCCGTTAAGCCCGCCATCTGGGGAGTACGGTCGCAAGGCTGAAACTTAAAGAAATTGGCGGGGGAGCACCACAACGG
GTGGAGCCTGCGGTTCAATCGGATTCAACGCCGGAAATCTCACCGGAGGCGACAGCGAGATGAAGGTCAGGCTGAAGACCTTACCGGATTAGCTGAGAGGTGGCGC
TXv5v6-0621787
CCAGCTGTAAACGATGCGGGCCAGGTGTTGGCATTACTGCGAGTGATGTCAGTGCCAAAGGGAAGCCGTTAAGCCCGCCATCTGGGAGTACGGTCGCAAGGCTGAAACTTAAAGAAATTGGCGGGGGAGCACCACAACGGGT
GGAGCCTGCGGTTCAATCGGATTCAACGCCGGAAAACTCACCGGAGGCGACAGCGAGATGAAGGTCAGGCTGAAGACCTTACCGGATTAGCTGAGAGGTGGCGC
>PTM28_CONSENSUS
CACGCTGTAAACGATGGGAACTAGGTGTAGCGGGTATTGATCCCTGCTGTGCCGAAGCTAACGCATTAAGTTCCCCGCCTGGGGAGTACGGTCGCAAGGCTAAAACTCAAAGGAATTGACGGGGGCCCGCACAAGCGGTGGA
GCATGTGGTTCAATTCGATGCAACGCGAAAAACCTTACCTGGGTTTGACATCCTTTGACAGTCYCTGAAAGGGGATCTTTCCGATTTATCGGGACAAAGTGACAGGTGCTGC
TXv5v6-0545759
CACGCTGTAAACGATGGGAACTAGGTGTAGCGGGTATTGATCCCTGCTGTGCCGAAGCTAACGCATTAAGTTCCCCGCCTGGGGAGTACGGTCGCAAGGCTAAAACTCAAAGGAATTGACGGGGGCCCGCACAAGCGGTGGA
GCATGTGGTTCAATTCGATGCAACGCGAAAAACCTTACCTGGGTTTGACATCCTTTGACAGTCTCTGAAAGGGGATCTTTCCGATTTATCGGGACAAAGTGACAGGTGCTGC
TXv5v6-0194637
CACGCTGTAAACGATGGGAACTAGGTGTAGCGGGTATTGATCCCTGCTGTGCCGAAGCTAACGCATTAAGTTCCCCGCCTGGGGAGTACGGTCGCAAGGCTAAAACTCAAAGGAATTGACGGGGGCCCGCACAAGCGGTGGA
GCATGTGGTTCAATTCGATGCAACGCGAAAAACCTTACCTGGGTTTGACATCCTTTGACAGTCCCTGAAAGGGGATCTTTCCGATTTATCGGGACAAAGTGACAGGTGCTGC
>PTM29_CONSENSUS
CACGCCCTAAACGATGGGCACTAGGTGCAGGGGGTGTTGACCCCTCCTGTGCCGCAGCTAACGCATTAAGTGCCCCGCCTGGGGAGTACGGCCGCAAGGTTGAAACTCAAAGGAATTGACGGGGGCCCGCACAAGCGGTGGA
GCATGTGGTTYAATTCGACGCAACGCGAAGAACCTTACCTGGGCTTGACATCCCGGGAACTCTGTGGAAACACGGAGGTGCCCCTTCGGGGGAACCTGGTGACAGGTGCTGC
TXv5v6-0045163
CACGCCCTAAACGATGGGCACTAGGTGCAGGGGGTGTTGACCCCTCCTGTGCCGCAGCTAACGCATTAAGTGCCCCGCCTGGGGAGTACGGCCGCAAGGTTGAAACTCAAAGGAATTGACGGGGGCCCGCACAAGCGGTGGA
GCATGTGGTTCAATTCGACGCAACGCGAAGAACCTTACCTGGGCTTGACATCCCGGGAACTCTGTGGAAACACGGAGGTGCCCCTTCGGGGGAACCTGGTGACAGGTGCTGC
TXv5v6-0045206
CACGCCCTAAACGATGGGCACTAGGTGCAGGGGGTGTTGACCCCTCCTGTGCCGCAGCTAACGCATTAAGTGCCCCGCCTGGGGAGTACGGCCGCAAGGTTGAAACTCAAAGGAATTGACGGGGGCCCGCACAAGCGGTGGA
GCATGTGGTTTAATTCGACGCAACGCGAAGAACCTTACCTGGGCTTGACATCCCGGGAACTCTGTGGAAACACGGAGGTGCCCCTTCGGGGGAACCTGGTGACAGGTGCTGC
>PTM30_CONSENSUS
CACGCCSTAAACAGTGGACACTAGATATGGGGAGTATCGACCCTTCTCGTGTCGAAGCTAACGCCTTAAGTGTCCCACCTGGGGACTACGATCGCAAGGTTAAAACTCAAAGGAATTGACGGGGGCCCGCACAAGCAGCGGAG
CGTGTGGTTTAATTCGATGCTACGCGAAGAACCTTACCAGGGCTTGACATGTCAGTAGTAGGAATCCGAAAGGAGGACGACCTGTATCCAGTCAGGAACTGTCACAGGTGCTGC
TXv5v6-0063016
CACGCCGTAAACAGTGGACACTAGATATGGGGAGTATCGACCCTTCTCGTGTCGAAGCTAACGCCTTAAGTGTCCCACCTGGGGACTACGATCGCAAGGTTAAAACTCAAAGGAATTGACGGGGGCCCGCACAAGCAGCGGAG
CGTGTGGTTTAATTCGATGCTACGCGAAGAACCTTACCAGGGCTTGACATGTCAGTAGTAGGAATCCGAAAGGAGGACGACCTGTATCCAGTCAGGAACTGTCACAGGTGCTGC
TXv5v6-1284822
CACGCCCTAAACAGTGGACACTAGATATGGGGAGTATCGACCCTTCTCGTGTCGAAGCTAACGCCTTAAGTGTCCCACCTGGGGACTACGATCGCAAGGTTAAAACTCAAAGGAATTGACGGGGGCCCGCACAAGCAGCGGAG
CGTGTGGTTTAATTCGATGCTACGCGAAGAACCTTACCAGGGCTTGACATGTCAGTAGTAGGAATCCGAAAGGAGGACGACCTGTATCCAGTCAGGAACTGTCACAGGTGCTGC
>PTM31_CONSENSUS
CTAGCTGTAAACGATGCGGGCTAGGTGTTGGCATTACTGCGAGTGATGCCAGTGCCGAAGGGAAGCCGTTAAGCCCGCCATCTGGGgAGTACGGTCGCAAGGCTGAAACTTAAAGGAATTGGCGGGGGAGCACTACAACGGG
TGGAGCCTGCGGTTCAATTGGATTCAACGCCGGaAAACTCACCGGAGGCGACAGCGAgATGAAGGTCAGGcTGAAGACCTTACcGGATTAGCTGAGAGGTGGCGC
TXv5v6-0258790
CTAGCTGTAAACGATGCGGGCTAGGTGTTGGCATTACTGCGAGTGATGCCAGTGCCGAAGGGAAGCCGTTAAGCCCGCCATCTGGGGAGTACGGTCGCAAGGCTGAAACTTAAAGGAATTGGCGGGGGAGCACTACAACGG
GTGGAGCCTGCGGTTCAATTGGATTCAACGCCGGGAAACTCACCGGAGGCGACAGCGAGATGAAGGTCAGGCTGAAGACCTTACCGGATTAGCTGAGAGGTGGCGC
TXv5v6-0717922
CTAGCTGTAAACGATGCGGGCTAGGTGTTGGCATTACTGCGAGTGATGCCAGTGCCGAAGGGAAGCCGTTAAGCCCGCCATCTGGGAGTACGGTCGCAAGGCTGAAACTTAAAGGAATTGGCGGGGGAGCACTACAACGGGT
GGAGCCTGCGGTTCAATTGGATTCAACGCCGGGAAACTCACCGGAGGCGACAGCGAGATGAAGGTCAGGCTGAAGACCTTACCGGATTAGCTGAGAGGTGGCGC
TXv5v6-0258776
CTAGCTGTAAACGATGCGGGCTAGGTGTTGGCATTACTGCGAGTGATGCCAGTGCCGAAGGGAAGCCGTTAAGCCCGCCATCTGGGGAGTACGGTCGCAAGGCTGAAACTTAAAGGAATTGGCGGGGGAGCACTACAACGG
GTGGAGCCTGCGGTTCAATTGGATTCAACGCCGGAAAACTCACCGGAGGCGACAGCGATATGAAGGTCAGGCTGAAGACCTTACCGGATTAGCTGAGAGGTGGCGC
TXv5v6-0258773
CTAGCTGTAAACGATGCGGGCTAGGTGTTGGCATTACTGCGAGTGATGCCAGTGCCGAAGGGAAGCCGTTAAGCCCGCCATCTGGGGAGTACGGTCGCAAGGCTGAAACTTAAAGGAATTGGCGGGGGAGCACTACAACGG
GTGGAGCCTGCGGTTCAATTGGATTCAACGCCGGAAAACTCACCGGAGGCGACAGCGAGATGAAGGTCAGGCTGAAGACCTTACCGGATTAGCTGAGAGGTGGCGC
TXv5v6-1691264
CTAGCTGTAAACGATGCGGGCTAGGTGTTGGCATTACTGCGAGTGATGCCAGTGCCGAAGGGAAGCCCGTTAAGCCCGCCATCTGGGGAGTACGGTCGCAAGGCTGAAACTTAAAGGAATTGGCGGGGGAGCACTACAACGG
GTGGAGCCTGCGGTTCAATTGGATTCAACGCCGGAAAACTCACCGGAGGCGACAGCGAGATGAAGGTCAGGCTGAAGACCTTACCGGATTAGCTGAGAGGTGGCGC
TXv5v6-0718915
CTAGCTGTAAACGATGCGGGCTAGGTGTTGGCATTACTGCGAGTGATGCCAGTGCCGAAGGGGAAGCCGTTAAGCCCGCCATCTGGGGAGTACGGTCGCAAGGCTGAAACTTAAAGGAATTGGCGGGGGAGCACTACAACG
GGTGGAGCCTGCGGTTCAATTGGATTCAACGCCGGAAAACTCACCGGAGGCGACAGCGAGATGAAGGTCAGGCTGAAGACCTTACCGGATTAGCTGAGAGGTGGCGC
TXv5v6-0258774
CTAGCTGTAAACGATGCGGGCTAGGTGTTGGCATTACTGCGAGTGATGCCAGTGCCGAAGGGAAGCCGTTAAGCCCGCCATCTGGGGAGTACGGTCGCAAGGCTGAAACTTAAAGGAATTGGCGGGGGAGCACTACAACGG
GTGGAGCCTGCGGTTCAATTGGATTCAACGCCGGAAAACTCACCGGAGGCGACAGCGAGATGAAGGTCAGGTTGAAGACCTTACTGGATTAGCTGAGAGGTGGCGC
>PTM32_CONSENSUS
CTAGCCGTAAACGATGGGCACTAGATGTTTCCGCTTTTAGCGGRGGTGTCGAAGCTAACGCATTAAGTGCCCCGCCTGGGGAGTACGGTCGCAAGGCTGAAACTCAAAGGAATTGACGGGGGCCCGCACAAGCGGTGGAGCA
TGTGGTTCAATTCGACGCAACGCGAAGAACCTTACCTGGGTTTGAACTGCTGGTGGTAARACCTCGAAAGRGGAATGATCCTGGCTTGCCAGGAAGCCAGCAGAGGTGCTGC
TXv5v6-0252248
CTAGCCGTAAACGATGGGCACTAGATGTTTCCGCTTTTAGCGGGGGTGTCGAAGCTAACGCATTAAGTGCCCCGCCTGGGGAGTACGGTCGCAAGGCTGAAACTCAAAGGAATTGACGGGGGCCCGCACAAGCGGTGGAGCA
TGTGGTTCAATTCGACGCAACGCGAAGAACCTTACCTGGGTTTGAACTGCTGGTGGTAAGACCTCGAAAGGGGAATGATCCTGGCTTGCCAGGAAGCCAGCAGAGGTGCTGC
TXv5v6-0689158
CTAGCCGTAAACGATGGGCACTAGATGTTTCCGCTTTTAGCGGAGGTGTCGAAGCTAACGCATTAAGTGCCCCGCCTGGGGAGTACGGTCGCAAGGCTGAAACTCAAAGGAATTGACGGGGGCCCGCACAAGCGGTGGAGCA
TGTGGTTCAATTCGACGCAACGCGAAGAACCTTACCTGGGTTTGAACTGCTGGTGGTAAAACCTCGAAAGAGGAATGATCCTGGCTTGCCAGGAAGCCAGCAGAGGTGCTGC
TXv5v6-0252247
CTAGCCGTAAACGATGGGCACTAGATGTTTCCGCTTTTAGCGGGGGTGTCGAAGCTAACGCATTAAGTGCCCCGCCTGGGGAGTACGGTCGCAAGGCTGAAACTCAAAGGAATTGACGGGGGCCCGCACAAGCGGTGGAGCA
TGTGGTTCAATTCGACGCAACGCGAAGAACCTTACCTGGGTTTGAACTGCTGGTGGTAAAACCTCGAAAGGGGAATGATCCTGGCTTGCCAGGAAGCCAGCAGAGGTGCTGC
>PTM33_CONSENSUS
CTAGCCGTAAACGATGGGCACTTGACGTAGGCGATAATAGTCTGCGTCGTAGCTAACGTGTTAAGTGCCCCGCCTGGGGAGTACGTTCGCAAGGATGAAACTCAAAGGAATTGACGGGGACCCGCACAAGCGGTGGAGGATG
TGGTTTAATTCGAGGCAACGCGAAGAACCTTACCTGGGCTTGACATACAGGAAGTAGGAMCCCGAAAGGGTAACGACCGGTAACCAATCCGGAGCCTGTACAGGTGTTGC
TXv5v6-0254691
CTAGCCGTAAACGATGGGCACTTGACGTAGGCGATAATAGTCTGCGTCGTAGCTAACGTGTTAAGTGCCCCGCCTGGGGAGTACGTTCGCAAGGATGAAACTCAAAGGAATTGACGGGGACCCGCACAAGCGGTGGAGGATG
TGGTTTAATTCGAGGCAACGCGAAGAACCTTACCTGGGCTTGACATACAGGAAGTAGGACCCCGAAAGGGTAACGACCGGTAACCAATCCGGAGCCTGTACAGGTGTTGC
TXv5v6-0254679
CTAGCCGTAAACGATGGGCACTTGACGTAGGCGATAATAGTCTGCGTCGTAGCTAACGTGTTAAGTGCCCCGCCTGGGGAGTACGTTCGCAAGGATGAAACTCAAAGGAATTGACGGGGACCCGCACAAGCGGTGGAGGATG
TGGTTTAATTCGAGGCAACGCGAAGAACCTTACCTGGGCTTGACATACAGGAAGTAGGAACCCGAAAGGGTAACGACCGGTAACCAATCCGGAGCCTGTACAGGTGTTGC
>PTM34_CONSENSUS
CTAGCTGTAAACGATGTGGACTTGGCGTTGGTGGGGTCAAATCCATCAGTGCCGKAGCTAACGCGATAAGTCCACCGCCTGGGGACTACGACCGCAAGGTTAAAACTCAAAGGAATTGGCGGGGGCCCGCACAAGCAGCGGA
GCGTGTGGTTTAATTCGATGCTACACGAAGAACCTTACCCGGGTTTGACATCCAGGTGGTAGGGAACCGAAAGGCGACCGACCCTTCGGGGAGCCTGGACAGGTGCTGC
TXv5v6-0262828
CTAGCTGTAAACGATGTGGACTTGGCGTTGGTGGGGTCAAATCCATCAGTGCCGGAGCTAACGCGATAAGTCCACCGCCTGGGGACTACGACCGCAAGGTTAAAACTCAAAGGAATTGGCGGGGGCCCGCACAAGCAGCGGA
GCGTGTGGTTTAATTCGATGCTACACGAAGAACCTTACCCGGGTTTGACATCCAGGTGGTAGGGAACCGAAAGGCGACCGACCCTTCGGGGAGCCTGGACAGGTGCTGC
TXv5v6-0262852
CTAGCTGTAAACGATGTGGACTTGGCGTTGGTGGGGTCAAATCCATCAGTGCCGTAGCTAACGCGATAAGTCCACCGCCTGGGGACTACGACCGCAAGGTTAAAACTCAAAGGAATTGGCGGGGGCCCGCACAAGCAGCGGA
GCGTGTGGTTTAATTCGATGCTACACGAAGAACCTTACCCGGGTTTGACATCCAGGTGGTAGGGAACCGAAAGGCGACCGACCCTTCGGGGAGCCTGGACAGGTGCTGC
>PTM35_CONSENSUS
CTAGCTGTAAACGATGGATACTAGATTTTGCAAGTTATTGCWAGATCGAAGCTAACGCATTAAGTATCCCGCCTGGGGAGTACGGYCGCAAGGCTAAAACTCAAAGGAATTGACGGGGACCCGCACAAGCAGTGGAGCATGT
GGTTTAATTCGATGCAACGCGAAGAACCTTACCTGGGCTTGAACTGTAGGCATTAGCCGCCTGAAAGGGTTGGTTATCCTCTTCGGAGGAACCTATAGAGGTGCTGC
TXv5v6-1434138
CTAGCTGTAAACGATGGATACTAGATTTTGCAAGTTATTGCAAGATCGAAGCTAACGCATTAAGTATCCCGCCTGGGGAGTACGGCCGCAAGGCTAAAACTCAAAGGAATTGACGGGGACCCGCACAAGCAGTGGAGCATGTG
GTTTAATTCGATGCAACGCGAAGAACCTTACCTGGGCTTGAACTGTAGGCATTAGCCGCCTGAAAGGGTTGGTTATCCTCTTCGGAGGAACCTATAGAGGTGCTGC
TXv5v6-0259077
CTAGCTGTAAACGATGGATACTAGATTTTGCAAGTTATTGCTAGATCGAAGCTAACGCATTAAGTATCCCGCCTGGGGAGTACGGTCGCAAGGCTAAAACTCAAAGGAATTGACGGGGACCCGCACAAGCAGTGGAGCATGTG
GTTTAATTCGATGCAACGCGAAGAACCTTACCTGGGCTTGAACTGTAGGCATTAGCCGCCTGAAAGGGTTGGTTATCCTCTTCGGAGGAACCTATAGAGGTGCTGC
TXv5v6-0722828
CTAGCTGTAAACGATGGATACTAGATTTTGCAAGTTATTGCAAGATCGAAGCTAACGCATTAAGTATCCCGCCTGGGGAGTACGGTCGCAAGGCTAAAACTCAAAGGAATTGACGGGGACCCGCACAAGCAGTGGAGCATGTG
GTTTAATTCGATGCAACGCGAAGAACCTTACCTGGGCTTGAACTGTAGGCATTAGCCGCCTGAAAGGGTTGGTTATCCTCTTCGGAGGAACCTATAGAGGTGCTGC
>PTM36_CONSENSUS
CTAGCTGTAAACGATGGATACTAGGTGTGGGAGGTATCGACCCCTTCTGTGCCGCAGCTAACGCATTAAGTATCCCGCCTGGGGAGTACGGTCGCAAGGCTGAAACTCAAAGGAATTGACGGGGGCCCGCACAAGCGGTGGA
GCATGTGGTTTAATTCGACGCAACGCGAAGAACCTTACCGGGACTTGACATTATYTTGCCCGTCTAAGAAATTAGATCTTCTTTCCTTTTAGGGAAGACGARATAACAGGTGGTGC
TXv5v6-1437489
CTAGCTGTAAACGATGGATACTAGGTGTGGGAGGTATCGACCCCTTCTGTGCCGCAGCTAACGCATTAAGTATCCCGCCTGGGGAGTACGGTCGCAAGGCTGAAACTCAAAGGAATTGACGGGGGCCCGCACAAGCGGTGGA
GCATGTGGTTTAATTCGACGCAACGCGAAGAACCTTACCGGGACTTGACATTATTTTGCCCGTCTAAGAAATTAGATCTTCTTTCCTTTTAGGGAAGACGAAATAACAGGTGGTGC
TXv5v6-0726865
CTAGCTGTAAACGATGGATACTAGGTGTGGGAGGTATCGACCCCTTCTGTGCCGCAGCTAACGCATTAAGTATCCCGCCTGGGGAGTACGGTCGCAAGGCTGAAACTCAAAGGAATTGACGGGGGCCCGCACAAGCGGTGGA
GCATGTGGTTTAATTCGACGCAACGCGAAGAACCTTACCGGGACTTGACATTATCTTGCCCGTCTAAGAAATTAGATCTTCTTTCCTTTTAGGGAAGACGAGATAACAGGTGGTGC
>PTM37_CONSENSUS
CACGCCSTAAACGGTGGACACTAGATATAGGARGTATCGACCCYTTCTGTGTCGAAGCTAACGCCTTAAGTGTCCCGCCTGGGKAGTACGGCCGCAAGGCTGAAACTCAAAGGAATTGACGGGGGCCCGCACAAGCAGCGGAG
CGTGTGGTTTAATTCGATGCTACRCGAAGAACCTTACCAGGGCTTGACATGRCAGAAGTAGGAATCCGAAAGGACGACGACCTGTATCCAGTCAGGAGCTGYCACAGGTGCTGC
TXv5v6-0489473
CACGCCGTAAACGGTGGACACTAGATATAGGAGGTATCGACCCCTTCTGTGTCGAAGCTAACGCCTTAAGTGTCCCGCCTGGGTAGTACGGCCGCAAGGCTGAAACTCAAAGGAATTGACGGGGGCCCGCACAAGCAGCGGA
GCGTGTGGTTTAATTCGATGCTACACGAAGAACCTTACCAGGGCTTGACATGGCAGAAGTAGGAATCCGAAAGGACGACGACCTGTATCCAGTCAGGAGCTGTCACAGGTGCTGC
TXv5v6-0059568
CACGCCCTAAACGGTGGACACTAGATATAGGAAGTATCGACCCTTTCTGTGTCGAAGCTAACGCCTTAAGTGTCCCGCCTGGGGAGTACGGCCGCAAGGCTGAAACTCAAAGGAATTGACGGGGGCCCGCACAAGCAGCGGA
GCGTGTGGTTTAATTCGATGCTACGCGAAGAACCTTACCAGGGCTTGACATGACAGAAGTAGGAATCCGAAAGGACGACGACCTGTATCCAGTCAGGAGCTGCCACAGGTGCTGC
>PTM38_CONSENSUS
CTAGCCGTAAACGATGGACACTTGACGTGGGCGATTTTAGTCTGCGTCGGAGCTAACGTATTAAGTGTCCCGCCTGGGGAGTACGTTCGCAAGGATGAAACTCAAAGGAATTGACGGGGACCCGCACAAGCGGTGGAGGATG
TGGTTTAATTCGAGGCAACGCGAAGAACCTTACCTGGGTTTGACATGCAGAAAGTAGGAGCCCGAAAGGGTRACAACCGGTAACCARTCCGGAATCTGCACAGGTGCTGC
TXv5v6-0678112
CTAGCCGTAAACGATGGACACTTGACGTGGGCGATTTTAGTCTGCGTCGGAGCTAACGTATTAAGTGTCCCGCCTGGGGAGTACGTTCGCAAGGATGAAACTCAAAGGAATTGACGGGGACCCGCACAAGCGGTGGAGGATG
TGGTTTAATTCGAGGCAACGCGAAGAACCTTACCTGGGTTTGACATGCAGAAAGTAGGAGCCCGAAAGGGTAACAACCGGTAACCAATCCGGAATCTGCACAGGTGCTGC
TXv5v6-0249051
CTAGCCGTAAACGATGGACACTTGACGTGGGCGATTTTAGTCTGCGTCGGAGCTAACGTATTAAGTGTCCCGCCTGGGGAGTACGTTCGCAAGGATGAAACTCAAAGGAATTGACGGGGACCCGCACAAGCGGTGGAGGATG
TGGTTTAATTCGAGGCAACGCGAAGAACCTTACCTGGGTTTGACATGCAGAAAGTAGGAGCCCGAAAGGGTGACAACCGGTAACCAGTCCGGAATCTGCACAGGTGCTGC
TXv5v6-0249046
CTAGCCGTAAACGATGGACACTTGACGTGGGCGATTTTAGTCTGCGTCGGAGCTAACGTATTAAGTGTCCCGCCTGGGGAGTACGTTCGCAAGGATGAAACTCAAAGGAATTGACGGGGACCCGCACAAGCGGTGGAGGATG
TGGTTTAATTCGAGGCAACGCGAAGAACCTTACCTGGGTTTGACATGCAGAAAGTAGGAGCCCGAAAGGGTGACAACCGGTAACCAATCCGGAATCTGCACAGGTGCTGC
>PTM39_CONSENSUS
CCAGCCGTAAACGATGCTCGCTATGTGTCAGGTACGGTGYGACCGTATCTGGTGCCGTAGGGAAGCCGTGAAGCGAGCCACCTGGGAAGTACGGYCGCAAGRCTGAAACTTAAAGGAATTGGCGGGGGAGCACTACAACGGT
GGAGCCTGCGGTTTAATTGGATTCAACGCCGGAAATCTTACCGGGKGAGACAGCARYATGAAGGTCAGGCTGAAGACCTTACCRGATYCGCTGAGAGGAAGTGC
TXv5v6-0231931
CCAGCCGTAAACGATGCTCGCTATGTGTCAGGTACGGTGTGACCGTATCTGGTGCCGTAGGGAAGCCGTGAAGCGAGCCACCTGGGAAGTACGGCCGCAAGGCTGAAACTTAAAGGAATTGGCGGGGGAGCACTACAACGG
GTGGAGCCTGCGGTTTAATTGGATTCAACGCCGGAAATCTTACCGGGGGAGACAGCAGCATGAAGGTCAGGCTGAAGACCTTACCAGATCCGCTGAGAGGAAGTGC
TXv5v6-0232006
CCAGCCGTAAACGATGCTCGCTATGTGTCAGGTACGGTGTGACCGTATCTGGTGCCGTAGGGAAGCCGTGAAGCGAGCCACCTGGGAAGTACGGTCGCAAGACTGAAACTTAAAGGAATTGGCGGGGGAGCACTACAACGGG
TGGAGCCTGCGGTTTAATTGGATTCAACGCCGGAAATCTTACCGGGGGAGACAGCAGCATGAAGGTCAGGCTGAAGACCTTACCAGATCCGCTGAGAGGAAGTGC
TXv5v6-0231898
CCAGCCGTAAACGATGCTCGCTATGTGTCAGGTACGGTGCGACCGTATCTGGTGCCGTAGGGAAGCCGTGAAGCGAGCCACCTGGGAAGTACGGTCGCAAGACTGAAACTTAAAGGAATTGGCGGGGGAGCACTACAACGG
GTGGAGCCTGCGGTTTAATTGGATTCAACGCCGGAAATCTTACCGGGTGAGACAGCAATATGAAGGTCAGGCTGAAGACCTTACCGGATTCGCTGAGAGGAAGTGC
>PTM40_CONSENSUS
CCAGCCCTAAACGATGTACACTTGGCATGCGYYRTATKRTGCGTGCCGTAGGTAACCTGTTAAGTGTACCGCCTGGGGAGTAYGCTCGCAAGGGTGAAACTCAAAGGAATTGACGGGGACCCGCACAAGCGGTGGAGGATGTG
GTTCAATTCGAGGCAACGCGAAGAACCTTACCTGGGCTTGACATGCTGATAGTACTRAACCGAAAGGTGAYGGATTCCACCTCTGGTGGAAAGTCAGCACAGGTGCTGC
TXv5v6-0217253
CCAGCCCTAAACGATGTACACTTGGCATGCGCTATATTGTGCGTGCCGTAGGTAACCTGTTAAGTGTACCGCCTGGGGAGTACGCTCGCAAGGGTGAAACTCAAAGGAATTGACGGGGACCCGCACAAGCGGTGGAGGATGTG
GTTCAATTCGAGGCAACGCGAAGAACCTTACCTGGGCTTGACATGCTGATAGTACTGAACCGAAAGGTGACGGATTCCACCTCTGGTGGAAAGTCAGCACAGGTGCTGC
TXv5v6-0217292
CCAGCCCTAAACGATGTACACTTGGCATGCGTCGTATGATGCGTGCCGTAGGTAACCTGTTAAGTGTACCGCCTGGGGAGTATGCTCGCAAGGGTGAAACTCAAAGGAATTGACGGGGACCCGCACAAGCGGTGGAGGATGT
GGTTCAATTCGAGGCAACGCGAAGAACCTTACCTGGGCTTGACATGCTGATAGTACTAAACCGAAAGGTGATGGATTCCACCTCTGGTGGAAAGTCAGCACAGGTGCTGC
>PTM41_CONSENSUS
CACGCAGTAAACGATGAACACTAGGTGTAGCGGGTATTGACCCCTGCTGTGCCGCAGTTAACGCATTAAGTGTTCCGCCTGGGGAGTACGACCGCAAGGTTAAAACTCAAAGGAATTGACGGGGGCCCGCACAAGCGGTGGA
GCATGTGGTTTAATTCGACGCAACGCGAAGAACCTTACCTGGATTTGACATCCcGGGAAgTCCCTTGAAAaAGGGATGTGCCCTTCGGGGAACCCGGTGACAGGTGCTGC
TXv5v6-0025886
CACGCAGTAAACGATGAACACTAGGTGTAGCGGGTATTGACCCCTGCTGTGCCGCAGTTAACGCATTAAGTGTTCCGCCTGGGGAGTACGACCGCAAGGTTAAAACTCAAAGGAATTGACGGGGGCCCGCACAAGCGGTGGA
GCATGTGGTTTAATTCGACGCAACGCGAAGAACCTTACCTGGATTTGACATCCTGGGAAGTCCCTTGAAAAAGGGATGTGCCCTTCGGGGAACCCGGTGACAGGTGCTGC
TXv5v6-0025873
CACGCAGTAAACGATGAACACTAGGTGTAGCGGGTATTGACCCCTGCTGTGCCGCAGTTAACGCATTAAGTGTTCCGCCTGGGGAGTACGACCGCAAGGTTAAAACTCAAAGGAATTGACGGGGGCCCGCACAAGCGGTGGA
GCATGTGGTTTAATTCGACGCAACGCGAAGAACCTTACCTGGATTTGACATCCCGGGAAGTCCCTTGAAAAAGGGATGTGCCCTTCGGGGAACCCGGTGACAGGTGCTGC
TXv5v6-0025863
CACGCAGTAAACGATGAACACTAGGTGTAGCGGGTATTGACCCCTGCTGTGCCGCAGTTAACGCATTAAGTGTTCCGCCTGGGGAGTACGACCGCAAGGTTAAAACTCAAAGGAATTGACGGGGGCCCGCACAAGCGGTGGA
GCATGTGGTTTAATTCGACGCAACGCGAAGAACCTTACCTGGATTTGACATCCCGGGAAATCCCTTGAAAAAGGGATGTGCCCTTCGGGGAACCCGGTGACAGGTGCTGC
TXv5v6-0025876
CACGCAGTAAACGATGAACACTAGGTGTAGCGGGTATTGACCCCTGCTGTGCCGCAGTTAACGCATTAAGTGTTCCGCCTGGGGAGTACGACCGCAAGGTTAAAACTCAAAGGAATTGACGGGGGCCCGCACAAGCGGTGGA
GCATGTGGTTTAATTCGACGCAACGCGAAGAACCTTACCTGGATTTGACATCCCGGGAAGTCCCTTGAAAGAGGGATGTGCCCTTCGGGGAACCCGGTGACAGGTGCTGC
>PTM42_CONSENSUS
CTAGCTGTAAACGATGGATACTAGGTGTGGGAGGTATCGACCCCTTCTGTGCCGcAGCTAACGCATTAAGTATCCCGCCTGGGGAGTACGGTCGCAAGGCTGAAACTCAAAGGAATTGACGGGGGCCCGCACAAGCGGTGGA
GCATGTGGTTTAATTCGACGCAACGCGAAGAACCTTACCGGGRCTTGACATTaTCTTGCCCGTCTAAGAAATTAGATCTTCTTCCTTTcGGAAGACGAGATAACAGGTGGTGC
TXv5v6-0726759
CTAGCTGTAAACGATGGATACTAGGTGTGGGAGGTATCGACCCCTTCTGTGCCGCAGCTAACGCATTAAGTATCCCGCCTGGGGAGTACGGTCGCAAGGCTGAAACTCAAAGGAATTGACGGGGGCCCGCACAAGCGGTGGA
GCATGTGGTTTAATTCGACGCAACGCGAAGAACCTTACCGGGACTTGACATTATCTTGCCCGTCTAAGAAATTAGATCTTCTTCCTTTCGGAAGACGAGATAACAGGTGGTGC
TXv5v6-0260150
CTAGCTGTAAACGATGGATACTAGGTGTGGGAGGTATCGACCCCTTCTGTGCCGTAGCTAACGCATTAAGTATCCCGCCTGGGGAGTACGGTCGCAAGGCTGAAACTCAAAGGAATTGACGGGGGCCCGCACAAGCGGTGGA
GCATGTGGTTTAATTCGACGCAACGCGAAGAACCTTACCGGGGCTTGACATTATCTTGCCCGTCTAAGAAATTAGATCTTCTTCCTTTCGGGGAAGACGAGATAACAGGTGGTGC
TXv5v6-0259561
CTAGCTGTAAACGATGGATACTAGGTGTGGGAGGTATCGACCCCTTCTGTGCCGCAGCTAACGCATTAAGTATCCCGCCTGGGGAGTACGGTCGCAAGGCTGAAACTCAAAGGAATTGACGGGGGCCCGCACAAGCGGTGGA
GCATGTGGTTTAATTCGACGCAACGCGAAGAACCTTACCGGGACTTGACATTATCTTGCCCGTCTAAGAAATTAGATCTTCTTCCTTTCGGGGAAGACGAGATAACAGGTGGTGC
TXv5v6-0259703
CTAGCTGTAAACGATGGATACTAGGTGTGGGAGGTATCGACCCCTTCTGTGCCGCAGCTAACGCATTAAGTATCCCGCCTGGGGAGTACGGTCGCAAGGCTGAAACTCAAAGGAATTGACGGGGGCCCGCACAAGCGGTGGA
GCATGTGGTTTAATTCGACGCAACGCGAAGAACCTTACCGGGGCTTGACATTGTCTTGCCCGTCTAAGAAATTAGATCTTCTTCCTTTTGGAAGACGAGATAACAGGTGGTGC
>PTM43_CONSENSUS
CTAGCTGTAAACGATGCTCGCTAGGTGTCAGACACGGTGCGACCGTGTTTGGTGCCGCAGGGAAGCCGTTAAGCGAGCCACCTGGGAAGTACGGTCGCAAGGCTGAAACTTAAAGGAATTGGCGGGGGAGCACTACAACGG
GTGGAGCCTGCGGTTTAATTGGATTCAACGCCGGAAAACTCACCGGGTGCGACAGCAAtATGTAGGTCAGGCTGAAGGTCTTACCTGAATCGCTGAGAGGAGGTGC
TXv5v6-0258903
CTAGCTGTAAACGATGCTCGCTAGGTGTCAGACACGGTGCGACCGTGTTTGGTGCCGCAGGGAAGCCGTTAAGCGAGCCACCTGGGAAGTACGGTCGCAAGGCTGAAACTTAAAGGAATTGGCGGGGGAGCACTACAACGG
GTGGAGCCTGCGGTTTAATTGGATTCAACGCCGGAAAACTCACCGGGTGCGACAGCAACATGTAGGTCAGGCTGAAGGTCTTACCTGAATCGCTGAGAGGAGGTGC
TXv5v6-1692076
CTAGCTGTAAACGATGCTCGCTAGGTGTCAGACACGGTGCGACCGTGTTTGGTGCCGCAGGGAAGCCCGTTAAGCGAGCCACCTGGGAAGTACGGTCGCAAGGCTGAAACTTAAAGGAATTGGCGGGGGAGCACTACAACGG
GTGGAGCCTGCGGTTTAATTGGATTCAACGCCGGAAAACTCACCGGGTGCGACAGCAATATGTAGGTCAGGCTGAAGGTCTTACCTGAATCGCTGAGAGGAGGTGC
TXv5v6-0258906
CTAGCTGTAAACGATGCTCGCTAGGTGTCAGACACGGTGCGACCGTGTTTGGTGCCGCAGGGAAGCCGTTAAGCGAGCCACCTGGGAAGTACGGTCGCAAGGCTGAAACTTAAAGGAATTGGCGGGGGAGCACTACAACGG
GTGGAGCCTGCGGTTTAATTGGATTCAACGCCGGAAAACTCACCGGGTGCGACAGCAATATGTAGGTCAGGCTGAAGGTCTTACCTGAATCGCTGAGAGGAGGTGC
TXv5v6-0719836
CTAGCTGTAAACGATGCTCGCTAGGTGTCAGACACGGTGCGACCGTGTTTGGTGCCGCAGGGAAGCCGTTAAGCGAGCCACCTGGGAAGTACGGTCGCAAGGCTGAAACTTAAAGGAATTGGCGGGGGAGCACTACAACGG
GTGGAGCCTGCGGTTTAATTGGATTCAACGCCGGAAAACTCACCGGGTGCGACAGCAATATGTAGGTCAGGCTGAAGGTCTTACCTGAATCGCTGAGAGGAGTGC
>PTM44_CONSENSUS
CTAGCTGTAAACGATGTGGACTTGGCGTTGGTGGGGTCAAATCCATCAGTGCCGKAGCTAACGCGATAAGTCCACCGCCTGGGGACTACGGTCGCAAGGCTAAAACTCAAAGGAATTGGCGGGGGCCCGCACAAGCAGCGGA
GCGTGTGGTTTAATTCGATGCTACACGAAGAACCTTACCCGGGTTTGACATCCAGGTGGTAGGGAACCGAAAGGCGACCGACCCTTCGGGGAGCCTGGACAGGTGCTGC
TXv5v6-0262835
CTAGCTGTAAACGATGTGGACTTGGCGTTGGTGGGGTCAAATCCATCAGTGCCGGAGCTAACGCGATAAGTCCACCGCCTGGGGACTACGGTCGCAAGGCTAAAACTCAAAGGAATTGGCGGGGGCCCGCACAAGCAGCGGA
GCGTGTGGTTTAATTCGATGCTACACGAAGAACCTTACCCGGGTTTGACATCCAGGTGGTAGGGAACCGAAAGGCGACCGACCCTTCGGGGAGCCTGGACAGGTGCTGC
TXv5v6-0262867
CTAGCTGTAAACGATGTGGACTTGGCGTTGGTGGGGTCAAATCCATCAGTGCCGTAGCTAACGCGATAAGTCCACCGCCTGGGGACTACGGTCGCAAGGCTAAAACTCAAAGGAATTGGCGGGGGCCCGCACAAGCAGCGGA
GCGTGTGGTTTAATTCGATGCTACACGAAGAACCTTACCCGGGTTTGACATCCAGGTGGTAGGGAACCGAAAGGCGACCGACCCTTCGGGGAGCCTGGACAGGTGCTGC
>PTM45_CONSENSUS
CTAGCTGTAAACGATGGATACTAGGTGTGGGAGGTATCGACCCCTTCTGTGCCGYAGCTAACGCATTAAGTATCCCGCCTGGGGAGTACGGTCGCAAGACTGAAACTCAAAGGAATTGACGGGGGCCCGCACAAGCGGTGGA
GCATGTGGTTTAATTCGACGCAACGCGAAGAACCTTACCGGGGCTTGACATTGTCTTGCCCGTTTAAGAAATTAAAYTTTCTTCCCTTTTAGGGAAGACAGGATAACAGGTGG
TGC
TXv5v6-0260001
CTAGCTGTAAACGATGGATACTAGGTGTGGGAGGTATCGACCCCTTCTGTGCCGTAGCTAACGCATTAAGTATCCCGCCTGGGGAGTACGGTCGCAAGACTGAAACTCAAAGGAATTGACGGGGGCCCGCACAAGCGGTGGA
GCATGTGGTTTAATTCGACGCAACGCGAAGAACCTTACCGGGGCTTGACATTGTCTTGCCCGTTTAAGAAATTAAACTTTCTTCCCTTTTAGGGAAGACAGGATAACAGGTGG
TGC
TXv5v6-1439641
CTAGCTGTAAACGATGGATACTAGGTGTGGGAGGTATCGACCCCTTCTGTGCCGTAGCTAACGCATTAAGTATCCCGCCTGGGGAGTACGGTCGCAAGACTGAAACTCAAAGGAATTGACGGGGGCCCGCACAAGCGGTGGA
GCATGTGGTTTAATTCGACGCAACGCGAAGAACCTTACCGGGGCTTGACATTGTCTTGCCCGTTTAAGAAATTAAATTTTCTTCCCTTTTAGGGAAGACAGGATAACAGGTGGTGC
TXv5v6-0725610
CTAGCTGTAAACGATGGATACTAGGTGTGGGAGGTATCGACCCCTTCTGTGCCGCAGCTAACGCATTAAGTATCCCGCCTGGGGAGTACGGTCGCAAGACTGAAACTCAAAGGAATTGACGGGGGCCCGCACAAGCGGTGGA
GCATGTGGTTTAATTCGACGCAACGCGAAGAACCTTACCGGGGCTTGACATTGTCTTGCCCGTTTAAGAAATTAAACTTTCTTCCCTTTTAGGGAAGACAGGATAACAGGTGGTGC
>PTM46_CONSENSUS
CTAGCTGTAAACGATGGATACTAGGTGTRGGAGGTATCGACCCCTTCTGTGCCGYAGCTAACGCATTAAGTATCCCGCCTGGGGAGTACGGTCGCAAGGCTGAAACTCAAAGGAATTGACGGGGGCCCGCACAAGCGGTGGA
GCATGTGGTTTAATTCGACGCAACGCGAAGAACCTTACCGGGACTTGACATTATCTTGCCCGTCTAAGAAATTAGATCTTCTTCCTTACSGAAGACAGGATAACAGGTGGTGC
TXv5v6-0259164
CTAGCTGTAAACGATGGATACTAGGTGTAGGAGGTATCGACCCCTTCTGTGCCGCAGCTAACGCATTAAGTATCCCGCCTGGGGAGTACGGTCGCAAGGCTGAAACTCAAAGGAATTGACGGGGGCCCGCACAAGCGGTGGA
GCATGTGGTTTAATTCGACGCAACGCGAAGAACCTTACCGGGACTTGACATTATCTTGCCCGTCTAAGAAATTAGATCTTCTTCCTTACCGAAGACAGGATAACAGGTGGTGC
TXv5v6-0729803
CTAGCTGTAAACGATGGATACTAGGTGTGGGAGGTATCGACCCCTTCTGTGCCGTAGCTAACGCATTAAGTATCCCGCCTGGGGAGTACGGTCGCAAGGCTGAAACTCAAAGGAATTGACGGGGGCCCGCACAAGCGGTGGA
GCATGTGGTTTAATTCGACGCAACGCGAAGAACCTTACCGGGACTTGACATTATCTTGCCCGTCTAAGAAATTAGATCTTCTTCCTTACGGAAGACAGGATAACAGGTGGTGC
```

### Table 4

In Table 4, below, lists unique V5V6 sequences (PTM 47 through 103) whose distributions among the samples were correlated with gasoline-range hydrocarbons. Primers (oligonucleotides) designed to amplify each sequence is indicated by bold text and shading. For ease of viewing, the reverse primer is shown not as its actual sequence (which is listed in Table 2), but as its reverse-complement. The term “V5V6” indicates sequences that include the fifth variable (V5) and sixth variable (V6) regions of the 16S rRNA gene.

In summary, PTM 47 through 103, the sequences of Table 4, are 57 sequences that did not group into “clades” having multiple species, or members (although, in one sense, that each define a “clade” but only having one member). PTM 03 to 46 have multiple members in their respective “clades”, and thus each have a true “consensus” sequence.

The methods used to design the PTM 03 to 46 clade primer/probes was different than for the PTM 46 to PTM 103 clade primer/probes. The analysis found 35 groups (clades) of sequences (clades PTM 12 to 46) with similarity within a group greater than 97% and 57 sequences (PTM 47 through 103) that did not cluster and were treated separately. Bioindicator primers were designed as described in Example 1 to the consensus sequence of the 35 groups (Table 3), and to each of the 57 unique un-grouped sequences (Table 4) resulting in 92 bioindicator probes (PTM12 through PTM103, Table 5).

TABLE 4
>TXv5v6-0774428
PTM47
(SEQ ID NO: 525)

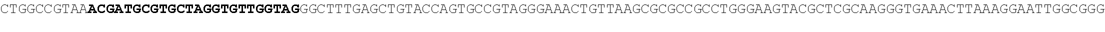

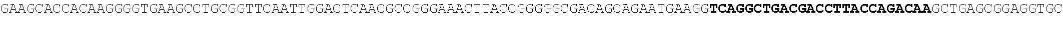

>TXv5v6-0220974
PTM48
(SEQ ID NO: 526)

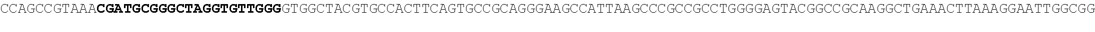

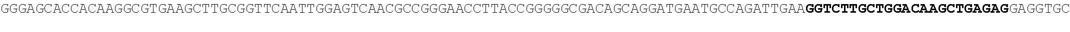

>TXv5v6-0206754
PTM49
(SEQ ID NO: 527)

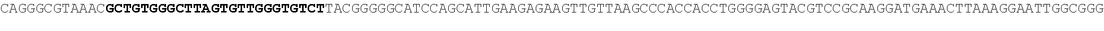

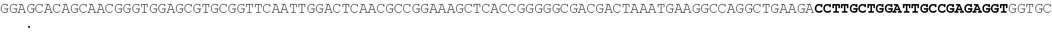

>TXv5v6-0266718
PTM50
(SEQ ID NO: 528)

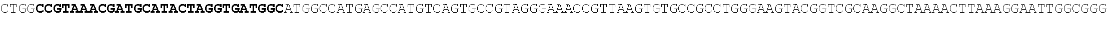

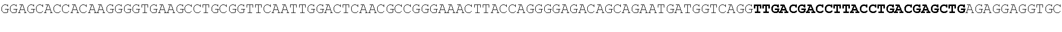

>TXv5v6-0771067
PTM51
(SEQ ID NO: 529)

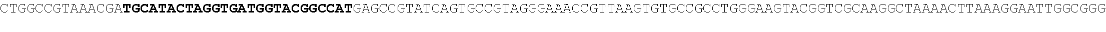

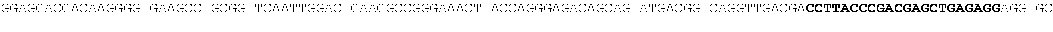

>TXv5v6-0220961
PTM52
(SEQ ID NO: 530)

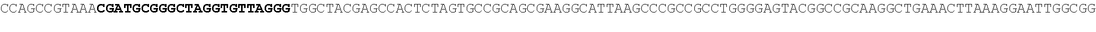

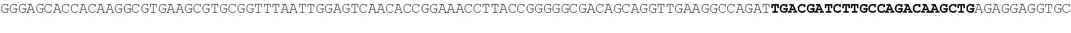

>TXv5v6-0207124
PTM53
(SEQ ID NO: 531)

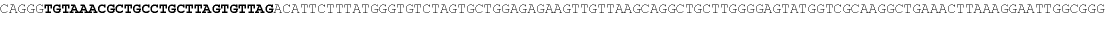

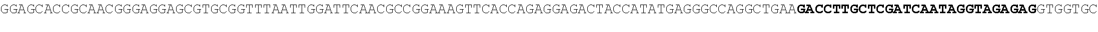

>TXv5v6-0206646
PTM54
(SEQ ID NO: 532)

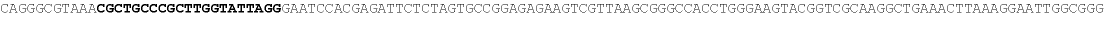

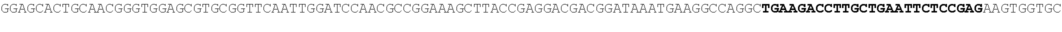

>TXv5v6-0208572
PTM55
(SEQ ID NO: 533)

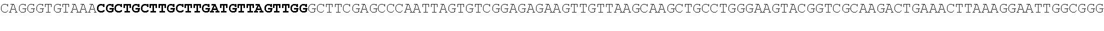

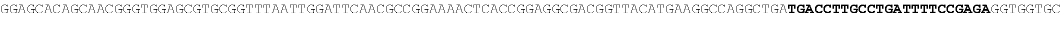

>TXv5v6-0242332
PTM56
(SEQ ID NO: 534)

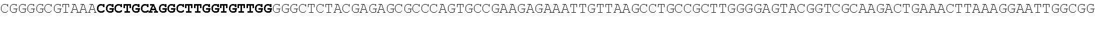

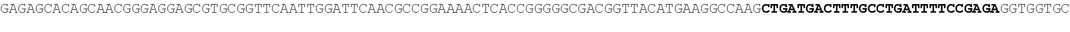

>TXv5v6-0206834
PTM57
(SEQ ID NO: 535)

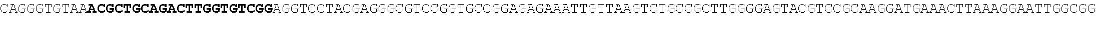

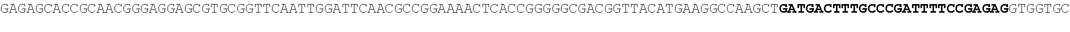

>TXv5v6-0206604
PTM58
(SEQ ID NO: 536)

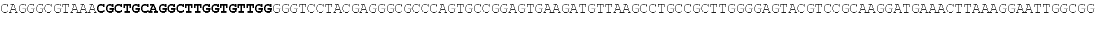

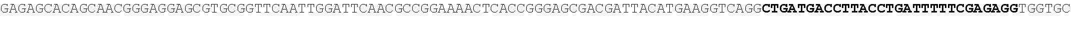

>TXv5v6-0257786
PTM59
(SEQ ID NO: 537)

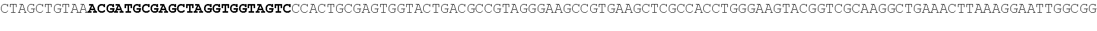

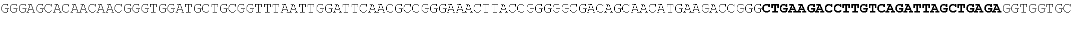

>TXv5v6-0258881
PTM60
(SEQ ID NO: 538)

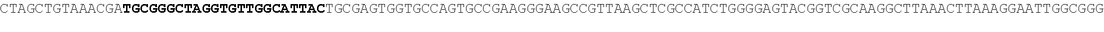

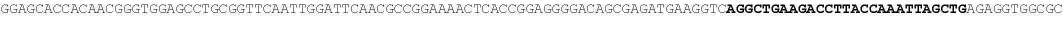

>TXv5v6-0257959
PTM61
(SEQ ID NO: 539)

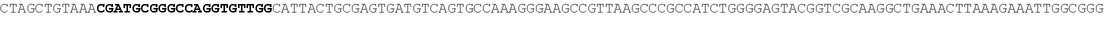

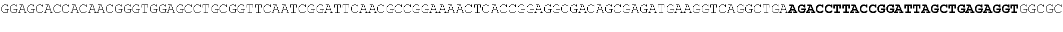

>TXv5v6-0220923
PTM62
(SEQ ID NO: 540)

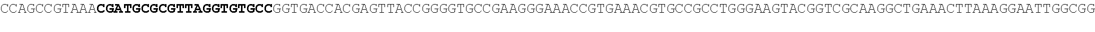

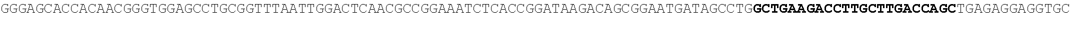

>TXv5v6-0256396
PTM63
(SEQ ID NO: 541)

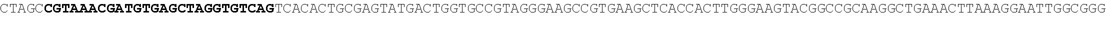

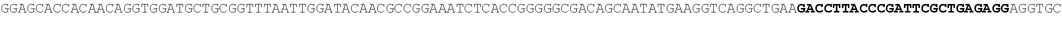

>TXv5v6-0256404
PTM64
(SEQ ID NO: 542)

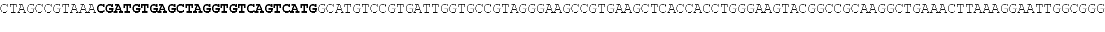

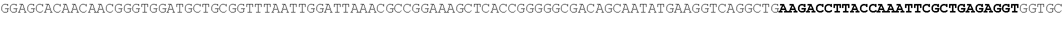

>TXv5v6-0600543
PTM65
(SEQ ID NO: 543)

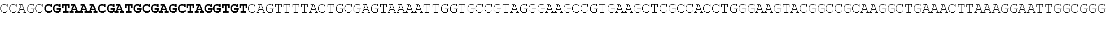

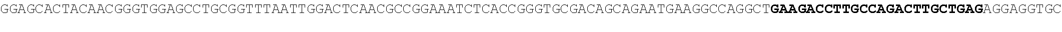

>TXv5v6-0248410
PTM66
(SEQ ID NO: 544)

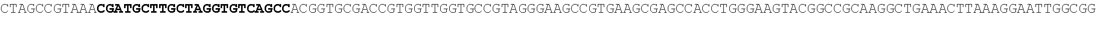

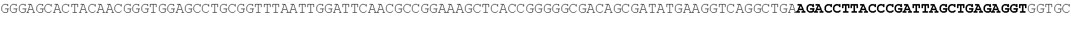

>TXv5v6-0237795
PTM67
(SEQ ID NO: 545)

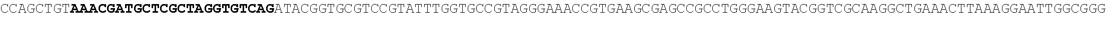

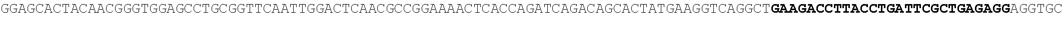

>TXv5v6-0210733
PTM68
(SEQ ID NO: 546)

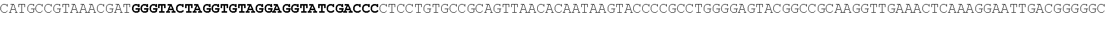

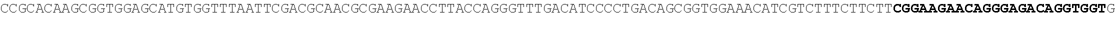

C
>TXv5v6-0195046
PTM69
(SEQ ID NO: 547)

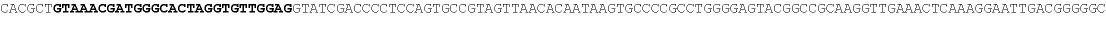

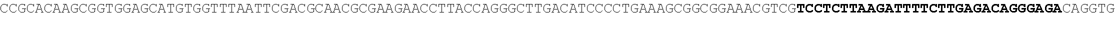

GTGC
>TXv5v6-1308235
PTM70
(SEQ ID NO: 548)

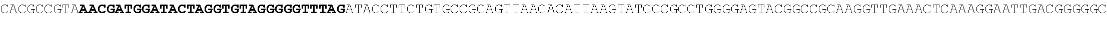

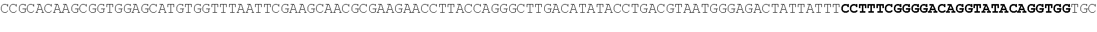

>TXv5v6-0543221
PTM71
(SEQ ID NO: 549)

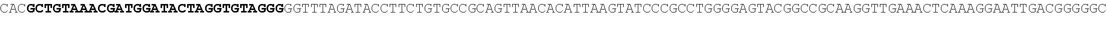

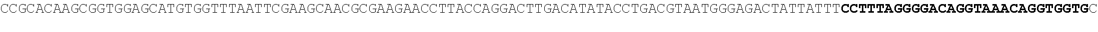

>TXv5v6-0257331
PTM72
(SEQ ID NO: 550)

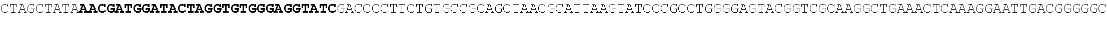

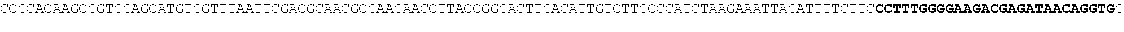

TGC
>TXv5v6-0591983
PTM73
(SEQ ID NO: 551)

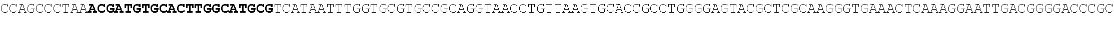

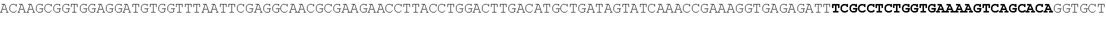

GC
>TXv5v6-1294019
PTM74
(SEQ ID NO: 552)

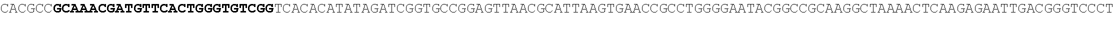

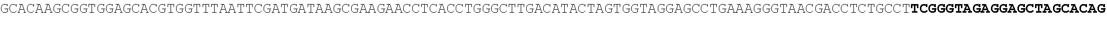

GTGATGC
>TXv5v6-1410009
PTM75
(SEQ ID NO: 553)

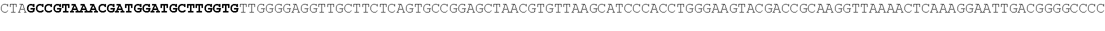

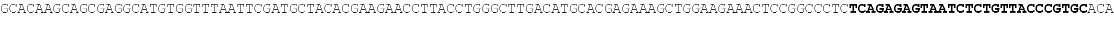

GGTGCTGC
>TXv5v6-1287141
PTM76
(SEQ ID NO: 554)

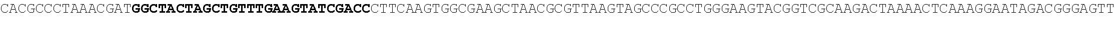

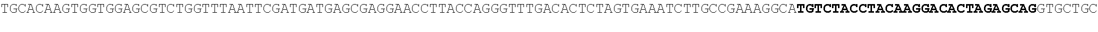

>TXv5v6-1336703
PTM77
(SEQ ID NO: 555)

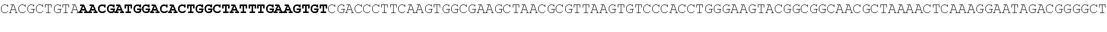

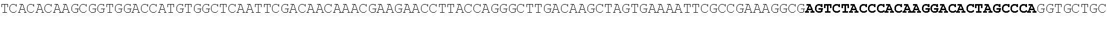

>TXv5v6-0062459
PTM78
(SEQ ID NO: 556)

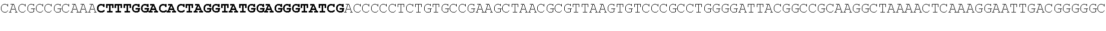

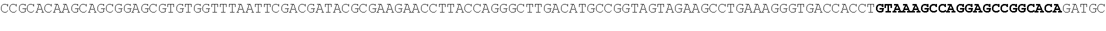

TGC
>TXv5v6-0062219
PTM79
(SEQ ID NO: 557)

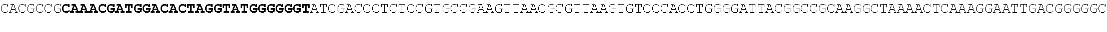

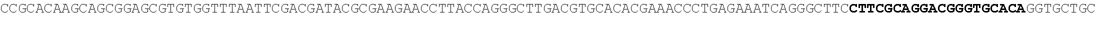

>TXv5v6-0059823
PTM80
(SEQ ID NO: 558)

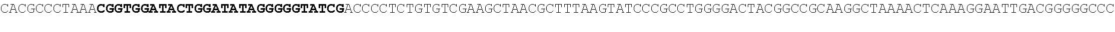

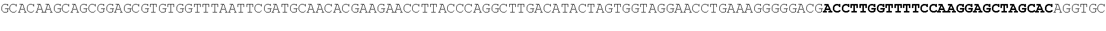

TGC
>TXv5v6-0179152
PTM81
(SEQ ID NO: 559)

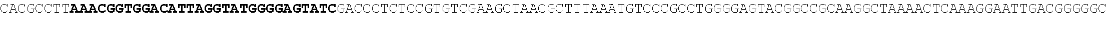

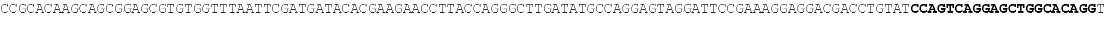

GCTGC
>TXv5v6-0059458
PTM82
(SEQ ID NO: 560)

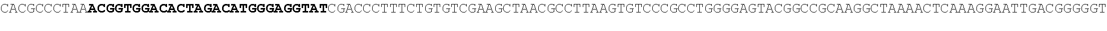

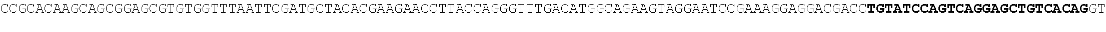

GCTGC
>TXv5v6-0059692
PTM83
(SEQ ID NO: 561)

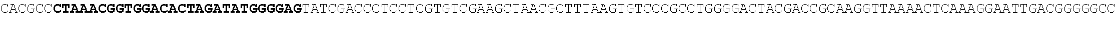

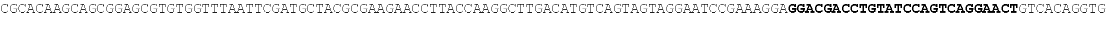

CTGC
>TXv5v6-0305895
PTM84
(SEQ ID NO: 562)

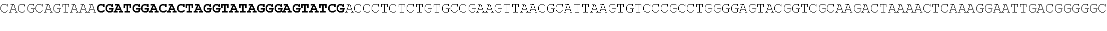

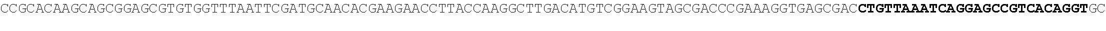

TGC
>TXv5v6-0060461
PTM85
(SEQ ID NO: 563)

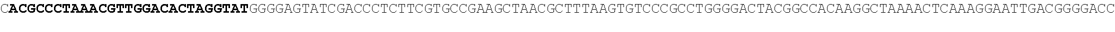

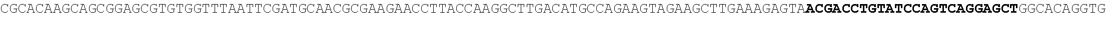

CTGC
>TXv5v6-0175746
PTM86
(SEQ ID NO: 564)

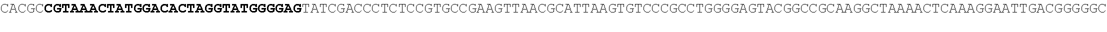

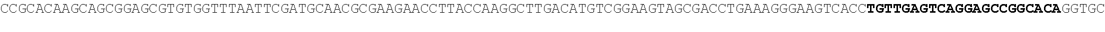

TGC
>TXv5v6-0250092
PTM87
(SEQ ID NO: 565)

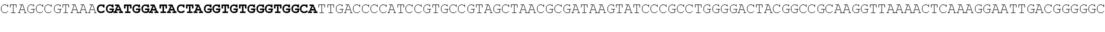

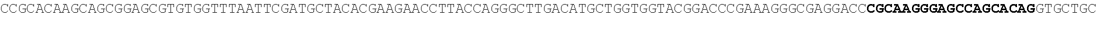

>TXv5v6-0252039
PTM88
(SEQ ID NO: 566)

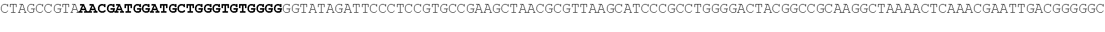

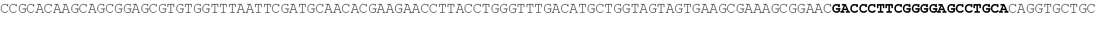

>TXv5v6-0257726
PTM89
(SEQ ID NO: 567)

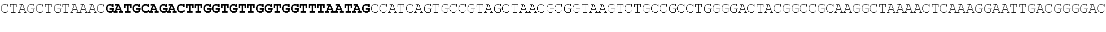

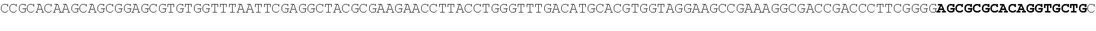

>TXv5v6-0120132
PTM90
(SEQ ID NO: 568)

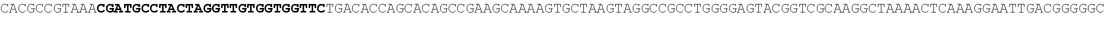

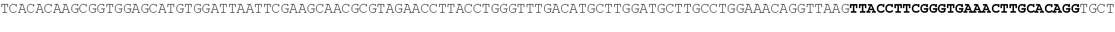

GC
>TXv5v6-1276382
PTM91
(SEQ ID NO: 569)

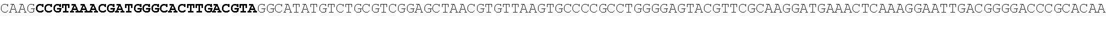

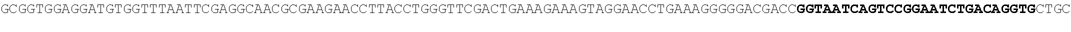

>TXv5v6-0722918
PTM92
(SEQ ID NO: 570)

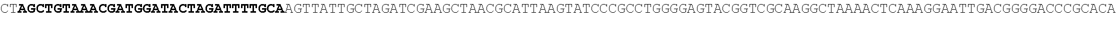

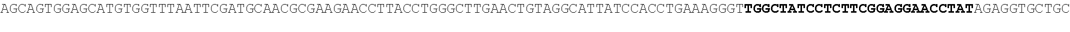

>TXv5v6-0690447
PTM93
(SEQ ID NO: 571)

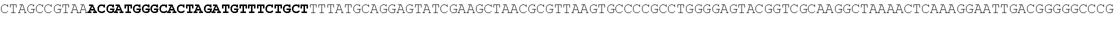

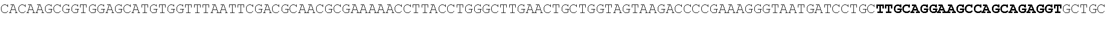

>TXv5v6-0690171
PTM94
(SEQ ID NO: 572)

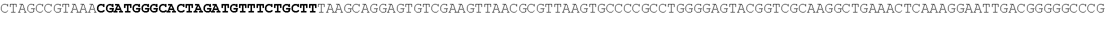

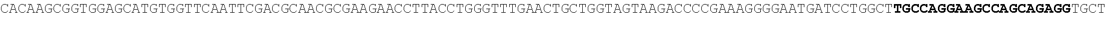

GC
>TXv5v6-0187739
PTM95
(SEQ ID NO: 573)

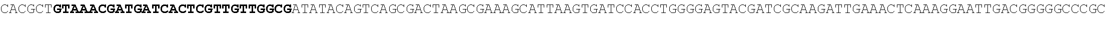

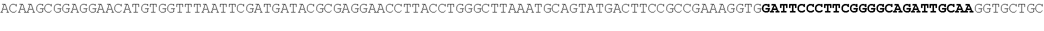

>TXv5v6-0404321
PTM96
(SEQ ID NO: 574)

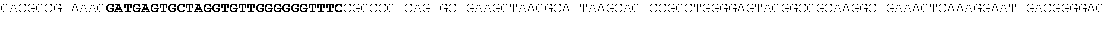

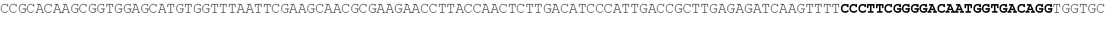

>TXv5v6-0168244
PTM97
(SEQ ID NO: 575)

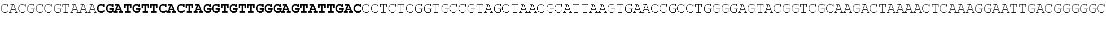

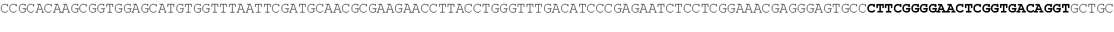

>TXv5v6-0168232
PTM98
(SEQ ID NO: 576)

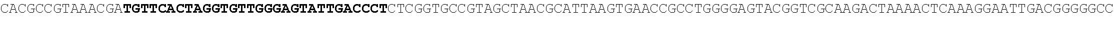

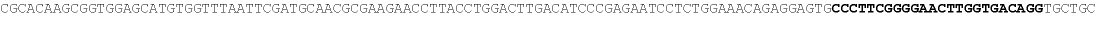

>TXv5v6-0183853
PTM99
(SEQ ID NO: 577)

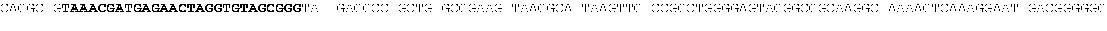

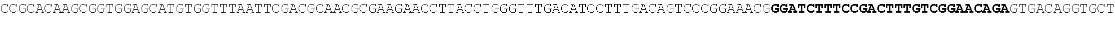

GC
>TXv5v6-0063999
PTM100
(SEQ ID NO: 578)

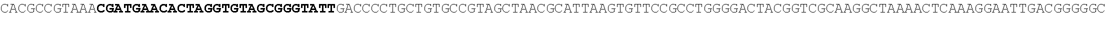

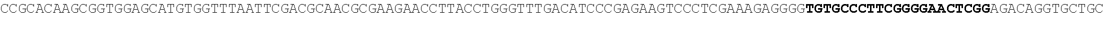

>TXv5v6-0176581
PTM101
(SEQ ID NO: 579)

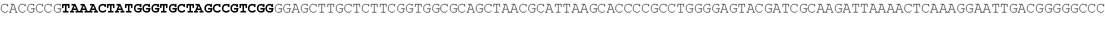

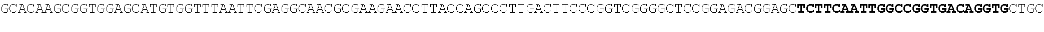

>TXv5v6-0255064
PTM102
(SEQ ID NO: 580)

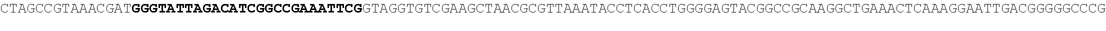

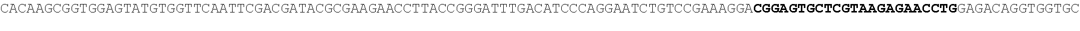

>TXv5v6-0138901
PTM103
(SEQ ID NO: 581)

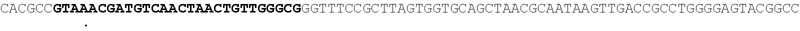

GCAAGGCTAAAACTCAAATGAATTGACGGGGGCCCGCACAAGCGGTGGAGCATGTGGTTTAATTCGATGCAACGCGAAGAACCTTACCTACC

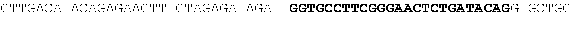

A number of embodiments of the invention have been described. Nevertheless, it will be understood that various modifications may be made without departing from the spirit and scope of the invention. Accordingly, other embodiments are within the scope of the following claims.

1. An isolated, synthetic or recombinant nucleic acid comprising or consisting of:
(a) a nucleic acid or a nucleic acid sequence as set forth in Table 1, Table 2, Table 3 or Table 4;
(b) a nucleic acid or a nucleic acid sequence having at least about 85%, 86%, 87%, 88%, 89%, 90%, 91%, 92%, 93%, 94%, 95%, 96%, 97%, 98% or 99% or complete (100%) sequence homology to a nucleic acid or a nucleic acid sequence as set forth in Table 1, Table 2, Table 3 or Table 4;
(c) a nucleic acid or a nucleic acid sequence having at least about 85%, 86%, 87%, 88%, 89%, 90%, 91%, 92%, 93%, 94%, 95%, 96%, 97%, 98% or 99% or complete (100%) sequence homology to a nucleic acid or a nucleic acid sequence: as set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165, SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192 SEQ ID NO:193, SEQ ID NO:194, SEQ ID NO:195, SEQ ID NO:196, SEQ ID NO:197, SEQ ID NO:198, SEQ ID NO:199 or SEQ ID NO:200 (hereinafter referenced as SEQ ID NO:1 to SEQ ID NO:200); or
(d) a nucleic acid or a nucleic acid sequence having at least about 85%, 86%, 87%, 88%, 89%, 90%, 91%, 92%, 93%, 94%, 95%, 96%, 97%, 98% or 99% or complete (100%) sequence homology to a nucleic acid or a nucleic acid sequence: as set forth in any one of SEQ ID NO:201 to SEQ ID NO:583,
and optionally the sequence identities are determined by analysis with a sequence comparison algorithm or by a visual inspection,
and optionally the sequence comparison algorithm is a BLAST version 2.2.2 algorithm where a filtering setting is set to blastall -p blastp -d “nr pataa”-F F, and all other options are set to default.

2. An isolated, synthetic or recombinant nucleic acid comprising or consisting of a nucleic acid sequence capable of specifically (selectively) hybridizing (hybridizes under stringent conditions to) to a nucleic acid of claim 1, or a nucleic acid sequence as set forth in Table 1, Table 2, Table 3 or Table 4, or a nucleic acid or nucleic acid sequence as set forth in any one of SEQ ID NO:1 to SEQ ID NO:200 or SEQ ID NO:201 to SEQ ID NO:583,
wherein optionally the stringent conditions include a wash step comprising a wash in 0.2×SSC at a temperature of about 65° C. for about 15 minutes.

3. The isolated, synthetic or recombinant nucleic acid of claim 2, wherein the nucleic acid sequence capable of specifically (selectively) hybridizing to (hybridizes under stringent conditions to) a nucleic acid of claim 1, or a nucleic acid sequence as set forth in Table 1, Table 2, Table 3 or Table 4, comprises or consists of:
(a) a member of an amplification primer pair, a polymerase chain reaction (PCR) primer pair, ligase chain reaction (LCR) pair, or a qPCR primer pair capable of amplifying a nucleic acid sequence as set forth in Table 2; or,
(b) a hybridization probe sequence capable of specifically (selectively) hybridizing to a nucleic acid or nucleic acid sequence of claim 1, or as set forth in Table 1, Table 2, Table 3 or Table 4, or a nucleic acid or nucleic acid sequence as set forth in any one of SEQ ID NO:1 to SEQ ID NO:200 or SEQ ID NO:201 to SEQ ID NO:583.

4. The isolated, synthetic or recombinant nucleic acid of claim 1, further comprising:
(a) a detectable moiety or an enzyme,
wherein optionally the detectable moiety comprises a radioactive probe, a fluorescent molecule (e.g., a fluorescent label or a fluorophore, e.g., a coumarin, resorufin, xanthene, benzoxanthene, cyanine or bodipy analog), a quantum dot or a colloidal quantum dot (QD) (e.g., a QDOT™ nanocrystal, Life Technologies, Carlsbad, Calif.), and/or an epitope or binding molecule (e.g. a ligand); or
(b) further comprising a solid or semi-solid surface, wherein optionally the nucleic acid is immobilized or conjugated or bound to, the solid or semi-solid surface,
wherein optionally the solid or semi-solid surface comprises or consists of an array, a biochip, a chip, a bead, a gel, a liposome, a fiber, a film, a membrane, a metal, a resin, a polymer, a ceramic, a glass, an electrode, a microelectrode, a graphitic particle, or a microparticle or a nanoparticle.

5-7. (canceled)

8. An amplification primer pair or amplification pair, a polymerase chain reaction (PCR) primer pair, a ligase chain reaction (LCR) pair, or a qPCR primer pair comprising or consisting of:
(a) a primer pair as set forth in Table 2, or one member of a primer pair as set forth in Table 2,
(b) a primer pair comprising or consisting of: SEQ ID NO:1 and SEQ ID NO:2; SEQ ID NO:3 and SEQ ID NO:4; SEQ ID NO:5 and SEQ ID NO:6; SEQ ID NO:7 and SEQ ID NO:8; SEQ ID NO:9 and SEQ ID NO:10; SEQ ID NO:11 and SEQ ID NO:12; SEQ ID NO:13 and SEQ ID NO:14; SEQ ID NO:15 and SEQ ID NO:16; SEQ ID NO:17 and SEQ ID NO:18; SEQ ID NO:19 and SEQ ID NO:20; SEQ ID NO:21 and SEQ ID NO:22; SEQ ID NO:23 and SEQ ID NO:24; SEQ ID NO:25 and SEQ ID NO:26; SEQ ID NO:27 and SEQ ID NO:28; SEQ ID NO:29 and SEQ ID NO:30; SEQ ID NO:31 and SEQ ID NO:32; SEQ ID NO:33 and SEQ ID NO:34; SEQ ID NO:35 and SEQ ID NO:36; SEQ ID NO:37 and SEQ ID NO:38; SEQ ID NO:39 and SEQ ID NO:40; SEQ ID NO:41 and SEQ ID NO:42; SEQ ID NO:43 and SEQ ID NO:44; SEQ ID NO:45 and SEQ ID NO:46; SEQ ID NO:47 and SEQ ID NO:48; SEQ ID NO:49 and SEQ ID NO:50; SEQ ID NO:51 and SEQ ID NO:52; SEQ ID NO:53 and SEQ ID NO:54; SEQ ID NO:55 and SEQ ID NO:56; SEQ ID NO:57 and SEQ ID NO:58; SEQ ID NO:59 and SEQ ID NO:60; SEQ ID NO:61 and SEQ ID NO:62, SEQ ID NO:63 and SEQ ID NO:64; SEQ ID NO:65 and SEQ ID NO:66; SEQ ID NO:67 and SEQ ID NO:68; SEQ ID NO:69 and SEQ ID NO:70; SEQ ID NO:71 and SEQ ID NO:72; SEQ ID NO:73 and SEQ ID NO:74; SEQ ID NO:75 and SEQ ID NO:76; SEQ ID NO:77 and SEQ ID NO:78; SEQ ID NO:79 and SEQ ID NO:80; SEQ ID NO:81 and SEQ ID NO:82; SEQ ID NO:83 and SEQ ID NO:84; SEQ ID NO:85 and SEQ ID NO:86; SEQ ID NO:87 and SEQ ID NO:88; SEQ ID NO:89 and SEQ ID NO:90; SEQ ID NO:91 and SEQ ID NO:92; SEQ ID NO:93 and SEQ ID NO:94; SEQ ID NO:95 and SEQ ID NO:96; SEQ ID NO:97 and SEQ ID NO:98; SEQ ID NO:99 and SEQ ID NO:100; SEQ ID NO:101 and SEQ ID NO:102; SEQ ID NO:103 and SEQ ID NO:104; SEQ ID NO:105 and SEQ ID NO:106; SEQ ID NO:107 and SEQ ID NO:108; SEQ ID NO:109 and SEQ ID NO:110; SEQ ID NO:111 and SEQ ID NO:112; SEQ ID NO:113 and SEQ ID NO:114; SEQ ID NO:115 and SEQ ID NO:116; SEQ ID NO:117 and SEQ ID NO:118; SEQ ID NO:119 and SEQ ID NO:120; SEQ ID NO:121 and SEQ ID NO:122; SEQ ID NO:123 and SEQ ID NO:124; SEQ ID NO:125 and SEQ ID NO:126; SEQ ID NO:127 and SEQ ID NO:128; SEQ ID NO:129 and SEQ ID NO:130; SEQ ID NO:131 and SEQ ID NO:132; SEQ ID NO:133 and SEQ ID NO:134; SEQ ID NO:135 and SEQ ID NO:136; SEQ ID NO:137 and SEQ ID NO:138; SEQ ID NO:139 and SEQ ID NO:140; SEQ ID NO:141 and SEQ ID NO:142; SEQ ID NO:143 and SEQ ID NO:144; SEQ ID NO:145 and SEQ ID NO:146; SEQ ID NO:147 and SEQ ID NO:148; SEQ ID NO:149 and SEQ ID NO:150; SEQ ID NO:151 and SEQ ID NO:152; SEQ ID NO:153 and SEQ ID NO:154; SEQ ID NO:155 and SEQ ID NO:156; SEQ ID NO:157 and SEQ ID NO:158; SEQ ID NO:159 and SEQ ID NO:160; SEQ ID NO:161 and SEQ ID NO:162; SEQ ID NO:163 and SEQ ID NO:164; SEQ ID NO:165 and SEQ ID NO:166; SEQ ID NO:167 and SEQ ID NO:168; SEQ ID NO:169 and SEQ ID NO:170; SEQ ID NO:171 and SEQ ID NO:172; SEQ ID NO:173 and SEQ ID NO:174; SEQ ID NO:175 and SEQ ID NO:176; SEQ ID NO:177 and SEQ ID NO:178; SEQ ID NO:179 and SEQ ID NO:180; SEQ ID NO:181 and SEQ ID NO:182; SEQ ID NO:183 and SEQ ID NO:184; SEQ ID NO:185 and SEQ ID NO:186; SEQ ID NO:187 and SEQ ID NO:188; SEQ ID NO:189 and SEQ ID NO:190; SEQ ID NO:191 and SEQ ID NO:192; SEQ ID NO:193 and SEQ ID NO:194; SEQ ID NO:195 and SEQ ID NO:196; SEQ ID NO:197 and SEQ ID NO:198; or, SEQ ID NO:199 and SEQ ID NO:200;
(c) all of the primer pairs as set forth in Table 2; or
(d) all of the primer pairs of (b).

9. The amplification primer pair or amplification pair, polymerase chain reaction (PCR) primer pair, ligase chain reaction (LCR) pair, or qPCR primer pair of claim 8, wherein:
(a) at least one member of the primer pair further comprises a detectable moiety;
(b) the detectable moiety comprises a radioactive probe, a fluorescent molecule (e.g., a fluorescent label or a fluorophore, e.g., a coumarin, resorufin, xanthene, benzoxanthene, cyanine or bodipy analog), a quantum dot or a colloidal quantum dot (QD) (e.g., a QDOT™ nanocrystal, Life Technologies, Carlsbad, Calif.), and/or an epitope or binding molecule (e.g. a ligand);
(c) at least one member of the primer pair, or both members of the primer pair, further comprise, or are immobilized or conjugated or bound to, a solid or a semi-solid surface;
(d) the amplification primer pair or amplification pair, polymerase chain reaction (PCR) primer pair, ligase chain reaction (LCR) pair, or qPCR primer pair of (c), wherein the solid or semi-solid surface comprises or consists of an array, a biochip, a chip, a bead, a gel, a liposome, a fiber, a film, a membrane, a metal, a resin, a polymer, a ceramic, a glass, an electrode, a microelectrode, a graphitic particle, or a microparticle or a nanoparticle.

10-12. (canceled)

13. An array, a biochip, a chip, a bead, a gel, a liposome, a fiber, a film, a membrane, a metal, a resin, a polymer, a ceramic, a glass, an electrode, a microelectrode, a graphitic particle, or a microparticle or a nanoparticle, comprising a nucleic acid of claim 1, or a plurality of or all of the nucleic acids of claim 1, or an amplification primer pair, polymerase chain reaction (PCR) primer pair, a ligase chain reaction (LCR) pair, or a qPCR primer pair comprising a nucleic acid of claim 1.

14. A product of manufacture, an array, a biochip, a chip, a bead, a gel, a liposome, a fiber, a film, a membrane, a metal, a resin, a polymer, a ceramic, a glass, an electrode, a microelectrode, a graphitic particle, or a microparticle or a nanoparticle, comprising a nucleic acid of claim 1, or a plurality of or all of the nucleic acids of claim 1, or an amplification primer pair, polymerase chain reaction (PCR) primer pair, a ligase chain reaction (LCR) pair, or a qPCR primer pair comprising a nucleic acid of claim 1.

15. A kit comprising a nucleic acid of claim 1, or a plurality of or all of the nucleic acids of claim 1, or an amplification primer pair, polymerase chain reaction (PCR) primer pair, a ligase chain reaction (LCR) pair, or a qPCR primer pair comprising a nucleic acid of claim 1,
wherein optionally the kit comprises or is a PCR, LCR or qPCR kit,
and optionally the nucleic acid, amplification primer pair, polymerase chain reaction (PCR) primer pair, ligase chain reaction (LCR) pair or qPCR primer pair is contained or stored in a solution, a test tube or a container.

16. A method of detecting, identifying, quantifying and/or indicating the presence of a hydrocarbon in a sample, comprising:
(1) (a) obtaining or providing one sample or a set of samples,
wherein optionally the sample is an aqueous sample, a fresh water sample or a sea water sample, or a sediment, sand, shale or mud, or a marine sediment, sand, shale or mud, or a solution,
or optionally the samples comprise fresh water, underground water or seawater, or a production water, or an aqueous sample or a marine sediment, sand, shale or mud are taken from or prepared from a core sample; and
(b) detecting, determining, quantifying and/or characterizing the presence of a nucleic acid in the sample or samples, wherein the detecting, determining, characterizing or quantifying (measuring) the presence of the nucleic acid in the sample or samples indicates the presence of, or quantifies or estimates the amount of, the hydrocarbon in the sample or solution,
and the nucleic acid detected, characterized or quantified comprises or consists of a nucleic acid of claim 1, and/or
the nucleic acid is detected, characterized or quantified using:
a nucleic acid of claim 1, or
an amplification primer pair, polymerase chain reaction (PCR) primer pair, ligase chain reaction (LCR) pair, or qPCR primer pair comprising a nucleic acid of claim 1, or
an array, a biochip, a chip, a bead, a gel, a liposome, a fiber, a film, a membrane, a metal, a resin, a polymer, a ceramic, a glass, an electrode, a microelectrode, a graphitic particle, or a microparticle or a nanoparticle comprising a nucleic acid of claim 1,
a product of manufacture, an array, a biochip, a chip, a bead, a gel, a liposome, a fiber, a film, a membrane, a metal, a resin, a polymer, a ceramic, a glass, an electrode, a microelectrode, a graphitic particle, or a microparticle or a nanoparticle comprising a nucleic acid of claim 1;
wherein optionally the determining, quantifying and/or characterizing the presence of a nucleic acid in the sample or samples is by a method comprising an amplification, a polymerase chain reaction (PCR), a qPCR and/or a hybridization;
wherein optionally identifying, quantifying and/or characterizing a nucleic acid in the sample or samples also by correlation identifies, quantifies or indicates the presence of a hydrocarbon in the solution.
wherein detecting, quantifying, determining and/or characterizing the nucleic acid in the sample or samples quantifies, identifies or detects the presence of the hydrocarbon in the sample; or
(2) the method of (1), wherein each test sample is assayed for the presence of a plurality of, or many independent, bioindicators that are positively correlated with the presence of one or more hydrocarbons,
wherein optionally the bioindicator comprises a nucleic acid of claim 1;
(3) the method of (1), wherein a test sample is assayed for the presence of one or more, or a plurality of, microbial bioindicator sequences or nucleic acids that are positively and negatively associated with the presence of a hydrocarbon,
wherein optionally the microbial bioindicator sequence or nucleic acid comprises a nucleic acid of claim 1;
(4) the method of any of (1) to (3), wherein an RNA is extracted from the sample or samples, and the RNA converted to a DNA prior to PCR amplification and/or hybridization,
wherein optionally the RNA is a ribosomal RNA, or
or optionally the RNA converted to a DNA using a reverse transcriptase enzyme; or
(5) the method of any of (1) to (4), further comprising characterizing and/or identifying one, all or substantially most of the microbes in the sample or samples,
wherein optionally the microbial composition is determined by a chemical or analytical method, and optionally the chemical or analytical method comprises a fatty acid methyl ester analysis, a membrane lipid analysis and/or a cultivation-dependent method.

17-20. (canceled)

21. A method of detecting the presence of a subsurface hydrocarbon, petroleum, oil or gas accumulation or deposit, or the presence of a petroleum or hydrocarbon seep, spill, pollutant or leak, comprising:
(1) (a) obtaining or providing one samples or a set of samples,
wherein optionally the sample or samples are from, or comprise, a marine sediment, shale, sand or mud, or an aqueous source, or seawater, fresh water or production fluid,
and optionally the sample or samples comprise a fresh water, underground water or seawater source, or a production water, or the marine sediment, sand or mud, or aqueous sample is taken from or prepared from a core sample, and optionally the seep is a thermogenic hydrocarbon seep or a macroseep or a microseep; and
(b) determining, detecting and/or characterizing the presence of a nucleic acid in the sample or samples, wherein the presence of a nucleic acid in the sample or samples indicates the presence of a subsurface hydrocarbon, petroleum, oil or gas accumulation or deposit, or a leak, pollutant, seep or spill,
and the nucleic acid detected, characterized or quantified comprises or consists of a nucleic acid of claim 1, and/or
the nucleic acid is detected, characterized or quantified using:
a nucleic acid of claim 1, or
an amplification primer pair, polymerase chain reaction (PCR) primer pair, ligase chain reaction (LCR) pair, or qPCR primer pair comprising a nucleic acid of claim 1, or
an array, a biochip, a chip, a bead, a gel, a liposome, a fiber, a film, a membrane, a metal, a resin, a polymer, a ceramic, a glass, an electrode, a microelectrode, a graphitic particle, or a microparticle or a nanoparticle comprising a nucleic acid of claim 1,
a product of manufacture, an array, a biochip, a chip, a bead, a gel, a liposome, a fiber, a film, a membrane, a metal, a resin, a polymer, a ceramic, a glass, an electrode, a microelectrode, a graphitic particle, or a microparticle or a nanoparticle comprising a nucleic acid of claim 1;
wherein optionally the detecting, quantifying, determining and/or characterizing the presence of a nucleic acid in the sample or samples is by a method comprising amplification, polymerase chain reaction (PCR), qPCR and/or hybridization;
wherein detecting, quantifying, determining and/or characterizing a nucleic acid in the sample or samples quantifies, identifies or detects the presence of a subsurface hydrocarbon, petroleum, oil or gas accumulation or deposit, or the presence of a petroleum or hydrocarbon seep, pollutant, spill or leak;
(2) the method of (1), wherein each sample is assayed for the presence of a plurality of, or many independent, bioindicators that are positively correlated with the presence of one or more hydrocarbons;
(3) the method of (1), wherein the sample is assayed for the presence of one or more, or a plurality of, microbial bioindicator sequences that are positively and negatively associated with the presence of hydrocarbons;
(4) the method of any of (1) to (3), wherein an RNA is extracted from samples and converted to a DNA prior to a PCR amplification and/or a hybridization,
wherein optionally the RNA is a ribosomal RNA; or
(5) the method of any of (1) to (4), further comprising characterizing and/or identifying one, all or substantially most of the microbes in the sample or samples, wherein optionally the microbial composition is determined by a chemical or analytical method, and optionally the chemical or analytical method comprises a fatty acid methyl ester analysis, a membrane lipid analysis and/or a cultivation-dependent method.

22-25. (canceled)

26. A kit comprising a nucleic acid of claim 1.

27. A kit comprising an array, a biochip, a chip, a bead, a gel, a liposome, a fiber, a film, a membrane, a metal, a resin, a polymer, a ceramic, a glass, an electrode, a microelectrode, a graphitic particle, or a microparticle or a nanoparticle of claim 13.

28. A kit comprising a product of manufacture, an array, a biochip, a chip, a bead, a gel, a liposome, a fiber, a film, a membrane, a metal, a resin, a polymer, a ceramic, a glass, an electrode, a microelectrode, a graphitic particle, or a microparticle or a nanoparticle of claim 14.

29. A kit comprising an isolated, synthetic or recombinant nucleic acid of claim 2.


SEQUENCE LISTING (619 sequences)

SEQ ID NO: 1 - DNA, 25 bp, Artificial Sequence
  Synthetic PTM03-834F
aggggatatcaactcctccgtgtcg

SEQ ID NO: 2 - DNA, 22 bp, Artificial Sequence
  Synthetic PTM03-1270R
atcactccgtggccacccgttg

SEQ ID NO: 3 - DNA, 22 bp, Artificial Sequence
  Synthetic PTM04-808F
gggcgtaaacgctgtgggctta

SEQ ID NO: 4 - DNA, 26 bp, Artificial Sequence
  Synthetic PTM04-1301R
tggatgggtttcgggattgccttcac

SEQ ID NO: 5 - DNA, 19 bp, Artificial Sequence
  Synthetic PTM05-811F
cgtaaacgctgcccgcttg

SEQ ID NO: 6 - DNA, 24 bp, Artificial Sequence
  Synthetic PTM05-1135R
tcgaagatagcaactaagagcgag

SEQ ID NO: 7 - DNA, 23 bp, Artificial Sequence
  Synthetic PTM06-820F
gctatgtgtcgggagatccacgt

SEQ ID NO: 8 - DNA, 24 bp, Artificial Sequence
  Synthetic PTM06-1267R
tcgggatcggtactctttgttccg

SEQ ID NO: 9 - DNA, 25 bp, Artificial Sequence
  Synthetic PTM07-820F-ALT
tgctagcttggtgttggataaccta

SEQ ID NO: 10 - DNA, 28 bp, Artificial Sequence
  Synthetic PTM07-1115R-ALT
cggacttgaaaatagcaactgaagatgg

SEQ ID NO: 11 - DNA, 25 bp, Artificial Sequence
  Synthetic PTM08-849F
ctctgtgtcgaagctaacgctttaa

SEQ ID NO: 12 - DNA, 25 bp, Artificial Sequence
  Synthetic PTM08-1142R
caggatttctgggcagtttcgtcag

SEQ ID NO: 13 - DNA, 20 bp, Artificial Sequence
  Synthetic PTM10-840F
tcgaccccttctgtgccgca

SEQ ID NO: 14 - DNA, 23 bp, Artificial Sequence
  Synthetic PTM10-1190R
accttcctccgcattatctgcga

SEQ ID NO: 15 - DNA, 25 bp, Artificial Sequence
  Synthetic PTM11-818F
gatgttcacttggtgtcggtcgcac

SEQ ID NO: 16 - DNA, 28 bp, Artificial Sequence
  Synthetic PTM11-1244R
ttgcaactctctgtaccttccattgtag

SEQ ID NO: 17 - DNA, 19 bp, Artificial Sequence
  Synthetic PTM12-851F
gccccagtgccgcagggaa

SEQ ID NO: 18 - DNA, 23 bp, Artificial Sequence
  Synthetic PTM12-1045R
ctctcagcttgtctggcaaggtc

SEQ ID NO: 19 - DNA, 24 bp, Artificial Sequence
  Synthetic PTM13-844F
acgtggttattcagtgccggagag

SEQ ID NO: 20 - DNA, 24 bp, Artificial Sequence
  Synthetic PTM13-1046R
cctctcagctagtccagcaaagtc

SEQ ID NO: 21 - DNA, 24 bp, Artificial Sequence
  Synthetic PTM14-819F
ctgcttgcttgatgttagttggct

SEQ ID NO: 22 - DNA, 28 bp, Artificial Sequence
  Synthetic PTM14-1042R
ctctcggaaaatcaggcaaggtcatcag

SEQ ID NO: 23 - DNA, 23 bp, Artificial Sequence
  Synthetic PTM15-817F
cgatgccagctatgtgtcggaag

SEQ ID NO: 24 - DNA, 24 bp, Artificial Sequence
  Synthetic PTM15-1046R
ctctcagctaatctggcaaggtcc

SEQ ID NO: 25 - DNA, 24 bp, Artificial Sequence
  Synthetic PTM16-810F
ccgtaaacgatgcaggctaggtgt

SEQ ID NO: 26 - DNA, 22 bp, Artificial Sequence
  Synthetic PTM16-1045R
ctctcagctcgtccagcaagac

SEQ ID NO: 27 - DNA, 23 bp, Artificial Sequence
  Synthetic PTM17-828F
ccagctgtaaacgatgcaggcta

SEQ ID NO: 28 - DNA, 25 bp, Artificial Sequence
  Synthetic PTM17-1050R
acctcctctcagcttgtctggtaag

SEQ ID NO: 29 - DNA, 26 bp, Artificial Sequence
  Synthetic PTM18-851F
ctaaacatcagtacctcctcgagagg

SEQ ID NO: 30 - DNA, 24 bp, Artificial Sequence
  Synthetic PTM18-1049R
actcctctcagcgtgtcaggtaag

SEQ ID NO: 31 - DNA, 22 bp, Artificial Sequence
  Synthetic PTM19-809F
gcagtaaacgatgcgggcyagg

SEQ ID NO: 32 - DNA, 26 bp, Artificial Sequence
  Synthetic PTM19-1048R
cacctctcagctaattcagcaaagtc

SEQ ID NO: 33 - DNA, 27 bp, Artificial Sequence
  Synthetic PTM20-844F
gatgctcgctaggtgttaaataccctg

SEQ ID NO: 34 - DNA, 27 bp, Artificial Sequence
  Synthetic PTM20-1049R
cttcctctcagcgaatttggtaaggtc

SEQ ID NO: 35 - DNA, 26 bp, Artificial Sequence
  Synthetic PTM21-833F
ggccgtaaacgatgcatactaggtga

SEQ ID NO: 36 - DNA, 21 bp, Artificial Sequence
  Synthetic PTM21-1051R
cacctcctctcagctcgtcgg

SEQ ID NO: 37 - DNA, 26 bp, Artificial Sequence
  Synthetic PTM22-849F
tactaggtgatggtacggctatgagc

SEQ ID NO: 38 - DNA, 24 bp, Artificial Sequence
  Synthetic PTM22-1050R
acctcctctcagctcgttgggtaa

SEQ ID NO: 39 - DNA, 27 bp, Artificial Sequence
  Synthetic PTM23-838F
gtaaatgatgtgggctaggtgcaaagc

SEQ ID NO: 40 - DNA, 25 bp, Artificial Sequence
  Synthetic PTM23-1038R
cttgtctggtaaggtcatcagcctg

SEQ ID NO: 41 - DNA, 24 bp, Artificial Sequence
  Synthetic PTM24-852F
aggtgtggcattactgcgagtgat

SEQ ID NO: 42 - DNA, 22 bp, Artificial Sequence
  Synthetic PTM24-1051R
cgccacctctcagctaatctgg

SEQ ID NO: 43 - DNA, 22 bp, Artificial Sequence
  Synthetic PTM25-809F
ggcgtaaacgatgtgggcttcg

SEQ ID NO: 44 - DNA, 25 bp, Artificial Sequence
  Synthetic PTM25-1053R
gcaccacctctctgcctattattcg

SEQ ID NO: 45 - DNA, 21 bp, Artificial Sequence
  Synthetic PTM26-809F
gctgtaaacgatgcgggccag

SEQ ID NO: 46 - DNA, 22 bp, Artificial Sequence
  Synthetic PTM26-1052R
cgccacctctcagctaatccag

SEQ ID NO: 47 - DNA, 23 bp, Artificial Sequence
  Synthetic PTM27-812F
gtaacgatgcgggccaggtgttg

SEQ ID NO: 48 - DNA, 21 bp, Artificial Sequence
  Synthetic PTM27-1052R
cgccacctctcagctaatccg

SEQ ID NO: 49 - DNA, 24 bp, Artificial Sequence
  Synthetic PTM28-829F
ggtgtagcgggtattgatccctgc

SEQ ID NO: 50 - DNA, 23 bp, Artificial Sequence
  Synthetic PTM28-1058R
cagcacctgtcactttgtcccga

SEQ ID NO: 51 - DNA, 21 bp, Artificial Sequence
  Synthetic PTM29-841F
gggcactaggtgcagggggtg

SEQ ID NO: 52 - DNA, 23 bp, Artificial Sequence
  Synthetic PTM29-1051R
tgtcaccaggttcccccgaaggg

SEQ ID NO: 53 - DNA, 27 bp, Artificial Sequence
  Synthetic PTM30-832F
cacgccstaaacagtggacactagata

SEQ ID NO: 54 - DNA, 24 bp, Artificial Sequence
  Synthetic PTM30-1061R
cagcacctgtgacagttcctgact

SEQ ID NO: 55 - DNA, 22 bp, Artificial Sequence
  Synthetic PTM31-806F
ctagctgtaaacgatgcgggct

SEQ ID NO: 56 - DNA, 25 bp, Artificial Sequence
  Synthetic PTM31-1040R
agctaatccggtaaggtcttcagcc

SEQ ID NO: 57 - DNA, 25 bp, Artificial Sequence
  Synthetic PTM32-807F
tagccgtaaacgatgggcactagat

SEQ ID NO: 58 - DNA, 20 bp, Artificial Sequence
  Synthetic PTM32-1054R
acctctgctggcttcctggc

SEQ ID NO: 59 - DNA, 23 bp, Artificial Sequence
  Synthetic PTM33-818F
gatgggcacttgacgtaggcgat

SEQ ID NO: 60 - DNA, 23 bp, Artificial Sequence
  Synthetic PTM33-1053R
cacctgtacaggctccggattgg

SEQ ID NO: 61 - DNA, 23 bp, Artificial Sequence
  Synthetic PTM34-839F
cgatgtggacttggcgttggtgg

SEQ ID NO: 62 - DNA, 20 bp, Artificial Sequence
  Synthetic PTM34-1056R
gcagcacctgtccaggctcc

SEQ ID NO: 63 - DNA, 30 bp, Artificial Sequence
  Synthetic PTM35-838F
gctgtaaacgatggatactagattttgcaa

SEQ ID NO: 64 - DNA, 26 bp, Artificial Sequence
  Synthetic PTM35-1032R
cgaagaggataaccaaccctttcagg

SEQ ID NO: 65 - DNA, 28 bp, Artificial Sequence
  Synthetic PTM36-808F
agctgtaaacgatggatactaggtgtgg

SEQ ID NO: 66 - DNA, 28 bp, Artificial Sequence
  Synthetic PTM36-1063R
gcaccacctgttatytcgtcttccctaa

SEQ ID NO: 67 - DNA, 24 bp, Artificial Sequence
  Synthetic PTM37-829F
cacgccctaaacggtggacactag

SEQ ID NO: 68 - DNA, 21 bp, Artificial Sequence
  Synthetic PTM37-1059R
gcacctgtggcagctcctgac

SEQ ID NO: 69 - DNA, 25 bp, Artificial Sequence
  Synthetic PTM38-808F
agccgtaaacgatggacacttgacg

SEQ ID NO: 70 - DNA, 25 bp, Artificial Sequence
  Synthetic PTM38-1031R
gttaccggttgtcaccctttcgggc

SEQ ID NO: 71 - DNA, 23 bp, Artificial Sequence
  Synthetic PTM39-838F
acgatgctcgctatgtgtcaggt

SEQ ID NO: 72 - DNA, 24 bp, Artificial Sequence
  Synthetic PTM39-1045R
ctctcagcggatctggtaaggtct

SEQ ID NO: 73 - DNA, 26 bp, Artificial Sequence
  Synthetic PTM40-834F
gccctaaacgatgtacacttggcatg

SEQ ID NO: 74 - DNA, 24 bp, Artificial Sequence
  Synthetic PTM40-1051R
cctgtgctgactttccaccagagg

SEQ ID NO: 75 - DNA, 22 bp, Artificial Sequence
  Synthetic PTM41-838F
ggtattgacccctgctgtgccg

SEQ ID NO: 76 - DNA, 23 bp, Artificial Sequence
  Synthetic PTM41-1042R
gggttccccgaagggcacatccc

SEQ ID NO: 77 - DNA, 23 bp, Artificial Sequence
  Synthetic PTM42-837F
aggtatcgaccccttctgtgccg

SEQ ID NO: 78 - DNA, 25 bp, Artificial Sequence
  Synthetic PTM42-1060R
gcaccacctgttatctcgtcttccg

SEQ ID NO: 79 - DNA, 22 bp, Artificial Sequence
  Synthetic PTM43-824F
cgctaggtgtcagacacggtgc

SEQ ID NO: 80 - DNA, 25 bp, Artificial Sequence
  Synthetic PTM43-1048R
tcctctcagcgattcaggtaagacc

SEQ ID NO: 81 - DNA, 24 bp, Artificial Sequence
  Synthetic PTM44-809F
gctgtaaacgatgtggacttggcg

SEQ ID NO: 82 - DNA, 20 bp, Artificial Sequence
  Synthetic PTM44-1045R
ccaggctccccgaagggtcg

SEQ ID NO: 83 - DNA, 23 bp, Artificial Sequence
  Synthetic PTM45-842F
aggtatcgaccccttctgtgccg

SEQ ID NO: 84 - DNA, 26 bp, Artificial Sequence
  Synthetic PTM45-1063R
gcaccacctgttatcctgtcttccct

SEQ ID NO: 85 - DNA, 21 bp, Artificial Sequence
  Synthetic PTM46-837F
cgaccccttctgtgccgtagc

SEQ ID NO: 86 - DNA, 25 bp, Artificial Sequence
  Synthetic PTM46-1060R
gcaccacctgttatcctgtcttcgg

SEQ ID NO: 87 - DNA, 24 bp, Artificial Sequence
  Synthetic PTM47-816F
acgatgcgtgctaggtgttggtag

SEQ ID NO: 88 - DNA, 25 bp, Artificial Sequence
  Synthetic PTM47-1037R
ttgtctggtaaggtcgtcagcctga

SEQ ID NO: 89 - DNA, 21 bp, Artificial Sequence
  Synthetic PTM48-817F
cgatgcgggctaggtgttggg

SEQ ID NO: 90 - DNA, 23 bp, Artificial Sequence
  Synthetic PTM48-1045R
ctctcagcttgtccagcaagacc

SEQ ID NO: 91 - DNA, 25 bp, Artificial Sequence
  Synthetic PTM49-818F
gctgtgggcttagtgttgggtgtct

SEQ ID NO: 92 - DNA, 22 bp, Artificial Sequence
  Synthetic PTM49-1046R
acctctcggcaatccagcaagg

SEQ ID NO: 93 - DNA, 27 bp, Artificial Sequence
  Synthetic PTM50-811F
cgtaaacgatgcatactaggtgatggc

SEQ ID NO: 94 - DNA, 24 bp, Artificial Sequence
  Synthetic PTM50-1041R
cagctcgtcaggtaaggtcgtcaa

SEQ ID NO: 95 - DNA, 26 bp, Artificial Sequence
  Synthetic PTM51-835F
tgcatactaggtgatggtacggccat

SEQ ID NO: 96 - DNA, 22 bp, Artificial Sequence
  Synthetic PTM51-1045R
cctctcagctcgtcgggtaagg

SEQ ID NO: 97 - DNA, 22 bp, Artificial Sequence
  Synthetic PTM52-817F
cgatgcgggctaggtgttaggg

SEQ ID NO: 98 - DNA, 23 bp, Artificial Sequence
  Synthetic PTM52-1041R
cagcttgtctggcaagatcgtca

SEQ ID NO: 99 - DNA, 26 bp, Artificial Sequence
  Synthetic PTM53-811F
tgtaaacgctgcctgcttagtgttag

SEQ ID NO: 100 - DNA, 27 bp, Artificial Sequence
  Synthetic PTM53-1049R
ctctctacctattgatcgagcaaggtc

SEQ ID NO: 101 - DNA, 21 bp, Artificial Sequence
  Synthetic PTM54-817F
cgctgcccgcttggtattagg

SEQ ID NO: 102 - DNA, 25 bp, Artificial Sequence
  Synthetic PTM54-1043R
ctcggagaattcagcaaggtcttca

SEQ ID NO: 103 - DNA, 24 bp, Artificial Sequence
  Synthetic PTM55-817F
cgctgcttgcttgatgttagttgg

SEQ ID NO: 104 - DNA, 26 bp, Artificial Sequence
  Synthetic PTM55-1044R
tctcggaaaatcaggcaaggtcatca

SEQ ID NO: 105 - DNA, 20 bp, Artificial Sequence
  Synthetic PTM56-817F
cgctgcaggcttggtgttgg

SEQ ID NO: 106 - DNA, 27 bp, Artificial Sequence
  Synthetic PTM56-1044R
tctcggaaaatcaggcaaagtcatcag

SEQ ID NO: 107 - DNA, 21 bp, Artificial Sequence
  Synthetic PTM57-816F
acgctgcagacttggtgtcgg

SEQ ID NO: 108 - DNA, 26 bp, Artificial Sequence
  Synthetic PTM57-1045R
ctctcggaaaatcgggcaaagtcatc

SEQ ID NO: 109 - DNA, 20 bp, Artificial Sequence
  Synthetic PTM58-817F
cgctgcaggcttggtgttgg

SEQ ID NO: 110 - DNA, 29 bp, Artificial Sequence
  Synthetic PTM58-1046R
cctctcgaaaaatcaggtaaggtcatcag

SEQ ID NO: 111 - DNA, 23 bp, Artificial Sequence
  Synthetic PTM59-816F
acgatgcgagctaggtggtagtc

SEQ ID NO: 112 - DNA, 27 bp, Artificial Sequence
  Synthetic PTM59-1044R
tctcagctaatctgacaaggtcttcag

SEQ ID NO: 113 - DNA, 23 bp, Artificial Sequence
  Synthetic PTM60-820F
tgcgggctaggtgttggcattac

SEQ ID NO: 114 - DNA, 27 bp, Artificial Sequence
  Synthetic PTM60-1041R
cagctaatttggtaaggtcttcagcct

SEQ ID NO: 115 - DNA, 20 bp, Artificial Sequence
  Synthetic PTM61-817F
cgatgcgggccaggtgttgg

SEQ ID NO: 116 - DNA, 26 bp, Artificial Sequence
  Synthetic PTM61-1047R
acctctcagctaatccggtaaggtct

SEQ ID NO: 117 - DNA, 20 bp, Artificial Sequence
  Synthetic PTM62-817F
cgatgcgcgttaggtgtgcc

SEQ ID NO: 118 - DNA, 23 bp, Artificial Sequence
  Synthetic PTM62-1039R
gctggtcaagcaaggtcttcagc

SEQ ID NO: 119 - DNA, 26 bp, Artificial Sequence
  Synthetic PTM63-811F
cgtaaacgatgtgagctaggtgtcag

SEQ ID NO: 120 - DNA, 24 bp, Artificial Sequence
  Synthetic PTM63-1046R
cctctcagcgaatcgggtaaggtc

SEQ ID NO: 121 - DNA, 25 bp, Artificial Sequence
  Synthetic PTM64-817F
cgatgtgagctaggtgtcagtcatg

SEQ ID NO: 122 - DNA, 27 bp, Artificial Sequence
  Synthetic PTM64-1047R
acctctcagcgaatttggtaaggtctt

SEQ ID NO: 123 - DNA, 23 bp, Artificial Sequence
  Synthetic PTM65-811F
cgtaaacgatgcgagctaggtgt

SEQ ID NO: 124 - DNA, 24 bp, Artificial Sequence
  Synthetic PTM65-1043R
ctcagcaagtctggcaaggtcttc

SEQ ID NO: 125 - DNA, 22 bp, Artificial Sequence
  Synthetic PTM66-817F
cgatgcttgctaggtgtcagcc

SEQ ID NO: 126 - DNA, 26 bp, Artificial Sequence
  Synthetic PTM66-1047R
acctctcagctaatcgggtaaggtct

SEQ ID NO: 127 - DNA, 23 bp, Artificial Sequence
  Synthetic PTM67-814F
aaacgatgctcgctaggtgtcag

SEQ ID NO: 128 - DNA, 27 bp, Artificial Sequence
  Synthetic PTM67-1046R
cctctcagcgaatcaggtaaggtcttc

SEQ ID NO: 129 - DNA, 28 bp, Artificial Sequence
  Synthetic PTM68-821F
gggtactaggtgtaggaggtatcgaccc

SEQ ID NO: 130 - DNA, 24 bp, Artificial Sequence
  Synthetic PTM68-1057R
accacctgtctccctgttcttccg

SEQ ID NO: 131 - DNA, 27 bp, Artificial Sequence
  Synthetic PTM69-819F
gtaaacgatgggcactaggtgttggag

SEQ ID NO: 132 - DNA, 30 bp, Artificial Sequence
  Synthetic PTM69-1052R
tctccctgtctcaagaaaatcttaagagga

SEQ ID NO: 133 - DNA, 30 bp, Artificial Sequence
  Synthetic PTM70-815F
aacgatggatactaggtgtagggggtttag

SEQ ID NO: 134 - DNA, 26 bp, Artificial Sequence
  Synthetic PTM70-1053R
ccacctgtatacctgtccccgaaagg

SEQ ID NO: 135 - DNA, 29 bp, Artificial Sequence
  Synthetic PTM71-809F
gctgtaaacgatggatactaggtgtaggg

SEQ ID NO: 136 - DNA, 28 bp, Artificial Sequence
  Synthetic PTM71-1055R
caccacctgtttacctgtcccctaaagg

SEQ ID NO: 137 - DNA, 29 bp, Artificial Sequence
  Synthetic PTM72-815F
aacgatggatactaggtgtgggaggtatc

SEQ ID NO: 138 - DNA, 27 bp, Artificial Sequence
  Synthetic PTM72-1058R
cacctgttatctcgtcttccccaaagg

SEQ ID NO: 139 - DNA, 21 bp, Artificial Sequence
  Synthetic PTM73-816F
acgatgtgcacttggcatgcg

SEQ ID NO: 140 - DNA, 23 bp, Artificial Sequence
  Synthetic PTM73-1050R
tgctgacttttcaccagaggcga

SEQ ID NO: 141 - DNA, 25 bp, Artificial Sequence
  Synthetic PTM74-812F
gcaaacgatgttcactgggtgtcgg

SEQ ID NO: 142 - DNA, 22 bp, Artificial Sequence
  Synthetic PTM74-1037R
ctgtgctagctcctctacccga

SEQ ID NO: 143 - DNA, 24 bp, Artificial Sequence
  Synthetic PTM75-809F
gccgtaaacgatggatgcttggtg

SEQ ID NO: 144 - DNA, 27 bp, Artificial Sequence
  Synthetic PTM75-1055R
gcacgggtaacagagattactctctga

SEQ ID NO: 145 - DNA, 27 bp, Artificial Sequence
  Synthetic PTM76-821F
ggctactagctgtttgaagtatcgacc

SEQ ID NO: 146 - DNA, 27 bp, Artificial Sequence
  Synthetic PTM76-1050R
ctgctctagtgtccttgtaggtagaca

SEQ ID NO: 147 - DNA, 28 bp, Artificial Sequence
  Synthetic PTM77-815F
aacgatggacactggctatttgaagtgt

SEQ ID NO: 148 - DNA, 26 bp, Artificial Sequence
  Synthetic PTM77-1049R
tgggctagtgtccttgtgggtagact

SEQ ID NO: 149 - DNA, 28 bp, Artificial Sequence
  Synthetic PTM78-817F
ctttggacactaggtatggagggtatcg

SEQ ID NO: 150 - DNA, 21 bp, Artificial Sequence
  Synthetic PTM78-1052R
tgtgccggctcctggctttac

SEQ ID NO: 151 - DNA, 28 bp, Artificial Sequence
  Synthetic PTM79-813F
caaacgatggacactaggtatggggggt

SEQ ID NO: 152 - DNA, 20 bp, Artificial Sequence
  Synthetic PTM79-1048R
tgtgcacccgtcctgcgaag

SEQ ID NO: 153 - DNA, 28 bp, Artificial Sequence
  Synthetic PTM80-817F
cggtggatactggatatagggggtatcg

SEQ ID NO: 154 - DNA, 26 bp, Artificial Sequence
  Synthetic PTM80-1052R
gtgctagctccttggaaaaccaaggt

SEQ ID NO: 155 - DNA, 30 bp, Artificial Sequence
  Synthetic PTM81-814F
aaacggtggacattaggtatggggagtatc

SEQ ID NO: 156 - DNA, 21 bp, Artificial Sequence
  Synthetic PTM81-1056R
cctgtgccagctcctgactgg

SEQ ID NO: 157 - DNA, 27 bp, Artificial Sequence
  Synthetic PTM82-816F
acggtggacactagacatgggaggtat

SEQ ID NO: 158 - DNA, 25 bp, Artificial Sequence
  Synthetic PTM82-1055R
ctgtgacagctcctgactggataca

SEQ ID NO: 159 - DNA, 28 bp, Artificial Sequence
  Synthetic PTM83-812F
ctaaacggtggacactagatatggggag

SEQ ID NO: 160 - DNA, 26 bp, Artificial Sequence
  Synthetic PTM83-1048R
agttcctgactggatacaggtcgtcc

SEQ ID NO: 161 - DNA, 28 bp, Artificial Sequence
  Synthetic PTM84-817F
cgatggacactaggtatagggagtatcg

SEQ ID NO: 162 - DNA, 26 bp, Artificial Sequence
  Synthetic PTM84-1055R
acctgtgacggctcctgatttaacag

SEQ ID NO: 163 - DNA, 27 bp, Artificial Sequence
  Synthetic PTM85-807F
acgccctaaacgttggacactaggtat

SEQ ID NO: 164 - DNA, 24 bp, Artificial Sequence
  Synthetic PTM85-1048R
agctcctgactggatacaggtcgt

SEQ ID NO: 165 - DNA, 29 bp, Artificial Sequence
  Synthetic PTM86-811F
cgtaaactatggacactaggtatggggag

SEQ ID NO: 166 - DNA, 22 bp, Artificial Sequence
  Synthetic PTM86-1052R
tgtgccggctcctgactcaaca

SEQ ID NO: 167 - DNA, 25 bp, Artificial Sequence
  Synthetic PTM87-817F
cgatggatactaggtgtgggtggca

SEQ ID NO: 168 - DNA, 19 bp, Artificial Sequence
  Synthetic PTM87-1049R
ctgtgctggctcccttgcg

SEQ ID NO: 169 - DNA, 23 bp, Artificial Sequence
  Synthetic PTM88-815F
aacgatggatgctgggtgtgggg

SEQ ID NO: 170 - DNA, 20 bp, Artificial Sequence
  Synthetic PTM88-1046R
tgcaggctccccgaagggtc

SEQ ID NO: 171 - DNA, 30 bp, Artificial Sequence
  Synthetic PTM89-818F
gatgcagacttggtgttggtggtttaatag

SEQ ID NO: 172 - DNA, 17 bp, Artificial Sequence
  Synthetic PTM89-1055R
cagcacctgtgcgcgct

SEQ ID NO: 173 - DNA, 26 bp, Artificial Sequence
  Synthetic PTM90-817F
cgatgcctactaggttgtggtggttc

SEQ ID NO: 174 - DNA, 26 bp, Artificial Sequence
  Synthetic PTM90-1054R
cctgtgcaagtttcacccgaaggtaa

SEQ ID NO: 175 - DNA, 25 bp, Artificial Sequence
  Synthetic PTM91-810F
ccgtaaacgatgggcacttgacgta

SEQ ID NO: 176 - DNA, 27 bp, Artificial Sequence
  Synthetic PTM91-1293R
cacctgtcagattccggactgattacc

SEQ ID NO: 177 - DNA, 30 bp, Artificial Sequence
  Synthetic PTM92-808F
agctgtaaacgatggatactagattttgca

SEQ ID NO: 178 - DNA, 26 bp, Artificial Sequence
  Synthetic PTM92-1043R
ataggttcctccgaagaggatagcca

SEQ ID NO: 179 - DNA, 25 bp, Artificial Sequence
  Synthetic PTM93-816F
acgatgggcactagatgtttctgct

SEQ ID NO: 180 - DNA, 21 bp, Artificial Sequence
  Synthetic PTM93-1053R
acctctgctggcttcctgcaa

SEQ ID NO: 181 - DNA, 25 bp, Artificial Sequence
  Synthetic PTM94-817F
cgatgggcactagatgtttctgctt

SEQ ID NO: 182 - DNA, 20 bp, Artificial Sequence
  Synthetic PTM94-1053R
cctctgctggcttcctggca

SEQ ID NO: 183 - DNA, 27 bp, Artificial Sequence
  Synthetic PTM95-812F
gtaaacgatgatcactcgttgttggcg

SEQ ID NO: 184 - DNA, 24 bp, Artificial Sequence
  Synthetic PTM95-1042R
gattcccttcggggcagattgcaa

SEQ ID NO: 185 - DNA, 27 bp, Artificial Sequence
  Synthetic PTM96-818F
gatgagtgctaggtgttggggggtttc

SEQ ID NO: 186 - DNA, 24 bp, Artificial Sequence
  Synthetic PTM96-1051R
cctgtcaccattgtccccgaaggg

SEQ ID NO: 187 - DNA, 30 bp, Artificial Sequence
  Synthetic PTM97-817F
cgatgttcactaggtgttgggagtattgac

SEQ ID NO: 188 - DNA, 23 bp, Artificial Sequence
  Synthetic PTM97-1053R
acctgtcaccgagttccccgaag

SEQ ID NO: 189 - DNA, 30 bp, Artificial Sequence
  Synthetic PTM98-820F
tgttcactaggtgttgggagtattgaccct

SEQ ID NO: 190 - DNA, 24 bp, Artificial Sequence
  Synthetic PTM98-1051R
cctgtcaccaagttccccgaaggg

SEQ ID NO: 191 - DNA, 27 bp, Artificial Sequence
  Synthetic PTM99-813F
taaacgatgagaactaggtgtagcggg

SEQ ID NO: 192 - DNA, 27 bp, Artificial Sequence
  Synthetic PTM99-1046R
tctgttccgacaaagtcggaaagatcc

SEQ ID NO: 193 - DNA, 27 bp, Artificial Sequence
  Synthetic PTM100-817F
cgatgaacactaggtgtagcgggtatt

SEQ ID NO: 194 - DNA, 21 bp, Artificial Sequence
  Synthetic PTM100-1044R
ccgagttccccgaagggcaca

SEQ ID NO: 195 - DNA, 24 bp, Artificial Sequence
  Synthetic PTM101-813F
taaactatgggtgctagccgtcgg

SEQ ID NO: 196 - DNA, 24 bp, Artificial Sequence
  Synthetic PTM101-1046R
cacctgtcaccggccaattgaaga

SEQ ID NO: 197 - DNA, 26 bp, Artificial Sequence
  Synthetic PTM102-821F
gggtattagacatcggccgaaattcg

SEQ ID NO: 198 - DNA, 23 bp, Artificial Sequence
  Synthetic PTM102-1040R
caggttctcttacgagcactccg

SEQ ID NO: 199 - DNA, 28 bp, Artificial Sequence
  Synthetic PTM103-812F
gtaaacgatgtcaactaactgttgggcg

SEQ ID NO: 200 - DNA, 25 bp, Artificial Sequence
  Synthetic PTM103-1046R
ctgtatcagagttcccgaaggcacc

SEQ ID NO: 201 - DNA, 22 bp, Artificial Sequence
  Synthetic PTM03-Reverse
caacgggtggccacggagtgat

SEQ ID NO: 202 - DNA, 26 bp, Artificial Sequence
  Synthetic PTM04-Reverse
gtgaaggcaatcccgaaacccatcca

SEQ ID NO: 203 - DNA, 24 bp, Artificial Sequence
  Synthetic PTM05-Reverse
ctcgctcttagttgctatcttcga

SEQ ID NO: 204 - DNA, 24 bp, Artificial Sequence
  Synthetic PTM06-Reverse
cggaacaaagagtaccgatcccga

SEQ ID NO: 205 - DNA, 28 bp, Artificial Sequence
  Synthetic PTM07-Reverse
ccatcttcagttgctattttcaagtccg

SEQ ID NO: 206 - DNA, 25 bp, Artificial Sequence
  Synthetic PTM08-Reverse
ctgacgaaactgcccagaaatcctg

SEQ ID NO: 207 - DNA, 23 bp, Artificial Sequence
  Synthetic PTM10-Reverse
tcgcagataatgcggaggaaggt

SEQ ID NO: 208 - DNA, 474 bp, Artificial Sequence
  Synthetic CONSENS_3
cacgccctaaacggtggatactagatayaggggatatcaactcctccgtgtcgaagctaacgctttaagtatcccgcctgggaactacggccgcaaggctaaaactcaaaggaattgacgggggcccgcacaagcagcggagcgtgtggtttaattcgatgcaacacgaagaaccttacccaggyttgacatgctagtggtaggaacctgaaagggagacgaccctggttttccagggagctagcacaggygctgcatggctgtcgtcagctcgtgccgtgaggtgttgggttaagtcccacaacgagcgcaacccccatcgtcagttgaatttctctgacgaaactgcccagaaatcctgggaggaaggaggggatgacgtcaagtcagyatggcccttatgcctggggcracacacacgctacaatgggtggtacaacgggtggccacggagygatccggagctaatcctca

SEQ ID NO: 209 - DNA, 474 bp, Artificial Sequence
  Synthetic 1625_Q993T
cacgccctaaacggtggatactagatataggggatatcaactcctccgtgtcgaagctaacgctttaagtatcccgcctgggaactacggccgcaaggctaaaactcaaaggaattgacgggggcccgcacaagcagcggagcgtgtggtttaattcgatgcaacacgaagaaccttacccaggcttgacatgctagtggtaggaacctgaaagggagacgaccctggttttccagggagctagcacaggtgctgcatggctgtcgtcagctcgtgccgtgaggtgttgggttaagtcccacaacgagcgcaacccccatcgtcagttgaatttctctgacgaaactgcccagaaatcctgggaggaaggaggggatgacgtcaagtcagtatggcccttatgcctggggcaacacacacgctacaatgggtggtacaacgggtggccacggagtgatccggagctaatcctca

SEQ ID NO: 210 - DNA, 474 bp, Artificial Sequence
  Synthetic 1637_T238T
cacgccctaaacggtggatactagatacaggggatatcaactcctccgtgtcgaagctaacgctttaagtatcccgcctgggaactacggccgcaaggctaaaactcaaaggaattgacgggggcccgcacaagcagcggagcgtgtggtttaattcgatgcaacacgaagaaccttacccaggtttgacatgctagtggtaggaacctgaaagggagacgaccctggttttccagggagctagcacaggcgctgcatggctgtcgtcagctcgtgccgtgaggtgttgggttaagtcccacaacgagcgcaacccccatcgtcagttgaatttctctgacgaaactgcccagaaatcctgggaggaaggaggggatgacgtcaagtcagcatggcccttatgcctggggcgacacacacgctacaatgggtggtacaacgggtggccacggagcgatccggagctaatcctca

SEQ ID NO: 211 - DNA, 474 bp, Artificial Sequence
  Synthetic 1638_U765T
cacgccctaaacggtggatactagatataggggatatcaactcctccgtgtcgaagctaacgctttaagtatcccgcctgggaactacggccgcaaggctaaaactcaaaggaattgacgggggcccgcacaagcagcggagcgtgtggtttaattcgatgcaacacgaagaaccttacccaggtttgacatgctagtggtaggaacctgaaagggagacgaccctggttttccagggagctagcacaggtgctgcatggctgtcgtcagctcgtgccgtgaggtgttgggttaagtcccacaacgagcgcaacccccatcgtcagttgaatttctctgacgaaactgcccagaaatcctgggaggaaggaggggatgacgtcaagtcagcatggcccttatgcctggggcgacacacacgctacaatgggtggtacaacgggtggccacggagcgatccggagctaatcctca

SEQ ID NO: 212 - DNA, 551 bp, Artificial Sequence
  Synthetic CONSENS_14
cagggcgtaaacgctgtgggcttagtgttaggtgtcccatgagggcccctagtgctggagagaagttgttaagcccacaacctgggaagtacggtcgcaaggctgaaacttaaaggaatcggcgggggagcacagcaacgggtggagcgtacggttcaattggattctacgccggaaatctcaccgggggcgacggctcgatgarggccaggctgatgaccttgccagatgtgccgagaggtggtgcatggccgccgtcagttcgtgccgcaaggtgttctgttaagtcagataacgaacgagaccctcacctttaattgctaccctttcctctgggagaggggcacattagagggaccgccactgctaaagtggaggaaggggggggcaacggtaggtcagtatgccccaaatctcccgggctacacgcgcgctacaaagaatgggacaatgggytccgacaccgagaggtgaaggcaatcccgaaacccatccatagttcggattgagggctgaaactcgccctcatgaagctggaatccgtagtaatc

SEQ ID NO: 213 - DNA, 550 bp, Artificial Sequence
  Synthetic 1639_V292T
cagggcgtaaacgctgtgggcttagtgttaggtgtcccatgagggcccctagtgctggagagaagttgttaagcccacaacctgggaagtacggtcgcaaggctgaaacttaaaggaatcggcgggggagcacagcaacgggtggagcgtacggttcaattggattctacgccggaaatctcaccgggggcgacggtcgatgaaggccaggctgatgaccttgctagatgtgccgagaggtggtgcatggccgccgtcagttcgtgccgcaaggtgttctgttaagtcagataacgaacgagaccctcacctttaattgctaccctttcctctgggagaggggcacattagagggaccgccactgctaaagtggagggaggggggggcaacggtaggtcagtatgccccaaatctcccgggctacacgcgcgctacaaagaatgggacaatgggttccgacaccgagaggtgaaggcaatcccgaaacccatccatagttcggattgagggctgaaactcgccctcatgaagctggaatccgtagtaatc

SEQ ID NO: 214 - DNA, 550 bp, Artificial Sequence
  Synthetic 1629_R760T
cagggcgtaaacgctgtgggcttagtgttaggtgtcccatgagggcccctagtgctggagagaagttgttaagcccacaacctgggaagtacggtcgcagggctgaaacttaaaggaatcggcgggggagcacagcaacgggtggagcgtacggttcaattggattctacgccggaaatctcaccgggggcgacggtcgatgaaggccaggctgatgaccttgccagatgtgccgagaggtggtgcatggccgccgtcagttcgtgccgcaaggtgttctgttaagtcagataacgaacgagaccctcacctttaattgctaccctttcctctgggagaggggcacattagagggaccgccactgctaaagtggaggaaggggggggcaacggtaggtcagtatgccccaaatctcccgggctacacgcgcgctacaaagaatgggacaatgggttccgacaccgagaggtgaaggcaatcccgaaacccatccatagttcggattgagggctgaaactcgccctcatgaagctggaatccgtagtaatc

SEQ ID NO: 215 - DNA, 550 bp, Artificial Sequence
  Synthetic 1629_R361T
cagggcgtaaacgctgtgggcttagtgttaggtgtcccatgagggcccctagtgctggagagaagttgttaagcccacaacctgggaagtacggtcgcaaggctgaaacttaaaggaatcggcgggggagcacagcaacgggtggagcgtacggttcaattggattctacgccggaaatctcaccgggggcgacggctcgatgagggccaggctgatgaccttgccagatgtgccgagaggtggtgcatggccgccgtcagttcgtgccgcaaggtgttctgttaagtcagataacgaacgagaccctcacctttaattgctaccctttcctctgggagaggggcacattagagggaccgccactgctaaagtggaggaaggggggggcaacggtaggtcagtatgccccaaatctcccgggctacacgcgcgctacaaagaatgggacaatgggttccgacaccgagaggtgaaggcaatcccgaaacccatccatagttcggattgagggctgaaactcgccctcatgaagctgaatccgtagtaatc

SEQ ID NO: 216 - DNA, 551 bp, Artificial Sequence
  Synthetic 1637_T237T
cagggcgtaaacgctgtgggcttagtgttaggtgtcccatgagggcccctagtgctggagagaagttgttaagcccacaacctgggaagtacggtcgcaaggctgaaacttaaaggaatcggcgggggagcacagcaacgggtggagcgtacggttcaattggattctacgccggaaatctcaccgggggcgacggctcgatgagggccaggctgatgaccttgccagatgtgccgagaggtggtgcatggccgccgtcagttcgtgccgcaaggtgttctgttaagtcagataacgaacgagaccctcacctttaattgctaccctttcctctgggagaggggcacattagagggaccgccactgctaaagtggaggaaggggggggcaacggtaggtcagtatgccccaaatctcccgggctacacgcgcgctacaaagaatgggacaatgggttccgacaccgagaggtgaaggcaatcccgaaacccatccatagttcggattgagggctgaaactcgccctcatgaagctggaatccgtagtaatc

SEQ ID NO: 217 - DNA, 551 bp, Artificial Sequence
  Synthetic 1637_T148T
cagggcgtaaacgctgtgggcttagtgttgggtgtcccatgagggcccctagtgctggagagaagttgttaagcccacaacctgggaagtacggtcgcaaggctgaaacttaaaggaatcggcgggggagcacagcaacgggtggagcgtacggttcaattggattctacgccggaaatctcaccgggggcgacggctcgatgagggccaggctgatgaccttgccagatgtgccgagaggtggtgcatggccgccgtcagttcgtgccgcaaggtgttctgttaagtcagataacgaacgagaccctcacctttaattgctaccctttcctctgggagaggggcacattagagggaccgccactgctaaagtggaggaaggggggggcaacggtaggtcagtatgccccaaatctcccgggctacacgcgcgctacaaagaatgggacaatgggttccgacaccgagaggtgaaggcaatcccgaaacccatccatagttcggattgagggctgaaactcgccctcatgaagctggaatccgtagtaatc

SEQ ID NO: 218 - DNA, 551 bp, Artificial Sequence; identical to SEQ ID NO: 216
  Synthetic 1639_V895T

SEQ ID NO: 219 - DNA, 551 bp, Artificial Sequence; identical to SEQ ID NO: 216
  Synthetic 1629_R065T

SEQ ID NO: 220 - DNA, 551 bp, Artificial Sequence; identical to SEQ ID NO: 216
  Synthetic 1638_U187T

SEQ ID NO: 221 - DNA, 551 bp, Artificial Sequence
  Synthetic 1629_R855T
cagggcgtaaacgctgtgggcttagtgttaggtgtcccatgagggcccctagtgctggagagaagttgttaagcccacaacctgggaagtacggtcgcaaggctgaaacttaaaggaatcggcgggggagcacagcaacgggtggagcgtacggttcaattggattctacgccggaaatctcaccgggggcgacggctcgatgaaggccaggctgatgaccttgccagatgtgccgagaggtggtgcatggccgccgtcagttcgtgccgcaaggtgttctgttaagtcagataacgaacgagaccctcacctttaattgctaccctttcctctgggagaggggcacattagagggaccgccactgctaaagtggaggaaggggggggcaacggtaggtcagtatgccccaaatctcccgggctacacgcgcgctacaaagaatgggacaatgggctccgacaccgagaggtgaaggcaatcccgaaacccatccatagttcggattgagggctgaaactcgccctcatgaagctggaatccgtagtaatc

SEQ ID NO: 222 - DNA, 551 bp, Artificial Sequence; identical to SEQ ID NO: 221
  Synthetic 1629_R061T

SEQ ID NO: 223 - DNA, 551 bp, Artificial Sequence
  Synthetic 1629_R018T
cagggcgtaaacgctgtgggcttagtgttaggtgtcccatgagggcccctagtgctggagagaagttgttaagcccacaacctgggaagtacggtcgcaaggctgaaacttaaaggaatcggcgggggagcacagcaacgggtggagcgtacggttcaattggattctacgccggaaatctcaccgggggcgacggctcgatgaaggccaggctgatgaccttgctagatgtgccgagaggtggtgcatggccgccgtcagttcgtgccgcaaggtgttctgttaagtcagataacgaacgagaccctcacctttaattgctaccctttcctctgggagaggggcacattagagggaccgccactgctaaagtggaggaaggggggggcaacggtaggtcagtatgccccaaatctcccgggctacacgcgcgctacaaagaatgggacaatgggttccgacaccgagaggtgaaggcaatcccgaaacccatccatagttcggattgagggctgaaactcgccctcatgaagctggaatccgtagtaatc

SEQ ID NO: 224 - DNA, 551 bp, Artificial Sequence
  Synthetic 1639_V551T
cagggcgtaaacgctgtgggcttagtgttaggtgtcccatgagggcccctagtgctgtagcgaaggtgttaagcccacaacctgggaagtacggtcgcaaggctgaaacttaaaggaatcggcgggggagcacagcaacgggtggagcgtgcggttcaattggattatacgccggaaagctcaccgggggcgacggctcgatgagggccaggctgatgaccttgccagatgtgccgagaggtggtgcatggccgccgtcagttcgtgccgcaaggtgttctgttaagtcagacaacgaacgagatccccgcccttaattgccaccccttcctctgggaagggagcacattagggggaccaccactgctaaagtggaggaagaggggggcaacggtaggtcagtatgccccaaatctcccgggctacacgcgcgctacaaagaatgggacaatgggctccgaccccgagaggggaaggcaatcccgaaacccatccatagttcggattgaggactgaaactcgtcctcatgaagctggaatccgtagtaatc

SEQ ID NO: 225 - DNA, 551 bp, Artificial Sequence
  Synthetic 1639_V652T
cagggcgtaaacgctgtgggcttagtgttaggtgtcccatgagggcccctagtgctggagagaagttgttaagcccacaacctgggaagtacggtcgcaaggctgaaacttaaaggaatcggcgggggagcacagcaacgggtggagcgtgcggttcaattggattatacgccggaaagctcaccgggggcgacggctcgatgagggccaggccgatgaccttgccagatgtgccgagaggtggtgcatggccgccgtcagttcgtgccgcaaggtgttctgttaagtcagataacgaacgagatcctcgcccttaattgccaccccttcctctgggaagggagcacattaaggggaccgccactgctaaagtggaggaaggggggggcaacggtaggtcagtatgccccaaatctcccgggctacacgcgcgctacaaaggatgggacaatgggctccgacaccgagaggtgaaggcaatcccgaaacccatccatagttcggattgaggactgaaactcgtcctcatgaagctggaatccgtagtaatc

SEQ ID NO: 226 - DNA, 551 bp, Artificial Sequence; identical to SEQ ID NO: 225
  Synthetic 1637_T014T

SEQ ID NO: 227 - DNA, 507 bp, Artificial Sequence
  Synthetic CONSENS_9
cagggcgtaaacgctgcccgcttggtattagggaacttacaagatttcctattgccggagagaagtcgttaagcgggccacctgggaagtacggtcgcaaggctgaaacttaaaggaattggcgggggagcaccgcaacgggtggagcgtgcggttcaattggatccaacgccggaaagcttaccgagggcgacggatagatgaaggccaggctaatgaccttgctagattttccgagaggtggtgcatggccatcgacagctcgtaccgtgaggcgttctgttaagtcagataacgagcgagaccctcgctcttagttgctatcttcgagtccgctcggggagcactctaagaggaccgctggtgctaaaccagaggaagaagggggcaacggtaggtcagtatgccctgaatccctcgggctacacgcgcgctacaaaggatgggacaatgggtttcgaccccgagagggggaggcaatcccgaaacctatccatagttcggatc

SEQ ID NO: 228 - DNA, 507 bp, Artificial Sequence
  Synthetic 1637_T012T
cagggcgtaaacgctgcccgcttggtattagggaacttacaagatttcctattgccggagagaagtcgttaagcgggccacctgggaagtacggtcgcaaggctgaaacttaaaggaattggcgggggagcaccgcaacgggtggagcgtgcggttcaattggatccaacgccggaaagcttaccgagggcgacggatagatgaaggccaggctaatgaccttgctagattttccgagaggtggtgcatggccatcgacagctcgtaccgtgaggcgttctgttaagtcagataacgagcgagaccctcgctcttagttgctatcttcgagtccgctcggggagcactctaagaggaccgctggtgctaaaccagaggaagaagggggcaacggtaggtcagtatgccctgaatccctcgggctacacgcgcgctacaaaggatgggacaatgggtttcgaccccgagagggggaggcaatcccgaaacctatccataattcggatc

SEQ ID NO: 229 - DNA, 507 bp, Artificial Sequence; identical to SEQ ID NO: 227
  Synthetic 1639_V878T

SEQ ID NO: 230 - DNA, 507 bp, Artificial Sequence; identical to SEQ ID NO: 227
  Synthetic 1638_U823T

SEQ ID NO: 231 - DNA, 507 bp, Artificial Sequence
  Synthetic 1638_U381T
cagggcgtaaacgctgcccgcttggtgttagggaacttacaagatttcctattgccggagagaagtcgttaagcgggccacctgggaagtacggtcgcaaggctgaaacttaaaggaattggcgggggagcaccgcaacgggtggagcgtgcggttcaattggatccaacgccggaaagcttaccgagggcgacggatagatgaaggccaggctaatgaccttgctagattttccgagaggtggtgcatggccatcgacagctcgtaccgtgaggcgttctgttaagtcagataacgagcgagaccctcgctcttagttgctatcttcgagtccgctcggggagcactctaagaggaccgctggtgctaaaccagaggaagaagggggcaacggtaggtcagtatgccctgaatccctcgggctacacgcgcgctacaaaggatgggacaatgggtttcgaccccgagagggggaggcaatcccgaaacctatccatagttcggatc

SEQ ID NO: 232 - DNA, 507 bp, Artificial Sequence
  Synthetic 1637_T010T
cagggcgtaaacgctgcccgcttggtattagggaacttacaagatttcctattgccggagagaagtcgttaagcgggccacctgggaagtacggtcgcaaggctgaaacttaaaggaattggcgggggagcaccgcaacgggtggagcgtgcggttcaattggatccaacgccggaaagcttaccgagggcgacggatagatgaaggccaggctaatgaccttgctagattctccgagaggtggtgcatggccatcgacagctcgtaccgtgaggcgttctgttaagtcagataacgagcgagaccctcgctcttagttgctatcttcgagtccgctcggggagcactctaagaggaccgctggtgctaaaccagaggaagaagggggcaacggtaggtcagtatgccctgaatccctcgggctacacgcgcgctacaaaggatgggacaatgggtttcgaccccgagagggggaggcaatcccgaaacctatccatagttcggatc

SEQ ID NO: 233 - DNA, 507 bp, Artificial Sequence; identical to SEQ ID NO: 227
  Synthetic 1639_V953T

SEQ ID NO: 234 - DNA, 507 bp, Artificial Sequence
  Synthetic 1638_U677T
cagggcgtaaacgctgcccgcttggtattagggaacttacaagatttcctattgccggagagaagtcgttaagcgggccacctgggaagtacggtcgcaaggctgaaacttaaaggaattggcgggggagcaccgcaacgggtggagcgtgcggttcaattggatccaacgccggaaagcttaccgagggcgacggatagatgaaggccaggctaatgaccttgctagattttccgagaggtggtgcatggccatcgacagctcgtaccgtgaggcgttctgttaagtcagataacgagcgagaccctcgctcttagttgctatcttcgagtccgctcggggagcactctaagaggaccgctggtgctaaaccagaggaagaagggggcaacggtaggtcagtatgccctgaatccctcgggctacacgcgcgctacaaaggatgggacaatgggtttcgaccccgagagggggaggcaatcccgaaacctatccatagttcgaatc

SEQ ID NO: 235 - DNA, 507 bp, Artificial Sequence; identical to SEQ ID NO: 227
  Synthetic 1639_V483T

SEQ ID NO: 236 - DNA, 507 bp, Artificial Sequence
  Synthetic 1639_V339T
cagggcgtaaacgctgcccgcttgatgttaggaaacctacaggatttcctagtgtcggagagaagtcgttaagcgggccacctgggaagtacggtcgcaaggctgaaacttaaaggaattggcgggggagcaccgcaacgggtggagcgtgcggttcaattggatccaacgccggaaagcttaccgagggcgacggataaatgaaggccaggctgatgaccttgctagattttccgagaggtggtgcatggccatcgacagctcgtaccgcgaggcgttctgttaagtcagataacgagcgagaccctcgctcttagttgctatcttcgagtccgctcgaagggcactctaagaggaccgctggtgctaaaccagaggaaggagggggcaacggtaggtcagtatgccctgaatccctcgggctacacgcgcgctacaaaggatgggacaatgggctccgaccccgagagggggaggcaatcccgaaacccatccatagttcgaatt

SEQ ID NO: 237 - DNA, 578 bp, Artificial Sequence
  Synthetic CONSENS_19
ccagccgtaaacgatgccagctatgtgtcgggagatccacgtgttcttccggtgccgtagggaagccgtgaagctggccacctgggaagtacggccgcaaggctgaaacttaaaggaattggcgggggagtactacaaccggtggagcttgcggtttaattggatacaacgccggaaatctcaccgggggcgacagcagtatgaaggccaggctgaggaccttgctagattagctgagaggaggtgcatggccgtcgtcagttcgtaccgtgaggcatcctgttaagtcaggcaacgggcgagacccgcggtcttaattgccagcatacccttcggggtgattgggtacaataagacgactgccagcgctaagctggaggaagaagcgggctacggtaggtcagcatgccccraatcccccgggctacacgcgtgctacaatggtcggaacaaagagtaccgatcccgaaagggaaaggtgatctcctaaacccgatcgaagttcggatcgaaggttgcaattcgccttcgtgaagttggaatcggtagtaatcgtgtctcaaaatgacacggtgaat

SEQ ID NO: 238 - DNA, 578 bp, Artificial Sequence
  Synthetic 1639_V271T
ccagccgtaaacgatgccagctatgtgtcgggagatccatgtgttcttccggtgccgtagggaagccgtgaagctggccacctgggaagtacggccgcaaggctgaaacttaaaggaattggcgggggagtactacaaccggtggagcttgcggtttaattggatacaacgccggaaatctcaccgggggcgacagcagtatgaaggccaggctgaggaccttgctagattagctgagaggaggtgcatggccgtcgtcagttcgtaccgtgaggcatcctgttaagtcaggcaacgggcgagacccgcggtcttaattgccagcatacccttcggggtgattgggtacaataagacgactgccagcgctaagctggaggaagaagcgggctacggtaggtcagcatgccccgaatcccccgggctacacgcgtgctacaatggtcggaacaaagagtaccgatcccgaaagggaaaggtgatctcctaaacccgatcgaagttcggatcgaaggttgcaattcgccttcgtgaagttggaatcggtagtaatcgtgtctcaaaatgacacggtgaat

SEQ ID NO: 239 - DNA, 578 bp, Artificial Sequence; identical to SEQ ID NO: 238
  Synthetic 1637_T413T

SEQ ID NO: 240 - DNA, 578 bp, Artificial Sequence; identical to SEQ ID NO: 238
  Synthetic 1639_V773T

SEQ ID NO: 241 - DNA, 578 bp, Artificial Sequence
  Synthetic 1638_U748T
ccagccgtaaacgatgccagctatgtgtcgggagatccatgtgttcttccggtgccgtagggaagccgtgaagctggccacctgggaagtacggccgcaaggctgaaacttaaaggaattggcgggggagtactacaaccggtggagcttgcggtttaattggatacaacgccggaaatctcaccgggggcgacagcagtatgaaggccaggctgaggaccttgctagattagctgagaggaggtgcatggccgtcgtcagttcgtaccgtgaggcatcctgttaagtcaggcaacgggcgagacccgcggtcttaattgccagcatacccttcggggtgattgggtacaataagacgactgccagcgctaagctggaggaagaagcgggctacggtaggtcagcatgccccgaatcccccgggctacacgcgtgccacaatggtcggaacaaagaataccgatcccgaaagggaaaggtgatctcctaaacccgatcgaagttcggatcgaaggttgcaattcgccttcgtgaagttggaatcggtagtaatcgtgtctcaaaatgacacggtgaat

SEQ ID NO: 242 - DNA, 578 bp, Artificial Sequence; identical to SEQ ID NO: 238
  Synthetic 1629_R151T

SEQ ID NO: 243 - DNA, 578 bp, Artificial Sequence
  Synthetic 1629_R125T
ccagccgtaaacgatgccagctatgtgtcgggagatccacgtgttcttccggtgccgtagggaagccgtgaagctggccacctgggaagtacggccgcaaggctgaaacttaaaggaattggcgggggagtactacaaccggtggagcttgcggtttaattggatacaacgccggaaatctcaccgggggcgacagcagtatgaaggccaggctgaggaccttgctagattagctgagaggaggtgcatggccgtcgtcagttcgtaccgtgaggcatcctgttaagtcaggcaacgggcgagacccgcggtcttaattgccagcatacccttcggggtgattgggtacaataagacgactgccagcgctaagctggaggaagaagcgggctacggtaggtcagcatgccccaaatcccccgggctacacgcgtgctacaatggtcggaacaaagagtaccgatcccgaaagggaaaggtgatctcctaaacccgatcgaagttcggatcgaaggttgcaattcgccttcgtgaagttggaatcggtagtaatcgtgtctcaaaatgacacggtgaat

SEQ ID NO: 244 - DNA, 578 bp, Artificial Sequence; identical to SEQ ID NO: 243
  Synthetic 1629_R924T

SEQ ID NO: 245 - DNA, 578 bp, Artificial Sequence
  Synthetic 1629_R782T
ccagccgtaaacgatgccagctatgtgtcgggagatccacgtgttcttccggtgccgtagggaagccgtgaagctggccacctgggaagtacggccgcaaggctgaaacttaaaggaattggcgggggagtactacaaccggtggagcttgcggtttaattggatacaacgccggaaatctcaccgggggcgacagcagtatgaaggccaggctgaggaccttgctagattagctgagaggaggtgcatggccgtcgtcagttcgtaccgtgaggcatcctgttaagtcaggcaacgggcgagacccgcggtcttaattgccagcatacccttcggggtgattgggtacaataagacgactgccagcgctaagctggaggaagaagcgggctacggtaggtcagcatgccccaaatcccccgggctacacgcgtgctacaatggtcggaacaaagagtaccaatcccgaaagggaaaggtgatctcctaaacccgatcgaagttcggatcgaaggttgcaattcgccttcgtgaagttggaatcggtagtaatcgtgtctcaaaatgacacggtgaat

SEQ ID NO: 246 - DNA, 578 bp, Artificial Sequence; identical to SEQ ID NO: 243
  Synthetic 1639_V537T

SEQ ID NO: 247 - DNA, 578 bp, Artificial Sequence
  Synthetic 1629_R839T
ccagccgtaaacgatgccagctatgtgtcgggagatccacgtgttctttcggtgccgtagggaagccgtgaagctggccacctgggaagtacggccgcaaggctgaaacttaaaggaattggcgggggagtactacaaccggtggagcttgcggtttaattggatacaacgccggaaatctcaccgggggcgacagcagtatgaaggccaggctgaggaccttgctagattagctgagaggaggtgcatggccgtcgtcagttcgtaccgtgaggcatcctgttaagtcaggcaacgggcgagacccgcggtcttaattgccagcatacccttcggggtgattgggtacaataagacgactgccagcgctaagctggaggaagaagcgggctacggtaggtcagcatgccccgaatcccccgggctacacgcgtgctacaatggtcggaacaaagagtaccgatcccgaaagggaaaggtgatctcctaaacccgatcgaagttcggatcgaaggttgcaattcgccttcgtgaagttggaatcggtagtaatcgtgtctcaaaatgacacggtgaat

SEQ ID NO: 248 - DNA, 578 bp, Artificial Sequence
  Synthetic 1629_R573T
ccagccgtaaacgatgccagctatgtgtcgggagatccacgtgttcttccggtgccgtagggaagccgtgaagctggccacctgggaagtacggccgcaaggctgaaacttaaaggaattggcgggggagtactacaaccggtggagcttgcggtttaattggatacaacgccggaaatctcaccgggggcgacagcagtatgaaggccaggctgaggaccttgctagattagctgagaggaggtgcatggccgtcgtcagttcgtaccgtgaggcatcctgttaagtcaggcaacgggcgagacccgcggtcttaattgccagcatacccttcggggtgattgggtacaataagacgactgccagcgctaagctggaggaagaagcgggctacggtaggtcagcatgccccgaatcccccgggctacacgcgtgctacaatggtcggaacaaagagtaccgatcccgaaagggaaaggtgatctcctaaacccgatcgaagttcggatcgaaggttgcaattcgccttcgtgaagttggaatcggtagtaatcgtgtctcaaaatgacacggtgaat

SEQ ID NO: 249 - DNA, 578 bp, Artificial Sequence
  Synthetic 1629_R984T
ccagccgtaaacgatgccagctatgtgtcgggagatccacgtgttcttccggtgccgtagggaagccgtgaagctggccacctgggaagtacggccgcaaggctgaaacttaaaggaattggcgggggagtactacaaccggtggagcttgcggtttaattggatacaacgccggaaatctcaccgggggcgacagcagtatgaaggccaggctgaggaccttgctagattagctgagaggaggtgcatggccgtcgtcagttcgtaccgtgaggcatcctgttaagtcaggcaacgggcgagacccgcggtcttaattgccagcatacccttcggggtgattgggtacaataagacgactgccagcgctaagctggaggaagaagcgggctacggtaggtcagcatgccccaaatcccccgggctacacgcgtgctacaatggtcggaacaaagagtaccgatcccgaaagggaaaggtgatctcctaaacccgatcaaagttcggatcgaaggttgcaattcgccttcgtgaagttggaatcggtagtaatcgtgtctcaaaatgacacggtgaat

SEQ ID NO: 250 - DNA, 578 bp, Artificial Sequence
  Synthetic 1629_R261T
ccagccgtaaacgatgccagctatgtgtcgggagatccacgtgttcttccggtgccgtagggaagccgtgaagctggccacctgggaagtacggccgcaaggctgaaacttaaaggaattggcgggggagtactacaaccggtggagcttgcggtttaattggatacaacgccggaaatctcaccgggggcgacagcagtatgaaggccaggctgaggaccttgctagattagctgagaggaggtgcatggccgtcgtcagttcgtaccgtgaggcatcctgttaagtcaggcaacgggcgagacccgcggtcttaattgccagcatacccttcggggtgattgggtacaataggacgactgccagcgctaagctggaggaagaagcgggctacggtaggtcagcatgccccgaatcccccgggctacacgcgtgctacaatggtcggaacaaagagtaccgatcccgaaagggaaaggtgatctcctaaacccgatcgaagttcggatcgaaggttgcaattcgccttcgtgaagttggaatcggtagtaatcgtgtctcaaaatgacacggtgaat

SEQ ID NO: 251 - DNA, 578 bp, Artificial Sequence
  Synthetic 1629_R616T
ccagccgtaaacgatgccagctatgtgtcgggagatccacgtgttcttccggtgccgtagggaagccgtgaagctggccacctgggaagtacggccgcaaggctgaaacttaaaggaattggcgggggagtactacaaccggtggagcttgcggtttaattggatacaacgccggaaatctcaccgggggcgacagcagtatgaaggccaggctgaggaccttgctagattagctgagaggaggtgcatggccgtcgtcagttcgtaccgtgaggcatcctgttaagtcaggcaacgggcgagacccgcggtcttaattgccagcatacccttcggggtgattgggtacaataagacgactgccagcgctaagctggaggaagaagcgggctacggcaggtcagcatgccccgaatcccccgggctacacgcgtgctacaatggtcggaacaaagagtaccgatcccgaaagggaaaggtgatctcctaaacccgatcgaagttcggatcgaaggttgcaattcgccttcgtgaagttggaatcggtagtaatcgtgtctcaaaatgacacggtgaat

SEQ ID NO: 252 - DNA, 578 bp, Artificial Sequence; identical to SEQ ID NO: 248
  Synthetic 1629_R027T

SEQ ID NO: 253 - DNA, 578 bp, Artificial Sequence
  Synthetic 1653_Y011T
ccagccgtaaacgatgccagctatgtgtcgggagatccacgtgttcttccggtgccgtagaaaagccgtgaagctggccacctgggaagtacggccgcaaggctgaaacttaaaggaattggcgggggagtactacaaccggtggagcttgcggtttaattggatacaacgccggaaatctcaccgggggcgacagcagtatgaaggccaggctgaggaccttgctagattagctgagaggaggtgcatggccgtcgtcagttcgtaccgtgaggcatcctgttaagtcaggcaacgggcgagacccgcggtcttaattgccagcatacccttcggggtgattgggtacaataagacgaccgccagcgctaagctggaggaagaagcgggctacggtaggtcagcatgccccaaatcccccgggctacacgcgtgctacaatggtcggaacaaagagtaccgatcccgaaagggaaaggtgatctcctaaacccgatcgaagttcggatcgaaggttgcaattcgccttcgtgaagttggaatcggtagtaatcgtgtctcaaaatgacacggtgaat

SEQ ID NO: 254 - DNA, 578 bp, Artificial Sequence; identical to SEQ ID NO: 248
  Synthetic 1629_R495T

SEQ ID NO: 255 - DNA, 578 bp, Artificial Sequence
  Synthetic 1629_R713T
ccagccgtaaacgatgccagctatgtgtcgggagatccacgtgttcttccggtgccgtagggaagccgtgaatctggccacctgggaagtacggccgcaaggctgaaacttaaaggaattggcgggggagtactacaaccggtggagcttgcggtttaattggatacaacgccggaaatctcaccgggggcgacagcagtatgaaggccaggctgaggaccttgctagattagctgagaggaggtgcatggccgtcgtcagttcgtaccgtgaggcatcctgttaagtcaggcaacgggcgagacccgcggtcttaattgccagcatacccttcggggtgattgggtacaataagacgactgccagcgctaagctggaggaagaagcgggctacggtaggtcagcatgccccaaatcccccgggctacacgcgtgctacaatggtcggaacaaagagtaccgatcccgaaagggaaaggtgatctcctaaacccgatcaaagttcggatcgaaggttgcaattcgccttcgtgaagttggaatcggtagtaatcgtgtctcaaaatgacacggtgaat

SEQ ID NO: 256 - DNA, 578 bp, Artificial Sequence
  Synthetic 1637_T212T
ccagccgtaaacgatgccagctatgtgtcgggagatccacgtgttcttccggtgccgtagggaagccgtgaagctggccacctgggaagtacggccgcaaggctgaaacttaaaggaattggcgggggagtactacaaccggtggagcttgcggtttaattggatacaacgccggaaatctcaccgggggcgacagcagtatgaaggccaggctgaggaccttgctagattagctgagaggaggtgcatggccgtcgtcagttcgtaccgtgaggcatcctgttaagttaggcaacgggcgagacccgcggtcttaattgccagcatacccttcggggtgattgggtacaataagacgactgccagcgctaagctggaggaagaagcgggctacggtaggtcagcatgccccaaatcccccgggctacacgcgtgctacaatggtcggaacaaagagtaccgatcccgaaagggaaaggtgatctcctaaacccgatcgaagttcggatcgaaggttgcaattcgccttcgtgaagttggaatcggtagtaatcgtgtctcaaaatgacacggtgaat

SEQ ID NO: 257 - DNA, 459 bp, Artificial Sequence
  Synthetic CONSENS_16
cagggtgtaaacgctgctagcttggtgttggataacccacgtggttattcagtgccggagagaagttgttaagctagctacctgggaagtacggtcgcaaggctgaaacttaaaggaattggcgggggagcactgcaacgggtggagcgtacggtttaattggattcaacgccgaaaacctcaccggaggcgacagtggatgaaggccaggctaaagactttgctggactagctgagaggtggtgcatggccatcggcagttcgtactgtaagcgttctgttaagtcagataacgaacaagaccccatcttcagttgctattttcaagtccgcttgaaaagcactctggagatactgcccgcgctaagtgggaggaaggagrgggccacggtaggtccgtattccccgaatcctccgggctacacgcgcgctacaaaggatgggacaatgggctccgac

SEQ ID NO: 258 - DNA, 458 bp, Artificial Sequence
  Synthetic 1637_T704T
cagggtgtaaacgctgctagcttggtgttggataacccacgtggttattcagtgccggagagaagttgttaagctagctacctgggaagtacggtcgcaaggctgaaacttaaaggaattggcgggggagcactgcaacgggtggagcgtacggtttaattggattcaacgccgaaaacctcaccggaggcgacagtggatgaaggccaggctaaagactttgctggactagctgagaggtggtgcatggccatcggcagttcgtactgtaagcgttctgttaagtcagataacgaacaagaccccatcttcagttgctattttcaagtccgcttgaaagcactctggagatactgcccgcgctaagtgggaggaaggagggggccacggtaggtccgtattccccgaatcctccgggctacacgcgcgctacaaaggatgggacaatgggctccgac

SEQ ID NO: 259 - DNA, 458 bp, Artificial Sequence; identical to SEQ ID NO: 258
  Synthetic 1638_U627T

SEQ ID NO: 260 - DNA, 458 bp, Artificial Sequence; identical to SEQ ID NO: 258
  Synthetic 1638_U364T

SEQ ID NO: 261 - DNA, 458 bp, Artificial Sequence
  Synthetic 1637_T983T
cagggtgtaaacgctgctagcttggtgttggataacccacgtggttattcagtgccggagagaagttgttaagctagctacctgggaagtacggtcgcaaggctgaaacttaaaggaattggcgggggagcactgcaacgggtggagcgtacggtttaattggattcaacgccgaaaacctcaccggaggcgacagtggatgaaggccaggctaaagactttgctggactagctgagaggtggtgcatggccatcggcagttcgtactgtaagcgttctgttaagtcagataacgaacaagaccccatcttcagttgctattttcaagtccgcttgaaagcactctggagatactgcccgcgctaagtgggaggaaggagagggccacggtaggtccgtattccccgaatcctccgggctacacgcgcgctacaaaggatgggacaatgggctccgac

SEQ ID NO: 262 - DNA, 458 bp, Artificial Sequence; identical to SEQ ID NO: 261
  Synthetic 1637_T915T

SEQ ID NO: 263 - DNA, 458 bp, Artificial Sequence
  Synthetic 1637_T686T
cagggtgtaaacgctgctagcttggtgttggataacccacgtggttattcagtgccggagagaagttgttaggctagctacctgggaagtacggtcgcaaggctgaaacttaaaggaattggcgggggagcactgcaatgggtggagcgtacggtttaattggattcaacgccgaaaacctcaccggaggcgacagtggatgaaggccaggctaaagactttgctggactagctgagaggtggtgcatggccatcggcagttcgtactgtaagcgttctgttaagtcagataacgaacaagaccccatcttcagttgctattttcaagtccgcttgaaagcactctggagatactgcccgcgctaagtgggaggaaggagagggccacggtaggtccgtattccccgaatcctccgggctacacgcgcgctacaaaggatgggacaatgggctccgac

SEQ ID NO: 264 - DNA, 458 bp, Artificial Sequence
  Synthetic 1638_U702T
cagggtgtaaacgctgctagcttggtgttggataacccacgtggttattcagtgccggagagaagttgttaagctagctacctgggaagtacggtcacaaggctgaaacttaaaggaattggcgggggagcactgcgacgggtggagcgtacggtttaattggattcaacgccgaaaacctcaccggaggcgacagtggatgaaggccaggctaaagactttgctggactagctgagaggtggtgcatggccatcggcagttcgtactgtaagcgttctgttaagtcagataacgaacaagaccccatcttcagttgctattttcaagtccgcttgaaagcactctggagatactgcccgcgctaagtgggaggaaggagagggccacggtaggtccgtattccccgaatcctccgggctacacgcgcgctacaaaggatgggacaacgggctccgac

SEQ ID NO: 265 - DNA, 458 bp, Artificial Sequence; identical to SEQ ID NO: 258
  Synthetic 1638_U701T

SEQ ID NO: 266 - DNA, 462 bp, Artificial Sequence
  Synthetic 1638_U208T
cagggtgtaaacgctgctagcttggtgttggataacccacgtggttattcagtgccggagagaagttgttaagctagctacctgggaagtacggtcgcaaggctgaaacttaaaggaattggcgggggagcactgcaacgggtggagcgtacggtttaattggattcaacgccgaaaacctcaccggaggcgacagctggatgaaggccaggctaaagactttgctggactagctgagaggtggtgcatggccatcggcagttcgtactgtaaagcgttctgttaagtcagataacgaacaagacccccatcttcagttgctattttcaagtccgcttgaaaagcactctggagatactgcccgcgctaagtgggaggaaggagagggccacggtaggtccgtattccccgaatcctccgggctacacgcgcgctacaaaggatgggacaatgggctccgac

SEQ ID NO: 267 - DNA, 461 bp, Artificial Sequence
  Synthetic 1639_V673T
cagggtgtaaacgctgctagcttggtgttggataacccacgtggttattcagtgccggagagaagttgttaagctagctacctgggaagtacggtcgcaaggctgaaacttaaaggaattggcgggggagcactgcaacgggtggagcgtacggtttaattggattcaacgccgaaaacctcaccggaggcgacagctggatgaaggccaggctaaagactttgctggactagctgagaggtggtgcatggccatcggcagttcgtactgtaaagcgttctgttaagtcagataacgaacaagaccccatcttcagttgctattttcaagtccgcttgaaaagcactctggagatactgcccgcgctaagtgggaggaaggagagggccacggtaggtccgtattccccgaatcctccgggctacacgcgcgctacaaaggatgggacaatgggctccgac

SEQ ID NO: 268 - DNA, 461 bp, Artificial Sequence; identical to SEQ ID NO: 267
  Synthetic 1638_U641T

SEQ ID NO: 269 - DNA, 462 bp, Artificial Sequence
  Synthetic 1629_R562T
cagggtgtaaacgctgctagcttggtgttggataacccacgtggttattcagtgccggagagaagttgttaagctagctacctgggaagtacggtcgcaaggctgaaacttaaaggaattggcgggggagcactgcaacgggtggagcgtacggtttaattggattcaacgccgaaaacctcaccggaggcgacagctggatgaaggccaggctaaagactttgctggactagctgagaggtggtgcatggccatcggcagttcgtactgtaaagcgttctgttaagtcagataacgaacaagacccccatcttcagttgctattttcaagtccgcttgaaaagcactctggagatactgcccgcgctaagtgggaggaaggagggggccacggtaggtccgtattccccgaatcctccgggctacacgcgcgctacaaaggatgggacaatgggctccgac

SEQ ID NO: 270 - DNA, 461 bp, Artificial Sequence
  Synthetic 1637_T741T
cagggtgtaaacgctgctagcttggtgttggataacccacgtggttattcagtgccggagagaagttgttaagctagctacctgggaagtacggtcgcaaggctgaaacttaaaggaattggcgggggagcactgcaacgggtggagcgtacggtttaattggattcaacgccgaaaacctcaccggaggcgacagctggatgaaggccaggctaaagactttgctggactagctgagaggtggtgcatggccatcggcagttcgtactgtaaagcgttctgttaagtcagataacgaacaagaccccatcttcagttgctattttcaagtccgcttgaaaagcactctggagatactgcccgcgctaagtgggaggaaggagggggccacggtaggtccgtattccccgaatcctccgggctacacgcgcgctacaaaggatgggacaatgggctccgac

SEQ ID NO: 271 - DNA, 462 bp, Artificial Sequence; identical to SEQ ID NO: 269
  Synthetic 1637_T788T

SEQ ID NO: 272 - DNA, 461 bp, Artificial Sequence; identical to SEQ ID NO: 270
  Synthetic 1629_R480T

SEQ ID NO: 273 - DNA, 458 bp, Artificial Sequence
  Synthetic 1653_Y067T
cagggtgtaaacgctgctagcttggtgttggataacccacgtggttattcagtgccggagagaagttgttaagctagctacctggggagtacggtcgcaaggctgaaacttaaaggaattggcgggggagcactgcaacgggtggagcgtacgatttaattggattcaacgccgaaaacctcaccggaggcgacagtggatgaaggccaggctaaagaccttgctggactagctgagaggtggtgcatggccatcggcagttcgtactgtaagcgttctgttaagtcagataacgaacaagaccccatcttcagttgctattttcaagtccgcttgaaagcactctggagatactgcccgcgctaagtgggaggaaggagagggccacggtaggtccgtattccccgaatcctccgggctacacgcgcgctacaaaggatgggacaatgggctccgac

SEQ ID NO: 274 - DNA, 567 bp, Artificial Sequence
  Synthetic CONSENS_20
cacgccctaaacggtggatactrgatataggggrtatcracycctcygtgtcgaagctaacgctttaagtatcccgcctgggractacggccgcaaggctaaaactcaaaggaattgacgggggcccgcacaagcagcggagcgtgtggtttaattcgatgcaacacgaaaaccttacccaggcttgacatrctagtggtaggaacctgaaagggrgacgaccyggttttccarggagctagcacaggtgctgcatggctrtcgtcagctcgtgccgtgaggtgttgggttaagtcccacaacgagcgcaaccccyatcgycagttgaatttttctgrcgaaactgcccagaaatcctgggaggaaggaggggatgacgtyaagtcagcatggcccttatgyctggggcracacacacgctacaatgggtggtacaryrggtkgcyacggagcaatccggagctaatccycaaagcaycctcagtagggattgcaggctgaaacccgcctgcatgaacgcggagttgctagtaaccgcaggtcagaatactgcggtgaatrcgtctc

SEQ ID NO: 275 - DNA, 569 bp, Artificial Sequence
  Synthetic 1637_T019T
cacgccctaaacggtggatactagatataggggatatcaactcctccgtgtcgaagctaacgctttaagtatcccgcctgggaactacggccgcaaggctaaaactcaaaggaattgacgggggcccgcacaagcagcggagcgtgtggtttaattcgatgcaacacgaaaaaccttacccaggcttgacatgctagtggtaggaacctgaaagggagacgaccctggttttccagggagctagcacaggtgctgcatggctatcgtcagctcgtgccgtgaggtgttgggttaagtcccacaacgagcgcaacccctattgccagttgaatttttctggcgaaactgcccagaaatcctgggaggaaggaggggatgacgttaagtcagcatggcccttatgtctggggcaacacacacgctacaatgggtggtacagcgggtggccacggagcaatccggagctaatcctcaaagcatcctcagtagggattgcaggctgaaacccgcctgcatgaacgcggagttgctagtaaccgcaggtcagaatactgcggtgaatgcgtctc

SEQ ID NO: 276 - DNA, 569 bp, Artificial Sequence
  Synthetic 1637_T401T
cacgccctaaacggtggatactagatataggggatatcaactcctccgtgtcgaagctaacgctttaagtatcccgcctgggaactacggccgcaaggctaaaactcaaaggaattgacgggggcccgcacaagcagcggagcgtgtggtttaattcgatgcaacacgaagaaccttacccaggcttgacatgctagtggtaggaacctgaaagggagacgaccctggttttccggggagctagcacaggtgctgcatggctatcgtcagctcgtgccgtgaggtgttgggttaagtcccacaacgagcgcaacccctatcgccagttgaatttttctggcgaaactgcccagaaatcctgggaggaaggaggggatgacgttaagtcagcatggcccttatgtctggggcaacacacacgctacaatgggtggtacagcgggtggccacggagcaatccggagctaatcctcaaagcatcctcagtagggattgcaggctgaaacccgcctgcatgaacgcggagttgctagtaaccgcaggtcagaatactgcggtgaatgcgtctc

SEQ ID NO: 277 - DNA, 572 bp, Artificial Sequence
  Synthetic 1629_R722T
cacgccctaaacggtggatactagatataggggatatcaactcctccgtgtcgaagctaacgctttaagtatcccgcctgggaactacggccgcaaggctaaaactcaaaggaattgacgggggcccgcacaagcagcggagcgtgtggtttaattcgatgcaacacgaaaaaccttacccaggcttgacatgctagtggtaggaacctgaaagggagacgaccctggttttccagggagctagcacaggtgctgcatggctatcgtcagctcgtgccgtgaggtgttgggttaagtcccacaacgagcgcaacccctatcgccagttgaatttttctggcgaaactgcccagaaatcctgggaggaaggaggggatgacgttaagtcagcatggcccttatgtctggggcaacacacacgctacaatgggtggtacagcgggtggccacggagcaatccggagctaatcctcaaagccatcctcagtagggattgcaggctgaaacccgcctgcatgaacgcggagttgctagtaaccgcaggtcagtaatactgcggtgaatgcgttctc

SEQ ID NO: 278 - DNA, 569 bp, Artificial Sequence
  Synthetic 1629_R791T
cacgccctaaacggtggatactagatataggggatatcaactcctccgtgtcgaagctaacgctttaagtatcccgcctgggaactacggccgcaaggctaaaactcaaaggaattgacgggggcccgcacaagcagcggagcgtgtggtttaattcgatgcaacacgaaaaaccttacccaggcttgacatgctagtggtaggaacctgaaagggagacgaccctggttttccagggagctagcacaggtgctgcatggctatcgtcagctcgtgccgtgaggtgttgggttaagtcccacaacgagcgcaacccctatcgccagttgaatttttctggcgaaactgcccagaaatcctgggaggaaggaggggatgacgttaagtcagcatggcccttatgtctggggcaacacacacgctacaatgggtggtacagcgggtggccacggagcaatccggagctaatcctcaaagcatcctcagtagggattgcaggctgaaacccgcctgcatgaacgcggagttgctagtaaccgcaggtcagaatactgcggtgaatgcgtctc

SEQ ID NO: 279 - DNA, 569 bp, Artificial Sequence
  Synthetic 1639_V401T
cacgccctaaacggtggatactagatataggggatatcaactcctccgtgtcgaagctaacgctttaagtatcccgcctgggaactacggccgcaaggctaaaactcaaaggaattgacgggggcccgcacaagcagcggagcgtgtggtttaattcgatgcaacacgaaaaaccttacccaggcttgacatgctagtggtaggaacctgaaagggagacgatcctggttttccagggagctagcacaggtgctgcatggctatcgtcagctcgtgccgtgaggtgttgggttaagtcccacaacgagcgcaacccctattgccagttgaatttttctggcgaaactgcccagaaatcctgggaggaaggaggggatgacgttaagtcagcatggcccttatgtctggggcaacacacacgctacaatgggtggtacagcgggtggccacggagcaatccggagctaatcctcaaagcatcctcagtagggattgcaggctgaaacccgcctgcatgaacgcggagttgctagtaaccgcaggtcagaatactgcggtgaatgcgtctc

SEQ ID NO: 280 - DNA, 569 bp, Artificial Sequence
  Synthetic 1638_U062T
cacgccctaaatggtggatactagatataggggatatcaactcctccgtgtcgaagctaacgctttaagtatcccgcctgggaactacggccgcaaggctaaaactcaaaggaattgacgggggcccgcacaagcagcggagcgtgtggtttaattcgatgcaacacgaaaaaccttacccaggcttgacatgctagtggcaggaacctgaaagggagacgaccctggttttccagggagctagcacaggtgctgcatggctatcgtcagctcgtgccgtgaggtgttgggttaagtcccacaacgagcgcaacccctatcgccagttgaatttttctggcgaaactgcccagaaatcctgggaggaaggaggggatgacgttaagtcagcatggcccttatgtctggggcaacacacacgctacaatgggtggtacagcgggtggccacggagcaatccggagctaatcctcaaagcatcctcagtagggattgcaggctgaaacccgcctgcatgaacgcggagttgctagtaaccgcaggtcagaatactgcggtgaatgcgtctc

SEQ ID NO: 281 - DNA, 569 bp, Artificial Sequence
  Synthetic 1629_R186T
cacgccctaaacggtggatactagatataggggatatcaactcctccgtgtcgaagctaacgctttaagtatcccgcctgggaactacggccgcaaggctaaaactcaaaggaattgacgggggcccgcacaagcagcggagcgtgtggtttaattcgatgcaacacgaataaccttacccaggcttgacatgctagtggtaggaacctgaaagggagacgacccggttttccagggagctagcacaggtgctgcatggctgtcgtcagctcgtgccgtgaggtgttgggttaagtcccacaacgagcgcaacccccatcgtcagttgaatttctctggcgaaactgcccagaaatcctgggaggaaggaggggatgacgtcaagtcagcatggcccttatgcctggggcaacacacacgctacaatgggtggtacaacgggtagccacggagcaatccggagctaatcctcaaagccatcctcagtagggattgcaggctgaaactcgcctgcatgaacgcggagttgctagtaaccgcaggtcagaatactgcggtgaatacgtctc

SEQ ID NO: 282 - DNA, 569 bp, Artificial Sequence; identical to SEQ ID NO: 281
  Synthetic 1639_V458T

SEQ ID NO: 283 - DNA, 569 bp, Artificial Sequence
  Synthetic 1639_V781T
cacgccctaaacggtggatactagatataggggatatcaactcctccgtgtcgaagctaacgctttaagtatcccgcctgggaactacggccacaaggctaaaactcaaaggaattgacgggggcccgcacaagcagcggagcgtgtggtttaattcgatgcaacacgaataaccttacccaggcttgacatgctagtggtaggaacctgaaagggagacgacccggttttccagggagctagcacaggtgctgcatggctgtcgtcagctcgtgccgtgaggtgttgggttaagtcccacaacgagcgcaacccccatcgtcagttgaatttctctggcgaaactgcccagaaatcctgggaggaaggaggggatgacgtcaagtcagcatggcccttatgcctggggcaacacacacgctacaatgggtggtacaacgggtagccacggagcaatccggagctaatcctcaaagccatcctcagtagggattgcaggctgaaactcgcctgcatgaacgcggagttgctagtaaccgcaggtcagaatactgcggtgaatacgtctc

SEQ ID NO: 284 - DNA, 569 bp, Artificial Sequence
  Synthetic 1638_U272T
cacgccctaaacggtggatactagatataggggatatcaactcctccgtgtcgaagctaacgctttaagtatcccgcctgggaactacggccgcaaggctaaaactcaaaggaattgacgggggcccgcacaagcagcggagcgtgtggtttaattcgatgcaacacgaataaccttacccaggcttgacatgctagtggtaggaacctgaaagggagacgacccggttttccagggagctagcacaggtgctgcatggctgtcgtcagctcgtgccgtgaggtgttgggttaagtcccacaacgagcgcaacccccatcgtcagttgaatttctctggcgaaactgcccagaaatcctgggaggaaggaggggatgacgtcaagtcagcatggcccttatgcctgaggcaacacacacgctacaatgggtggtacaacgggtagccacggagcaatccggagctaatcctcaaagccatcctcagtagggattgcaggctgaaactcgcctgcatgaacgcggagttgctagtaaccgcaggtcagaatactgcggtgaatacgtctc

SEQ ID NO: 285 - DNA, 568 bp, Artificial Sequence
  Synthetic 1629_R746T
cacgccctaaacggtggatactggatatagggggtatcgacccctctgtgtcgaagctaacgccttaagtatcccgcctggggactacggccgcaaggctaaaactcaaaggaattgacgggggcccgcacaagcagcggagcgtgtggtttaattcgatgcaacacgaagaaccttacccaggcttgacatactagtggtaggaacctgaaagggggacgacctggttttccaaggagctagcacaggtgctgcatggctgtcgtcagctcgtgccgtgaggtgttgggttaagtcccacaacgagcgcaacccccatcgtcagttgaatttttctgacgaaactgcccagaaatcctgggaggaaggaggggatgacgtcaagtcagcatggcccttatgcctggggcgacacacacgctacaatgggtggtacaataggttgctacggagcaatccggagctaatccccaaagcaccctcagtagggattgcaggctgaaacccgcctgcatgaacgcggagttgctagtaaccgcaggtcagaatactgcggtgaatacgtctc

SEQ ID NO: 286 - DNA, 568 bp, Artificial Sequence
  Synthetic 1639_V420T
cacgccctaaacggtggatactggatatagggggtatcgacccctctgtgtcgaagctaacgccttaagtatcccgcctggggactacggccgcaaggctaaaactcaaaggaattgacgggggcccgcacaagcagcggagcgtgtggtttaattcgatgcaacacgaagaaccttacccaggcttgacatactagtggtaggaacctgaaagggggacgacctggttttccaaggagctagcacaggtgctgcatggctgtcgtcagctcgtgccgtgaggtgttgggttaagtcccacaacgagcgcaatccccatcgtcagttgaatttttctgacgaaactgcccagaaatcctgggaggaaggaggggatgacgtcaagccagcatggcccttatgcctggggcgacacacacgctacaatgggtggtacaataggttgctacggagcaatccggagctaatccccaaagcaccctcagtagggattgcaggctgaaacccgcctgcatgaacgcggagttgctagtaaccgcaggtcagaatactgcggtgaatacgtctc

SEQ ID NO: 287 - DNA, 568 bp, Artificial Sequence; identical to SEQ ID NO: 285
  Synthetic 1639_V415T

SEQ ID NO: 288 - DNA, 568 bp, Artificial Sequence
  Synthetic 1639_V265T
cacgccctaaacggtggatactggatatagggggtatcgacccctctgtgtcgaagctaacgccttaagtatcccgcctggggactacggccgcaaggctaaaactcaaagaaattgacgggggcccgcacaagcagcggagcgtgtggtttaattcgatgcaacacgaagaaccttacccaggcttgacatactagtggtaggaacctgaaagggggacgacctggttttccaaggagctagcacaggtgctgcatggctgtcgtcagctcgtgccgtgaggtgttgggttaagtcccacaacgagcgcaacccccatcgtcagttgaatttttctgacgaaactgcccagaaatcctgggaggaaggaggggatgacgtcaagtcagcatggcccttatgcctggggcgacacacacgctacaatgggtggtacaataggttgctacggagcaatccggagctaatccccaaagcaccctcagtagggattgcaggctgaaacccgcctgcatgaacgcggagttgctagtaaccgcaggtcagaatactgcggtgaatacgtctc

SEQ ID NO: 289 - DNA, 568 bp, Artificial Sequence
  Synthetic 1629_R130T
cacgccctaaacggtggatactggatatagggggtatcgaccctctgtgtcgaagctaacgctttaagtatcccgcctggggactacggccgcaaggctaaaactcaaaggaattgacgggggcccgcacaagcagcggagcgtgtggtttaattcgatgcaacacgaagacccttacccaggcttgacatactagtggtaggaacctgaaagggggacgaccttggttttccaaggagctagcacaggtgctgcatggctgtcgtcagctcgtgccgtgaggtgttgggttaagtcccacaacgagcgcaacccccatcgtcagttgaatttttctgacgaaactgcccagaaatcctgggaggaaggaggggatgacgtcaagtcagcatggcccttatgcctggggcgacacacacgctacaatgggtggtacaataggttgctacggagcaatccggagctaatccccaaagcaccctcagtagggattgcaggctgaaacccgcctgcatgaacgcggagttgctagtaaccgcaggtcagaatactgcggtgaatacgtctc

SEQ ID NO: 290 - DNA, 568 bp, Artificial Sequence
  Synthetic 1637_T261T
cacgccctaaacggtggatactggatatagggggtatcgacccctctgtgtcgaagctaacgctttaagtatcccgcctggggactacggccgcaaggctaaaactcaaaggaattgacgggggcccgcacaagcagcggagcgtgtggtttaattcgatgcaacacgaagaaccttacccaggcttgacatactagtggtaggaacctgaaagggggacgacctggttttccaaggagctagcacaggtgctgcatggctgtcgtcagctcgtgccgtgaggtgttgggttaagtcccacaacgagcgcaacccccatcgtcagttgaatttttctgacgaaactgcccagaaatcctgggaggaaggaggggatgacgtcaagtcagcatggcccttatgcctggggcgacacacacgctacaatgggtggtacaataggttgctacggagcaatccggagctaatccccaaagcaccctcagtagggattgcaggctgaaacccgcctgcatgaacgcggagttgctagtaaccgcaggtcagaatactgcggtgaatacgtctc

SEQ ID NO: 291 - DNA, 567 bp, Artificial Sequence
  Synthetic 1638_U215T
cacgccctaaacggtggatactggatatagggggtatcgaccctctgtgtcgaggctaacgctttaagtatcccgcctggggactacggccgcaaggctaaaactcaaaggaattgatgggggcccgcacaagcagcggagcgtgtggtttaattcgatgcaacacgaagaaccttacccaggcttgacatactagtggtaggaacctgaaagggggacgacctggttttccaaggagctagcacaggtgctgcatggctgtcgtcagctcgtgccgtgaggtgttgggttaagtcccacaacgagcgcaacccccatcgtcagttgaatttttctgacgaaactgcccagaaatcctgggaggaaggaggggatgacgtcaagtcagcatggcccttatgcctggggcgacacacacgctacaatgggtggtacaataggttgctacggagcaatccggaactaatccccaaagcaccctcagtagggattgcaggctgaaacccgcctgcatgaacgcggagttgctagtaaccgcaggtcagaatactgcggtgaatacgtctc

SEQ ID NO: 292 - DNA, 568 bp, Artificial Sequence; identical to SEQ ID NO: 290
  Synthetic 1629_R214T

SEQ ID NO: 293 - DNA, 568 bp, Artificial Sequence; identical to SEQ ID NO: 290
  Synthetic 1629_R523T

SEQ ID NO: 294 - DNA, 568 bp, Artificial Sequence; identical to SEQ ID NO: 290
  Synthetic 1639_V221T

SEQ ID NO: 295 - DNA, 587 bp, Artificial Sequence
  Synthetic 1653_Y162T
ctagctgtaaacgatggatactaggtgtgggaggtatcgaccccttctgtgccgcagctaacgcattaagtatcccgcctggggagtacggtcgcaaggctgaaactcaaaggaattgacgggggcccgcacaagcggtggagcatgtggtttaattcgacgcaacgcgaagaaccttaccgggacttgacattatcttgcccgtctaagaaattagatcttcttccttaccgaagacgagataacaggtggtgcatggttgtcgtcagctcgtgtcgtgagatgttgggttaagtcccacaacgagcgcaacccttatgcttagttgctaacttgttttacaagtgcactctaagcagactgtcgcagataatgcggaggaaggtggggatgacgtcaaatcatcatgccccttacgtcccgggctacacacgtgctacaatggtctgtacagagggtagcgaaagagcgatcttaagccaatcccaaaaagcaggccccagttcggattggaggctgcaactcgcctccatgaagtaggaatcgctagtaatcgcggatcagcatgccgcggtgaatacgtcccg

SEQ ID NO: 296 - DNA, 588 bp, Artificial Sequence
  Synthetic 1629_R376T
ctagctgtaaacgatggatactaggtgtgggaggtatcgaccccttctgtgccgcagctaacgcattaagtatcccgcctggggagtacggtcgcaaggctgaaactcaaaggaattgacgggggcccgcacaagcggtggagcatgtggtttaattcgacgcaacgcgaagaaccttaccgggacttgacattatcttgcccgtctaagaaattagatcttcttccttaccgaagacgagataacaggtggtgcatggttgtcgtcagctcgtgtcgtgagatgttgggttaagtcccacaacgagcgcaacccttatgcttagttgctaacttgttttacaagtgcactctaagcagactgtcgcagataatgcggaggaaggtggggatgacgtcaaatcatcatgccccttacgtcccgggctacacacgtgctacaatggtctgtacagagggtagcgaaagagcgatcttaagccaatcccaaaaagcaggccccagttcggattggaggctgcaactcgcctccatgaagtaggaatcgctagtaatcgcggatcagcatgccgcggtgaatacgtcccgg

SEQ ID NO: 297 - DNA, 587 bp, Artificial Sequence; identical to SEQ ID NO: 295
  Synthetic 1637_T173T

SEQ ID NO: 298 - DNA, 488 bp, Artificial Sequence
  Synthetic 1629_R657T
ctagctgtaaacgatggatactaggtgtgggaggtatcgaccccttctgtgccgcagctaacgcattaagtatcccgcctggggagtacggtcgcaaggccccgtctaagaaattagatcttcttccttaccgaagacgagataacaggtggtgcatggttgtcgtcagctcgtgtcgtgagatgttgggttaagtcccacaacgagcgcaacccttatgcttagttgctaacttgttttacaagtgcactctaagcagactgtcgcagataatgcggaggaaggtggggatgacgtcaaatcatcatgccccttacgtcccgggctacacacgtgctacaatggtctgtacagagggtagcgaaagagcgatcttaagccaatcccaaaaagcaggccccagttcggattggaggctgcaactcgcctccatgaagtaggaatcgctagtaatcgcggatcagcatgccgcggtgaatacgtcccgg

SEQ ID NO: 299 - DNA, 587 bp, Artificial Sequence; identical to SEQ ID NO: 295
  Synthetic 1637_T463T

SEQ ID NO: 300 - DNA, 587 bp, Artificial Sequence; identical to SEQ ID NO: 295
  Synthetic 1629_R388T

SEQ ID NO: 301 - DNA, 28 bp, Artificial Sequence
  Synthetic PTM11-Reverse
ctacaatggaaggtacagagagttgcaa

SEQ ID NO: 302 - DNA, 533 bp, Artificial Sequence
  Synthetic CONSENS_10
cacgccctaaacgatgttcacttggtgtcggtcgcacatacagatcggtgccggagctaacgcgttaagtgaaccgcctggggagtacggtcgcaaggctaaaactcaagagaattgacgggtccccgcacaagcggtggagcacgtggtttaattcgatgataagcgaagaacctcacctgggcttgacatgctagtggtaggaaccrgaaacggkgacgaccctgccttcgggtagggagctwgcacaggtgatgcatggctgtcgtcagctcgtgtcgtgggacgtagggttaagtcccgaaacgagcgcaacccctgtcgtcagttgccagcggataatgccggggactctgacgagactgctggtgaatagccggaggaaggaggggaygacgtcaagtcatcatgtcccttatgcccagggcgacacacatgctacaatggaaggtacagagagttgcaataccgtaaggtggagctaatcccaaaaagccttcccyagttcggattgaggtctgcaactcgacctc

SEQ ID NO: 303 - DNA, 533 bp, Artificial Sequence; identical to SEQ ID NO: 302
  Synthetic AT425_EubG

SEQ ID NO: 304 - DNA, 533 bp, Artificial Sequence
  Synthetic 1638_U231T
cacgccctaaacgatgttcacttggtgtcggtcgcacatacagatcggtgccggagctaacgcgttaagtgaaccgcctggggagtacggtcgcaaggctaaaactcaagagaattgacgggtccccgcacaagcggtggagcacgtggtttaattcgatgataagcgaagaacctcacctgggcttgacatgctagtggtaggaaccggaaacggggacgaccctgccttcgggtagggagcttgcacaggtgatgcatggctgtcgtcagctcgtgtcgtgggacgtagggttaagtcccgaaacgagcgcaacccctgtcgtcagttgccagcggataatgccggggactctgacgagactgctggtgaatagccggaggaaggaggggatgacgtcaagtcatcatgtcccttatgcccagggcgacacacatgctacaatggaaggtacagagagttgcaataccgtaaggtggagctaatcccaaaaagccttccccagttcggattgaggtctgcaactcgacctc

SEQ ID NO: 305 - DNA, 533 bp, Artificial Sequence; identical to SEQ ID NO: 304
  Synthetic 1638_U387T

SEQ ID NO: 306 - DNA, 533 bp, Artificial Sequence
  Synthetic 1644_W005T
cacgccctaaacgatgttcacttggtgtcggtcgcacatacagatcggtgccggagctaacgcgttaagtgaaccgcctggggagtacggtcgcaaggctaaaactcaagagaattgacgggtccccgcacaagcggtggagcacgtggtttaattcgatgataagcgaagaacctcacctgggcttgacatgctagtggtaggaaccggaaacggggacgaccctgccttcgggtagggagctagcacaggtgatgcatggctgtcgtcagctcgtgtcgtgggacgtagggttaagtcccgaaacgagcgcaacccctgtcgtcagttgccagcggataatgccggggactctgacgagactgctggtgaatagccggaggaaggaggggatgacgtcaagtcatcatgtcccttatacccagggcgacacacatgctacaatggaaggtacagagagttgcaataccgtaaggtggagctaatcccaaaaagccttccccagttcggattgaggtctgcaactcgacctc

SEQ ID NO: 307 - DNA, 533 bp, Artificial Sequence
  Synthetic 1637_T043T
cacgccctaaacgatgttcacttggtgtcggtcgcacatacagatcggtgccggagctaacgcgttaagtgaaccgcctggggagtacggtcgcaaggctaaaactcaagagaattgacgggtccccgcacaagcggtggagcacgtggtttaattcgatgataagcgaagaacctcacctgggcttgacatgctagtggtaggaaccggaaacggtgacgaccctgccttcgggtagggagctagcacaggtgatgcatggctgtcgtcagctcgtgtcgtgggacgtagggttaagtcccgaaacgagcgcaacccctgtcgtcagttgccagcggataatgccggggactctgacgagactgctggtgaatagccggaggaaggaggggatgacgtcaagtcatcatgtcccttatgcccagggcgacacacatgctacaatggaaggtacaaagagttgcaataccgtaaggtggagctaatcccaaaaagccttccccagttcggattgaggtctgcaactcgacctc

SEQ ID NO: 308 - DNA, 533 bp, Artificial Sequence
  Synthetic 1656_Z801T
cacgccctaaacgatgttcacttggtgtcggtcgcacatacagatcggtgccggagctaacgcgttaagtgaaccgcctggggagtacggtcgcaaggctaaaactcaagagaattgacgggtccccgcacaagcggtggagcacgtggtttaattcgatgataagcgaagaacctcacctgggcttgacatgctagtggtaggaaccggaaacggtgacgaccctgccttcgggtagggagcttgcacaggtgatgcatggctgtcgtcagctcgtgtcgtgggacgtagggttaagccccgaaacgagcgcaacccctgtcgtcagttgccagcggataatgccggggactctgacgagactgctggtgaatagccggaggaaggaggggatgacgtcaagtcatcatgtcccttatgcccagggcgacacacatgctacaatggaaggtacagagagttgcaataccgtaaggtggagctaatcccaaaaagccttccccagttcggattgaggtctgcaactcgacctc

SEQ ID NO: 309 - DNA, 533 bp, Artificial Sequence
  Synthetic 1638_U767T
cacgccctaaacgatgttcacttggtgtcggtcgcacatacagatcggtgccggagctaacgcgttaagtgaaccgcctggggagtacggtcgcaaggctaaaactcaagagaattgacgggtccccgcacaagcggtggagcacgtggtttaattcgatgataagcgaagaacctcacctgggcttgacatgctagtggtaggaaccggaaacggtgacgaccctgccttcgggtagggagcttgcacaggtgatgcatggctgtcgtcagctcgtgtcgtgggacgtagggttaagtcccgaaacgagcgcaacccctgtcgtcagttgccagcggataatgccggggactctgacgagactgctggtgaatagccggaggaaggaggggatgacgtcaagtcatcatgtcccttatgcccagggcgacacacatgctacaatggaaggtacagagagttgcaataccgtaaggtggagctaatcccaaaaagccttccccagttcggattgaggtctgcaactcgacctc

SEQ ID NO: 310 - DNA, 533 bp, Artificial Sequence
  Synthetic 1639_V275T
cacgccctaaacgatgttcacttggtgtcggtcgcacatacagatcggtgccggagctaacgcgttaagtgaaccgcctggggagtacggtcgcaaggctaaaactcaagagaattgacgggtccccgcacaagcggtggagcacgtggtttaattcgatgataagcgaagaacctcacctgggcttgacatgctagtggtaggaaccggaaacggtgacgaccctgccttcgggtagggagctagcacaggtgatgcatggctgtcgtcagctcgtgtcgtgggacgtagggttaagtcccgaaacgagcgcaacccctgtcgtcagttgccagcggataatgccggggactctgacgagactgctggtgaatagccggaggaaggaggggatgacgtcaagtcgtcatgtcccttatgcccagggcgacacacatgctacaatggaaggtacagagagttgcaataccgtaaggtggagctaatcccaaaaagccttccccagttcggattgaggtctgcaactcgacctc

SEQ ID NO: 311 - DNA, 533 bp, Artificial Sequence
  Synthetic 1639_V047T
cacgccctaaacgatgttcacttggtgtcggtcgcacatacagatcggtgccggagctaacgcgttaagtgaaccgcctggggagtacggtcgcaaggctaaaactcaagagaattgacgggtccccgcacaagcggtggagcacgtggtttaattcgatgataagcgaagaacctcacctgggcttgacatgctagtggtaggaaccagaaacggtgacgaccctgccttcgggtagggagctagcacaggtgatgcatggctgtcgtcagctcgtgtcgtgggacgtagggttaagtcccgaaacgagcgcaacccctgtcgtcagttgccagcggataatgccggggactctgacgagactgctggtgaatagccggaggaaggaggggacgacgtcaagtcatcatgtcccttatgcccagggcgacacacacgctacaatggaaggtacagagagttgcaataccgtaaggtggagctaatcccaaaaagccttccctagttcggattgaggtctgcaactcgacctc

SEQ ID NO: 312 - DNA, 533 bp, Artificial Sequence
  Synthetic 1629_R758T
cacgccctaaacgatgttcacttggtgtcggtcgcacatacagatcggtgccggagctaacgcgttaagtgaaccgcctggggagtacggtcgcaaggctaaaactcaagagaattgacgggtccccgcacaagcggtggagcacgtggtttaattcgatgataagcgaagaacctcacctgggcttgacatgctagtggtaggaaccagaaacggtgacgaccctgccttcgggtagggagctagcacaggtgatgcatggctgtcgtcagctcgtgtcgtgggacgtagggttaagtcccgaaacgagcgcaacccctgtcgtcagttgccagcggataatgccggggactctgacgagactgctggtgaatagccggaggaaggaggggacgacgtcaagtcatcatgtcccttatgcccagggcgacacacatgctacaatggaaggtacagagagttgcaataccgtaaggtggagctaatcccaaaaagccttccctagttcggattgaggtctgcaactcgacctc

SEQ ID NO: 313 - DNA, 533 bp, Artificial Sequence; identical to SEQ ID NO: 312
  Synthetic 1629_R488T

SEQ ID NO: 314 - DNA, 23 bp, Artificial Sequence
  Synthetic
gaccttgccagacaagctgagag

SEQ ID NO: 315 - DNA, 246 bp, Artificial Sequence
  Synthetic CONSENS_3
ccagccgtaaacgatgcacgctaggtgtgggtcggccacgagccgccccagtgccgcagggaagccrttaagcgtgccgcctggggagtacggccgcaaggctgaaacttaaaggaattggcgggggagcaccaccaggcgtgaagcctgcggtttaattggagtcaacgccgggaaccttaccgggagcgacagcagagtgaaggccaggctgaagaccttgccagacaagctgagaggaggtgc

SEQ ID NO: 316 - DNA, 246 bp, Artificial Sequence
  Synthetic TXv5v6-0593770
ccagccgtaaacgatgcacgctaggtgtgggtcggccacgagccgccccagtgccgcagggaagccgttaagcgtgccgcctggggagtacggccgcaaggctgaaacttaaaggaattggcgggggagcaccaccaggcgtgaagcctgcggtttaattggagtcaacgccgggaaccttaccgggagcgacagcagagtgaaggccaggctgaagaccttgccagacaagctgagaggaggtgc

SEQ ID NO: 317 - DNA, 246 bp, Artificial Sequence
  Synthetic TXv5v6-0219684
ccagccgtaaacgatgcacgctaggtgtgggtcggccacgagccgccccagtgccgcagggaagccattaagcgtgccgcctggggagtacggccgcaaggctgaaacttaaaggaattggcgggggagcaccaccaggcgtgaagcctgcggtttaattggagtcaacgccgggaaccttaccgggagcgacagcagagtgaaggccaggctgaagaccttgccagacaagctgagaggaggtgc

SEQ ID NO: 318 - DNA, 24 bp, Artificial Sequence
  Synthetic reverse complement of reverse primer
gactttgctggactagctgagagg

SEQ ID NO: 319 - DNA, 247 bp, Artificial Sequence
  Synthetic CONSENS_0208415
cagggtgtaaacgctgctagcttggtgttggataaccyacgtggttattcagtgccggagagaagttgttaagctagctacctgggaagtacggtcgcaaggctgaaacttaaaggaattggcgggggagcactgcaacgggtggagcgtacggtttaattggattcaacgccgaaaacctcaccggaggcgacagctgratgaaggccaggctaaagactttgctggactagctgagaggtggtgc

SEQ ID NO: 320 - DNA, 247 bp, Artificial Sequence
  Synthetic TXv5v6-0208415
cagggtgtaaacgctgctagcttggtgttggataacccacgtggttattcagtgccggagagaagttgttaagctagctacctgggaagtacggtcgcaaggctgaaacttaaaggaattggcgggggagcactgcaacgggtggagcgtacggtttaattggattcaacgccgaaaacctcaccggaggcgacagctgaatgaaggccaggctaaagactttgctggactagctgagaggtggtgc

SEQ ID NO: 321 - DNA, 247 bp, Artificial Sequence
  Synthetic TXv5v6-0208460
cagggtgtaaacgctgctagcttggtgttggataacctacgtggttattcagtgccggagagaagttgttaagctagctacctgggaagtacggtcgcaaggctgaaacttaaaggaattggcgggggagcactgcaacgggtggagcgtacggtttaattggattcaacgccgaaaacctcaccggaggcgacagctggatgaaggccaggctaaagactttgctggactagctgagaggtggtgc

SEQ ID NO: 322 - DNA, 28 bp, Artificial Sequence
  Synthetic reverse complement of reverse primer
ctgatgaccttgcctgattttccgagag

SEQ ID NO: 323 - DNA, 247 bp, Artificial Sequence
  Synthetic CONSENS_0208552
cagggtgtaaacgctgcttgcttgatgttagttgggctccgagcccaaytagtgtcggagagaagttgttaagcaagctgcctgggaagtacggtcgcaagrctgaaacttaaaggaattggcgggggagcacagcaacgggtggagcgtgcggtttaattggattcaacgccggaaaactcaccggaggcgacggttacatgaaggccaggctgatgaccttgcctgattttccgagaggtggtgc

SEQ ID NO: 324 - DNA, 247 bp, Artificial Sequence
  Synthetic TXv5v6-0208552
cagggtgtaaacgctgcttgcttgatgttagttgggctccgagcccaattagtgtcggagagaagttgttaagcaagctgcctgggaagtacggtcgcaagactgaaacttaaaggaattggcgggggagcacagcaacgggtggagcgtgcggtttaattggattcaacgccggaaaactcaccggaggcgacggttacatgaaggccaggctgatgaccttgcctgattttccgagaggtggtgc

SEQ ID NO: 325 - DNA, 247 bp, Artificial Sequence
  Synthetic TXv5v6-0208531
cagggtgtaaacgctgcttgcttgatgttagttgggctccgagcccaactagtgtcggagagaagttgttaagcaagctgcctgggaagtacggtcgcaaggctgaaacttaaaggaattggcgggggagcacagcaacgggtggagcgtgcggtttaattggattcaacgccggaaaactcaccggaggcgacggttacatgaaggccaggctgatgaccttgcctgattttccgagaggtggtgc

SEQ ID NO: 326 - DNA, 24 bp, Artificial Sequence
  Synthetic reverse complement of reverse primer
ggaccttgccagattagctgagag

SEQ ID NO: 327 - DNA, 245 bp, Artificial Sequence
  Synthetic CONSENS_0217476
ccagccgtaaacgatgccagctatgtgtcggaagatccagtgttcttccggtgtcgtagggaagccgtgaagctggccacctgggaagtacggccgcaaggctgaaacttaaaggaattggcgggggagtactacaaccggtggagcttgcggtttaattggatacaacgccggaaatctaccgggggcgacagcagtatgaaggccaggctgaggaccttgcyagaytagctgagaggaggtgc

SEQ ID NO: 328 - DNA, 245 bp, Artificial Sequence
  Synthetic TXv5v6-0217476
ccagccgtaaacaatgccagctatgtgtcggaagatccagtgttcttccggtgttgtagggaagccgtgaagctggccacctgggaagtacggccgcaaggctgaaacttaaaggaattggcgggggagtactacaaccggtggagcttgcggtttaattggatacaacgccggaaatctaccgggggcgacagcagtatgaaggccaggctgaggaccttgccagactagctgagaggaggtgc

SEQ ID NO: 329 - DNA, 245 bp, Artificial Sequence
  Synthetic TXv5v6-0219822
ccagccgtaaacgatgccagctatgtgtcggaagatccagcgttcttccggtgtcgtagggaagccgtgaagctggccacctgggaagtacggccgcaaggctgaaacttaaaggaattggcgggggagtactacaaccggtggagcttgcggtttaattggatacaacgccggaaatctaccgggggcgacagcagtatgaaggccaggctgaggaccttgccagattagctgagaggaggtgc

SEQ ID NO: 330 - DNA, 245 bp, Artificial Sequence
  Synthetic TXv5v6-0219861
ccagccgtaaacgatgccagctatgtgtcggaagatccagtgttcttccggtgtcgtagggaagccgtgaagctggccacctgggaagtacggccgcaaggctgaaacttaaaggaattggcgggggagtactacaaccggtggagcttgcggtttaattggatacaacgccggaaatctaccgggggcgacagcagtatgaaggccaggctgaggaccttgccagattagctgagaggaggtgc

SEQ ID NO: 331 - DNA, 245 bp, Artificial Sequence
  Synthetic TXv5v6-0219863
ccagccgtaaacgatgccagctatgtgtcggaagatccagtgttcttccggtgtcgtagggaagccgtgaagctggccacctgggaagtacggccgcaaggctgaaacttaaaggaattggcgggggagtactacaaccggtggagcttgcggtttaattggatacaacgccggaaatctaccgggggcgacagcagtatgaaggccaggctgaggaccttgctagattagctgagaggaggtgc

SEQ ID NO: 332 - DNA, 245 bp, Artificial Sequence
  Synthetic TXv5v6-0219845
ccagccgtaaacgatgccagctatgtgtcggaagatccagtgttcttccggtgccgtagggaagccgtgaagctggccacctgggaagtacggccgcaaggctgaaacttaaaggaattggcgggggagtactacaaccggtggagcttgcggtttaattggatacaacgccggaaatctaccgggggcgacagcagtatgaaggccaggctgaggaccttgctggactagctgagaggaggtgc

SEQ ID NO: 333 - DNA, 22 bp, Artificial Sequence
  Synthetic reverse complement of reverse primer
gtcttgctggacgagctgagag

SEQ ID NO: 334 - DNA, 245 bp, Artificial Sequence
  Synthetic CONSENS_0219799
ccagccgtaaacgatgcaggctaggtgtgggttggccacgtgccgctcagtgccacagggaagccattaagcctgccgcctggggagtacggycgcaaggctgaaacttaaaggaattggcgggggagcaccaccaggcgtgaagcctgcggtttaattggagtcaacgccgggaaccttaccgggagcgacagcagagtgaaggccaggttgaaggtcttgcyggacgagctgagaggaggtgc

SEQ ID NO: 335 - DNA, 246 bp, Artificial Sequence
  Synthetic TXv5v6-0219799
ccagccgtaaacgatgcaggctaggtgtgggttggccacgtgccgactcagtgccacagggaagccattaagcctgctgcctggggagtacggtcgcaaggctgaaacttaaaggaattggcgggagagcaccaccaggcgtgaagcctgcggtttaattggagtcaacgccgggaaccttaccgggagcgacagcagagtgaaagccaggttgaaggtcttgctggacgagctgagaggaggtgc

SEQ ID NO: 336 - DNA, 246 bp, Artificial Sequence
  Synthetic TXv5v6-0219794
ccagccgtaaacgatgcaggctaggtgtggggtggccacgtgccgcctcagtgccacagggaagccattaagcctgccgcctggggagtacggtcgcaaggctgaaacttaaaggaattggcgggggagcaccaccaggcgtgaagcctgcggtttaattggagtcaacgccgggaaccttaccgggagcgacagcagagtgaaggccaggttgaaggtcttgccggacgagctgagaggaggtgc

SEQ ID NO: 337 - DNA, 246 bp, Artificial Sequence
  Synthetic TXv5v6-0596935
ccagccgtaaacgatgcaggctaggtgtgggttggccacgtgccagctcagtgccacagggaagccattaagcctgccgcctggggagtacggccgcaaggctgaaacttaaaggaattggcgggggagcaccaccaggcgtgaagcctgcggtttaattggagtcaacgccgggaatcttaccgggagcgacagcagaatgaaggccaggttgaaggtcttgctggacgagctgagaggtggtgc

SEQ ID NO: 338 - DNA, 246 bp, Artificial Sequence
  Synthetic TXv5v6-0219795
ccagccgtaaacgatgcaggctaggtgtgggtcggccacgcgccgcctcagtgccacagggaagccattaagcctgccgcctggggagtacggccgcaaggctgaaacttaaaggaattggcgggggagcaccaccaggcgtgaagcctgcggtttaattggagtcaacgccgggaaccttaccgggagcgacagcagagtgaaggccaggctgaaggtcttgccagacgagctgagaggaggtgc

SEQ ID NO: 339 - DNA, 25 bp, Artificial Sequence
  Synthetic reverse complement of reverse primer
cttaccagacaagctgagaggaggt

SEQ ID NO: 340 - DNA, 245 bp, Artificial Sequence
  Synthetic CONSENS_0235530
ccagctgtaaacgatgcaggctaggtgtggcgcggctacgtgccgctcagtgccgcagggaagccgttaagcctgccgcctgggaagtacggccgcaaggctgaaacttaaaggaattggcgggggagcaccacaaggkgtgaagcttgcggtttaattggagtcaacgccggaaatctcaccgggggcgacagcagaatgaaggtcagattgaaggtcttaccagacaagctgagaggaggtgc

SEQ ID NO: 341 - DNA, 245 bp, Artificial Sequence
  Synthetic TXv5v6-0235530
ccagctgtaaacgatgcaggctaggtgtggcgcggctacgtgccgctcagtgccgcagggaagccgttaagcctgccgcctgggaagtacggccgcaaggctgaaacttaaaggaattggcgggggagcaccacaaggggtgaagcttgcggtttaattggagtcaacgccggaaatctcaccgggggcgacagcagaatgaaggtcagattgaaggtcttaccagacaagctgagaggaggtgc

SEQ ID NO: 342 - DNA, 245 bp, Artificial Sequence
  Synthetic TXv5v6-0235545
ccagctgtaaacgatgcaggctaggtgtggcgcggctacgtgccgctcagtgccgcagggaagccgttaagcctgccgcctgggaagtacggccgcaaggctgaaacttaaaggaattggcgggggagcaccacaaggtgtgaagcttgcggtttaattggagtcaacgccggaaatctcaccgggggcgacagcagaatgaaggtcagattgaaggtcttaccagacaagctgagaggaggtgc

SEQ ID NO: 343 - DNA, 24 bp, Artificial Sequence
  Synthetic reverse complement of reverse primer
cttacctgacacgctgagaggagt

SEQ ID NO: 344 - DNA, 247 bp, Artificial Sequence
  Synthetic CONSENS_0242586
ctagcagtaaacactgcacactaaacattagtacctcytcgagaggtattggtgctgwagggaagccgaagagtgtgctacctgggaagtatagycgcaaggccgaaacttaaaggaatwggcggggagacactacaacrggtgacgcgtgcggttcaattagattatacaccgtgaacctcaccaggagcgacagcagaatgaaggtcagtctgaagggcttacctgacacgctgagaggagttgc

SEQ ID NO: 345 - DNA, 247 bp, Artificial Sequence
  Synthetic TXv5v6-0242586
ctagcagtaaacactgcacactaaacattagtacctcctcgagaggtattggtgctgtagcgaaggcgaagagtgtgctacctgggaagtatagccgcaaggccgaaacttaaaggaataggcggggaggcactacaacgggtgacgcgtgcggttcaattagattatacaccgtgaacctcaccaggagcgatagcagaatgaaggtcagtctgaagggcttacctgacacgctaagaggagttgc

SEQ ID NO: 346 - DNA, 247 bp, Artificial Sequence
  Synthetic TXv5v6-0242630
ctagcagtaaacactgcacactaaacattagtacctcttcgagaggtattggtgctgtagcgaaggcgaagagtgtgctacctgggaagtatagccgcaaggccgaaacttaaaggaataggcggggaggcactacaacgggtgacgcgtgcggttcaattagattatacaccgtgaacctcaccaggagcgatagcagaatgaaggtcagtctgaagggcttacctgacacgctaagaggagttgc

SEQ ID NO: 347 - DNA, 247 bp, Artificial Sequence
  Synthetic TXv5v6-0647404
ctagcagtaaacactgcacactaaacattagtacctcttcgagaggtattggtgctgtagcgaaggcgaagagtgtgctacctgggaagtatagccgcaaggccgaaacttaaaggaataggcggggagacactacaacgggtgacgcgtgcggttcaattagattatacaccgtgaacctcaccaggagcgatagcagaatgaaggtcagtctgaagggcttacctgacacgctaagaggagttgc

SEQ ID NO: 348 - DNA, 247 bp, Artificial Sequence
  Synthetic TXv5v6-0242596
ctagcagtaaacactgcacactaaacattagtacctcctcgagaggtattggtgctgtagggaagccgaagagtgtgctacctgggaagtatagccgcaaggccgaaacttaaaggaataggcggggagacactacaacgggtgacgcgtgcggttcaattagattatacaccgtgaacctcaccaggagcgatagcagaatgaaggtcagtctgaagggcttacctgacacgctaagaggagttgc

SEQ ID NO: 349 - DNA, 247 bp, Artificial Sequence
  Synthetic TXv5v6-0242606
ctagcagtaaacactgcacactaaacattagtacctcctcgagaggtattggtgctgtagggaagccgaagagtgtgctacctgggaagtatagccgcaaggccgaaacttaaaggaataggcggggagacactacaacgggtgacgcgtgcggttcaattagattctacaccgtgaacctcaccaggagcgacagcaggatgaaggtcagtctgaagggcttacctgacacgctgagaggagttgc

SEQ ID NO: 350 - DNA, 247 bp, Artificial Sequence
  Synthetic TXv5v6-0642293
ctagcagtaaacactgcacactaaacatcagtacctcttcgagaggcattggtgctgcagggaagccgaagagtgtgctacctgggaagtatagccgcaaggccgaaacttaaaggaataggcggggaggcactacaacgggtgacgcgtgcggttcaattagattatacaccgtgaacctcaccaggagcgacagcagaatgaaggtcagtctgaagggcttacctgacacgctgagaggagttgc

SEQ ID NO: 351 - DNA, 247 bp, Artificial Sequence
  Synthetic TXv5v6-0651560
ctagcagtaaactctgcacactaaacattagtacctcttcgagaggtattagtgctgaagggaagccgaagagtgtgctacctgggaagtatagccgcaaggccgaaacttaaaggaattggcggggagacactacaacaggtgacgcgtgcggttcaattagattatacaccgtgaacctcaccaggagcgacagcagaatgaaggtcagtctaaagggcttacctgacacgctgagaggagttgc

SEQ ID NO: 352 - DNA, 247 bp, Artificial Sequence
  Synthetic TXv5v6-0644101
ctagcagtaaacactgcacactaaacattagtacctcctcgagaggtattggtgctgaagggaagccgaagagtgtgctacctgggaagtatagccgcaaggccgaaacttaaaggaattggcggggagacactacaacaggtgacgcgtgcggttcaattagattatacaccgtgaacctcaccaggagcgacagcaggatgaaggtcagtctgaagggcttacctgacacgctgagaggagttgc

SEQ ID NO: 353 - DNA, 247 bp, Artificial Sequence
  Synthetic TXv5v6-0242619
ctagcagtaaacactgcacactaaacattagtacctcctcgagaggtattggtgctgtagggaagccgaagagtgtgctacctgggaagtatagtcgcaaggccgaaacttaaaggaattggcggggagacactacaacgggtgacgcgtgcggttcaattagattatacaccgtgaacctcaccaggagcgacagcagaatgaaggtcagtctgaagggcttacctaacacgctgagaggagttgc

SEQ ID NO: 354 - DNA, 247 bp, Artificial Sequence
  Synthetic TXv5v6-0646437
ctagcagtaaacactgcacactaaacattagtacctcctcgagaggtattggtgctgtagggaagccgaagagtgtgctacctgggaagtatagtcgcaaggccgaaacttaaaggaattggcggggagacactacaacgggtgacgcgtgcggttcaattagattatacaccgtgaacctcaccaggggcgacagcagaatgaaggtcagtctgaagggcttacctgacacgctgagaggagttgc

SEQ ID NO: 355 - DNA, 247 bp, Artificial Sequence
  Synthetic TXv5v6-0641596
ctagcagtaaacactgcacactaaacatcagtacctcctcgagaggtattggtgctgaagggaagccgaagagtgtgctacctgggaagtatagtcgcaaggccgaaacttaaaggaattggcggggagacactacaacaggtgacgcgtgcggttcaattagattatacaccgtgaacctcaccaggagcgacagcagaatgaaggtcagtctgaagggcttacctgacacgctgagaggagttgc

SEQ ID NO: 356 - DNA, 247 bp, Artificial Sequence
  Synthetic TXv5v6-0644254
ctagcagtaaacactgcacactaaacattagtacctcctcgagaggtattggtgctgaagggaagccgaagagtgtgctacctgggaagtatagtcgcaaggccgaaacttaaaggaattggcggggagacactacaacaggtgacgcgtgcggttcaattagattatacaccgtgaaccttaccaggaccgacagcagaatgaaggtcagtctaaagggcttacctgacacgctgagaggagctgc

SEQ ID NO: 357 - DNA, 247 bp, Artificial Sequence
  Synthetic TXv5v6-0643665
ctagcagtaaacactgcacactaaacattagtacctcctcgagaggtattagtgctgaagggaagccgaagagtgtgctacctgggaagtatagtcgcaaggccgaaacttaaaggaattggcggggaggcactacaacgggtgacgcgtgcggttcaattagattatacaccgtgaacctcaccaggagcgacagcagaatgaaggtcagtctgaagggcttacctgacacgctgagaggagctgc

SEQ ID NO: 358 - DNA, 247 bp, Artificial Sequence
  Synthetic TXv5v6-0647677
ctagcagtaaacactgcacactaaacattagtacctcttcgggaggtattagtgctgaagggaagccaaagagtgtgctacctgggaagtatagtcgcaaggctgaaacttaaaggaattggcggggagacactacaacaggtgacgcgtgcggttcaattagattatacaccgtgaatctcaccaggaccgacagcagaatgaaggtcagtctgaagggcttacctgacacgctgagaggagttgc

SEQ ID NO: 359 - DNA, 26 bp, Artificial Sequence
  Synthetic reverse complement of reverse primer
gactttgctgaattagctgagaggtg

SEQ ID NO: 360 - DNA, 247 bp, Artificial Sequence
  Synthetic CONSENS_0242690
ctagcagtaaacgatgcgggcyaggtgttagtatcactgcgagtggtactagtgtcgaagggaagccgttaagcccgccatctgggaagtacggtcgcaaggctgaaacttaaaggaattggcgggggagcactacaacgggtggagcttgcggtttaattggattcaacgccgtgaatcttaccggggaagacagcaagatgaaagccaagctaaagactttgctgaattagctgagaggtggtgc

SEQ ID NO: 361 - DNA, 247 bp, Artificial Sequence
  Synthetic TXv5v6-0242690
ctagcagtaaacgatgcgggccaggtgttagtatcactgcgagtggtactagtgtcgaagggaagccgttaagcccgccatctgggaagtacggtcgcaaggctgaaacttaaaggaattggcgggggagcactacaacgggtggagcttgcggtttaattggattcaacgccgtgaatcttaccggggaagacagcaagatgaaagccaagctaaagactttgctgaattagctgagaggtggtgc

SEQ ID NO: 362 - DNA, 247 bp, Artificial Sequence
  Synthetic TXv5v6-0242726
ctagcagtaaacgatgcgggctaggtgttagtatcactgcgagtggtactagtgtcgaagggaagccgttaagcccgccatctgggaagtacggtcgcaaggctgaaacttaaaggaattggcgggggagcactacaacgggtggagcttgcggtttaattggattcaacgccgtgaatcttaccggggaagacagcaagatgaaagccaagctaaagactttgctgaattagctgagaggtggtgc

SEQ ID NO: 363 - DNA, 27 bp, Artificial Sequence
  Synthetic reverse complement of reverse primer
gaccttaccaaattcgctgagaggaag

SEQ ID NO: 364 - DNA, 246 bp, Artificial Sequence
  Synthetic CONSENS_0248376
ctagccgtaaacgatgctcgctaggtgttaaataccctgggagggtatttagtgtcgtaaggaagccgtgaagcgagccacctgggaagtacggtcgcaaggctgaaacttaaaggaattggcgggggagcacaacaacgggtggatgctgcggtttaattggattcaacgccggaaatcttaccggaggcgacagaatatgaaggtcaggttgaagaccttaccaaattcgctgagaggaagtgc

SEQ ID NO: 365 - DNA, 246 bp, Artificial Sequence; identical to SEQ ID NO: 364
  Synthetic TXv5v6-0248376

SEQ ID NO: 366 - DNA, 246 bp, Artificial Sequence; identical to SEQ ID NO: 364
  Synthetic TXv5v6-0671483

SEQ ID NO: 367 - DNA, 21 bp, Artificial Sequence
  Synthetic reverse complement of reverse primer
ccgacgagctgagaggaggtg

SEQ ID NO: 368 - DNA, 246 bp, Artificial Sequence
  Synthetic CONSENS_0266750
ctggccgtaaacgatgcatactaggtgatggtacggccatgagccgtatcagtgccgtagggaaaccgttaagtgtgccgcctgggaagtacggtcgcaaggctaaaacttaaaggaattggcggggagcaccacaaggggtgaagcctgcggttcaattggactcaacgccgggaaacttaccaggggagacagcagtatgamggtcaggytgacgaccttaccyracgagctgagaggaggtgc

SEQ ID NO: 369 - DNA, 247 bp, Artificial Sequence
  Synthetic TXv5v6-0266750
ctggccgtaaacgatgcatactaggtgatggtacggccatgagccgtatcagtgccgtagggaaaccgttaagtgtgccgcctgggaagtacggtcgcaaggctaaaacttaaaggaattggcgggggagcaccacaaggggtgaagcctgcggttcaattggactcaacgccgggaaacttaccaggggagacagcagtatgacggtcaggctgacgaccttacccaacgagctgagaggaggtgc

SEQ ID NO: 370 - DNA, 247 bp, Artificial Sequence
  Synthetic TXv5v6-0771140
ctggccgtaaacgatgcatactaggtgatggtacggccatgagccgtatcagtgccgtagggaaaccgttaagtgtgccgcctgggaagtacggtcgcaaggctaaaacttaaaggaattggcgggggagcaccacaaggggtgaagcctgcggttcaattggactcaacgccgggaaacttaccaggggagacagcagtatgaaggtcaggttgacgaccttacctgacgagctgagaggaggtgc

SEQ ID NO: 371 - DNA, 246 bp, Artificial Sequence
  Synthetic TXv5v6-0770570
ctggccgtaaacgatgcatactaggtgatggtacggccatgagccgtatcagtgccgtagggaaaccgttaagtgtgccgcctgggaagtacggtcgcaaggctaaaacttaaaggaattggcggggagcaccacaaggggtgaagcctgcggttcaattggactcaacgccgggaaacttaccaggggagacagcagtatgacggtcaggttgacgaccttacccgacgagctgagaggaggtgc

SEQ ID NO: 372 - DNA, 24 bp, Artificial Sequence
  Synthetic reverse complement of reverse primer
ttacccaacgagctgagaggaggt

SEQ ID NO: 373 - DNA, 247 bp, Artificial Sequence
  Synthetic CONSENS_0266796
ctggccgtaaacgatgcatactaggtgatggtacggctatgagccgtrtcagtgccgtagggaaaccgttaagtgtgccgcctgggaagtacggtcgcaaggctaaaacttaaaggaattggcgggggagcaccacaaggggtgaagcctgcggttcaattggactcaacgccgggaaacttaccaggggagacagcagwatgmcggtcaggttgacgaccttaccyracgagctgagaggaggtgc

SEQ ID NO: 374 - DNA, 247 bp, Artificial Sequence
  Synthetic TXv5v6-0266796
ctggccgtaaacgatgcatactaggtgatggtacggctatgagccgtgtcagtgccgtagggaaaccgttaagtgtgccgcctgggaagtacggtcgcaaggctaaaacttaaaggaattggcgggggagcaccacaaggggtgaagcctgcggttcaattggactcaacgccgggaaacttaccaggggagacagcagaatgccggtcaggttgacgaccttacctaacgagctgagaggaggtgc

SEQ ID NO: 375 - DNA, 247 bp, Artificial Sequence
  Synthetic TXv5v6-0772899
ctggccgtaaacgatgcatactaggtgatggtacggctatgagccgtatcagtgccgtagggaaaccgttaagtgtgccgcctgggaagtacggtcgcaaggctaaaacttaaaggaattggcgggggagcaccacaaggggtgaagcctgcggttcaattggactcaacgccgggaaacttaccaggggagacagcagtatgacggtcaggttgacgaccttacccgacgagctgagaggaggtgc

SEQ ID NO: 376 - DNA, 25 bp, Artificial Sequence
  Synthetic reverse complement of reverse primer
caggctgatgaccttaccagacaag

SEQ ID NO: 377 - DNA, 247 bp, Artificial Sequence
  Synthetic CONSENS_0283719
ctgggcgtaaatgatgtgggctaggtgcaaagctacctaagyggtagcttggtgccgatgggaagccgttaagcccaccgcctggggagtacggtcgcaaggctgaaacttaaaggaattggcgggkgagcaccacaaggggtggaggctgcggtttaattggattcaacgccgggaaactcaccgggggcgacagcagtatgaaggtcaggctgatgaccttaccagacaagctgagaggaggtgc

SEQ ID NO: 378 - DNA, 247 bp, Artificial Sequence
  Synthetic TXv5v6-0283719
ctgggcgtaaatgatgtgggctaggtgcaaagctacctaagtggtagcttggtgccgatgggaagccgttaagcccaccgcctggggagtacggtcgcaaggctgaaacttaaaggaattggcgggggagcaccacaaggggtggaggctgcggtttaattggattcaacgccgggaaactcaccgggggcgacagcagtatgaaggtcaggctgatgaccttaccagacaagctgagaggaggtgc

SEQ ID NO: 379 - DNA, 247 bp, Artificial Sequence
  Synthetic TXv5v6-0283712
ctgggcgtaaatgatgtgggctaggtgcaaagctacctaagcggtagcttggtgccgatgggaagccgttaagcccaccgcctggggagtacggtcgcaaggctgaaacttaaaggaattggcgggtgagcaccacaaggggtggaggctgcggtttaattggattcaacgccgggaaactcaccgggggcgacagcagtatgaaggtcaggctgatgaccttaccagacaagctgagaggaggtgc

SEQ ID NO: 380 - DNA, 247 bp, Artificial Sequence
  Synthetic TXv5v6-0788889
ctgggcgtaaatgatgtgggctaggtgcaaagctacctaagtggtagcttggtgccgatgggaagccgttaagcccaccgcctggggagtacggtcgcaaggctgaaacttaaaggaattggcgggtgagcaccacaaggggtggaggctgcggtttaattggattcaacgccgggaaactcaccgggggcgacagcagtatgaaggtcaggctgatgaccttaccagacaagctgagaggaggtgc

SEQ ID NO: 381 - DNA, 22 bp, Artificial Sequence
  Synthetic reverse complement of reverse primer
ccagattagctgagaggtggcg

SEQ ID NO: 382 - DNA, 247 bp, Artificial Sequence
  Synthetic CONSENS_0714814
ctagctgtaaacgatgcrggcyaggtgttggcattactgcgagtgatgccagtgccgaagggaagccgttaagccygccatctggggagtacggtcgcaaggctgaaacttaaaggaattggcgggggagcacyacaacgggtggagcytgcggttcaattggattcaacgccggaaamctcaccggrggmgacagcgakatgaaggtcaggctgaagaccttaccrrattagctgagaggtggcgc

SEQ ID NO: 383 - DNA, 247 bp, Artificial Sequence
  Synthetic TXv5v6-0714814
ctagctgtaaacgatgcgggccaggtgttggcattactgcgagtgatgccagtgccgaagggaagccgttaagcccgccatctggggagtacggtcgcaaggctgaaacttaaaggaattggcgggggagcaccacaacgggtggagcttgcggttcaattggattcaacgccggaaaactcaccgggggagacagcgagatgaaggtcaggctgaagaccttaccaaattagctgagaggtggcgc

SEQ ID NO: 384 - DNA, 247 bp, Artificial Sequence
  Synthetic TXv5v6-0257743
ctagctgtaaacgatgcaggctaggtgttggcattactgcgagtgatgccagtgccgaagggaagccgttaagcctgccatctggggagtacggtcgcaaggctgaaacttaaaggaattggcgggggagcactacaacgggtggagcctgcggttcaattggattcaacgccggaaacctcaccggaggcgacagcgatatgaaggtcaggctgaagaccttaccggattagctgagaggtggcgc

SEQ ID NO: 385 - DNA, 25 bp, Artificial Sequence
  Synthetic reverse complement of reverse primer
cgaataataggcagagaggtggtgc

SEQ ID NO: 386 - DNA, 248 bp, Artificial Sequence
  Synthetic CONSENS_1349302
cagggcgtaaacgatgtgggcttcgyattgaagaccgtatggttttcagtgctggaacgaaggcgttaagcccaccgcctgggaagtacggccgcaaggctgaaacttaaaggaattgacgggggagcacagcaacgggaggagcgtgcggttcaattggattcaacgccggaaaactcaccggaggagactgccagatgtgggccaagctgaagactttgctcgaataataggcagagaggtggtgc

SEQ ID NO: 387 - DNA, 248 bp, Artificial Sequence
  Synthetic TXv5v6-1349302
cagggcgtaaacgatgtgggcttcgtattgaagaccgtatggttttcagtgctggaacgaaggcgttaagcccaccgcctgggaagtacggccgcaaggctgaaacttaaaggaattgacgggggagcacagcaacgggaggagcgtgcggttcaattggattcaacgccggaaaactcaccggaggagactgccagatgtgggccaagctgaagactttgctcgaataataggcagagaggtggtgc

SEQ ID NO: 388 - DNA, 248 bp, Artificial Sequence
  Synthetic TXv5v6-1349224
cagggcgtaaacgatgtgggcttcgcattgaagaccgtatggttttcagtgctggaacgaaggcgttaagcccaccgcctgggaagtacggccgcaaggctgaaacttaaaggaattgacgggggagcacagcaacgggaggagcgtgcggttcaattggattcaacgccggaaaactcaccggaggagactgccagatgtgggccaagctgaagactttgctcgaataataggcagagaggtggtgc

SEQ ID NO: 389 - DNA, 22 bp, Artificial Sequence
  Synthetic reverse complement of reverse primer
ctggattagctgagaggtggcg

SEQ ID NO: 390 - DNA, 247 bp, Artificial Sequence
  Synthetic CONSENS_1689428
ctagctgtaaacgatgcgggccaggtgttggcattactgcgagtgatgtcagtgccgaagggaagccgttaagcccgccatctggggagtacggtcgcaaggctgaaacttaaaggaattggcgggggagcaccacaacgggtggagcctgcggttcaattggattcaacgccggraaactcaccggaggcgacagcaagatgaaggtcaggctgaagaccttacyggattagctgagaggtggcgc

SEQ ID NO: 391 - DNA, 248 bp, Artificial Sequence
  Synthetic TXv5v6-1689428
ctagctgtaaacgatgcgggccaggtgttggcattactgcgagtgatgtcagtgccgaagggaagcccgttaagcccgccatctggggagtacggtcgcaaggctgaaacttaaaggaattggcgggggagcaccacaacgggtggagcctgcggttcaattggattcaacgccgggaaactcaccggaggcgacagcaatatgaaggtcaggctgaagaccttaccagattagctgagaggtggcgc

SEQ ID NO: 392 - DNA, 248 bp, Artificial Sequence
  Synthetic TXv5v6-1425443
ctagctgtaaacgatgcgggccaggtgttggcattactgcgagtgatgtcagtgccgaagggaagcccgttaagcccgccatctggggagtacggtcgcaaggctgaaacttaaaggaattggcgggggagcaccacaacgggtggagcctgcggttcaattggattcaacgccgggaaactcaccggaggcgacagcaagatgaaggtcaggctgaagaccttactggattagctgagaggtggcgc

SEQ ID NO: 393 - DNA, 248 bp, Artificial Sequence
  Synthetic TXv5v6-1688200
ctagctgtaaacgatgcgggccaggtgttgacattactgcgagtgatgtcagtgccgaagggaagcccgttaagcccgccatctggggagtacggtcgcaaggctgaaacttaaaggaattggcgggggagcaccacaacgggtggagcctgcggttcaattggattcaacgccgggaaactcaccggaggcgacagcaagatgaaggtcaggctgaagaccttaccggattagctgagaggtggcgc

SEQ ID NO: 394 - DNA, 247 bp, Artificial Sequence
  Synthetic TXv5v6-0257863
ctagctgtaaacgatgcgggccaggtgttgacattactgcgagtgatgtcagtgccgaagggaagccgttaagcccgccatctggggagtacggtcgcaaggctgaaacttaaaggaattggcgggggagcaccacaacgggtggagcctgcggttcaattggattcaacgccgggaaactcaccggaggcgacagcaagatgaaggtcaggctgaagaccttaccggattagctgagaggtggcgc

SEQ ID NO: 395 - DNA, 247 bp, Artificial Sequence
  Synthetic TXv5v6-0716397
ctagctgtaaacgatgcgggccaggtgttggcattactgcgagtgatgtcagtgccgaagggaagccgttaagcccgccatctggggagtacggtcgcaaggctgaaacttaaaggaattggcgggggagcaccacaacgggtggagcctgcggttcaattggattcaacgccgggaaactcaccggaggcgacagcaagatgaaggtcaggctgaagaccttacccgattagctgagaggtggcgc

SEQ ID NO: 396 - DNA, 247 bp, Artificial Sequence
  Synthetic TXv5v6-0258422
ctagctgtaaacgatgcgggccaggtgttggcattactgcgagtgatgtcagtgccgaagggaagccgttaagcccgccatctggggagtacggtcgcaaggctgaaacttaaaggaattggcgggggagcaccacaacgggtggagcctgcggttcaattggattcaacgccgggaaactcaccggaggcgacagcaatatgaaggtcaggctgaagaccttaccagattagctgagaggtggcgc

SEQ ID NO: 397 - DNA, 247 bp, Artificial Sequence
  Synthetic TXv5v6-0258367
ctagctgtaaacgatgcgggccaggtgttggcattactgcgagtgatgtcagtgccgaagggaagccgttaagcccgccatctggggagtacggtcgcaaggctgaaacttaaaggaattggcgggggagcaccacaacgggtggagcctgcggttcaattggattcaacgccgggaaactcaccggaggcgacagcaagatgaaagtcaggctgaagaccttactggattagctgagaggtggcgc

SEQ ID NO: 398 - DNA, 247 bp, Artificial Sequence
  Synthetic TXv5v6-0258396
ctagctgtaaacgatgcgggccaggtgttggcattactgcgagtgatgtcagtgccgaagggaagccgttaagcccgccatctggggagtacggtcgcaaggctgaaacttaaaggaattggcgggggagcaccacaacgggtggagcctgcggttcaattggattcaacgccgggaaactcaccggaggcgacagcaagatgaaggtcaggctgaagaccttactggattagctgagaggtggcgc

SEQ ID NO: 399 - DNA, 248 bp, Artificial Sequence
  Synthetic TXv5v6-1689332
ctagctgtaaacgatgcgggccaggtgttggcattactgcgagtgatgtcagtgccgaagggaagcccgttaagcccgccatctggggagtacggtcgcaaggctgaaacttaaaggaattggcgggggagcaccacaacgggtggagcctgcggttcaattggattcaacgccgggaaactcaccggaggcgacagcaagatgaaggtcaggctgaagaccttaccggattagctgagaggtggcgc

SEQ ID NO: 400 - DNA, 248 bp, Artificial Sequence
  Synthetic TXv5v6-0715252
ctagctgtaaacgatgcgggccaggtgttggcattactgcgagtgatgtcagtgccgaagggaagcccgttaagcccgccatctggggagtacggtcgcaaggctgaaacttaaaggaattggcgggggagcaccacaacgggtggagcctgcggttcaattggattcaacgccgggaaactcaccggaggcgacagcaagatgaaggtcaggctgaagaccttaccagattagctgagaggtggcgc

SEQ ID NO: 401 - DNA, 247 bp, Artificial Sequence
  Synthetic TXv5v6-0258423
ctagctgtaaacgatgcgggccaggtgttggcattactgcgagtgatgtcagtgccgaagggaagccgttaagcccgccatctggggagtacggtcgcaaggctgaaacttaaaggaattggcgggggagcaccacaacgggtggagcctgcggttcaattggattcaacgccgggaaactcaccggaggcgacagcaatatgaaggtcaggctgaagaccttaccggattagctgagaggtggcgc

SEQ ID NO: 402 - DNA, 247 bp, Artificial Sequence
  Synthetic TXv5v6-0258384
ctagctgtaaacgatgcgggccaggtgttggcattactgcgagtgatgtcagtgccgaagggaagccgttaagcccgccatctggggagtacggtcgcaaggctgaaacttaaaggaattggcgggggagcaccacaacgggtggagcctgcggttcaattggattcaacgccgggaaactcaccggaggcgacagcaagatgaaggtcaggctgaagaccttaccggattagctgagaggtggcgc

SEQ ID NO: 403 - DNA, 247 bp, Artificial Sequence
  Synthetic TXv5v6-0258379
ctagctgtaaacgatgcgggccaggtgttggcattactgcgagtgatgtcagtgccgaagggaagccgttaagcccgccatctggggagtacggtcgcaaggctgaaacttaaaggaattggcgggggagcaccacaacgggtggagcctgcggttcaattggattcaacgccgggaaactcaccggaggcgacagcaagatgaaggtcaggctgaagaccttaccagattagctgagaggtggcgc

SEQ ID NO: 404 - DNA, 248 bp, Artificial Sequence
  Synthetic TXv5v6-1425442
ctagctgtaaacgatgcgggccaggtgttggcattactgcgagtgatgtcagtgccgaagggaagcccgttaagcccgccatctggggagtacggtcgcaaggctgaaacttaaaggaattggcgggggagcaccacaacgggtggagcctgcggttcaattggattcaacgccgggaaactcaccggaggcgacagcaagatgaaagtcaggctgaagaccttactggattagctgagaggtggcgc

SEQ ID NO: 405 - DNA, 247 bp, Artificial Sequence
  Synthetic TXv5v6-0258269
ctagctgtaaacgatgcgggccaggtgttggcattactgcgagtgatgtcagtgccgaagggaagccgttaagcccgccatctggggagtacggtcgcaaggctgaaacttaaaggaattggcgggggagcaccacaacgggtggagcctgcggttcaattggattcaacgccggaaaactcaccggaggcgacagcaagatgaaggtcaggctgaagaccttaccggattagctgagaggtggcgc

SEQ ID NO: 406 - DNA, 248 bp, Artificial Sequence
  Synthetic TXv5v6-1689136
ctagctgtaaacgatgcgggccaggtgttggcattactgcgagtgatgtcagtgccgaagggaagcccgttaagcccgccatctggggagtacggtcgcaaggctgaaacttaaaggaattggcgggggagcaccacaacgggtggagcctgcggttcaattggattcaacgccggaaaactcaccggaggcgacagcaagatgaaggtcaggctgaagaccttaccggattagctgagaggtggcgc

SEQ ID NO: 407 - DNA, 247 bp, Artificial Sequence
  Synthetic TXv5v6-0258307
ctagctgtaaacgatgcgggccaggtgttggcattactgcgagtgatgtcagtgccgaagggaagccgttaagcccgccatctggggagtacggtcgcaaggctgaaacttaaaggaattggcgggggagcaccacaacgggtggagcctgcggttcaattggattcaacgccggaaaactcaccggaggcgacagcaatatgaaggtcaggctgaagaccttaccggattagctgagaggtggcgc

SEQ ID NO: 408 - DNA, 248 bp, Artificial Sequence
  Synthetic TXv5v6-1689106
ctagctgtaaacgatgcgggccaggtgttggcattactgcgagtgatgtcagtgccgaagggaagcccgttaagcccgccatctggggagtacggtcgcaaggctgaaacttaaaggaattggcgggggagcaccacaacgggtggagcctgcggttcaattggattcaacgccggaaaactcaccggaggcgacagcaagatgaaagtcaggctgaagaccttactggattagctgagaggtggcgc

SEQ ID NO: 409 - DNA, 247 bp, Artificial Sequence
  Synthetic TXv5v6-0258247
ctagctgtaaacgatgcgggccaggtgttggcattactgcgagtgatgtcagtgccgaagggaagccgttaagcccgccatctggggagtacggtcgcaaggctgaaacttaaaggaattggcgggggagcaccacaacgggtggagcctgcggttcaattggattcaacgccggaaaactcaccggaggcgacagcaagatgaaagtcaggctgaagaccttactggattagctgagaggtggcgc

SEQ ID NO: 410 - DNA, 247 bp, Artificial Sequence
  Synthetic TXv5v6-0258276
ctagctgtaaacgatgcgggccaggtgttggcattactgcgagtgatgtcagtgccgaagggaagccgttaagcccgccatctggggagtacggtcgcaaggctgaaacttaaaggaattggcgggggagcaccacaacgggtggagcctgcggttcaattggattcaacgccggaaaactcaccggaggcgacagcaagatgaaggtcaggctgaagaccttactggattagctgagaggtggcgc

SEQ ID NO: 411 - DNA, 247 bp, Artificial Sequence
  Synthetic TXv5v6-0258315
ctagctgtaaacgatgcgggccaggtgttggcattactgcgagtgatgtcagtgccgaagggaagccgttaagcccgccatctggggagtacggtcgcaaggctgaaacttaaaggaattggcgggggagcaccacaacgggtggagcctgcggttcaattggattcaacgccggaaaactcaccggaggcgacagcgagatgaaggtcaggctgaagaccttaccggattagctgagaggtggcgc

SEQ ID NO: 412 - DNA, 21 bp, Artificial Sequence
  Synthetic reverse complement of reverse primer
cggattagctgagaggtggcg

SEQ ID NO: 413 - DNA, 247 bp, Artificial Sequence
  Synthetic CONSENS_1671056
ccagctgtaaacgatgcgggccaggtgttggcattactgcgagtgatgtcagtgccaaagggaagccgttaagcccgccatctggggagtacggtcgcaaggctgaaacttaaagaaattggcgggggagcaccacaacgggtggagcctgcggttcaatyggattcaacgccggaaaactcaccggaggcgacagcgagatgaaggtcaggctgaagaccttaccggattagctgagaggtggcgc

SEQ ID NO: 414 - DNA, 248 bp, Artificial Sequence
  Synthetic TXv5v6-1671056
ccagctgtaaacgatgcgggccaggtgttggcattactgcgagtgatgtcagtgccaaagggaagcccgttaagcccgccatctggggagtacggtcgcaaggctgaaacttaaagaaattggcgggggagcaccacaacgggtggagcctgcggttcaattggattcaacgccggaaaactcaccggaggcgacagcgagatgaaggtcaggctgaagaccttaccagattagctgagaggtggcgc

SEQ ID NO: 415 - DNA, 247 bp, Artificial Sequence
  Synthetic TXv5v6-0237067
ccagctgtaaacgatgcgggccaggtgttggcattactgcgagtgatgtcagtgccaaagggaagccgttaagcccgccatctggggagtacggtcgcaaggctgaaacttaaagaaattggcgggggagcaccacaacgggtggagcctgcggttcaattggattcaacgccggaaaactcaccggaggcgacagcgagatgaaggtcaggctgaagaccttaccagattagctgagaggtggcgc

SEQ ID NO: 416 - DNA, 247 bp, Artificial Sequence
  Synthetic TXv5v6-1672136
ccagctgtaaacgatgcgggccaggtgttggcattactgcgagtgatgtcagtgccaaagggaagccgttaagcccgccatctggggagtacggtcgcaaggctgaaacttaaagaaattggcgggggagcaccacaacgggtggagcctgcggttcaattggattcaacgccggaaaactcaccggaggcgacagcgagatgaaggtcaggctgaagaccttaccggattagctgagaggtggcgc

SEQ ID NO: 417 - DNA, 247 bp, Artificial Sequence
  Synthetic TXv5v6-0237299
ccagctgtaaacgatgcgggccaggtgttggcattactgcgagtgatgtcagtgccaaagggaagccgttaagcccgccatctggggagtacggtcgcaaggctgaaacttaaaggaattggcgggggagcaccacaacgggtggagcctgcggttcaattggattcaacgccggaaaactcaccggaggcgacagcgagatgaaggtcaggctgaagaccttaccggattagctgagaggtggcgc

SEQ ID NO: 418 - DNA, 247 bp, Artificial Sequence
  Synthetic TXv5v6-0237037
ccagctgtaaacgatgcgggccaggtgttggcattactgcgagtgatgtcagtgccaaagggaagccgttaagcccgccatctggggagtacggtcgcaaggctgaaacttaaagaaattggcgggggagcaccacaacgggtggagcctgcggttcaattggattcaacgccggaaaactcaccggaggcaacagcgagatgaaggtcaggctgaagaccttaccggattagctgagaggtggcgc

SEQ ID NO: 419 - DNA, 248 bp, Artificial Sequence
  Synthetic TXv5v6-1376733
ccagctgtaaacgatgcgggccaggtgttggcattactgcgagtgatgtcagtgccaaagggaagcccgttaagcccgccatctggggagtacggtcgcaaggctgaaacttaaagaaattggcgggggagcaccacaacgggtggagcctgcggttcaattggattcaacgccggaaaactcaccggaggcgacagcgagatgaaggtcaggctgaagaccttaccggattagctgagaggtggcgc

SEQ ID NO: 420 - DNA, 247 bp, Artificial Sequence
  Synthetic TXv5v6-0237185
ccagctgtaaacgatgcgggccaggtgttggcattactgcgagtgatgtcagtgccaaagggaagccgttaagcccgccatctggggagtacggtcgcaaggctgaaacttaaagaaattggcgggggagcaccacaacgggtggagcttgcggttcaattggattcaacgccggaaaactcaccggaggcgacagcgagatgaaggtcaggctgaagaccttaccggattagctgagaggtggcgc

SEQ ID NO: 421 - DNA, 247 bp, Artificial Sequence; identical to SEQ ID NO: 416
  Synthetic TXv5v6-0237083

SEQ ID NO: 422 - DNA, 246 bp, Artificial Sequence
  Synthetic TXv5v6-1377062
ccagctgtaaacgatgcgggccaggtgttggcattactgcgagtgatgtcagtgccaaagggaagccgttaagcccgccatctgggagtacggtcgcaaggctgaaacttaaagaaattggcgggggagcaccacaacgggtggagcttgcggttcaattggattcaacgccggaaaactcaccggaggcgacagcgagatgaaggtcaggctgaagaccttaccggattagctgagaggtggcgc

SEQ ID NO: 423 - DNA, 246 bp, Artificial Sequence
  Synthetic TXv5v6-0236558
ccagctgtaaacgatgcgggccaggtgttggcattactgcgagtgatgtcagtgccaaagggaagccgttaagcccgccatctgggagtacggtcgcaaggctgaaacttaaagaaattggcgggggagcaccacaacgggtggagcctgcggttcaattggattcaacgccggaaaactcaccggaggcgacagcgagatgaaggtcaggctgaagaccttaccggattagctgagaggtggcgc

SEQ ID NO: 424 - DNA, 247 bp, Artificial Sequence
  Synthetic TXv5v6-0237291
ccagctgtaaacgatgcgggccaggtgttggcattactgcgagtgatgtcagtgccaaagggaagccgttaagcccgccatctggggagtacggtcgcaaggctgaaacttaaaggaattggcgggggagcaccacaacgggtggagcctgcggttcaatcggattcaacgccggaaaactcaccggaggcgacagcaagatgaaggtcaggctgaagaccttaccagattagctgagaggtggcgc

SEQ ID NO: 425 - DNA, 247 bp, Artificial Sequence
  Synthetic TXv5v6-0236906
ccagctgtaaacgatgcgggccaggtgttggcattactgcgagtgatgtcagtgccaaagggaagccgttaagcccgccatctggggagtacggtcgcaaggctgaaacttaaagaaattggcgggggagcaccacaacgggtggagcctgcggttcaatcggattcaacgccggaaaactcaccggaggcgacagcgagatgaaggtcaggctgaagaccttaccggattagctgagaggtggcgc

SEQ ID NO: 426 - DNA, 247 bp, Artificial Sequence
  Synthetic TXv5v6-0236917
ccagctgtaaacgatgcgggccaggtgttggcattactgcgagtgatgtcagtgccaaagggaagccgttaagcccgccatctggggagtacggtcgcaaggctgaaacttaaagaaattggcgggggagcaccacaacgggtggagcctgcggttcaatcggattcaacgccggaaaactcaccggaggcgacagcgagatgaaggtcaggctgaagaccttactggattagctgagaggtggcgc

SEQ ID NO: 427 - DNA, 247 bp, Artificial Sequence
  Synthetic TXv5v6-0624771
ccagctgtaaacgatgcgggccaggtgttggtattactgcgagtgatgtcagtgccaaagggaagccgttaagcccgccatctggggagtacggtcgcaaggctgaaacttaaagaaattggcgggggagcaccacaacgggtggagcctgcggttcaatcggattcaacgccggaaaactcaccggaggcgacagcgagatgaaggtcaggctgaagaccttaccggattagctgagaggtggcgc

SEQ ID NO: 428 - DNA, 247 bp, Artificial Sequence
  Synthetic TXv5v6-0236386
ccagctgtaaacgatgcgggccaggtgttgacattactgcgagtgatgtcagtgccaaagggaagccgttaagcccgccatctggggagtacggtcgcaaggctgaaacttaaagaaattggcgggggagcaccacaacgggtggagcctgcggttcaatcggattcaacgccggaaaactcaccggaggcgacagcgagatgaaggtcaggctgaagaccttaccggattagctgagaggtggcgc

SEQ ID NO: 429 - DNA, 247 bp, Artificial Sequence
  Synthetic TXv5v6-0236838
ccagctgtaaacgatgcgggccaggtgttggcattactgcgagtgatgtcagtgccaaagggaagccgttaagcccgccatctggggagtacggtcgcaaggctgaaacttaaagaaattggcgggggagcaccacaacgggtggagcctgcggttcaatcggattcaacgccggaaaactcaccggaggcgacagcaagatgaaggtcaggctgaagaccttaccggattagctgagaggtggcgc

SEQ ID NO: 430 - DNA, 247 bp, Artificial Sequence
  Synthetic TXv5v6-0236818
ccagctgtaaacgatgcgggccaggtgttggcattactgcgagtgatgtcagtgccaaagggaagccgttaagcccgccatctggggagtacggtcgcaaggctgaaacttaaagaaattggcgggggagcaccacaacgggtggagcctgcggttcaatcggattcaacgccggaaaactcaccggaggcgacagcaagatgaaggtcaggctgaagaccttaccagattagctgagaggtggcgc

SEQ ID NO: 431 - DNA, 247 bp, Artificial Sequence
  Synthetic TXv5v6-0236985
ccagctgtaaacgatgcgggccaggtgttggcattactgcgagtgatgtcagtgccaaagggaagccgttaagcccgccatctggggagtacggtcgcaaggctgaaacttaaagaaattggcgggggagcaccacaacgggtggagcctgcggttcaatcggattcaacgccggaaatctcaccggaggcgacagcgagatgaaggtcaggctgaagaccttaccggattagctgagaggtggcgc

SEQ ID NO: 432 - DNA, 246 bp, Artificial Sequence
  Synthetic TXv5v6-0621787
ccagctgtaaacgatgcgggccaggtgttggcattactgcgagtgatgtcagtgccaaagggaagccgttaagcccgccatctgggagtacggtcgcaaggctgaaacttaaagaaattggcgggggagcaccacaacgggtggagcctgcggttcaatcggattcaacgccggaaaactcaccggaggcgacagcgagatgaaggtcaggctgaagaccttaccggattagctgagaggtggcgc

SEQ ID NO: 433 - DNA, 23 bp, Artificial Sequence
  Synthetic reverse complement of reverse primer
tcgggacaaagtgacaggtgctg

SEQ ID NO: 434 - DNA, 254 bp, Artificial Sequence
  Synthetic CONSENS_0545759
cacgctgtaaacgatgggaactaggtgtagcgggtattgatccctgctgtgccgaagctaacgcattaagttccccgcctggggagtacggtcgcaaggctaaaactcaaaggaattgacgggggcccgcacaagcggtggagcatgtggttcaattcgatgcaacgcgaaaaaccttacctgggtttgacatcctttgacagtcyctgaaaggggatctttccgatttatcgggacaaagtgacaggtgctgc

SEQ ID NO: 435 - DNA, 254 bp, Artificial Sequence
  Synthetic TXv5v6-0545759
cacgctgtaaacgatgggaactaggtgtagcgggtattgatccctgctgtgccgaagctaacgcattaagttccccgcctggggagtacggtcgcaaggctaaaactcaaaggaattgacgggggcccgcacaagcggtggagcatgtggttcaattcgatgcaacgcgaaaaaccttacctgggtttgacatcctttgacagtctctgaaaggggatctttccgatttatcgggacaaagtgacaggtgctgc

SEQ ID NO: 436 - DNA, 254 bp, Artificial Sequence
  Synthetic TXv5v6-0194637
cacgctgtaaacgatgggaactaggtgtagcgggtattgatccctgctgtgccgaagctaacgcattaagttccccgcctggggagtacggtcgcaaggctaaaactcaaaggaattgacgggggcccgcacaagcggtggagcatgtggttcaattcgatgcaacgcgaaaaaccttacctgggtttgacatcctttgacagtccctgaaaggggatctttccgatttatcgggacaaagtgacaggtgctgc

SEQ ID NO: 437 - DNA, 23 bp, Artificial Sequence
  Synthetic reverse complement of reverse primer
cccttcgggggaacctggtgaca

SEQ ID NO: 438 - DNA, 254 bp, Artificial Sequence
  Synthetic CONSENS_0045163
cacgccctaaacgatgggcactaggtgcagggggtgttgacccctcctgtgccgcagctaacgcattaagtgccccgcctggggagtacggccgcaaggttgaaactcaaaggaattgacgggggcccgcacaagcggtggagcatgtggttyaattcgacgcaacgcgaagaaccttacctgggcttgacatcccgggaactctgtggaaacacggaggtgccccttcgggggaacctggtgacaggtgctgc

SEQ ID NO: 439 - DNA, 254 bp, Artificial Sequence
  Synthetic TXv5v6-0045163
cacgccctaaacgatgggcactaggtgcagggggtgttgacccctcctgtgccgcagctaacgcattaagtgccccgcctggggagtacggccgcaaggttgaaactcaaaggaattgacgggggcccgcacaagcggtggagcatgtggttcaattcgacgcaacgcgaagaaccttacctgggcttgacatcccgggaactctgtggaaacacggaggtgccccttcgggggaacctggtgacaggtgctgc

SEQ ID NO: 440 - DNA, 254 bp, Artificial Sequence
  Synthetic TXv5v6-0045206
cacgccctaaacgatgggcactaggtgcagggggtgttgacccctcctgtgccgcagctaacgcattaagtgccccgcctggggagtacggccgcaaggttgaaactcaaaggaattgacgggggcccgcacaagcggtggagcatgtggtttaattcgacgcaacgcgaagaaccttacctgggcttgacatcccgggaactctgtggaaacacggaggtgccccttcgggggaacctggtgacaggtgctgc

SEQ ID NO: 441 - DNA, 24 bp, Artificial Sequence
  Synthetic reverse complement of reverse primer
agtcaggaactgtcacaggtgctg

SEQ ID NO: 442 - DNA, 257 bp, Artificial Sequence
  Synthetic CONSENS_0063016
cacgccstaaacagtggacactagatatggggagtatcgacccttctcgtgtcgaagctaacgccttaagtgtcccacctggggactacgatcgcaaggttaaaactcaaaggaattgacgggggcccgcacaagcagcggagcgtgtggtttaattcgatgctacgcgaagaaccttaccagggcttgacatgtcagtagtaggaatccgaaaggaggacgacctgtatccagtcaggaactgtcacaggtgctgc

SEQ ID NO: 443 - DNA, 257 bp, Artificial Sequence
  Synthetic TXv5v6-0063016
cacgccgtaaacagtggacactagatatggggagtatcgacccttctcgtgtcgaagctaacgccttaagtgtcccacctggggactacgatcgcaaggttaaaactcaaaggaattgacgggggcccgcacaagcagcggagcgtgtggtttaattcgatgctacgcgaagaaccttaccagggcttgacatgtcagtagtaggaatccgaaaggaggacgacctgtatccagtcaggaactgtcacaggtgctgc

SEQ ID NO: 444 - DNA, 257 bp, Artificial Sequence
  Synthetic TXv5v6-1284822
cacgccctaaacagtggacactagatatggggagtatcgacccttctcgtgtcgaagctaacgccttaagtgtcccacctggggactacgatcgcaaggttaaaactcaaaggaattgacgggggcccgcacaagcagcggagcgtgtggtttaattcgatgctacgcgaagaaccttaccagggcttgacatgtcagtagtaggaatccgaaaggaggacgacctgtatccagtcaggaactgtcacaggtgctgc

SEQ ID NO: 445 - DNA, 25 bp, Artificial Sequence
  Synthetic reverse complement of reverse primer
ggctgaagaccttaccggattagct

SEQ ID NO: 446 - DNA, 247 bp, Artificial Sequence
  Synthetic CONSENS_0258790
ctagctgtaaacgatgcgggctaggtgttggcattactgcgagtgatgccagtgccgaagggaagccgttaagcccgccatctggggagtacggtcgcaaggctgaaacttaaaggaattggcgggggagcactacaacgggtggagcctgcggttcaattggattcaacgccggaaaactcaccggaggcgacagcgagatgaaggtcaggctgaagaccttaccggattagctgagaggtggcgc

SEQ ID NO: 447 - DNA, 247 bp, Artificial Sequence
  Synthetic TXv5v6-0258790
ctagctgtaaacgatgcgggctaggtgttggcattactgcgagtgatgccagtgccgaagggaagccgttaagcccgccatctggggagtacggtcgcaaggctgaaacttaaaggaattggcgggggagcactacaacgggtggagcctgcggttcaattggattcaacgccgggaaactcaccggaggcgacagcgagatgaaggtcaggctgaagaccttaccggattagctgagaggtggcgc

SEQ ID NO: 448 - DNA, 246 bp, Artificial Sequence
  Synthetic TXv5v6-0717922
ctagctgtaaacgatgcgggctaggtgttggcattactgcgagtgatgccagtgccgaagggaagccgttaagcccgccatctgggagtacggtcgcaaggctgaaacttaaaggaattggcgggggagcactacaacgggtggagcctgcggttcaattggattcaacgccgggaaactcaccggaggcgacagcgagatgaaggtcaggctgaagaccttaccggattagctgagaggtggcgc

SEQ ID NO: 449 - DNA, 247 bp, Artificial Sequence
  Synthetic TXv5v6-0258776
ctagctgtaaacgatgcgggctaggtgttggcattactgcgagtgatgccagtgccgaagggaagccgttaagcccgccatctggggagtacggtcgcaaggctgaaacttaaaggaattggcgggggagcactacaacgggtggagcctgcggttcaattggattcaacgccggaaaactcaccggaggcgacagcgatatgaaggtcaggctgaagaccttaccggattagctgagaggtggcgc

SEQ ID NO: 450 - DNA, 247 bp, Artificial Sequence; identical to SEQ ID NO: 446
  Synthetic TXv5v6-0258773

SEQ ID NO: 451 - DNA, 247 bp, Artificial Sequence; identical to SEQ ID NO: 446
  Synthetic TXv5v6-1691264

SEQ ID NO: 452 - DNA, 247 bp, Artificial Sequence; identical to SEQ ID NO: 446
  Synthetic TXv5v6-0718915

SEQ ID NO: 453 - DNA, 247 bp, Artificial Sequence
  Synthetic TXv5v6-0258774
ctagctgtaaacgatgcgggctaggtgttggcattactgcgagtgatgccagtgccgaagggaagccgttaagcccgccatctggggagtacggtcgcaaggctgaaacttaaaggaattggcgggggagcactacaacgggtggagcctgcggttcaattggattcaacgccggaaaactcaccggaggcgacagcgagatgaaggtcaggttgaagaccttactggattagctgagaggtggcgc

SEQ ID NO: 454 - DNA, 20 bp, Artificial Sequence
  Synthetic reverse complement of reverse primer
gccaggaagccagcagaggt

SEQ ID NO: 455 - DNA, 254 bp, Artificial Sequence
  Synthetic CONSENS_0252248
ctagccgtaaacgatgggcactagatgtttccgcttttagcggrggtgtcgaagctaacgcattaagtgccccgcctggggagtacggtcgcaaggctgaaactcaaaggaattgacgggggcccgcacaagcggtggagcatgtggttcaattcgacgcaacgcgaagaaccttacctgggtttgaactgctggtggtaaracctcgaaagrggaatgatcctggcttgccaggaagccagcagaggtgctgc

SEQ ID NO: 456 - DNA, 254 bp, Artificial Sequence
  Synthetic TXv5v6-0252248
ctagccgtaaacgatgggcactagatgtttccgcttttagcgggggtgtcgaagctaacgcattaagtgccccgcctggggagtacggtcgcaaggctgaaactcaaaggaattgacgggggcccgcacaagcggtggagcatgtggttcaattcgacgcaacgcgaagaaccttacctgggtttgaactgctggtggtaagacctcgaaaggggaatgatcctggcttgccaggaagccagcagaggtgctgc

SEQ ID NO: 457 - DNA, 254 bp, Artificial Sequence
  Synthetic TXv5v6-0689158
ctagccgtaaacgatgggcactagatgtttccgcttttagcggaggtgtcgaagctaacgcattaagtgccccgcctggggagtacggtcgcaaggctgaaactcaaaggaattgacgggggcccgcacaagcggtggagcatgtggttcaattcgacgcaacgcgaagaaccttacctgggtttgaactgctggtggtaaaacctcgaaagaggaatgatcctggcttgccaggaagccagcagaggtgctgc

SEQ ID NO: 458 - DNA, 254 bp, Artificial Sequence
  Synthetic TXv5v6-0252247
ctagccgtaaacgatgggcactagatgtttccgcttttagcgggggtgtcgaagctaacgcattaagtgccccgcctggggagtacggtcgcaaggctgaaactcaaaggaattgacgggggcccgcacaagcggtggagcatgtggttcaattcgacgcaacgcgaagaaccttacctgggtttgaactgctggtggtaaaacctcgaaaggggaatgatcctggcttgccaggaagccagcagaggtgctgc

SEQ ID NO: 459 - DNA, 23 bp, Artificial Sequence
  Synthetic reverse complement of reverse primer
ccaatccggagcctgtacaggtg

SEQ ID NO: 460 - DNA, 252 bp, Artificial Sequence
  Synthetic CONSENS_0254691
ctagccgtaaacgatgggcacttgacgtaggcgataatagtctgcgtcgtagctaacgtgttaagtgccccgcctggggagtacgttcgcaaggatgaaactcaaaggaattgacggggacccgcacaagcggtggaggatgtggtttaattcgaggcaacgcgaagaaccttacctgggcttgacatacaggaagtaggamcccgaaagggtaacgaccggtaaccaatccggagcctgtacaggtgttgc

SEQ ID NO: 461 - DNA, 252 bp, Artificial Sequence
  Synthetic TXv5v6-0254691
ctagccgtaaacgatgggcacttgacgtaggcgataatagtctgcgtcgtagctaacgtgttaagtgccccgcctggggagtacgttcgcaaggatgaaactcaaaggaattgacggggacccgcacaagcggtggaggatgtggtttaattcgaggcaacgcgaagaaccttacctgggcttgacatacaggaagtaggaccccgaaagggtaacgaccggtaaccaatccggagcctgtacaggtgttgc

SEQ ID NO: 462 - DNA, 252 bp, Artificial Sequence
  Synthetic TXv5v6-0254679
ctagccgtaaacgatgggcacttgacgtaggcgataatagtctgcgtcgtagctaacgtgttaagtgccccgcctggggagtacgttcgcaaggatgaaactcaaaggaattgacggggacccgcacaagcggtggaggatgtggtttaattcgaggcaacgcgaagaaccttacctgggcttgacatacaggaagtaggaacccgaaagggtaacgaccggtaaccaatccggagcctgtacaggtgttgc

SEQ ID NO: 463 - DNA, 20 bp, Artificial Sequence
  Synthetic reverse complement of reverse primer
ggagcctggacaggtgctgc

SEQ ID NO: 464 - DNA, 251 bp, Artificial Sequence
  Synthetic CONSENS_0262828
ctagctgtaaacgatgtggacttggcgttggtggggtcaaatccatcagtgccgkagctaacgcgataagtccaccgcctggggactacgaccgcaaggttaaaactcaaaggaattggcgggggcccgcacaagcagcggagcgtgtggtttaattcgatgctacacgaagaaccttacccgggtttgacatccaggtggtagggaaccgaaaggcgaccgacccttcggggagcctggacaggtgctgc

SEQ ID NO: 465 - DNA, 251 bp, Artificial Sequence
  Synthetic TXv5v6-0262828
ctagctgtaaacgatgtggacttggcgttggtggggtcaaatccatcagtgccggagctaacgcgataagtccaccgcctggggactacgaccgcaaggttaaaactcaaaggaattggcgggggcccgcacaagcagcggagcgtgtggtttaattcgatgctacacgaagaaccttacccgggtttgacatccaggtggtagggaaccgaaaggcgaccgacccttcggggagcctggacaggtgctgc

SEQ ID NO: 466 - DNA, 251 bp, Artificial Sequence
  Synthetic TXv5v6-0262852
ctagctgtaaacgatgtggacttggcgttggtggggtcaaatccatcagtgccgtagctaacgcgataagtccaccgcctggggactacgaccgcaaggttaaaactcaaaggaattggcgggggcccgcacaagcagcggagcgtgtggtttaattcgatgctacacgaagaaccttacccgggtttgacatccaggtggtagggaaccgaaaggcgaccgacccttcggggagcctggacaggtgctgc

SEQ ID NO: 467 - DNA, 26 bp, Artificial Sequence
  Synthetic reverse complement of reverse primer
cctgaaagggttggttatcctcttcg

SEQ ID NO: 468 - DNA, 249 bp, Artificial Sequence
  Synthetic CONSENS_1434138
ctagctgtaaacgatggatactagattttgcaagttattgcwagatcgaagctaacgcattaagtatcccgcctggggagtacggycgcaaggctaaaactcaaaggaattgacggggacccgcacaagcagtggagcatgtggtttaattcgatgcaacgcgaagaaccttacctgggcttgaactgtaggcattagccgcctgaaagggttggttatcctcttcggaggaacctatagaggtgctgc

SEQ ID NO: 469 - DNA, 249 bp, Artificial Sequence
  Synthetic TXv5v6-1434138
ctagctgtaaacgatggatactagattttgcaagttattgcaagatcgaagctaacgcattaagtatcccgcctggggagtacggccgcaaggctaaaactcaaaggaattgacggggacccgcacaagcagtggagcatgtggtttaattcgatgcaacgcgaagaaccttacctgggcttgaactgtaggcattagccgcctgaaagggttggttatcctcttcggaggaacctatagaggtgctgc

SEQ ID NO: 470 - DNA, 249 bp, Artificial Sequence
  Synthetic TXv5v6-0259077
ctagctgtaaacgatggatactagattttgcaagttattgctagatcgaagctaacgcattaagtatcccgcctggggagtacggtcgcaaggctaaaactcaaaggaattgacggggacccgcacaagcagtggagcatgtggtttaattcgatgcaacgcgaagaaccttacctgggcttgaactgtaggcattagccgcctgaaagggttggttatcctcttcggaggaacctatagaggtgctgc

SEQ ID NO: 471 - DNA, 249 bp, Artificial Sequence
  Synthetic TXv5v6-0722828
ctagctgtaaacgatggatactagattttgcaagttattgcaagatcgaagctaacgcattaagtatcccgcctggggagtacggtcgcaaggctaaaactcaaaggaattgacggggacccgcacaagcagtggagcatgtggtttaattcgatgcaacgcgaagaaccttacctgggcttgaactgtaggcattagccgcctgaaagggttggttatcctcttcggaggaacctatagaggtgctgc

SEQ ID NO: 472 - DNA, 28 bp, Artificial Sequence
  Synthetic reverse complement of reverse primer
ttagggaagacgarataacaggtggtgc

SEQ ID NO: 473 - DNA, 258 bp, Artificial Sequence
  Synthetic CONSENS_1437489
ctagctgtaaacgatggatactaggtgtgggaggtatcgaccccttctgtgccgcagctaacgcattaagtatcccgcctggggagtacggtcgcaaggctgaaactcaaaggaattgacgggggcccgcacaagcggtggagcatgtggtttaattcgacgcaacgcgaagaaccttaccgggacttgacattatyttgcccgtctaagaaattagatcttctttccttttagggaagacgarataacaggtggtgc

SEQ ID NO: 474 - DNA, 258 bp, Artificial Sequence
  Synthetic TXv5v6-1437489
ctagctgtaaacgatggatactaggtgtgggaggtatcgaccccttctgtgccgcagctaacgcattaagtatcccgcctggggagtacggtcgcaaggctgaaactcaaaggaattgacgggggcccgcacaagcggtggagcatgtggtttaattcgacgcaacgcgaagaaccttaccgggacttgacattattttgcccgtctaagaaattagatcttctttccttttagggaagacgaaataacaggtggtgc

SEQ ID NO: 475 - DNA, 258 bp, Artificial Sequence
  Synthetic TXv5v6-0726865
ctagctgtaaacgatggatactaggtgtgggaggtatcgaccccttctgtgccgcagctaacgcattaagtatcccgcctggggagtacggtcgcaaggctgaaactcaaaggaattgacgggggcccgcacaagcggtggagcatgtggtttaattcgacgcaacgcgaagaaccttaccgggacttgacattatcttgcccgtctaagaaattagatcttctttccttttagggaagacgagataacaggtggtgc

SEQ ID NO: 476 - DNA, 21 bp, Artificial Sequence
  Synthetic reverse complement of reverse primer
gtcaggagctgccacaggtgc

SEQ ID NO: 477 - DNA, 257 bp, Artificial Sequence
  Synthetic CONSENS_0489473
cacgccstaaacggtggacactagatataggargtatcgacccyttctgtgtcgaagctaacgccttaagtgtcccgcctgggkagtacggccgcaaggctgaaactcaaaggaattgacgggggcccgcacaagcagcggagcgtgtggtttaattcgatgctacrcgaagaaccttaccagggcttgacatgrcagaagtaggaatccgaaaggacgacgacctgtatccagtcaggagctgycacaggtgctgc

SEQ ID NO: 478 - DNA, 257 bp, Artificial Sequence
  Synthetic TXv5v6-0489473
cacgccgtaaacggtggacactagatataggaggtatcgaccccttctgtgtcgaagctaacgccttaagtgtcccgcctgggtagtacggccgcaaggctgaaactcaaaggaattgacgggggcccgcacaagcagcggagcgtgtggtttaattcgatgctacacgaagaaccttaccagggcttgacatggcagaagtaggaatccgaaaggacgacgacctgtatccagtcaggagctgtcacaggtgctgc

SEQ ID NO: 479 - DNA, 257 bp, Artificial Sequence
  Synthetic TXv5v6-0059568
cacgccctaaacggtggacactagatataggaagtatcgaccctttctgtgtcgaagctaacgccttaagtgtcccgcctggggagtacggccgcaaggctgaaactcaaaggaattgacgggggcccgcacaagcagcggagcgtgtggtttaattcgatgctacgcgaagaaccttaccagggcttgacatgacagaagtaggaatccgaaaggacgacgacctgtatccagtcaggagctgccacaggtgctgc

SEQ ID NO: 480 - DNA, 25 bp, Artificial Sequence
  Synthetic reverse complement of reverse primer
gcccgaaagggtgacaaccggtaac

SEQ ID NO: 481 - DNA, 252 bp, Artificial Sequence
  Synthetic CONSENS_0678112
ctagccgtaaacgatggacacttgacgtgggcgattttagtctgcgtcggagctaacgtattaagtgtcccgcctggggagtacgttcgcaaggatgaaactcaaaggaattgacggggacccgcacaagcggtggaggatgtggtttaattcgaggcaacgcgaagaaccttacctgggtttgacatgcagaaagtaggagcccgaaagggtracaaccggtaaccartccggaatctgcacaggtgctgc

SEQ ID NO: 482 - DNA, 252 bp, Artificial Sequence
  Synthetic TXv5v6-0678112
ctagccgtaaacgatggacacttgacgtgggcgattttagtctgcgtcggagctaacgtattaagtgtcccgcctggggagtacgttcgcaaggatgaaactcaaaggaattgacggggacccgcacaagcggtggaggatgtggtttaattcgaggcaacgcgaagaaccttacctgggtttgacatgcagaaagtaggagcccgaaagggtaacaaccggtaaccaatccggaatctgcacaggtgctgc

SEQ ID NO: 483 - DNA, 252 bp, Artificial Sequence
  Synthetic TXv5v6-0249051
ctagccgtaaacgatggacacttgacgtgggcgattttagtctgcgtcggagctaacgtattaagtgtcccgcctggggagtacgttcgcaaggatgaaactcaaaggaattgacggggacccgcacaagcggtggaggatgtggtttaattcgaggcaacgcgaagaaccttacctgggtttgacatgcagaaagtaggagcccgaaagggtgacaaccggtaaccagtccggaatctgcacaggtgctgc

SEQ ID NO: 484 - DNA, 252 bp, Artificial Sequence
  Synthetic TXv5v6-0249046
ctagccgtaaacgatggacacttgacgtgggcgattttagtctgcgtcggagctaacgtattaagtgtcccgcctggggagtacgttcgcaaggatgaaactcaaaggaattgacggggacccgcacaagcggtggaggatgtggtttaattcgaggcaacgcgaagaaccttacctgggtttgacatgcagaaagtaggagcccgaaagggtgacaaccggtaaccaatccggaatctgcacaggtgctgc

SEQ ID NO: 485 - DNA, 24 bp, Artificial Sequence
  Synthetic reverse complement of reverse primer
agaccttaccagatccgctgagag

SEQ ID NO: 486 - DNA, 246 bp, Artificial Sequence
  Synthetic CONSENS_0231931
ccagccgtaaacgatgctcgctatgtgtcaggtacggtgygaccgtatctggtgccgtagggaagccgtgaagcgagccacctgggaagtacggycgcaagrctgaaacttaaaggaattggcgggggagcactacaacggtggagcctgcggtttaattggattcaacgccggaaatcttaccgggkgagacagcaryatgaaggtcaggctgaagaccttaccrgatycgctgagaggaagtgc

SEQ ID NO: 487 - DNA, 247 bp, Artificial Sequence
  Synthetic TXv5v6-0231931
ccagccgtaaacgatgctcgctatgtgtcaggtacggtgtgaccgtatctggtgccgtagggaagccgtgaagcgagccacctgggaagtacggccgcaaggctgaaacttaaaggaattggcgggggagcactacaacgggtggagcctgcggtttaattggattcaacgccggaaatcttaccgggggagacagcagcatgaaggtcaggctgaagaccttaccagatccgctgagaggaagtgc

SEQ ID NO: 488 - DNA, 246 bp, Artificial Sequence
  Synthetic TXv5v6-0232006
ccagccgtaaacgatgctcgctatgtgtcaggtacggtgtgaccgtatctggtgccgtagggaagccgtgaagcgagccacctgggaagtacggtcgcaagactgaaacttaaaggaattggcgggggagcactacaacggtggagcctgcggtttaattggattcaacgccggaaatcttaccgggggagacagcagcatgaaggtcaggctgaagaccttaccagatccgctgagaggaagtgc

SEQ ID NO: 489 - DNA, 246 bp, Artificial Sequence
  Synthetic TXv5v6-0231898
ccagccgtaaacgatgctcgctatgtgtcaggtacggtgcgaccgtatctggtgccgtagggaagccgtgaagcgagccacctgggaagtacggtcgcaagactgaaacttaaaggaattggcgggggagcactacaacggtggagcctgcggtttaattggattcaacgccggaaatcttaccgggtgagacagcaatatgaaggtcaggctgaagaccttaccggattcgctgagaggaagtgc

SEQ ID NO: 490 - DNA, 24 bp, Artificial Sequence
  Synthetic reverse complement of reverse primer
cctctggtggaaagtcagcacagg

SEQ ID NO: 491 - DNA, 252 bp, Artificial Sequence
  Synthetic CONSENS_0217253
ccagccctaaacgatgtacacttggcatgcgyyrtatkrtgcgtgccgtaggtaacctgttaagtgtaccgcctggggagtaygctcgcaagggtgaaactcaaaggaattgacggggacccgcacaagcggtggaggatgtggttcaattcgaggcaacgcgaagaaccttacctgggcttgacatgctgatagtactraaccgaaaggtgayggattccacctctggtggaaagtcagcacaggtgctgc

SEQ ID NO: 492 - DNA, 252 bp, Artificial Sequence
  Synthetic TXv5v6-0217253
ccagccctaaacgatgtacacttggcatgcgctatattgtgcgtgccgtaggtaacctgttaagtgtaccgcctggggagtacgctcgcaagggtgaaactcaaaggaattgacggggacccgcacaagcggtggaggatgtggttcaattcgaggcaacgcgaagaaccttacctgggcttgacatgctgatagtactgaaccgaaaggtgacggattccacctctggtggaaagtcagcacaggtgctgc

SEQ ID NO: 493 - DNA, 252 bp, Artificial Sequence
  Synthetic TXv5v6-0217292
ccagccctaaacgatgtacacttggcatgcgtcgtatgatgcgtgccgtaggtaacctgttaagtgtaccgcctggggagtatgctcgcaagggtgaaactcaaaggaattgacggggacccgcacaagcggtggaggatgtggttcaattcgaggcaacgcgaagaaccttacctgggcttgacatgctgatagtactaaaccgaaaggtgatggattccacctctggtggaaagtcagcacaggtgctgc

SEQ ID NO: 494 - DNA, 23 bp, Artificial Sequence
  Synthetic reverse complement of reverse primer
gggatgtgcccttcggggaaccc

SEQ ID NO: 495 - DNA, 252 bp, Artificial Sequence
  Synthetic CONSENS_0025886
cacgcagtaaacgatgaacactaggtgtagcgggtattgacccctgctgtgccgcagttaacgcattaagtgttccgcctggggagtacgaccgcaaggttaaaactcaaaggaattgacgggggcccgcacaagcggtggagcatgtggtttaattcgacgcaacgcgaagaaccttacctggatttgacatcccgggaagtcccttgaaaaagggatgtgcccttcggggaacccggtgacaggtgctgc

SEQ ID NO: 496 - DNA, 252 bp, Artificial Sequence
  Synthetic TXv5v6-0025886
cacgcagtaaacgatgaacactaggtgtagcgggtattgacccctgctgtgccgcagttaacgcattaagtgttccgcctggggagtacgaccgcaaggttaaaactcaaaggaattgacgggggcccgcacaagcggtggagcatgtggtttaattcgacgcaacgcgaagaaccttacctggatttgacatcctgggaagtcccttgaaaaagggatgtgcccttcggggaacccggtgacaggtgctgc

SEQ ID NO: 497 - DNA, 252 bp, Artificial Sequence; identical to SEQ ID NO: 495
  Synthetic TXv5v6-0025873

SEQ ID NO: 498 - DNA, 252 bp, Artificial Sequence
  Synthetic TXv5v6-0025863
cacgcagtaaacgatgaacactaggtgtagcgggtattgacccctgctgtgccgcagttaacgcattaagtgttccgcctggggagtacgaccgcaaggttaaaactcaaaggaattgacgggggcccgcacaagcggtggagcatgtggtttaattcgacgcaacgcgaagaaccttacctggatttgacatcccgggaaatcccttgaaaaagggatgtgcccttcggggaacccggtgacaggtgctgc

SEQ ID NO: 499 - DNA, 252 bp, Artificial Sequence
  Synthetic TXv5v6-0025876
cacgcagtaaacgatgaacactaggtgtagcgggtattgacccctgctgtgccgcagttaacgcattaagtgttccgcctggggagtacgaccgcaaggttaaaactcaaaggaattgacgggggcccgcacaagcggtggagcatgtggtttaattcgacgcaacgcgaagaaccttacctggatttgacatcccgggaagtcccttgaaagagggatgtgcccttcggggaacccggtgacaggtgctgc

SEQ ID NO: 500 - DNA, 25 bp, Artificial Sequence
  Synthetic reverse complement of reverse primer
cggaagacgagataacaggtggtgc

SEQ ID NO: 501 - DNA, 255 bp, Artificial Sequence
  Synthetic CONSENS_0726759
ctagctgtaaacgatggatactaggtgtgggaggtatcgaccccttctgtgccgcagctaacgcattaagtatcccgcctggggagtacggtcgcaaggctgaaactcaaaggaattgacgggggcccgcacaagcggtggagcatgtggtttaattcgacgcaacgcgaagaaccttaccgggrcttgacattatcttgcccgtctaagaaattagatcttcttcctttcggaagacgagataacaggtggtgc

SEQ ID NO: 502 - DNA, 255 bp, Artificial Sequence
  Synthetic TXv5v6-0726759
ctagctgtaaacgatggatactaggtgtgggaggtatcgaccccttctgtgccgcagctaacgcattaagtatcccgcctggggagtacggtcgcaaggctgaaactcaaaggaattgacgggggcccgcacaagcggtggagcatgtggtttaattcgacgcaacgcgaagaaccttaccgggacttgacattatcttgcccgtctaagaaattagatcttcttcctttcggaagacgagataacaggtggtgc

SEQ ID NO: 503 - DNA, 255 bp, Artificial Sequence
  Synthetic TXv5v6-0260150
ctagctgtaaacgatggatactaggtgtgggaggtatcgaccccttctgtgccgtagctaacgcattaagtatcccgcctggggagtacggtcgcaaggctgaaactcaaaggaattgacgggggcccgcacaagcggtggagcatgtggtttaattcgacgcaacgcgaagaaccttaccggggcttgacattatcttgcccgtctaagaaattagatcttcttcctttcggaagacgagataacaggtggtgc

SEQ ID NO: 504 - DNA, 255 bp, Artificial Sequence; identical to SEQ ID NO: 502
  Synthetic TXv5v6-0259561

SEQ ID NO: 505 - DNA, 255 bp, Artificial Sequence
  Synthetic TXv5v6-0259703
ctagctgtaaacgatggatactaggtgtgggaggtatcgaccccttctgtgccgcagctaacgcattaagtatcccgcctggggagtacggtcgcaaggctgaaactcaaaggaattgacgggggcccgcacaagcggtggagcatgtggtttaattcgacgcaacgcgaagaaccttaccggggcttgacattgtcttgcccgtctaagaaattagatcttcttccttttggaagacgagataacaggtggtgc

SEQ ID NO: 506 - DNA, 25 bp, Artificial Sequence
  Synthetic reverse complement of reverse primer
ggtcttacctgaatcgctgagagga

SEQ ID NO: 507 - DNA, 247 bp, Artificial Sequence
  Synthetic CONSENS_0258903
ctagctgtaaacgatgctcgctaggtgtcagacacggtgcgaccgtgtttggtgccgcagggaagccgttaagcgagccacctgggaagtacggtcgcaaggctgaaacttaaaggaattggcgggggagcactacaacgggtggagcctgcggtttaattggattcaacgccggaaaactcaccgggtgcgacagcaatatgtaggtcaggctgaaggtcttacctgaatcgctgagaggaggtgc

SEQ ID NO: 508 - DNA, 247 bp, Artificial Sequence
  Synthetic TXv5v6-0258903
ctagctgtaaacgatgctcgctaggtgtcagacacggtgcgaccgtgtttggtgccgcagggaagccgttaagcgagccacctgggaagtacggtcgcaaggctgaaacttaaaggaattggcgggggagcactacaacgggtggagcctgcggtttaattggattcaacgccggaaaactcaccgggtgcgacagcaacatgtaggtcaggctgaaggtcttacctgaatcgctgagaggaggtgc

SEQ ID NO: 509 - DNA, 247 bp, Artificial Sequence; identical to SEQ ID NO: 507
  Synthetic TXv5v6-1692076

SEQ ID NO: 510 - DNA, 247 bp, Artificial Sequence; identical to SEQ ID NO: 507
  Synthetic TXv5v6-0258906

SEQ ID NO: 511 - DNA, 243 bp, Artificial Sequence
  Synthetic TXv5v6-0719836
ctagctgtaaacgatgctcgctaggtgtcagacacggtgcgaccgtgtttggtgccgcagggaagccgttaagcgagccacctgggaagtacggtcgcaaggctgaaacttaaaggaattggcgggggagcactacaacgggtggagcctgcggtttaattggattcaacgccggaaaactcaccgggtgcgacagcaatatgtaggtcaggctgaaggtcttacctgaatcgctgagaggag

SEQ ID NO: 512 - DNA, 20 bp, Artificial Sequence
  Synthetic reverse complement of reverse primer
cgacccttcggggagcctgg

SEQ ID NO: 513 - DNA, 251 bp, Artificial Sequence
  Synthetic CONSENS_0262835
ctagctgtaaacgatgtggacttggcgttggtggggtcaaatccatcagtgccgkagctaacgcgataagtccaccgcctggggactacggtcgcaaggctaaaactcaaaggaattggcgggggcccgcacaagcagcggagcgtgtggtttaattcgatgctacacgaagaaccttacccgggtttgacatccaggtggtagggaaccgaaaggcgaccgacccttcggggagcctggacaggtgctgc

SEQ ID NO: 514 - DNA, 251 bp, Artificial Sequence
  Synthetic TXv5v6-026283
ctagctgtaaacgatgtggacttggcgttggtggggtcaaatccatcagtgccggagctaacgcgataagtccaccgcctggggactacggtcgcaaggctaaaactcaaaggaattggcgggggcccgcacaagcagcggagcgtgtggtttaattcgatgctacacgaagaaccttacccgggtttgacatccaggtggtagggaaccgaaaggcgaccgacccttcggggagcctggacaggtgctgc

SEQ ID NO: 515 - DNA, 251 bp, Artificial Sequence
  Synthetic TXv5v6-0262867
ctagctgtaaacgatgtggacttggcgttggtggggtcaaatccatcagtgccgtagctaacgcgataagtccaccgcctggggactacggtcgcaaggctaaaactcaaaggaattggcgggggcccgcacaagcagcggagcgtgtggtttaattcgatgctacacgaagaaccttacccgggtttgacatccaggtggtagggaaccgaaaggcgaccgacccttcggggagcctggacaggtgctgc

SEQ ID NO: 516 - DNA, 26 bp, Artificial Sequence
  Synthetic reverse complement of reverse primer
agggaagacaggataacaggtggtgc

SEQ ID NO: 517 - DNA, 258 bp, Artificial Sequence
  Synthetic CONSENS_0260001
ctagctgtaaacgatggatactaggtgtgggaggtatcgaccccttctgtgccgyagctaacgcattaagtatcccgcctggggagtacggtcgcaagactgaaactcaaaggaattgacgggggcccgcacaagcggtggagcatgtggtttaattcgacgcaacgcgaagaaccttaccggggcttgacattgtcttgcccgtttaagaaattaaaytttcttcccttttagggaagacaggataacaggtggtgc

SEQ ID NO: 518 - DNA, 258 bp, Artificial Sequence
  Synthetic TXv5v6-0260001
ctagctgtaaacgatggatactaggtgtgggaggtatcgaccccttctgtgccgtagctaacgcattaagtatcccgcctggggagtacggtcgcaagactgaaactcaaaggaattgacgggggcccgcacaagcggtggagcatgtggtttaattcgacgcaacgcgaagaaccttaccggggcttgacattgtcttgcccgtttaagaaattaaactttcttcccttttagggaagacaggataacaggtggtgc

SEQ ID NO: 519 - DNA, 258 bp, Artificial Sequence
  Synthetic TXv5v6-1439641
ctagctgtaaacgatggatactaggtgtgggaggtatcgaccccttctgtgccgtagctaacgcattaagtatcccgcctggggagtacggtcgcaagactgaaactcaaaggaattgacgggggcccgcacaagcggtggagcatgtggtttaattcgacgcaacgcgaagaaccttaccggggcttgacattgtcttgcccgtttaagaaattaaattttcttcccttttagggaagacaggataacaggtggtgc

SEQ ID NO: 520 - DNA, 258 bp, Artificial Sequence
  Synthetic TXv5v6-0725610
ctagctgtaaacgatggatactaggtgtgggaggtatcgaccccttctgtgccgcagctaacgcattaagtatcccgcctggggagtacggtcgcaagactgaaactcaaaggaattgacgggggcccgcacaagcggtggagcatgtggtttaattcgacgcaacgcgaagaaccttaccggggcttgacattgtcttgcccgtttaagaaattaaactttcttcccttttagggaagacaggataacaggtggtgc

SEQ ID NO: 521 - DNA, 25 bp, Artificial Sequence
  Synthetic reverse complement of reverse primer
ccgaagacaggataacaggtggtgc

SEQ ID NO: 522 - DNA, 255 bp, Artificial Sequence
  Synthetic CONSENS_0259164
ctagctgtaaacgatggatactaggtgtrggaggtatcgaccccttctgtgccgyagctaacgcattaagtatcccgcctggggagtacggtcgcaaggctgaaactcaaaggaattgacgggggcccgcacaagcggtggagcatgtggtttaattcgacgcaacgcgaagaaccttaccgggacttgacattatcttgcccgtctaagaaattagatcttcttccttacsgaagacaggataacaggtggtgc

SEQ ID NO: 523 - DNA, 255 bp, Artificial Sequence
  Synthetic TXv5v6-0259164
ctagctgtaaacgatggatactaggtgtaggaggtatcgaccccttctgtgccgcagctaacgcattaagtatcccgcctggggagtacggtcgcaaggctgaaactcaaaggaattgacgggggcccgcacaagcggtggagcatgtggtttaattcgacgcaacgcgaagaaccttaccgggacttgacattatcttgcccgtctaagaaattagatcttcttccttaccgaagacaggataacaggtggtgc

SEQ ID NO: 524 - DNA, 255 bp, Artificial Sequence
  Synthetic TXv5v6-0729803
ctagctgtaaacgatggatactaggtgtgggaggtatcgaccccttctgtgccgtagctaacgcattaagtatcccgcctggggagtacggtcgcaaggctgaaactcaaaggaattgacgggggcccgcacaagcggtggagcatgtggtttaattcgacgcaacgcgaagaaccttaccgggacttgacattatcttgcccgtctaagaaattagatcttcttccttacggaagacaggataacaggtggtgc

SEQ ID NO: 525 - DNA, 247 bp, Artificial Sequence
  Synthetic TXv5v6-0774428
ctggccgtaaacgatgcgtgctaggtgttggtagggctttgagctgtaccagtgccgtagggaaactgttaagcgcgccgcctgggaagtacgctcgcaagggtgaaacttaaaggaattggcggggaagcaccacaaggggtgaagcctgcggttcaattggactcaacgccgggaaacttaccgggggcgacagcagaatgaaggtcaggctgacgaccttaccagacaagctgagcggaggtgc

SEQ ID NO: 526 - DNA, 247 bp, Artificial Sequence
  Synthetic TXv5v6-0220974
ccagccgtaaacgatgcgggctaggtgttggggtggctacgtgccacttcagtgccgcagggaagccattaagcccgccgcctggggagtacggccgcaaggctgaaacttaaaggaattggcgggggagcaccacaaggcgtgaagcttgcggttcaattggagtcaacgccgggaaccttaccgggggcgacagcaggatgaatgccagattgaaggtcttgctggacaagctgagaggaggtgc

SEQ ID NO: 527 - DNA, 246 bp, Artificial Sequence
  Synthetic TXv5v6-0206754
cagggcgtaaacgctgtgggcttagtgttgggtgtcttacgggggcatccagcattgaagagaagttgttaagcccaccacctggggagtacgtccgcaaggatgaaacttaaaggaattggcgggggagcacagcaacgggtggagcgtgcggttcaattggactcaacgccggaaagctcaccgggggcgacgactaaatgaaggccaggctgaagaccttgctggattgccgagaggtggtgc

SEQ ID NO: 528 - DNA, 247 bp, Artificial Sequence
  Synthetic TXv5v6-0266718
ctggccgtaaacgatgcatactaggtgatggcatggccatgagccatgtcagtgccgtagggaaaccgttaagtgtgccgcctgggaagtacggtcgcaaggctaaaacttaaaggaattggcgggggagcaccacaaggggtgaagcctgcggttcaattggactcaacgccgggaaacttaccaggggagacagcagaatgatggtcaggttgacgaccttacctgacgagctgagaggaggtgc

SEQ ID NO: 529 - DNA, 246 bp, Artificial Sequence
  Synthetic TXv5v6-0771067
ctggccgtaaacgatgcatactaggtgatggtacggccatgagccgtatcagtgccgtagggaaaccgttaagtgtgccgcctgggaagtacggtcgcaaggctaaaacttaaaggaattggcgggggagcaccacaaggggtgaagcctgcggttcaattggactcaacgccgggaaacttaccagggagacagcagtatgacggtcaggttgacgaccttacccgacgagctgagaggaggtgc

SEQ ID NO: 530 - DNA, 247 bp, Artificial Sequence
  Synthetic TXv5v6-0220961
ccagccgtaaacgatgcgggctaggtgttagggtggctacgagccactctagtgccgcagcgaaggcattaagcccgccgcctggggagtacggccgcaaggctgaaacttaaaggaattggcgggggagcaccacaaggcgtgaagcgtgcggtttaattggagtcaacaccggaaaccttaccgggggcgacagcaggttgaaggccagattgacgatcttgccagacaagctgagaggaggtgc

SEQ ID NO: 531 - DNA, 251 bp, Artificial Sequence
  Synthetic TXv5v6-0207124
cagggtgtaaacgctgcctgcttagtgttagacattctttatgggtgtctagtgctggagagaagttgttaagcaggctgcttggggagtatggtcgcaaggctgaaacttaaaggaattggcgggggagcaccgcaacgggaggagcgtgcggtttaattggattcaacgccggaaagttcaccagaggagactaccatatgagggccaggctgaagaccttgctcgatcaataggtagagaggtggtgc

SEQ ID NO: 532 - DNA, 247 bp, Artificial Sequence
  Synthetic TXv5v6-0206646
cagggcgtaaacgctgcccgcttggtattagggaatccacgagattctctagtgccggagagaagtcgttaagcgggccacctgggaagtacggtcgcaaggctgaaacttaaaggaattggcgggggagcactgcaacgggtggagcgtgcggttcaattggatccaacgccggaaagcttaccgaggacgacggataaatgaaggccaggctgaagaccttgctgaattctccgagaagtggtgc

SEQ ID NO: 533 - DNA, 247 bp, Artificial Sequence
  Synthetic TXv5v6-0208572
cagggtgtaaacgctgcttgcttgatgttagttgggcttcgagcccaattagtgtcggagagaagttgttaagcaagctgcctgggaagtacggtcgcaagactgaaacttaaaggaattggcgggggagcacagcaacgggtggagcgtgcggtttaattggattcaacgccggaaaactcaccggaggcgacggttacatgaaggccaggctgatgaccttgcctgattttccgagaggtggtgc

SEQ ID NO: 534 - DNA, 247 bp, Artificial Sequence
  Synthetic TXv5v6-0242332
cggggcgtaaacgctgcaggcttggtgttgggggctctacgagagcgcccagtgccgaagagaaattgttaagcctgccgcttggggagtacggtcgcaagactgaaacttaaaggaattggcgggagagcacagcaacgggaggagcgtgcggttcaattggattcaacgccggaaaactcaccgggggcgacggttacatgaaggccaagctgatgactttgcctgattttccgagaggtggtgc

SEQ ID NO: 535 - DNA, 247 bp, Artificial Sequence
  Synthetic TXv5v6-0206834
cagggtgtaaacgctgcagacttggtgtcggaggtcctacgagggcgtccggtgccggagagaaattgttaagtctgccgcttggggagtacgtccgcaaggatgaaacttaaaggaattggcgggagagcaccgcaacgggaggagcgtgcggttcaattggattcaacgccggaaaactcaccgggggcgacggttacatgaaggccaagctgatgactttgcccgattttccgagaggtggtgc

SEQ ID NO: 536 - DNA, 247 bp, Artificial Sequence
  Synthetic TXv5v6-0206604
cagggcgtaaacgctgcaggcttggtgttgggggtcctacgagggcgcccagtgccggagtgaagatgttaagcctgccgcttggggagtacgtccgcaaggatgaaacttaaaggaattggcgggagagcacagcaacgggaggagcgtgcggttcaattggattcaacgccggaaaactcaccgggagcgacgattacatgaaggtcaggctgatgaccttacctgatttttcgagaggtggtgc

SEQ ID NO: 537 - DNA, 247 bp, Artificial Sequence
  Synthetic TXv5v6-0257786
ctagctgtaaacgatgcgagctaggtggtagtcccactgcgagtggtactgacgccgtagggaagccgtgaagctcgccacctgggaagtacggtcgcaaggctgaaacttaaaggaattggcgggggagcacaacaacgggtggatgctgcggtttaattggattcaacgccgggaaacttaccgggggcgacagcaacatgaagaccgggctgaagaccttgtcagattagctgagaggtggtgc

SEQ ID NO: 538 - DNA, 247 bp, Artificial Sequence
  Synthetic TXv5v6-0258881
ctagctgtaaacgatgcgggctaggtgttggcattactgcgagtggtgccagtgccgaagggaagccgttaagctcgccatctggggagtacggtcgcaaggcttaaacttaaaggaattggcgggggagcaccacaacgggtggagcctgcggttcaattggattcaacgccggaaaactcaccggaggggacagcgagatgaaggtcaggctgaagaccttaccaaattagctgagaggtggcgc

SEQ ID NO: 539 - DNA, 247 bp, Artificial Sequence
  Synthetic TXv5v6-0257959
ctagctgtaaacgatgcgggccaggtgttggcattactgcgagtgatgtcagtgccaaagggaagccgttaagcccgccatctggggagtacggtcgcaaggctgaaacttaaagaaattggcgggggagcaccacaacgggtggagcctgcggttcaatcggattcaacgccggaaaactcaccggaggcgacagcgagatgaaggtcaggctgaagaccttaccggattagctgagaggtggcgc

SEQ ID NO: 540 - DNA, 247 bp, Artificial Sequence
  Synthetic TXv5v6-0220923
ccagccgtaaacgatgcgcgttaggtgtgccggtgaccacgagttaccggggtgccgaagggaaaccgtgaaacgtgccgcctgggaagtacggtcgcaaggctgaaacttaaaggaattggcgggggagcaccacaacgggtggagcctgcggtttaattggactcaacgccggaaatctcaccggataagacagcggaatgatagcctggctgaagaccttgcttgaccagctgagaggaggtgc

SEQ ID NO: 541 - DNA, 247 bp, Artificial Sequence
  Synthetic TXv5v6-0256396
ctagccgtaaacgatgtgagctaggtgtcagtcacactgcgagtatgactggtgccgtagggaagccgtgaagctcaccacttgggaagtacggccgcaaggctgaaacttaaaggaattggcgggggagcaccacaacaggtggatgctgcggtttaattggatacaacgccggaaatctcaccgggggcgacagcaatatgaaggtcaggctgaagaccttacccgattcgctgagaggaggtgc

SEQ ID NO: 542 - DNA, 247 bp, Artificial Sequence
  Synthetic TXv5v6-0256404
ctagccgtaaacgatgtgagctaggtgtcagtcatggcatgtccgtgattggtgccgtagggaagccgtgaagctcaccacctgggaagtacggccgcaaggctgaaacttaaaggaattggcgggggagcacaacaacgggtggatgctgcggtttaattggattaaacgccggaaagctcaccgggggcgacagcaatatgaaggtcaggctgaagaccttaccaaattcgctgagaggtggtgc

SEQ ID NO: 543 - DNA, 247 bp, Artificial Sequence
  Synthetic TXv5v6-0600543
ccagccgtaaacgatgcgagctaggtgtcagttttactgcgagtaaaattggtgccgtagggaagccgtgaagctcgccacctgggaagtacggccgcaaggctgaaacttaaaggaattggcgggggagcactacaacgggtggagcctgcggtttaattggactcaacgccggaaatctcaccgggtgcgacagcagaatgaaggccaggctgaagaccttgccagacttgctgagaggaggtgc

SEQ ID NO: 544 - DNA, 247 bp, Artificial Sequence
  Synthetic TXv5v6-0248410
ctagccgtaaacgatgcttgctaggtgtcagccacggtgcgaccgtggttggtgccgtagggaagccgtgaagcgagccacctgggaagtacggccgcaaggctgaaacttaaaggaattggcgggggagcactacaacgggtggagcctgcggtttaattggattcaacgccggaaagctcaccgggggcgacagcgatatgaaggtcaggctgaagaccttacccgattagctgagaggtggtgc

SEQ ID NO: 545 - DNA, 247 bp, Artificial Sequence
  Synthetic TXv5v6-0237795
ccagctgtaaacgatgctcgctaggtgtcagatacggtgcgtccgtatttggtgccgtagggaaaccgtgaagcgagccgcctgggaagtacggtcgcaaggctgaaacttaaaggaattggcgggggagcactacaacgggtggagcctgcggttcaattggactcaacgccggaaaactcaccagatcagacagcactatgaaggtcaggctgaagaccttacctgattcgctgagaggaggtgc

SEQ ID NO: 546 - DNA, 254 bp, Artificial Sequence
  Synthetic TXv5v6-0210733
catgccgtaaacgatgggtactaggtgtaggaggtatcgacccctcctgtgccgcagttaacacaataagtaccccgcctggggagtacggccgcaaggttgaaactcaaaggaattgacgggggcccgcacaagcggtggagcatgtggtttaattcgacgcaacgcgaagaaccttaccagggtttgacatcccctgacagcggtggaaacatcgtctttcttcttcggaagaacagggagacaggtggtgc

SEQ ID NO: 547 - DNA, 257 bp, Artificial Sequence
  Synthetic TXv5v6-0195046
cacgctgtaaacgatgggcactaggtgttggaggtatcgacccctccagtgccgtagttaacacaataagtgccccgcctggggagtacggccgcaaggttgaaactcaaaggaattgacgggggcccgcacaagcggtggagcatgtggtttaattcgacgcaacgcgaagaaccttaccagggcttgacatcccctgaaagcggcggaaacgtcgtcctcttaagattttcttgagacagggagacaggtggtgc

SEQ ID NO: 548 - DNA, 251 bp, Artificial Sequence
  Synthetic TXv5v6-1308235
cacgccgtaaacgatggatactaggtgtagggggtttagataccttctgtgccgcagttaacacattaagtatcccgcctggggagtacggccgcaaggttgaaactcaaaggaattgacgggggcccgcacaagcggtggagcatgtggtttaattcgaagcaacgcgaagaaccttaccagggcttgacatatacctgacgtaatgggagactattatttcctttcggggacaggtatacaggtggtgc

SEQ ID NO: 549 - DNA, 251 bp, Artificial Sequence
  Synthetic TXv5v6-0543221
cacgctgtaaacgatggatactaggtgtagggggtttagataccttctgtgccgcagttaacacattaagtatcccgcctggggagtacggccgcaaggttgaaactcaaaggaattgacgggggcccgcacaagcggtggagcatgtggtttaattcgaagcaacgcgaagaaccttaccaggacttgacatatacctgacgtaatgggagactattatttcctttaggggacaggtaaacaggtggtgc

SEQ ID NO: 550 - DNA, 257 bp, Artificial Sequence
  Synthetic TXv5v6-0257331
ctagctataaacgatggatactaggtgtgggaggtatcgaccccttctgtgccgcagctaacgcattaagtatcccgcctggggagtacggtcgcaaggctgaaactcaaaggaattgacgggggcccgcacaagcggtggagcatgtggtttaattcgacgcaacgcgaagaaccttaccgggacttgacattgtcttgcccatctaagaaattagattttcttccctttggggaagacgagataacaggtggtgc

SEQ ID NO: 551 - DNA, 255 bp, Artificial Sequence
  Synthetic TXv5v6-0591983
ccagccctaaacgatgtgcacttggcatgcgtcataatttggtgcgtgccgcaggtaacctgttaagtgcaccgcctggggagtacgctcgcaagggtgaaactcaaaggaattgacggggacccgcacaagcggtggaggatgtggtttaattcgaggcaacgcgaagaaccttacctggacttgacatgctgatagtatcaaaccgaaaggtgagagatttcgcctctggtgaaaagtcagcacaggtgctgc

SEQ ID NO: 552 - DNA, 260 bp, Artificial Sequence
  Synthetic TXv5v6-1294019
cacgccgcaaacgatgttcactgggtgtcggtcacacatatagatcggtgccggagttaacgcattaagtgaaccgcctggggaatacggccgcaaggctaaaactcaagagaattgacgggtccctgcacaagcggtggagcacgtggtttaattcgatgataagcgaagaacctcacctgggcttgacatactagtggtaggagcctgaaagggtaacgacctctgccttcgggtagaggagctagcacaggtgatgc

SEQ ID NO: 553 - DNA, 261 bp, Artificial Sequence
  Synthetic TXv5v6-1410009
ctagccgtaaacgatggatgcttggtgttggggaggttgcttctcagtgccggagctaacgtgttaagcatcccacctgggaagtacgaccgcaaggttaaaactcaaaggaattgacggggccccgcacaagcagcgaggcatgtggtttaattcgatgctacacgaagaaccttacctgggcttgacatgcacgagaaagctggaagaaactccggccctctcagagagtaatctctgttacccgtgcacaggtgctgc

SEQ ID NO: 554 - DNA, 252 bp, Artificial Sequence
  Synthetic TXv5v6-1287141
cacgccctaaacgatggctactagctgtttgaagtatcgacccttcaagtggcgaagctaacgcgttaagtagcccgcctgggaagtacggtcgcaagactaaaactcaaaggaatagacgggagtttgcacaagtggtggagcgtctggtttaattcgatgatgagcgaggaaccttaccagggtttgacactctagtgaaatcttgccgaaaggcatgtctacctacaaggacactagagcaggtgctgc

SEQ ID NO: 555 - DNA, 252 bp, Artificial Sequence
  Synthetic TXv5v6-1336703
cacgctgtaaacgatggacactggctatttgaagtgtcgacccttcaagtggcgaagctaacgcgttaagtgtcccacctgggaagtacggcggcaacgctaaaactcaaaggaatagacggggcttcacacaagcggtggaccatgtggctcaattcgacaacaaacgaagaaccttaccagggcttgacaagctagtgaaaattcgccgaaaggcgagtctacccacaaggacactagcccaggtgctgc

SEQ ID NO: 556 - DNA, 255 bp, Artificial Sequence
  Synthetic TXv5v6-0062459
cacgccgcaaactttggacactaggtatggagggtatcgaccccctctgtgccgaagctaacgcgttaagtgtcccgcctggggattacggccgcaaggctaaaactcaaaggaattgacgggggcccgcacaagcagcggagcgtgtggtttaattcgacgatacgcgaagaaccttaccagggcttgacatgccggtagtagaagcctgaaagggtgaccacctgtaaagccaggagccggcacagatgctgc

SEQ ID NO: 557 - DNA, 251 bp, Artificial Sequence
  Synthetic TXv5v6-0062219
cacgccgcaaacgatggacactaggtatggggggtatcgaccctctccgtgccgaagttaacgcgttaagtgtcccacctggggattacggccgcaaggctaaaactcaaaggaattgacgggggcccgcacaagcagcggagcgtgtggtttaattcgacgatacgcgaagaaccttaccagggcttgacgtgcacacgaaaccctgagaaatcagggcttccttcgcaggacgggtgcacaggtgctgc

SEQ ID NO: 558 - DNA, 256 bp, Artificial Sequence
  Synthetic TXv5v6-0059823
cacgccctaaacggtggatactggatatagggggtatcgacccctctgtgtcgaagctaacgctttaagtatcccgcctggggactacggccgcaaggctaaaactcaaaggaattgacgggggcccgcacaagcagcggagcgtgtggtttaattcgatgcaacacgaagaaccttacccaggcttgacatactagtggtaggaacctgaaagggggacgaccttggttttccaaggagctagcacaggtgctgc

SEQ ID NO: 559 - DNA, 257 bp, Artificial Sequence
  Synthetic TXv5v6-0179152
cacgccttaaacggtggacattaggtatggggagtatcgaccctctccgtgtcgaagctaacgctttaaatgtcccgcctggggagtacggccgcaaggctaaaactcaaaggaattgacgggggcccgcacaagcagcggagcgtgtggtttaattcgatgatacacgaagaaccttaccagggcttgatatgccaggagtaggattccgaaaggaggacgacctgtatccagtcaggagctggcacaggtgctgc

SEQ ID NO: 560 - DNA, 257 bp, Artificial Sequence
  Synthetic TXv5v6-0059458
cacgccctaaacggtggacactagacatgggaggtatcgaccctttctgtgtcgaagctaacgccttaagtgtcccgcctggggagtacggccgcaaggctaaaactcaaaggaattgacgggggtccgcacaagcagcggagcgtgtggtttaattcgatgctacacgaagaaccttaccagggtttgacatggcagaagtaggaatccgaaaggaggacgacctgtatccagtcaggagctgtcacaggtgctgc

SEQ ID NO: 561 - DNA, 257 bp, Artificial Sequence
  Synthetic TXv5v6-0059692
cacgccctaaacggtggacactagatatggggagtatcgaccctcctcgtgtcgaagctaacgctttaagtgtcccgcctggggactacgaccgcaaggttaaaactcaaaggaattgacgggggcccgcacaagcagcggagcgtgtggtttaattcgatgctacgcgaagaaccttaccaaggcttgacatgtcagtagtaggaatccgaaaggaggacgacctgtatccagtcaggaactgtcacaggtgctgc

SEQ ID NO: 562 - DNA, 255 bp, Artificial Sequence
  Synthetic TXv5v6-0305895
cacgcagtaaacgatggacactaggtatagggagtatcgaccctctctgtgccgaagttaacgcattaagtgtcccgcctggggagtacggtcgcaagactaaaactcaaaggaattgacgggggcccgcacaagcagcggagcgtgtggtttaattcgatgcaacacgaagaaccttaccaaggcttgacatgtcggaagtagcgacccgaaaggtgagcgacctgttaaatcaggagccgtcacaggtgctgc

SEQ ID NO: 563 - DNA, 257 bp, Artificial Sequence
  Synthetic TXv5v6-0060461
cacgccctaaacgttggacactaggtatggggagtatcgaccctcttcgtgccgaagctaacgctttaagtgtcccgcctggggactacggccacaaggctaaaactcaaaggaattgacggggacccgcacaagcagcggagcgtgtggtttaattcgatgcaacgcgaagaaccttaccaaggcttgacatgccagaagtagaagcttgaaagagtaacgacctgtatccagtcaggagctggcacaggtgctgc

SEQ ID NO: 564 - DNA, 255 bp, Artificial Sequence
  Synthetic TXv5v6-0175746
cacgccgtaaactatggacactaggtatggggagtatcgaccctctccgtgccgaagttaacgcattaagtgtcccgcctggggagtacggccgcaaggctaaaactcaaaggaattgacgggggcccgcacaagcagcggagcgtgtggtttaattcgatgcaacgcgaagaaccttaccaaggcttgacatgtcggaagtagcgacctgaaagggaagtcacctgttgagtcaggagccggcacaggtgctgc

SEQ ID NO: 565 - DNA, 251 bp, Artificial Sequence
  Synthetic TXv5v6-0250092
ctagccgtaaacgatggatactaggtgtgggtggcattgaccccatccgtgccgtagctaacgcgataagtatcccgcctggggactacggccgcaaggttaaaactcaaaggaattgacgggggcccgcacaagcagcggagcgtgtggtttaattcgatgctacacgaagaaccttaccagggcttgacatgctggtggtacggacccgaaagggcgaggacccgcaagggagccagcacaggtgctgc

SEQ ID NO: 566 - DNA, 251 bp, Artificial Sequence
  Synthetic TXv5v6-0252039
ctagccgtaaacgatggatgctgggtgtggggggtatagattccctccgtgccgaagctaacgcgttaagcatcccgcctggggactacggccgcaaggctaaaactcaaacgaattgacgggggcccgcacaagcagcggagcgtgtggtttaattcgatgcaacacgaagaaccttacctgggtttgacatgctggtagtagtgaagcgaaagcggaacgacccttcggggagcctgcacaggtgctgc

SEQ ID NO: 567 - DNA, 251 bp, Artificial Sequence
  Synthetic TXv5v6-0257726
ctagctgtaaacgatgcagacttggtgttggtggtttaatagccatcagtgccgtagctaacgcggtaagtctgccgcctggggactacggccgcaaggctaaaactcaaaggaattgacggggacccgcacaagcagcggagcgtgtggtttaattcgaggctacgcgaagaaccttacctgggtttgacatgcacgtggtaggaagccgaaaggcgaccgacccttcggggagcgcgcacaggtgctgc

SEQ ID NO: 568 - DNA, 255 bp, Artificial Sequence
  Synthetic TXv5v6-0120132
cacgccgtaaacgatgcctactaggttgtggtggttctgacaccagcacagccgaagcaaaagtgctaagtaggccgcctggggagtacggtcgcaaggctaaaactcaaaggaattgacgggggctcacacaagcggtggagcatgtggattaattcgaagcaacgcgtagaaccttacctgggtttgacatgcttggatgcttgcctggaaacaggttaagttaccttcgggtgaaacttgcacaggtgctgc

SEQ ID NO: 569 - DNA, 248 bp, Artificial Sequence
  Synthetic TXv5v6-1276382
caagccgtaaacgatgggcacttgacgtaggcatatgtctgcgtcggagctaacgtgttaagtgccccgcctggggagtacgttcgcaaggatgaaactcaaaggaattgacggggacccgcacaagcggtggaggatgtggtttaattcgaggcaacgcgaagaaccttacctgggttcgactgaaagaaagtaggaacctgaaagggggacgaccggtaatcagtccggaatctgacaggtgctgc

SEQ ID NO: 570 - DNA, 249 bp, Artificial Sequence
  Synthetic TXv5v6-0722918
ctagctgtaaacgatggatactagattttgcaagttattgctagatcgaagctaacgcattaagtatcccgcctggggagtacggtcgcaaggctaaaactcaaaggaattgacggggacccgcacaagcagtggagcatgtggtttaattcgatgcaacgcgaagaaccttacctgggcttgaactgtaggcattatccacctgaaagggttggctatcctcttcggaggaacctatagaggtgctgc

SEQ ID NO: 571 - DNA, 253 bp, Artificial Sequence
  Synthetic TXv5v6-0690447
ctagccgtaaacgatgggcactagatgtttctgcttttatgcaggagtatcgaagctaacgcgttaagtgccccgcctggggagtacggtcgcaaggctaaaactcaaaggaattgacgggggcccgcacaagcggtggagcatgtggtttaattcgacgcaacgcgaaaaaccttacctgggcttgaactgctggtagtaagaccccgaaagggtaatgatcctgcttgcaggaagccagcagaggtgctgc

SEQ ID NO: 572 - DNA, 254 bp, Artificial Sequence
  Synthetic TXv5v6-0690171
ctagccgtaaacgatgggcactagatgtttctgctttaagcaggagtgtcgaagttaacgcgttaagtgccccgcctggggagtacggtcgcaaggctgaaactcaaaggaattgacgggggcccgcacaagcggtggagcatgtggttcaattcgacgcaacgcgaagaaccttacctgggtttgaactgctggtagtaagaccccgaaaggggaatgatcctggcttgccaggaagccagcagaggtgctgc

SEQ ID NO: 573 - DNA, 245 bp, Artificial Sequence
  Synthetic TXv5v6-0187739
cacgctgtaaacgatgatcactcgttgttggcgatatacagtcagcgactaagcgaaagcattaagtgatccacctggggagtacgatcgcaagattgaaactcaaaggaattgacgggggcccgcacaagcggaggaacatgtggtttaattcgatgatacgcgaggaaccttacctgggcttaaatgcagtatgacttccgccgaaaggtggattcccttcggggcagattgcaaggtgctgc

SEQ ID NO: 574 - DNA, 252 bp, Artificial Sequence
  Synthetic TXv5v6-0404321
cacgccgtaaacgatgagtgctaggtgttggggggtttccgcccctcagtgctgaagctaacgcattaagcactccgcctggggagtacggccgcaaggctgaaactcaaaggaattgacggggacccgcacaagcggtggagcatgtggtttaattcgaagcaacgcgaagaaccttaccaactcttgacatcccattgaccgcttgagagatcaagttttcccttcggggacaatggtgacaggtggtgc

SEQ ID NO: 575 - DNA, 253 bp, Artificial Sequence
  Synthetic TXv5v6-0168244
cacgccgtaaacgatgttcactaggtgttgggagtattgaccctctcggtgccgtagctaacgcattaagtgaaccgcctggggagtacggtcgcaagactaaaactcaaaggaattgacgggggcccgcacaagcggtggagcatgtggtttaattcgatgcaacgcgaagaaccttacctgggtttgacatcccgagaatctcctcggaaacgagggagtgcccttcggggaactcggtgacaggtgctgc

SEQ ID NO: 576 - DNA, 252 bp, Artificial Sequence
  Synthetic TXv5v6-0168232
cacgccgtaaacgatgttcactaggtgttgggagtattgaccctctcggtgccgtagctaacgcattaagtgaaccgcctggggagtacggtcgcaagactaaaactcaaaggaattgacgggggcccgcacaagcggtggagcatgtggtttaattcgatgcaacgcgaagaaccttacctggacttgacatcccgagaatcctctggaaacagaggagtgcccttcggggaacttggtgacaggtgctgc

SEQ ID NO: 577 - DNA, 255 bp, Artificial Sequence
  Synthetic TXv5v6-0183853
cacgctgtaaacgatgagaactaggtgtagcgggtattgacccctgctgtgccgaagttaacgcattaagttctccgcctggggagtacggccgcaaggctaaaactcaaaggaattgacgggggcccgcacaagcggtggagcatgtggtttaattcgacgcaacgcgaagaaccttacctgggtttgacatcctttgacagtcccggaaacgggatctttccgactttgtcggaacagagtgacaggtgctgc

SEQ ID NO: 578 - DNA, 252 bp, Artificial Sequence
  Synthetic TXv5v6-0063999
cacgccgtaaacgatgaacactaggtgtagcgggtattgacccctgctgtgccgtagctaacgcattaagtgttccgcctggggactacggtcgcaaggctaaaactcaaaggaattgacgggggcccgcacaagcggtggagcatgtggtttaattcgacgcaacgcgaagaaccttacctgggtttgacatcccgagaagtccctcgaaagaggggtgtgcccttcggggaactcggagacaggtgctgc

SEQ ID NO: 579 - DNA, 245 bp, Artificial Sequence
  Synthetic TXv5v6-0176581
cacgccgtaaactatgggtgctagccgtcggggagcttgctcttcggtggcgcagctaacgcattaagcaccccgcctggggagtacgatcgcaagattaaaactcaaaggaattgacgggggcccgcacaagcggtggagcatgtggtttaattcgaggcaacgcgaagaaccttaccagcccttgacttcccggtcggggctccggagacggagctcttcaattggccggtgacaggtgctgc

SEQ ID NO: 580 - DNA, 249 bp, Artificial Sequence
  Synthetic TXv5v6-0255064
ctagccgtaaacgatgggtattagacatcggccgaaattcggtaggtgtcgaagctaacgcgttaaatacctcacctggggagtacggccgcaaggctgaaactcaaaggaattgacgggggcccgcacaagcggtggagtatgtggttcaattcgacgatacgcgaagaaccttaccgggatttgacatcccaggaatctgtccgaaaggacggagtgctcgtaagagaacctggagacaggtggtgc

SEQ ID NO: 581 - DNA, 248 bp, Artificial Sequence
  Synthetic TXv5v6-0138901
cacgccgtaaacgatgtcaactaactgttgggcgggtttccgcttagtggtgcagctaacgcaataagttgaccgcctggggagtacggccgcaaggctaaaactcaaatgaattgacgggggcccgcacaagcggtggagcatgtggtttaattcgatgcaacgcgaagaaccttacctacccttgacatacagagaactttctagagatagattggtgccttcgggaactctgatacaggtgctgc

SEQ ID NO: 582 - DNA, 570 bp, Artificial Sequence
  Synthetic Consensus_GOM13
ccggattagawacccbggtagtcctatgcygtaaacgatgctcactaagtgttaggtaatgcaagacrttrtctagtgccgaagcgaaagcgttaagtgagccgcctgggaagtacgttcgcaagaatgaaacttaaaggaattggcgggggcctactacaagaagtggagcctgcggtttaattggactcaactccgggaarctcacctgggccgyaacrtgratgattgtcctgctgaagacactrcttgaygygttactggaggtgcatggccatcgtcagttcgtgccgtgaggtgtcctgttaagtcaggcaacgaacgagatcccyrccgctaattgccagcgagamcwgktcgtcggggacattagcgggactgctcgcgaaaaagtgagaggaaggaagggccaacggtaggtcagtatgccccgatatgcccagggctacacgcgggctacaatggckrgtacagagggttccwacaccgaaaggtgacggyaatctccaaamycgtctcagttgggattgygggctgcaactcgcccrcatgaacttggaatttctagta

SEQ ID NO: 583 - DNA, 585 bp, Artificial Sequence
  Synthetic Consensus_SAGMEG
gattagawacccgggtagtcctagctgtaaagcatgcgggccaggtgtctagcgctccttgagggcgctakgtgccggagggaagccgttaagcccgccgcctgggaagtacggcgcaaggctgaaacttaaagaaattggcgggggagcaccacaagrggtggracctgcggttcaattggattcaacgccggamaactcaccaggggcgacagytggttgamggccagrttgacgayyttgcysgactagctgagaggtggtgcatggccatcgtcagctcgtaccgtgaggcgtcctgttaagtcaggcaacgagcgagatcctcgcccytagttgccatcggtggraagccgggcactctagggggaccgctggcgctaagtcagaggaaggagagggcgacggtaggtcagtatgccccgaatcccctgggctacacgcgggtyacaatgcgcaggacaatgagatgcaaccccgtaaggggragccaarcccmtaaacctgcgctcggttcggatcgagggctgtaactcgccctcgtgaagctggaatcyctagtaatcgcgtgccaacaccgcgcgg

SEQ ID NO: 584 - DNA, 587 bp, Artificial Sequence; identical to SEQ ID NO: 295
  Synthetic CONSENS_26

SEQ ID NO: 585 - DNA, 588 bp, Artificial Sequence; identical to SEQ ID NO: 296
  Synthetic 1639_V360T

SEQ ID NO: 586 - DNA, 588 bp, Artificial Sequence
  Synthetic 1629_R586T
ctagctgtaaacgatggatactaggtgtgggaggtatcgaccccttctgtgccgcagctaacgcattaagtatcccgcctggggagtacggtcgcaaggctgaaactcaaaggaattgacgggggcccgcacaagcggtggagcatgtggtttaattcgacgcaacgcgaagaaccttaccgggacttgacattatcttgcccgtctaagaaattagatcttcttccttaccgaagacgagataacaggtggtgcatggttgtcgtcagctcgtgtcgtgagatgttgggttaagtcccacaacgagcgcaacccttatgcttagttgctaacttgttttacaagtgcactctaagcagactgccgcagataatgcggaggaaggtggggatgacgtcaaatcatcatgccccttacgtcccgggctacacacgtgctacaatggcctgtacagagggtagcgaaagagcgatcttaagccaatcccaaaaagcaggccccagttcggattggaggctgcaactcgcctccatgaagtaggaatcgctagtaatcgcggatcagcatgccgcggtgaatacgtcccgg

SEQ ID NO: 587 - DNA, 587 bp, Artificial Sequence; identical to SEQ ID NO: 295
  Synthetic 1639_V549T

SEQ ID NO: 588 - DNA, 588 bp, Artificial Sequence; identical to SEQ ID NO: 296
  Synthetic 1653_Y411T

SEQ ID NO: 589 - DNA, 587 bp, Artificial Sequence
  Synthetic 1629_R165T
ctagctgtaaacgatggatactaggtgtgggaggtatcgaccccttctgtgccgcagctaacgcattaagtatcccgcctggggagtacggtcgcaaggctgaaactcaaaggaattgacgggggcccgcacaagcggtggagcatgtggtttaattcgacgcaacgcgaagaaccttaccgggacttgacattatcttgcccgtctaagaaattagatcttcttccttaccgaagacgagataacaggtggtgcatggttgtcgtcagctcgtgtcgtgagatgttgggttaagtcccacaacgagcgcaacccttatgcttagttgctaacttgttttacaagtgcactctaagcagactgtcgcagataatgcggaggaaggtggggatgacgtcaaatcatcatgccccttacgtcccgggctacacacgtgctacaatggtctgtacagagggtagcgaaagagcgatcttaagccaatcccaaaaggcaggccccagttcggattggaggctgcaactcgcctccatgaagtaggaatcgctagtaatcgcggatcagcatgccgcggtgaatacgtcccg

SEQ ID NO: 590 - DNA, 587 bp, Artificial Sequence; identical to SEQ ID NO: 295
  Synthetic 1639_V005T

SEQ ID NO: 591 - DNA, 587 bp, Artificial Sequence; identical to SEQ ID NO: 295
  Synthetic 1637_T485T

SEQ ID NO: 592 - DNA, 587 bp, Artificial Sequence; identical to SEQ ID NO: 295
  Synthetic 1639_V287T

SEQ ID NO: 593 - DNA, 587 bp, Artificial Sequence; identical to SEQ ID NO: 295
  Synthetic 1639_V279T

SEQ ID NO: 594 - DNA, 587 bp, Artificial Sequence; identical to SEQ ID NO: 295
  Synthetic 1639_V350T

SEQ ID NO: 595 - DNA, 587 bp, Artificial Sequence; identical to SEQ ID NO: 295
  Synthetic 1629_R985T

SEQ ID NO: 596 - DNA, 587 bp, Artificial Sequence
  Synthetic 1653_Y262T
ctagctgtaaacgatggatactaggtgtgggaggtatcgaccccttctgtgccgcagctaacgcattaagtatcccgcctggggagtacggtcgcaaggctgaaactcgaaggaattgacgggggcccgcacaagcggtggagcatgtggtttaattcgacgcaacgcgaagaaccttaccgggacttgacattatcttgcccgtctaagaaattagatcttcttccttaccgaagacaagataacaggtggtgcatggttgtcgtcagctcgtgtcgtgagatgttgggttaagtcccacaacgagcgcaacccttatgcttagttgctaacttgttttacaagtgcactctaagcagactgtcgcagataatgcggaggaaggtggggatgacgtcaaatcatcatgccccttacgtcccgggctacacacgtgctacaatggtctgtacagagggtagcgaaagagcgatcttaagccaatcccaaaaagcaggccccagttcggattggaggctgcaactcgcctccatgaagtaggaatcgctagtaatcgcggatcagcatgccgcggtgaatacgtcccg

SEQ ID NO: 597 - DNA, 588 bp, Artificial Sequence; identical to SEQ ID NO: 296
  Synthetic 1629_R817T

SEQ ID NO: 598 - DNA, 588 bp, Artificial Sequence
  Synthetic 1629_R069T
ctagctgtaaacgatggatactaggtgtgggaggtatcgaccccttctgtgccgcagctaacgcattaagtatcccgcctggggagtacggtcgcaaggctgaaactcaaaggaattgacgggggcccgcacaagcggtggagcatgtggtttaattcgacgcaacgcgaagaaccttaccgggacttgacattatcttgcccgtctaagaaattagatcttcttccttaccgaagacgagataacaggtggtgcatggttgtcgtcagctcgtgtcgtgagatgttgggttaagtcccacaacgagcgcaacccttatgcttagttgctaacttgttttacaagtgcactctaagcagactgtcgcagataatgcggaggaaggtggggatgacgtcaaatcatcgtgccccttacgtcccgggctacacacgtgctacaatggtctgtacagagggtagcgaaagagcgatcttaagccaatcccaaaaagcaggccccagttcggattggaggctgcaactcgcctccatgaagtaggaatcgctagtaatcgcggatcagcatgccgcggtgaatacgtcccgg

SEQ ID NO: 599 - DNA, 587 bp, Artificial Sequence
  Synthetic 1629_R531T
ctagctgtaaacgatggatactaggtgtgggaggtatcgaccccttctgtgccgcagctaacgcattaagtatcccgcctggggagtacggtcgcaaggccgaaactcaaaggaattgacgggggcccgcacaagcggtggagcatgtggtttaattcgacgcaacgcgaagaaccttaccgggacttgacattatcttgcccgtctaagaaattagatcttcttccttaccgaagacgagataacaggtggtgcatggttgtcgtcagctcgtgtcgtgagatgttgggttaagtcccacaacgagtgcaacccttatgcttagttgctaacttgttttacaagtgcactctaagcagactgtcgcagataatgcggaggaaggtggggatgacgtcaaatcatcatgccccttacgtcccgggctacacacgtgctacaatggtctgtacagagggtagcgaaagagcgatcttaagccaatcccaaaaagcaggccccagttcggattggaggctgcaactcgcctccatgaagtaggaatcgctagtaatcgcggatcagcatgccgcggtgaatacgtcccg

SEQ ID NO: 600 - DNA, 588 bp, Artificial Sequence; identical to SEQ ID NO: 296
  Synthetic 1653_Y212T

SEQ ID NO: 601 - DNA, 587 bp, Artificial Sequence; identical to SEQ ID NO: 295
  Synthetic 1664_A211T

SEQ ID NO: 602 - DNA, 587 bp, Artificial Sequence; identical to SEQ ID NO: 295
  Synthetic 1629_R948T

SEQ ID NO: 603 - DNA, 587 bp, Artificial Sequence; identical to SEQ ID NO: 295
  Synthetic 1629_R747T

SEQ ID NO: 604 - DNA, 587 bp, Artificial Sequence; identical to SEQ ID NO: 295
  Synthetic 1629_R148T

SEQ ID NO: 605 - DNA, 23 bp, Artificial Sequence
  Synthetic PTM06
gctatgtgtcgggagatccacgt

SEQ ID NO: 606 - DNA, 246 bp, Artificial Sequence
  Synthetic TXv5v60644101
ctagcagtaaacactgcacactaaacattagtacctcctcgagaggtattggtgctgaagggaagccgaagagtgtgctacctgggaagtatagccgcaggccgaaacttaaaggaattggcggggagacactacaacaggtgacgcgtgcggttcaattagattatacaccgtgaacctcaccaggagcgacagcaggatgaaggtcagtctgaagggcttacctgacacgctgagaggagttgc

SEQ ID NO: 607 - DNA, 247 bp, Artificial Sequence
  Synthetic TXv5v60248376
ctagccgtaaacgatgctcgctaggtgttaaataccctgggagggtatttagtgtcgtaaggaagccgtgaagcgagccacctgggaagtacggtcgcaaggctgaaacttaaaggaattggcgggggagcacaacaacgggtggatgctgcggtttaattggattcaacgccggaaatcttaccggaggcgacagcaatatgaaggtcaggttgaagaccttaccaaattcgctgagaggaagtgc

SEQ ID NO: 608 - DNA, 248 bp, Artificial Sequence
  Synthetic TXv5v60671483
ctagccgtaaacgatgctcgctaggtgttaaataccctgggagggtatttagtgtcgtaaggaagccgtgaagcgagccacctgggaagtacggtcgcaaggctgaaacttaaagggaattggcgggggagcacaacaacgggtggatgctgcggtttaattggattcaacgccggaaatcttaccggaggcgacagcaatatgaaggtcaggttgaagaccttaccaaattcgctgagaggaagtgc

SEQ ID NO: 609 - DNA, 248 bp, Artificial Sequence
  Synthetic TXv5v6-1672136
ccagctgtaaacgatgcgggccaggtgttggcattactgcgagtgatgtcagtgccaaaggggaagccgttaagcccgccatctggggagtacggtcgcaaggctgaaacttaaagaaattggcgggggagcaccacaacgggtggagcctgcggttcaattggattcaacgccggaaaactcaccggaggcgacagcgagatgaaggtcaggctgaagaccttaccggattagctgagaggtggcgc

SEQ ID NO: 610 - DNA, 248 bp, Artificial Sequence
  Synthetic TXv5v6-0237299
ccagctgtaaacgatgcgggccaggtgttggcattactgcgagtgatgtcagtgccaaagggaagccgttaagcccgccatctggggagtacggtcgcaaggctgaaacttaaaggaaattggcgggggagcaccacaacgggtggagcctgcggttcaattggattcaacgccggaaaactcaccggaggcgacagcgagatgaaggtcaggctgaagaccttaccggattagctgagaggtggcgc

SEQ ID NO: 611 - DNA, 248 bp, Artificial Sequence
  Synthetic TXv5v6-0237291
ccagctgtaaacgatgcgggccaggtgttggcattactgcgagtgatgtcagtgccaaagggaagccgttaagcccgccatctggggagtacggtcgcaaggctgaaacttaaaggaaattggcgggggagcaccacaacgggtggagcctgcggttcaatcggattcaacgccggaaaactcaccggaggcgacagcaagatgaaggtcaggctgaagaccttaccagattagctgagaggtggcgc

SEQ ID NO: 612 - DNA, 248 bp, Artificial Sequence
  Synthetic TXv5v6-1691264
ctagctgtaaacgatgcgggctaggtgttggcattactgcgagtgatgccagtgccgaagggaagcccgttaagcccgccatctggggagtacggtcgcaaggctgaaacttaaaggaattggcgggggagcactacaacgggtggagcctgcggttcaattggattcaacgccggaaaactcaccggaggcgacagcgagatgaaggtcaggctgaagaccttaccggattagctgagaggtggcgc

SEQ ID NO: 613 - DNA, 248 bp, Artificial Sequence
  Synthetic TXv5v6-0718915
ctagctgtaaacgatgcgggctaggtgttggcattactgcgagtgatgccagtgccgaaggggaagccgttaagcccgccatctggggagtacggtcgcaaggctgaaacttaaaggaattggcgggggagcactacaacgggtggagcctgcggttcaattggattcaacgccggaaaactcaccggaggcgacagcgagatgaaggtcaggctgaagaccttaccggattagctgagaggtggcgc

SEQ ID NO: 614 - DNA, 247 bp, Artificial Sequence
  Synthetic TXv5v6-0232006
ccagccgtaaacgatgctcgctatgtgtcaggtacggtgtgaccgtatctggtgccgtagggaagccgtgaagcgagccacctgggaagtacggtcgcaagactgaaacttaaaggaattggcgggggagcactacaacgggtggagcctgcggtttaattggattcaacgccggaaatcttaccgggggagacagcagcatgaaggtcaggctgaagaccttaccagatccgctgagaggaagtgc

SEQ ID NO: 615 - DNA, 247 bp, Artificial Sequence
  Synthetic TXv5v6-0231898
ccagccgtaaacgatgctcgctatgtgtcaggtacggtgcgaccgtatctggtgccgtagggaagccgtgaagcgagccacctgggaagtacggtcgcaagactgaaacttaaaggaattggcgggggagcactacaacgggtggagcctgcggtttaattggattcaacgccggaaatcttaccgggtgagacagcaatatgaaggtcaggctgaagaccttaccggattcgctgagaggaagtgc

SEQ ID NO: 616 - DNA, 257 bp, Artificial Sequence
  Synthetic TXv5v6-0260150
ctagctgtaaacgatggatactaggtgtgggaggtatcgaccccttctgtgccgtagctaacgcattaagtatcccgcctggggagtacggtcgcaaggctgaaactcaaaggaattgacgggggcccgcacaagcggtggagcatgtggtttaattcgacgcaacgcgaagaaccttaccggggcttgacattatcttgcccgtctaagaaattagatcttcttcctttcggggaagacgagataacaggtggtgc

SEQ ID NO: 617 - DNA, 257 bp, Artificial Sequence
  Synthetic TXv5v6-0259561
ctagctgtaaacgatggatactaggtgtgggaggtatcgaccccttctgtgccgcagctaacgcattaagtatcccgcctggggagtacggtcgcaaggctgaaactcaaaggaattgacgggggcccgcacaagcggtggagcatgtggtttaattcgacgcaacgcgaagaaccttaccgggacttgacattatcttgcccgtctaagaaattagatcttcttcctttcggggaagacgagataacaggtggtgc

SEQ ID NO: 618 - DNA, 248 bp, Artificial Sequence
  Synthetic TXv5v6-1692076
ctagctgtaaacgatgctcgctaggtgtcagacacggtgcgaccgtgtttggtgccgcagggaagcccgttaagcgagccacctgggaagtacggtcgcaaggctgaaacttaaaggaattggcgggggagcactacaacgggtggagcctgcggtttaattggattcaacgccggaaaactcaccgggtgcgacagcaatatgtaggtcaggctgaaggtcttacctgaatcgctgagaggaggtgc

SEQ ID NO: 619 - DNA, 246 bp, Artificial Sequence
  Synthetic TXv5v6-0719836
ctagctgtaaacgatgctcgctaggtgtcagacacggtgcgaccgtgtttggtgccgcagggaagccgttaagcgagccacctgggaagtacggtcgcaaggctgaaacttaaaggaattggcgggggagcactacaacgggtggagcctgcggtttaattggattcaacgccggaaaactcaccgggtgcgacagcaatatgtaggtcaggctgaaggtcttacctgaatcgctgagaggagtgc